(19) United States
(12) Patent Application Publication
(10) Pub. No.: US 2024/0390298 A1
(43) Pub. Date: Nov. 28, 2024

(54) CYCLOHEXYL BETA-HYDROXY ALKYL AMINES AND MEDICAL USES THEREOF

(71) Applicant: ATROGI AB, Solna (SE)

(72) Inventors: Tore Bengtsson, Vaxholm (SE); Benjamin Pelcman, Stockholm (SE)

(21) Appl. No.: 18/693,571

(22) PCT Filed: Sep. 23, 2022
(86) PCT No.: PCT/EP2022/076473

§ 371 Date: Mar. 20, 2024

(30) Foreign Application Priority Data
Sep. 23, 2021 (GB) 2113594.2

(51) Int. Cl. A61K 31/137; A61K 31/44; A61P 3/10; C07C 215/30; C07C 311/07; C07D 213/61

(52) U.S. Cl. CPC A61K 31/137; A61K 31/44; A61P 3/10; C07C 215/30; C07C 311/07; C07D 213/61

(57) ABSTRACT

There is provided herein compounds of formula I and pharmaceutically acceptable salts thereof, wherein X¹, X², Z, the ring containing Q¹ to Q⁵, m and r have meanings as provided in the description. There is also provided medical uses of such compounds.

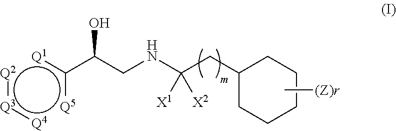

### FIELD OF THE INVENTION

The present invention relates to novel compounds and compositions, and their use in medicine, such as in the treatment of hyperglycaemia and disorders characterised by hyperglycaemia, such as type 2 diabetes. In particular, the invention relates to novel compounds, compositions and methods for the treatment of conditions such as type 2 diabetes through activation of the β₂-adrenergic receptor. Importantly, such compounds are thought to have a beneficial side-effect profile as they do not exert their effect through significant cAMP release.

### BACKGROUND OF THE INVENTION

The listing or discussion of an apparently prior-published document in this specification should not necessarily be taken as an acknowledgement that the document is part of the state of the art or is common general knowledge.

Hyperglycaemia, or high blood sugar is a condition in which an excessive amount of glucose circulates in the blood plasma. If not treated, hyperglycaemia can be a serious problem, potentially developing into life-threatening conditions such as ketoacidosis. For example, chronic hyperglycemia may cause injury to the heart, and is strongly associated with heart attacks and death in subjects with no coronary heart disease or history of heart failure. There are various causes of hyperglycaemia, including diabetes and severe insulin resistance.

Severe insulin resistance (SIR) is a condition wherein the patent experiences very low levels of (or, in extreme cases, no significant) response to insulin. There are several syndromes characterized by SIR, including Rabson-Mendenhall syndrome, Donohue's syndrome (leprechaunism), Type A and Type B syndromes of insulin resistance, the HAIR-AN (hyperandrogenism, insulin resistance, and acanthosis nigricans) syndrome, pseudoacromegaly, and lipodystrophy. The majority of these conditions have genetic causes, such as mutations in the insulin receptor gene. The prevalence for Donohue's syndrome, Rabson-Mendenhall syndrome and Type A syndrome of insulin resistance, has been reported to vary from about 50 reported cases to 1 in 100,000. However, since some diseases are severe and extremely rare, it is likely that many patients do not get diagnosed before they die, particularly in less developed areas of the world. Thus, the exact number of patients with these syndromes is difficult to assess.

The current standard for hyperglycaemia treatment in patients having SIR is a controlled diet, supplemented with drugs affecting insulin receptor sensitivity, such as metformin, or insulin supplement. However, particularly for disorders caused by mutations in the insulin receptor gene, this treatment is not sufficiently effective and ultimately proves unsuccessful.

Diabetes comprises two distinct diseases, type 1 (or insulin-dependent diabetes) and type 2 (insulin-independent diabetes), both of which involve the malfunction of glucose homeostasis. Type 2 diabetes affects more than 400 million people in the world and the number is rising rapidly. Complications of type 2 diabetes include severe cardiovascular problems, kidney failure, peripheral neuropathy, blindness and, in the later stages of the disease, even loss of limbs and, ultimately death. Type 2 diabetes is characterized by insulin resistance in skeletal muscle and adipose tissue, and there is presently no definitive cure. Most treatments used today are focused on remedying dysfunctional insulin signalling or inhibiting glucose output from the liver but many of those treatments have several drawbacks and side effects. There is thus a great interest in identifying novel insulin-independent ways to treat type 2 diabetes.

In type 2 diabetes, the insulin-signalling pathway is blunted in peripheral tissues such as adipose tissue and skeletal muscle. Methods for treating type 2 diabetes typically include lifestyle changes, as well as insulin injections or oral medications to regulate glucose homeostasis. People with type 2 diabetes in the later stages of the disease develop ‘beta-cell failure’ i.e. the inability of the pancreas to release insulin in response to high blood glucose levels. In the later stages of the disease patients often require insulin injections in combination with oral medications to manage their diabetes. Further, most common drugs have side effects including downregulation or desensitization of the insulin pathway and/or the promotion of lipid incorporation in adipose tissue, liver and skeletal muscle. There is thus a great interest in identifying novel ways to treat metabolic diseases including type 2 diabetes that do not include these side effects.

Following a meal, increased blood glucose levels stimulate insulin release from the pancreas. Insulin mediates normalization of the blood glucose levels. Important effects of insulin on glucose metabolism include facilitation of glucose uptake into skeletal muscle and adipocytes, and an increase of glycogen storage in the liver. Skeletal muscle and adipocytes are responsible for insulin-mediated glucose uptake and utilization in the fed state, making them very important sites for glucose metabolism.

The signalling pathway downstream from the insulin receptor has been difficult to understand in detail. In brief, control of glucose uptake by insulin involves activation of the insulin receptor (IR), the insulin receptor substrate (IRS), the phosphoinositide 3-kinase (PI3K) and thus stimulation of phosphatidylinositol (3,4,5)-triphosphate (PIP3), the mammalian target of rapamycin (also called the mechanistic target of rapamycin, mTOR), Akt/PKB (Akt) and TBC1D4 (AS160), leading to translocation of the glucose transporter 4 (GLUT4) to the plasma membrane. Akt activation is considered necessary for GLUT4 translocation.

It should be noted that skeletal muscles constitute a major part of the body weight of mammals and have a vital role in the regulation of systemic glucose metabolism, being responsible for up to 85% of whole-body glucose disposal. Glucose uptake in skeletal muscles is regulated by several intra- and extracellular signals. Insulin is the most well studied mediator but others also exist. For example, AMP activated kinase (AMPK) functions as an energy sensor in the cell, which can increase glucose uptake and fatty acid oxidation. Due to the great influence skeletal muscles have on glucose homeostasis it is plausible that additional mechanisms exist. In the light of the increased prevalence of type 2 diabetes, it is of great interest to find and characterize novel insulin-independent mechanisms to increase glucose uptake in muscle cells.

Blood glucose levels may be regulated by both insulin and catecholamines, but they are released in the body in response to different stimuli. Whereas insulin is released in response to the rise in blood sugar levels (e.g. after a meal), epinephrine and norepinephrine are released in response to various internal and external stimuli, such as exercise, emotions and stress, and also for maintaining tissue homeostasis. Insulin is an anabolic hormone that stimulates many processes involved in growth including glucose uptake, glycogen and triglyceride formation, whereas catecholamines are mainly catabolic.

Although insulin and catecholamines normally have opposing effects, it has been shown that they have similar actions on glucose uptake in skeletal muscle (Nevzorova et al., Br. J. Pharmacol, 137, 9, (2002)). In particular, it has been reported that catecholamines stimulate glucose uptake via adrenergic receptors (Nevzorova et al., Br. J. Pharmacol, 147, 446, (2006); Hutchinson, Bengtsson, Endocrinology 146, 901, (2005)) to supply muscle cells with an energy-rich substrate. Thus it is likely that in mammals, including humans, the adrenergic and the insulin systems can work independently to regulate the energy needs of skeletal muscle in different situations. Since insulin also stimulates many anabolic processes, including some that promote undesired effects such as stimulation of lipid incorporation into tissues, leading to e.g. obesity, it would be beneficial to be able to stimulate glucose uptake by other means; for example, by stimulation of the adrenergic receptors (ARs).

All ARs are G protein-coupled receptors (GPCRs) located in the cell membrane and characterized by an extracellular N-terminus, followed by seven transmembrane α-helices (TM-1 to TM-7) connected by three intracellular (IL-1 to IL-3) and three extracellular loops (EL-1 to EL-3), and finally an intracellular C-terminus. There are three different classes of ARs, with distinct expression patterns and pharmacological profiles: α₁-, α₂- and β-ARs. The α₁-ARs comprise the α_{1A}, α_{1B} and α_{1D} subtypes while α₂-ARs are divided into α_{2A}, α_{2B} and α_{2C}. The β-ARs are also divided into the subtypes β₁, β₂, and β₃, of which β₂-AR is the major isoform in skeletal muscle cells. ARs are G protein coupled receptors (GPCRs) that signal through classical secondary messengers such as cyclic adenosine monophosphate (cAMP) and phospholipase C (PLC).

Many effects occurring downstream of ARs in skeletal muscles have been attributed to classical secondary messenger signalling, such as increase in cAMP levels, PLC activity and calcium levels. Stimulation involving the classical secondary messengers has many effects in different tissues. For example, it increases heart rate, blood flow, airflow in lungs and release of glucose from the liver, which all can be detrimental or be considered unwanted side effects if stimulation of ARs should be considered as a type 2 diabetes treatment. Adverse effects of classical AR agonists are, for example, tachycardia, palpitation, tremor, sweats, agitation and increased glucose levels in the blood (glucose output from the liver). It would thus be beneficial to be able to activate ARs without activating these classical secondary messengers, such as cAMP, to increase glucose uptake in peripheral tissues without stimulating the unwanted side effects.

Glucose uptake is mainly stimulated via facilitative glucose transporters (GLUT) that mediate glucose uptake into most cells. GLUTs are transporter proteins that mediate transport of glucose and/or fructose over the plasma membrane down the concentration gradient. There are fourteen known members of the GLUT family, named GLUT1-14, divided into three classes (Class I, Class II and Class III) dependent on their substrate specificity and tissue expression. GLUT1 and GLUT4 are the most intensively studied isoforms and, together with GLUT2 and GLUT3, belong to Class I which mainly transports glucose (in contrast to Class II that also transports fructose). GLUT1 is ubiquitously expressed and is responsible for basal glucose transport. GLUT4 is only expressed in peripheral tissues such as skeletal muscle, cardiac muscle and adipose tissues. GLUT4 has also been reported to be expressed in, for example, the brain, kidney, and liver. GLUT4 is the major isoform involved in insulin stimulated glucose uptake. The mechanism whereby insulin signalling increases glucose uptake is mainly via GLUT4 translocation from intracellular storage to the plasma membrane. It is known that GLUT4 translocation is induced by stimulation of the β₂-adrenergic receptor.

Thus, a possible treatment of a condition involving dysregulation of glucose homeostasis or glucose uptake in a mammal, such as type 2 diabetes, would involve the activation of the β₂-adrenergic receptor leading to GLUT4 translocation to the plasma membrane and promotion of glucose uptake into skeletal muscle leading to normalization of whole body glucose homeostasis. In addition, it would be advantageous if the treatment does not involve signalling through cAMP as this would lead to a favourable side-effect profile.

### DESCRIPTION OF THE INVENTION

We have now surprisingly found that certain cyclohexyl beta-hydroxy alkyl amines acting as agonists at the β₂-adrenergic receptor increase glucose uptake in skeletal muscle.

In addition, we have found that this effect is not mediated through significant cAMP release, such that many of the commonly described side effects seen with traditional β₂-adrenergic agonists (e.g. tachycardia, palpitation, tremor, sweats, agitation, and the like) can be reduced.

The use of such compounds in medicine represents a promising strategy for the treatment of conditions as described herein, such as those characterized by high blood sugar levels (i.e. hyperglycaemia), such as type 2 diabetes.

### Compounds of the Invention

In a first aspect of the invention, there is provided a compound of formula I

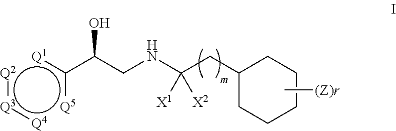

or a pharmaceutically acceptable salt thereof, wherein:
the ring comprising Q¹ to Q⁵ represents:
phenyl optionally substituted with one or more Y¹, or
a 5- or 6-membered heteroaryl optionally substituted with one or more Y²;
X¹ and X² each independently represent H or C₁₋₃ alkyl optionally substituted with one or more fluoro;
each Y¹ independently represents halo, R^{a1}, —CN or —N₃;
each Y² independently represent halo, R^{a2}, —CN or —N₃;
each Z independently represents halo, R^{a3}, —CN, —N₃, —N(R^{b3})R^{c3}, —OR^{d3}, —S(O)ₚR^{e3}, —S(O)_{q}N(R^{f3})R^{g3} or —N(R^{h3})S(O)ₜRⁱ³;
each R^{a1}, R^{a2}, R^{a3}, R^{e3} and Rⁱ³ represents C₁₋₆ alkyl, C₂₋₆ alkenyl, or C₂₋₆ alkynyl each optionally substituted by one or more groups independently selected from halo and G¹;
each R^{b3}, R^{c3}, R^{d3}, R^{f3}, R^{g3} and R^{h3} independently represents
H,
C₁₋₆ alkyl, C₂₋₆ alkenyl, C₂₋₆ alkynyl each optionally substituted by one or more groups independently selected from halo and G²;
or alternatively any of R^{b3} and R^{c3}, and/or R^{f3} and R^{g3} may be linked together to form, together with the nitrogen atom to which they are attached, a 4- to 6-membered ring, which ring optionally contains one further heteroatom and which ring optionally is substituted by one or more groups independently selected from halo, C₁₋₃ alkyl optionally substituted by one or more halo, and ═O;
each G¹ and G² independently represents R^{a4}, —CN, —N₃, —N(R^{b4})R^{c4}, —OR^{d4}, —S(O)ₚR^{e4}, —S(O)_{q}N(R^{f4})R^{g4}, or ═O;
each R^{a4} independently represents phenyl or 5- or 6-membered heteroaryl, each optionally substituted by one or more group selected from halo, —CN or C₁₋₃ alkyl optionally substituted by one or more halo or —CN;
each R^{b4}, R^{c4}, R^{d4}, R^{f4} and R^{g4} independently represents H, or C₁₋₆ alkyl, C₂₋₆ alkenyl or C₂₋₆ alkynyl optionally substituted by one or more halo or —CN;
each R^{e4} independently represents C₁₋₆ alkyl, C₂₋₆ alkenyl, C₂₋₆ alkynyl each optionally substituted by one or more halo or —CN;
or alternatively any of R^{b4} and R^{c4} and/or R^{f4} and R^{g4} may be linked together to form, together with the nitrogen atom to which they are attached, a 4- to 6-membered ring, which ring optionally contains one further heteroatom and which ring optionally is substituted by one or more groups independently selected from halo, C₁₋₃ alkyl optionally substituted by one or more halo, and ═O;
each p independently represents 0, 1 or 2;
each q independently represents 1 or 2;
each t independently represents 1 or 2;
m represents 0 to 3;
r represents 0 to 6,
which compounds (including pharmaceutically acceptable salts) may be referred to herein as “compounds of the invention”.

For the avoidance of doubt, the skilled person will understand that references herein to compounds of particular aspects of the invention (such as the first aspect of the invention, e.g. compounds of formula I) will include references to all embodiments and particular features thereof, which embodiments and particular features may be taken in combination to form further embodiments.

Unless indicated otherwise, all technical and scientific terms used herein have the same meaning as commonly understood by one of ordinary skill in the art to which this invention pertains.

Pharmaceutically acceptable salts include acid addition salts and base addition salts. Such salts may be formed by conventional means, for example by reaction of a free acid or a free base form of a compound of the invention with one or more equivalents of an appropriate acid or base, optionally in a solvent, or in a medium in which the salt is insoluble, followed by removal of said solvent, or said medium, using standard techniques (e.g. in vacuo, by freeze-drying or by filtration). Salts may also be prepared by exchanging a counter-ion of a compound of the invention in the form of a salt with another counter-ion, for example using a suitable ion exchange resin.

Particular acid addition salts that may be mentioned include carboxylate salts (e.g. formate, acetate, trifluoroacetate, propionate, isobutyrate, heptanoate, decanoate, caprate, caprylate, stearate, acrylate, caproate, propiolate, ascorbate, citrate, glucuronate, glutamate, glycolate, α-hydroxybutyrate, lactate, tartrate, phenylacetate, mandelate, phenylpropionate, phenylbutyrate, benzoate, chlorobenzoate, methylbenzoate, hydroxybenzoate, methoxybenzoate, dinitrobenzoate, o-acetoxy-benzoate, salicylate, nicotinate, isonicotinate, cinnamate, oxalate, malonate, succinate, suberate, sebacate, fumarate, malate, maleate, hydroxymaleate, hippurate, phthalate or terephthalate salts), halide salts (e.g. chloride, bromide or iodide salts), sulphonate salts (e.g. benzenesulphonate, methyl-, bromo- or chloro-benzenesulphonate, xylenesulphonate, methanesulphonate, ethanesulphonate, propanesulphonate, hydroxy-ethanesulphonate, 1- or 2-naphthalene-sulphonate or 1,5-naphthalenedisulphonate salts) or sulphate, pyrosulphate, bisulphate, sulphite, bisulphite, phosphate, monohydrogenphosphate, dihydrogenphosphate, metaphosphate, pyrophosphate or nitrate salts, and the like.

Particular base addition salts that may be mentioned include salts formed with alkali metals (such as Na and K salts), alkaline earth metals (such as Mg and Ca salts), organic bases (such as ethanolamine, diethanolamine, triethanolamine, tromethamine and lysine) and inorganic bases (such as ammonia and aluminium hydroxide). More particularly, base addition salts that may be mentioned include Mg, Ca and, most particularly, K and Na salts.

Particular pharmaceutically acceptable salts that may be mentioned include acetate salts.

For the avoidance of doubt, compounds of the first aspect of the invention may exist as solids, and thus the scope of the invention includes all amorphous, crystalline and part crystalline forms thereof, and may also exist as oils. Where compounds of the first aspect of the invention exist in crystalline and part crystalline forms, such forms may include solvates, which are included in the scope of the invention. Compounds of the first aspect of the invention may also exist in solution.

Compounds of the first aspect of the invention may contain double bonds and may thus exist as E (entgegen) and Z (zusammen) geometric isomers about each individual double bond. All such isomers and mixtures thereof are included within the scope of the invention.

Compounds of the first aspect of the invention may also exhibit tautomerism. All tautomeric forms and mixtures thereof are included within the scope of the invention.

Compounds of the first aspect of the invention may also contain one or more asymmetric carbon atoms and may therefore exhibit optical and/or diastereoisomerism. Diastereoisomers may be separated using conventional techniques, e.g. chromatography or fractional crystallisation. The various stereoisomers (i.e. enantiomers) may be isolated by separation of a racemic or other mixture of the compounds using conventional, e.g. fractional crystallisation or HPLC, techniques. Alternatively the desired optical isomers may be obtained from appropriate optically active starting materials under conditions which will not cause racemisation or epimerisation (i.e. a ‘chiral pool’ method), by reaction of the appropriate starting material with a ‘chiral auxiliary’ which can subsequently be removed at a suitable stage, by derivatisation (i.e. a resolution, including a dynamic resolution); for example, with a homochiral acid followed by separation of the diastereomeric derivatives by conventional means such as chromatography, or by reaction with an appropriate chiral reagent or chiral catalyst all under conditions known to the skilled person. All stereoisomers and mixtures thereof are included within the scope of the invention.

As used herein, references to halo and/or halogen groups will each independently refer to fluoro, chloro, bromo and iodo (for example, fluoro (F) and chloro (Cl), such as F).

Unless otherwise specified, C_{1-z} alkyl groups (where z is the upper limit of the range) defined herein may be straight-chain or, when there is a sufficient number (i.e. a minimum of three) of carbon atoms, be branched-chain and/or cyclic (so forming a C_{3-z}-cycloalkyl group). When there is a sufficient number (i.e. a minimum of four) of carbon atoms, such groups may also be part cyclic. Part cyclic alkyl groups that may be mentioned include cyclopropylmethyl and cyclohexylethyl. When there is a sufficient number of carbon atoms, such groups may also be multicyclic (e.g. bicyclic or tricyclic) or spirocyclic.

For the avoidance of doubt, alkyl groups may be linear (otherwise referred to as straight-chained), branched (otherwise referred to as branched-chain) and/or cyclic. More particularly, alkyl groups may be linear (otherwise referred to as straight-chained) or branched (otherwise referred to as branched-chain).

Unless otherwise specified, C_{2-z} alkenyl groups (where z is the upper limit of the range) defined herein may be straight-chain or, when there is a sufficient number (i.e. a minimum of three) of carbon atoms, be branched-chain.

Unless otherwise specified, C_{2-z} alkynyl groups (where z is the upper limit of the range) defined herein may be straight-chain or, when there is a sufficient number (i.e. a minimum of four) of carbon atoms, be branched-chain.

For the avoidance of doubt, the skilled person will understand that the term alkyl will refer to saturated hydrocarbon moieties, whereas the term alkenyl will refer to unsaturated hydrocarbon moieties containing at least one carbon-carbon double bond and the term alkynyl will refer to unsaturated hydrocarbon moieties containing at least one carbon-carbon triple bond.

As described herein, the ring comprising Q¹ to Q⁵ (which may be referred to as ring Q) represents a phenyl or a 5- or 6-membered heteroaryl optionally substituted with one or more Y.

As such, the skilled person will understand that representing Q¹ to Q⁵ the ring may comprise, in addition to carbon atoms, one or more heteroatom, so as to form suitable heteroaryl groups as known to those skilled in the art. Moreover, the skilled person will understand that where the ring containing Q¹ to Q⁵ is 5-membered, one of Q¹ to Q⁵ (e.g. Q⁵) will represent a direct bond (i.e. that group will not be present).

For the avoidance of doubt, the depiction of the ring containing the Q¹ to Q⁵ groups with a circle therein (for example, such as in formula I) will be understood to indicate that the ring is aromatic.

For the avoidance of doubt, as used herein, references to heteroatoms will take their normal meaning as understood by one skilled in the art. Particular heteroatoms that may be mentioned include phosphorus, selenium, tellurium, silicon, boron, oxygen, nitrogen and sulphur (e.g. oxygen, nitrogen and sulphur).

For the avoidance of doubt, references to polycyclic (e.g. bicyclic or tricyclic) groups (e.g. when employed in the context of cycloalkyl groups) will refer to ring systems wherein at least two scissions would be required to convert such rings into a straight chain, with the minimum number of such scissions corresponding to the number of rings defined (e.g. the term bicyclic may indicate that a minimum of two scissions would be required to convert the rings into a straight chain). For the avoidance of doubt, the term bicyclic (e.g. when employed in the context of alkyl groups) may refer to groups in which the second ring of a two-ring system is formed between two adjacent atoms of the first ring, and may also refer to groups in which two non-adjacent atoms are linked by an alkylene group, which later groups may be referred to as bridged.

The present invention also embraces isotopically-labelled compounds of the present invention which are identical to those recited herein, but for the fact that one or more atoms are replaced by an atom having an atomic mass or mass number different from the atomic mass or mass number usually found in nature (or the most abundant one found in nature). All isotopes of any particular atom or element as specified herein are contemplated within the scope of the compounds of the invention. Hence, the compounds of the invention also include deuterated compounds, i.e. in which one or more hydrogen atoms are replaced by the hydrogen isotope deuterium.

For the avoidance of doubt, in cases in which the identity of two or more substituents in a compound of the invention may be the same, the actual identities of the respective substituents are not in any way interdependent. For example, in the situation in which two or more Y groups are present, those Y groups may be the same or different. Similarly, where two or more Y groups are present and each represent halo, the halo groups in question may be the same or different. Likewise, when more than one R is present and each independently represents C₁₋₆ alkyl substituted by one or more G group, the identities of each G are in no way interdependent.

The skilled person will appreciate that compounds of the invention that are the subject of this invention include those that are stable. That is, compounds of the invention include those that are sufficiently robust to survive isolation, e.g. from a reaction mixture, to a useful degree of purity.

All embodiments of the invention and particular features mentioned herein may be taken in isolation or in combination with any other embodiments and/or particular features mentioned herein (hence describing more particular embodiments and particular features as disclosed herein) without departing from the disclosure of the invention.

For the avoidance of doubt, the ring comprising Q¹ to Q⁵ (also referred to herein as ring Q) represents a phenyl optionally substituted with one or more Y¹, or a 5- or 6-membered heteroaryl optionally substituted with one or more Y².

Various heteroaryl groups will be well-known to those skilled in the art, such as pyridinyl, pyridonyl, pyrrolyl, furanyl, thiophenyl, oxadiazolyl, thiadiazolyl, thiazolyl, oxazolyl, pyrazolyl, triazolyl, tetrazolyl, isoxazolyl, isothiazolyl, imidazolyl and the like. The oxides of heteroaryl/heteroaromatic groups are also embraced within the scope of the invention (e.g. the N-oxide).

In particular embodiments, when representing a heteroaryl, the ring comprising Q¹ to Q⁵ as defined herein may comprise one or more (e.g. one or two, such as one) heteroatoms, which may be selected from O, S and N (e.g. from O and N, such as N). For example, ring Q as defined herein may comprise one heteroatom, which may be selected from O, S and N (e.g. O and N, such as N).

In particular embodiments, where representing heteroaryl, the ring comprising Q¹ to Q⁵ as defined herein may be a 6-membered heteroaryl. As such, the ring comprising Q¹ to Q⁵ may represent phenyl optionally substituted with one or more (e.g. one) Y¹, or a 5- or 6-membered heteroaryl optionally substituted with one or more (e.g. one) Y².

More particular heteroaryl groups representing the ring comprising Q¹ to Q⁵ that may be include pyridinyl, such as pyridin-3-yl (referring to standard numbering wherein the N atom represents the 1-position).

In particular embodiments, the ring comprising Q¹ to Q⁵ may represent phenyl optionally substituted with one or more (e.g. one) Y¹, or pyridyl optionally substituted with one or more (e.g. one) Y².

Thus, in particular embodiments, the compound of formula I may be represented as a compound of formula IA or IB

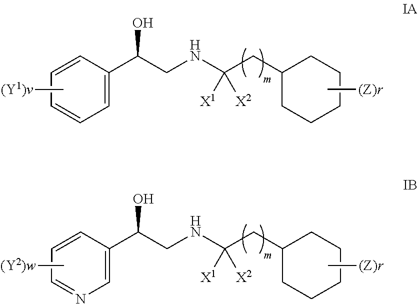

wherein Z, Y¹, Y², X¹, X², m and r are as defined for compounds of formula I (including all embodiments thereof), v represents 0 to 5 and w represents 0 to 4.

For the avoidance of doubt, the ring comprising Q¹ to Q⁵ may be substituted by a number of Y¹ or Y² groups, as defined herein, as appropriate in the circumstances. The skilled person will understand that the (maximum) number and position of such substituents will be dictated by the nature of the ring, such as by the size of the ring and the level of saturation thereof. Moreover, the skilled person will understand that such substituents may be present on suitable moieties comprised within ring Q, e.g. suitable C (carbon) moieties.

In particular embodiments, the ring comprising Q¹ to Q⁵ is substituted with up to two (i.e. 0 to 2) Y¹ or Y² groups, as appropriate.

In more particular embodiments, the ring comprising Q¹ to Q⁵ is substituted with up to one (i.e. 0 or 1) Y¹ or Y² groups, as appropriate.

In more particular embodiments, the ring comprising Q¹ to Q⁵ is (i.e. is required to be) substituted with one Y¹ or Y² group, as appropriate (i.e. in compounds of formula IA, v is 1, and in compounds of formula IB, w is 1).

In particular embodiments, taking the point of attachment to the essential —CH(OH)— moiety as the 1-position, at least one (or, when only one such group is present, the) Y¹ or Y² group, as appropriate, may be located in the 2- or 3-position (which may also be referred to as the ortho and meta positions, respectively).

In particular embodiments, the ring comprising Q¹ to Q⁵ may represent:
phenyl substituted with one or more (e.g. one) Y¹, or
6-membered heteroaryl substituted with one or more (e.g. one) Y².

In more particular embodiments, the ring comprising Q¹ to Q⁵ may represent:
phenyl substituted with one or more (e.g. one) Y¹, or
pyridyl (also referred to as pyridinyl) substituted with one or more (e.g. one) Y².

For example, the ring comprising Q¹ to Q⁵ may represent:
phenyl substituted with one Y¹ (e.g. in the 2- or 3-position using standard numbering) or pyridin-3-yl substituted with one Y² (e.g. in the 5-position using standard numbering, which may also be referred as the 3-position when the point of attachment to the essential —CH(OH)— moiety is taken as the 1-position)

Thus, in particular embodiments, the compounds of formula IA and IB may be represented as a compounds of formula IA′ and IB′, respectively

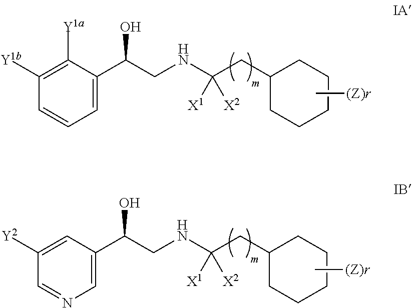

wherein Z, Y², X¹, X², m and r are as defined for compounds of formula I (such as compounds of formula IA and IB; including all embodiments thereof), and wherein one of Y^{1a} and Y^{1b} represents Y¹ and the other represents H.

In particular embodiments, each Y¹ independently represents R^{a1}, halo (e.g. F or Cl) or —CN.

In particular embodiments, R^{a1} represents C₁₋₃ alkyl (such as linear or branched C₁₋₃ alkyl, e.g. linear C₁₋₃ alkyl) optionally substituted by one or more halo.

In more particular embodiments, R^{a1} represents C₁ alkyl (i.e. methyl) optionally substituted by one or more halo. For example, R^{a1} may represent —CH₃ or —CF₃.

In more particular embodiments, each Y¹ independently represents halo (e.g. F or Cl) or —CN.

In more particular embodiments, each Y¹ independently represents Cl or F.

In more particular embodiments, each Y¹ independently represents F.

In more particular embodiments, each Y¹ independently represents —CN.

In particular embodiments, each Y² independently represents R^{a2}, halo (e.g. F or Cl) or —CN.

In particular embodiments, R^{a2} represents C₁₋₃ alkyl (such as linear or branched C₁₋₃ alkyl, e.g. linear C₁₋₃ alkyl) optionally substituted by one or more halo.

In more particular embodiments, R^{a2} represents C₁ alkyl (i.e. methyl) optionally substituted by one or more halo. For example, R^{a1} may represent —CH₃ or —CF₃.

In more particular embodiments, each Y² independently represents Cl or F.

In more particular embodiments, each Y² independently represents F.

In more particular embodiments, each Y² independently represents —CN.

For the avoidance of doubt, as indicated herein, embodiments of the invention will include combinations of embodiments as described herein.

In particular embodiments, each Y¹ and Y² independently represents R^{a1}, halo (e.g. F or Cl) or —CN.

For example, in certain embodiments that may be mentioned:
each Y¹ independently represents R^{a1}, halo (e.g. F or Cl) or —CN;
R^{a1} represents C₁₋₃ alkyl (such as linear or branched C₁₋₃ alkyl, e.g. linear C₁₋₃ alkyl) optionally substituted by one or more halo;
Y² independently represents R^{a2}, halo (e.g. F or Cl) or —CN; and
R^{a2} represents C₁₋₃ alkyl (such as linear or branched C₁₋₃ alkyl, e.g. linear C₁₋₃ alkyl) optionally substituted by one or more halo.

In certain embodiments, each Y¹ and Y² represents Cl or F (particularly, F).

In particular embodiments, X¹ and X² each independently represent H or C₁ alkyl (i.e. methyl) optionally substituted with one or more fluoro.

In certain embodiments, X¹ and X² each independently represent C₁ alkyl (i.e. methyl) optionally substituted with one or more fluoro.

In certain embodiments, X¹ and X² each represent C₁ alkyl (i.e. methyl).

In particular embodiments, X¹ and X² represent the same group.

In more particular embodiments, either:
X¹ and X² both represent H; or
X¹ and X² both represent methyl (i.e. —CH₃).

In particular embodiments, r represents at least 1 (i.e. there is a requirement that at least one Z group is present). In further embodiments r represents up to 5, such as up to 4, up to 3 or up to 2.

For example, r may represent 1 to 6, such as 1 to 5, 1 to 4 or 1 to 3.

In more particular embodiments, r represents 1 or 2.

In more particular embodiments, r represents 1.

In particular embodiments (e.g. where r represents 1), the Z group is present in the 3- or 4-position of the cyclohexyl (relative to the point of attachment to the essential core of the compound).

Thus, in certain embodiments the essential cyclohexyl group can be depicted as follows:

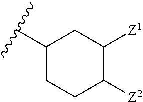

wherein the wavy line indicates the point of attachment to the essential core of the compound, and one of Z¹ and Z² represents Z and the other represents H.

For example, in certain embodiments that may be mentioned, Z² represents Z and Z¹ represents H.

In particular embodiments, each Z independently represents R^{a3}, —CN, —N₃, —N(R^{b3})R^{c3}, —OR^{d3}, —S(O)ₚR^{e3}, —S(O)_{q}N(R^{f3})R^{g3} or —N(R^{h3})S(O)ₜRⁱ³.

In more particular embodiments, each Z independently represents R^{a3}, —CN —N(R^{b3})R^{c3}, —OR^{d3}, —S(O)_{q}N(R^{f3})R^{g3} or —N(R^{h3})S(O)ₜRⁱ³.

In more particular embodiments, each Z independently represents R^{a3}, —N(R^{b3})R^{c3}, —OR^{d3} or —N(R^{h3})S(O)ₜRⁱ³.

In particular embodiments, each t represents 2.

In particular embodiments, where representing Z, R^{a3} represents C₁₋₆ alkyl (e.g. C₁₋₃ alkyl, such as C₁ alkyl) optionally substituted by one or more (e.g. one or two) G¹ groups.

For example, in certain such embodiments, Z² represents R^{a3}, —N(R^{b3})R^{c3}, —OR^{d3}, or —N(R^{h3})S(O)ₜRⁱ³, and Z¹ represents H. In other such embodiments, Z¹ represents —OR^{d3} and Z² represents H.

In particular embodiments, each R^{a4} independently represents phenyl optionally substituted by one or more halo.

In particular embodiments, when Z represents —N(R^{b3})R^{c3}, R^{b3} represents H. In alternative embodiments, R^{b3} represents C₁₋₆ alkyl (such as methyl).

In particular embodiments, when Z represents —N(R^{b3})R^{c3}, R^{b3} and/or (e.g. and) R^{c3} independently represents H or C₁₋₆ alkyl (e.g. C₁₋₅ alkyl, such as neo-pentyl, methyl(cyclobutyl), or methyl(cyclopropyl); or C₁₋₃ alkyl, such as methyl)), optionally substituted by one or more (e.g. one, two, three or four, such as two) groups independently selected from halo (such as fluoro) or G².

In certain such embodiments, G² is selected from R^{a4}, —N(R^{b4})R^{c4}, —OR^{d4}, and ═O (i.e. oxy), particularly wherein R^{a4} is phenyl, R^{b4} is methyl, R^{c4} is methyl, and R^{d4} is butyl (such as tert-butyl).

In particular embodiments, when Z represents —OR^{d3}, R^{d3} represents H or C₁₋₃ alkyl (such as methyl or ethyl) optionally substituted by one or more G² (such as one or two) such as wherein G² represents R^{a4} (particularly wherein R^{a4} represents phenyl), ═O (i.e. oxy), —OR^{d4} (particularly wherein R^{d4} is H or C₁₋₃ alkyl (such as methyl or ethyl)).

In particular embodiments, when Z represents —N(R^{h3})S(O)ₜRⁱ³, R^{h3} represents H. In alternative embodiments, R^{h3} represents C₁₋₆ alkyl (such as methyl).

In particular such embodiments, when Z represents —N(R^{h3})S(O)ₜRⁱ³, Rⁱ³ represents C₁₋₆ alkyl (such as methyl, propyl, n-propyl, i-butyl or cyclopropyl), optionally substituted by one or more groups independently selected from halo (such as fluoro) and G¹.

In particular such embodiments, t represents 2.

In particular embodiments, when Z represents —N(R^{h3})S(O)ₜRⁱ³, t represents 2, R^{h3} represents H or Me (such as H) and Rⁱ³ represents C₁₋₆ alkyl (e.g. C₁₋₄ alkyl, such as methyl, ethyl, propyl, n-propyl, i-butyl or cyclopropyl), optionally substituted by one or more groups independently selected from halo (such as fluoro) and G¹.

In particular embodiments, when Z represents —S(O)_{q}N(R^{f3})R^{g3}, R^{f3} and/or (e.g. and) R^{g3} independently represent H. In alternative embodiments, R^{f3} and/or (e.g. and) R^{g3} independently represent C₁₋₆ alkyl (such as methyl).

In particular embodiments:
R^{a3} represents C₁ alkyl optionally substituted by one or more (e.g. one or two, such as two) groups independently selected from G¹, such as wherein G¹ is selected from R^{d4} and ═O (i.e. oxy), particularly wherein R^{d4} is H or methyl;
R^{b3} represents H and R^{c3} represents C₁₋₆ alkyl (e.g. C₁₋₃ alkyl, such as methyl) optionally substituted by one or more (e.g. one or two, such as two) groups independently selected from G², such as wherein G² is selected from R^{a4} and ═O (i.e. oxy), particularly wherein R^{a4} is phenyl;
R^{d3} represents H or C₁ alkyl optionally substituted by one G², such as wherein G² represents R^{a4}, particularly wherein R^{a4} represents phenyl; and/or (e.g. and)
R^{h3} represents H and t represents 2, particularly wherein Rⁱ³ represents C₁ alkyl (i.e. methyl).

For example, in particular embodiments, each Z independently represents —OMe, —OBn (i.e. —OCH₂Ph), —OH, —NHC(O)Me, —NHC(O)Ph, —NHS(O)₂Me, —C(O)OH or —C(O)OMe.

In certain such embodiments, Z² represents —OMe, —OBn, —OH, —NHC(O)Me, —NHC(O)Ph, —NHS(O)₂Me, —C(O)OH or —C(O)OMe, and Z¹ represents H.

In other such embodiments, Z¹ represents —OMe and Z² represents H.

In particular embodiments each Z independently represents —CN, —OMe, —OBn (i.e. —OCH₂Ph), —OH, —OCH₂COOEt, —OCH₂COOH, —NH₂, —NHC(O)OtBu, —NHC(O)Me, —NHC(O)tBu, —NHC(O)(c-Bu), —NHC(O)(c-Pr), —NHC(O)CF₃, —NHC(O)NMe₂, —NHC(O)Ph, —NHS(O)₂Me, —NHS(O)₂CF₃, —NHS(O)₂(n-Pr), —NHS(O)₂(c-Pr), —NHS(O)₂(i-Bu), —NHS(O)₂(Et), —N(Me)C(O)Me, —N(Me)S(O)₂Me, —S(O)₂NHMe, —S(O)₂NMe₂, —SO₂NH₂, —C(O)OH or —C(O)OMe.

In certain such embodiments, Z² represents —CN, —OMe, —OBn, —OH, —OCH₂COOEt, —OCH₂COOH, —NH₂, —NHC(O)O^{t}Bu, —NHC(O)Me, —NHC(O)tBu, —NHC(O)(c-Bu), —NHC(O)(c-Pr), —NHC(O)CF₃, —NHC(O)NMe₂, —NHC(O)Ph, —NHS(O)₂Me, —NHS(O)₂CF₃, —NHS(O)₂(n-Pr), —NHS(O)₂(c-Pr), —NHS(O)₂(i-Bu), —NHS(O)₂(Et), —N(Me)C(O)Me, —N(Me)S(O)₂Me, —S(O)₂NHMe, —S(O)₂NMe₂, —SO₂NH₂, —C(O)OH or —C(O)OMe. In certain such embodiments, Z¹ represents H.

In particular embodiments, m represents 0 to 2 (i.e. m represents 0, 1 or 2).

In certain embodiments, m represents 0.

In certain embodiments, m represents 1.

In certain embodiments, m represents 2.

As described herein, compounds of the first aspect of the invention may also contain one or more asymmetric carbon atoms and may therefore exhibit optical and/or diastereoisomerism. Moreover, it has been found that certain such optical and/or diastereoisomers may show increased utility in the treatment of conditions as described herein, e.g. hyperglycaemia or disorders characterized by hyperglycaemia (such as type 2 diabetes).

The skilled person will understand that the stereochemical designation of the carbon bearing the essential hydroxy substituent may depend on the presence and position of heteroatoms in the ring comprising Q¹ to Q⁵. For example, the ring comprising Q¹ to Q⁵ is phenyl, the compound of formula I is such that the carbon substituted with the essential —OH group may be described as being in the (R) configuration, as understood by those skilled in the art.

The skilled person will understand that, in addition to the carbon bearing the essential hydroxy group, compounds of the invention may comprise further stereocenters. For the avoidance of doubt, unless specified, the stereochemistry at all stereocenters (including stereochemistry present in positions other than the carbon bearing the essential hydroxy group) may be in either configuration (i.e. in the R or S configuration), or may be present in compounds as a mixture thereof (e.g. a racemic mixture).

Thus, in a particular embodiment, the compound of formula I is a compound of formula IC or ID

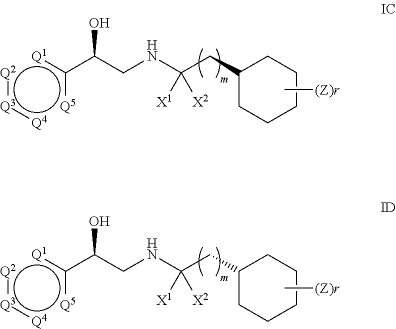

wherein ring Q, X¹, X², Z, r and m are as described herein (i.e. as described in the first aspect of the invention, including all embodiments and particular features, and combinations thereof).

In more particular embodiments, the compound of formula IC is a compound of formula IE or a compound of formula IF, and the compound of formula ID is a compound of formula IG or a compound of formula IH

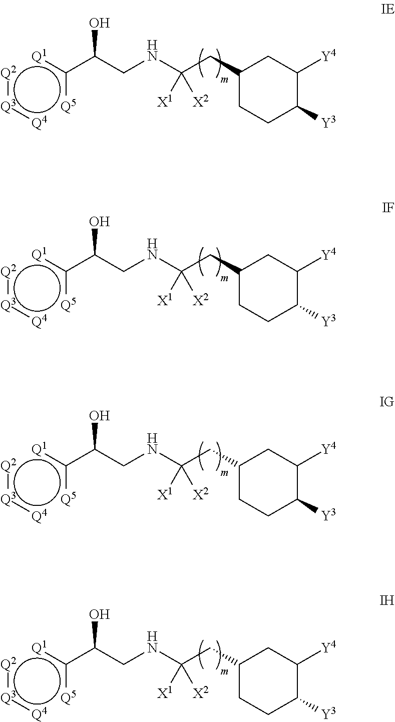

wherein ring Q, X¹, X², Y³, Y⁴ and m are as described herein (i.e. as described in the first aspect of the invention, including all embodiments and particular features, and combinations thereof).

In more particular embodiments, the compound of formula IC is a compound of formula IN or a compound of formula IJ, and the compound of formula ID is a compound of formula IL or a compound of formula IM

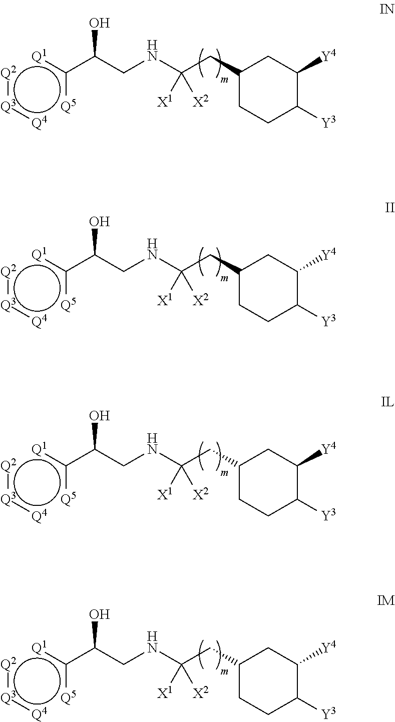

wherein ring Q, X¹, X², Y³, Y⁴ and m are as described herein (i.e. as described in the first aspect of the invention, including all embodiments and particular features, and combinations thereof).

The skilled person will understand that references to specific stereoisomer(s) of a compound of formula I (e.g. in the case of compounds of formula I, where the carbon substituted by the essential —OH group is in the R configuration will refer to the specific stereoisomer present in the substantial absence of the other (corresponding) stereoisomer(s) (e.g. in the case of compounds of formula I, where the carbon substituted by the essential —OH group is in the opposite configuration, i.e. the S configuration).

As used herein, references to the substantial absence of the corresponding opposite stereoisomer will refer to the desired stereoisomer (e.g. in the case of compounds of formula I, where the carbon substituted by the essential —OH group is in the (R) configuration) being present at a purity of at least 80% (e.g. at least 90%, such as at least 95%) relative to the opposite stereoisomer (e.g. in the case of compounds of formula I, where the carbon substituted by the essential —OH group is in the S configuration). Alternatively, in such instances, compounds may be indicated to be present in the substantial absence of the compound in the other configuration (i.e. (S) configuration), which may indicate that the compound in the relevant configuration is present in an enantiomeric excess (e.e.), or when two or more stereogenic centres are defined, in a diastereomeric excess (d.e.), of at least 90% (such as at least 95%, at least 98% or, particularly, at least 99%, for example at least 99.9%).

For the avoidance of doubt, where the stereochemistry of more than one position is specified, the compound will be present in the substantial absence of all other diastereoisomers.

For the avoidance of doubt, where the stereochemistry of a particular position is not specified, compounds of the invention will include compounds wherein that position has either available stereochemical configuration, and mixtures (e.g. racemic mixtures) thereof. Thus, compounds referred to as having a specific stereochemistry at a defined position (e.g. in the case of compounds of formula I, the carbon substituted by the essential —OH group being in the R configuration) may also have stereochemistry at one or more other positions, and so may exist as mixtures of enantiomers or diastereoisomers in relation to the stereochemistry at those positions.

### Medical Uses

As indicated herein, the compounds of the invention, and therefore compositions and kits comprising the same, are useful as pharmaceuticals.

Thus, according to a second aspect of the invention there is provided a compound of the first aspect of the invention, as hereinbefore defined (i.e. a compound as defined in the first aspect of the invention, including all embodiments and particular features thereof), for use in medicine (i.e. for use as a pharmaceutical, which may be described as use as a medicament).

Compounds described herein are β₂ adrenergic receptor agonists and therefore suitable in treating diseases such as those described herein. Such activity may be observed in compounds of the invention by identifying compounds which stimulate the uptake of glucose in skeletal muscle cells, which activity may be confirmed to be mediated by activation of the β₂ receptor by observation that such activity is prevented or diminished in the presence of a (e.g. selective) β₂ adrenergic receptor antagonist (such in the biological example provided herein).

Thus, in a third aspect of the invention, there is provided a compound of the first aspect of the invention, as hereinbefore defined, for use in treating a disease or disorder the treatment of which is mediated by activation of the β₂ adrenergic receptor.

In an alternative third aspect of the invention, there is provided the use of a compound of the first aspect of the invention in the manufacture of a medicament for use in treating a disease or disorder the treatment of which is mediated by activation of the β₂ adrenergic receptor.

In a further alternative third aspect of the invention, there is provided a method of treating a disease or disorder the treatment of which is mediated by activation of the β₂ adrenergic receptor comprising administering to a patient in need thereof a therapeutically effective amount of a compound of the first aspect of the invention.

For the avoidance of doubt, references to compounds as defined in the first aspect of the invention will include references to compounds of formula I (including all embodiments thereof) and pharmaceutically acceptable salts thereof.

As indicated herein, the compounds of the invention act by inducing uptake of glucose in skeletal muscle cells, thus allowing for the reduction of blood glucose levels in vivo. Thus, compounds of the invention may be of particular use in treating hyperglycaemia or a disorder characterized by hyperglycaemia.

In a particular embodiment of the third aspect of the invention, there is provided a compound of the first aspect of the invention, as hereinbefore defined, for use in the treatment of hyperglycaemia or a disorder characterized by hyperglycaemia.

In an alternative embodiment of the third aspect of the invention, there is provided the use of a compound of the first aspect of the invention in the manufacture of a medicament for use in the treatment of hyperglycaemia or a disorder characterized by hyperglycaemia.

In a further alternative embodiment of the third aspect of the invention, there is provided a method of treating hyperglycaemia or a disorder characterized by hyperglycaemia comprising administering to a patient in need thereof a therapeutically effective amount of a compound of the first aspect of the invention.

For the avoidance of doubt, the term “hyperglycaemia” as used herein will be understood by those skilled in the art to refer to a condition wherein an excessive amount of glucose circulates in blood plasma of the subject experiencing the same. In particular, it may refer to a subject (e.g a human subject) having blood glucose levels higher than about 10.0 mmol/L (such as higher than about 11.1 mmol/L, e.g. higher than about 15 mmol/L), although it may also refer to a subject (e.g. a human subject) having blood glucose levels higher than about 7 mmol/L for an extended period of time (e.g. for greater than 24 hours, such as for greater than 48 hours).

The skilled person will understand that references to the treatment of a particular condition (or, similarly, to treating that condition) take their normal meanings in the field of medicine. In particular, the terms may refer to achieving a reduction in the severity of one or more clinical symptom associated with the condition. For example, in the case of type 2 diabetes, the term may refer to achieving a reduction of blood glucose levels. In particular embodiments, in the case of treating hyperglycaemia or conditions characterised by hyperglycaemia, the term may refer to achieving a reduction of blood glucose levels (for example, to or below about 10.0 mmol/mL (e.g. to levels in the range of from about 4.0 mmol/L to about 10.0 mmol/L), such as to or below about 7.5 mmol/mL (e.g. to levels in the range of from about 4.0 mmol/L to about 7.5 mmol/L) or to or below about 6 mmol/mL (e.g. to levels in the range of from about 4.0 mmol/L to about 6.0 mmol/L)).

As used herein, references to patients will refer to a living subject being treated, including mammalian (e.g. human) patients. Thus, in particular embodiments of the first aspect of the invention, the treatment is in a mammal (e.g. a human).

As used herein, the term therapeutically effective amount will refer to an amount of a compound that confers a therapeutic effect on the treated patient. The effect may be objective (i.e. measurable by some test or marker) or subjective (i.e. the subject gives an indication of and/or feels an effect).

Although compounds of the first aspect of the invention may possess pharmacological activity as such, certain pharmaceutically-acceptable (e.g. “protected”) derivatives of compounds of the invention may exist or be prepared which may not possess such activity, but may be administered parenterally or orally and thereafter be metabolised in the body to form compounds of the invention. Such compounds (which may possess some pharmacological activity, provided that such activity is appreciably lower than that of the active compounds to which they are metabolised) may therefore be described as “prodrugs” of compounds of the invention.

As used herein, references to prodrugs will include compounds that form a compound of the invention, in an experimentally-detectable amount, within a predetermined time, following enteral or parenteral administration (e.g. oral or parenteral administration). All prodrugs of the compounds of the first aspect of the invention are included within the scope of the invention.

For the avoidance of doubt, the compounds of the first aspect of the invention are useful because they possess pharmacological activity, and/or are metabolised in the body following oral or parenteral administration to form compounds that possess pharmacological activity. In particular, as described herein, compounds of the first aspect of the invention are useful in the treatment of hyperglycaemia or disorders characterized by hyperglycaemia (such as type 2 diabetes), which terms will be readily understood by one of skill in the art (as described herein).

In a particular embodiment, the treatment is of a disorder (which may also be referred to as a condition or disease) characterised by hyperglycaemia.

In particular embodiments of the first aspect of the invention, the disorder is type 2 diabetes, such as type 2 diabetes of a sub-type selected from the list consisting of maturity-onset diabetes in the young (MODY), ketosis-prone diabetes in adults, latent autoimmune diabetes of adults (LADA), and gestational diabetes.

In further embodiments, the disorder is type 1 diabetes, particularly wherein the treatment further comprises treatment with insulin (or a derivative and/or functional mimetic thereof).

In particular embodiments, compounds of the invention (i.e. compounds of formula I, including all embodiments thereof) are for use in the treatment of type 2 diabetes (or useful in the manufacture of a medicament for such treatment, or useful in a method for such treatment, as described herein).

In further particular embodiments, the treatment of type 2 diabetes is in a non-obese patient.

For the avoidance of doubt, the skilled person will understand that patients with a Body Mass Index (BMI) of greater than 30 are considered to be obese.

In particular embodiments, the treatment may be of hyperglycaemia in a patent who is at risk of developing type 2 diabetes, which condition may be defined as pre-diabetes. Thus, compounds of the invention may be useful in the prevention of type 2 diabetes (e.g. in a patient having pre-diabetes).

As used herein, the term prevention (and, similarly, preventing) includes references to the prophylaxis of the disease or disorder (and vice-versa). As such, references to prevention may also be references to prophylaxis, and vice versa. In particular, the term may refer to achieving a reduction in the likelihood of the patient (or healthy subject) developing the condition (for example, at least a 10% reduction, such as at least a 20%, 30% or 40% reduction, e.g. at least a 50% reduction).

In more particular embodiments, the type 2 diabetes is characterised by the patient displaying severe insulin resistance (SIR).

In further embodiments, the treatment may be of hyperglycaemia in a patient having type 1 diabetes. Thus, compounds of the invention may be useful in the treatment of hyperglycaemia in type 1 diabetes.

The skilled person will understand that compounds of the invention may be useful in treating hyperglycaemia in patients having impaired insulin production, such as in patients having cystic fibrosis. Thus, in further embodiments, the disorder characterized by hyperglycaemia is cystic fibrosis-related diabetes.

In particular embodiments that may be mentioned, the disorder characterised by hyperglycaemia is (or is characterized by) severe insulin resistance (SIR), which may be understood by those in the art to refer to disorders wherein typically the subject has normal, or in some cases increased, insulin production but significantly reduced insulin sensitivity. In particular instances, such patients may be non-obese (e.g. being of a healthy weight). Thus, in particular embodiments, such treatments are performed in patients who are not defined as being obese (e.g. in patients who are defined as being of a healthy weight).

For example, SIR may be identified in a patient based in said patient having fasting insulin >150 pmol/L and/or a peak insulin on glucose tolerance testing of >1,500 pmol/L, particularly in individuals with a BMI <30 kg/m² (which patient may otherwise have normal glucose tolerance).

More particularly, SIR may be characterised by the patient having no significant response to the presence of insulin, which may result from a defect (e.g. a genetic defect) in the function of the insulin receptor.

Particular disorders that may be characterised by SIR include: Rabson-Mendenhall syndrome, Donohue's syndrome (leprechaunism), Type A and Type B syndromes of insulin resistance, the HAIR-AN (hyperandrogenism, insulin resistance, and acanthosis nigricans) syndromes, pseudoacromegaly, and lipodystrophy.

More particular disorders that may be characterised by SIR include Donohue's syndrome and Type A syndrome of insulin resistance and, yet more particularly, Rabson-Mendenhall syndrome.

The skilled person will understand that treatment with compounds of the first aspect of the invention may further comprise (i.e. be combined with) further (i.e. additional/other) treatment(s) for the same condition. In particular, treatment with compounds of the invention may be combined with other means for the treatment of type 2 diabetes, such as treatment with one or more other therapeutic agent that is useful in the treatment of type 2 diabetes as known to those skilled in the art, such as therapies comprising requiring the patient to undergo a change of diet and/or undertake exercise regiments, and/or surgical procedures designed to promote weight loss (such as gastric band surgery).

In particular, treatment with compounds of the invention may be performed in combination with (e.g. in a patient who is also being treated with) one or more (e.g. one) additional compounds (i.e. therapeutic agents) that:
(i) are capable of reducing blood sugar levels; and/or
(ii) are insulin sensitizers; and/or
(iii) enhance insulin release,
all of which are described herein below.

In alternative embodiments, compounds of the first aspect of the invention (i.e. compounds of the invention) may be useful in the treatment of a non-alcoholic fatty liver disease (NAFLD).

Non-alcoholic fatty liver disease (NAFLD) is defined by excessive fat accumulation in the form of triglycerides (steatosis) in the liver (designated as an accumulation of greater than 5% of hepatocytes histologically). It is the most common liver disorder in developed countries (for example, affecting around 30% of US adults) and most patients are asymptomatic. If left untreated, the condition may progressively worsen and may ultimately lead to cirrhosis of the liver. NAFLD is particularly prevalent in obese patents, with around 80% thought to have the disease.

A sub-group of NAFLD patients (for example, between 2 and 5% of US adults) exhibit liver cell injury and inflammation in addition to excessive fat accumulation. This condition, designated as non-alcoholic steatohepatitis (NASH), is virtually indistinguishable histologically from alcoholic steatohepatitis. While the simple steatosis seen in NAFLD does not directly correlate with increased short-term morbidity or mortality, progression of this condition to NASH dramatically increases the risks of cirrhosis, liver failure and hepatocellular carcinoma. Indeed, NASH is now considered to be one of the main causes of cirrhosis (including cryptogenic cirrhosis) in the developed world.

The exact cause of NASH has yet to be elucidated, and it is almost certainly not the same in every patient. It is most closely related to insulin resistance, obesity, and the metabolic syndrome (which includes diseases related to diabetes mellitus type 2, insulin resistance, central (truncal) obesity, hyperlipidaemia, low high-density lipoprotein (HDL) cholesterol, hypertriglyceridemia, and hypertension). However, not all patients with these conditions have NASH, and not all patients with NASH suffer from one of these conditions. Nevertheless, given that NASH is a potentially fatal condition, leading to cirrhosis, liver failure and hepatocellular carcinoma, there exists a clear need for an effective treatment.

In particular embodiments, compounds of the invention (i.e. compounds of formula I, including all embodiments thereof) are for use in the treatment of a non-alcoholic fatty liver disease (or useful in the manufacture of a medicament for such treatment, or useful in a method for such treatment, as described herein).

The process by which the triglyceride fat accumulates in liver cells is called steatosis (i.e. hepatic steatosis). The skilled person will understand that the term “steatosis” encompasses the abnormal retention of fat (i.e. lipids) within a cell. Thus, in particular embodiments of the first aspect of the invention, the treatment or prevention is of a fatty liver disease which is characterized by steatosis.

During steatosis, excess lipids accumulate in vesicles that displace the cytoplasm of the cell. Overtime, the vesicles can grow large enough to distort the nucleus, and the condition is known as macrovesicular steatosis. Otherwise, the condition may be referred to as microvesicular steatosis. Steatosis is largely harmless in mild cases; however, large accumulations of fat in the liver can cause significant health issues. Risk factors associated with steatosis include diabetes mellitus, protein malnutrition, hypertension, obesity, anoxia, sleep apnea and the presence of toxins within the cell.

As described herein, fatty liver disease is most commonly associated with alcohol or a metabolic syndrome (for example, diabetes, hypertension, obesity or dyslipidemia). Therefore, depending on the underlying cause, fatty liver disease may be diagnosed as alcohol-related fatty liver disease or non-alcoholic fatty liver disease (NAFLD).

Particular diseases or conditions that are associated with fatty liver disease that are not related to alcohol include metabolic conditions such as diabetes, hypertension, obesity, dyslipidemia, abetalipoproteinemia, glycogen storage diseases, Weber-Christian disease, acute fatty liver of pregnancy, and lipodystrophy. Other non-alcohol related factors related to fatty liver diseases include malnutrition, total parenteral nutrition, severe weight loss, refeeding syndrome, jejunoileal bypass, gastric bypass, polycystic ovary syndrome and diverticulosis.

The compounds of the invention have been found to be particularly useful in the treatment or prevention of NAFLD, which may be referred to as a fatty liver disease which is not alcohol related. A fatty liver disease which is “not alcohol related” may be diagnosed wherein alcohol consumption of the patient is not considered to be a main causative factor. A typical threshold for diagnosing a fatty liver disease as “not alcohol related” is a daily consumption of less than 20 g for female subjects and less than 30 g for male subjects.

If left untreated, subjects suffering from fatty liver disease may begin to experience inflammation of the the liver (hepatitis). It has been postulated that one of the possible causes of this inflammation may be lipid peroxidative damage to the membranes of the liver cells. Inflammation of a fatty liver can lead to a number of serious conditions and it is therefore desirable to treat or prevent fatty liver disease before inflammation occurs. Thus, in particular embodiments of the first aspect of the invention, the treatment or prevention is of a NAFLD which is associated with inflammation.

Non-alcoholic steatohepatitis (NASH) is the most aggressive form of NAFLD, and is a condition in which excessive fat accumulation (steatosis) is accompanied by inflammation of the liver. If advanced, NASH can lead to the development of scar tissue in the liver (fibrosis) and, eventually, cirrhosis. As described above, the compounds of the invention have been found to be useful in the treatment or prevention of NAFLD, particularly when accompanied by inflammation of the liver. It follows that the compounds of the invention are also useful in the treatment or prevention of NASH. Therefore, in a further embodiment of the first aspect of the invention, the treatment or prevention is of non-alcoholic steatohepatitis (NASH).

The skilled person will understand that treatment with compounds of the first aspect of the invention may further comprise (i.e. be combined with) further (i.e. additional/other) treatment(s) for the same condition. In particular, treatment with compounds of the invention may be combined with other means for the treatment of a fatty liver disease, as described herein, such as treatment with one or more other therapeutic agent that is useful in the treatment of a fatty liver disease as known to those skilled in the art; for example, therapies comprising requiring the patient to undergo a change of diet and/or undertake exercise regiments, and/or surgical procedures designed to promote weight loss (such as gastric band surgery).

In particular, treatment with compounds of the invention may be performed in combination with (e.g. in a patient who is also being treated with) one or more (e.g. one) additional compounds (i.e. therapeutic agents) that are capable of reducing the level of fat (e.g. triglycerides) in the liver.

References to treatment of a fatty liver disease may refer to achieving a therapeutically significant reduction of fat (e.g. triglycerides levels) in liver cells (such as a reduction of at least 5% by weight, e.g. a reduction of at least 10%, or at least 20% or even 25%).

As described herein, compounds of the invention may be of use in treating a disease or disorder the treatment of which is mediated by activation of the β₂ adrenergic receptor.

In particular embodiments, the compounds of the first aspect of the invention may be understood to positively modulate the β₂ adrenergic receptor, which compounds may be referred to as a β₂-adrenergic receptor agonist.

The skilled person will appreciate what is meant by “β₂ adrenergic receptor” (or “β₂-AR”). Such receptors are known in the art and have been reviewed in, e.g., Johnson. M., J. Allergy Clin. Immunol., 117, 18-24 (2006). For the avoidance of doubt, adrenergic receptors are a class of G protein-coupled receptors which bind and are activated by their endogenous ligands, the catecholamines, adrenaline and noradrenaline. The adrenergic receptor falls into five types: α₁, α₂, β₁, β₂ and β₃. These subtypes are expressed in distinct patterns and involved in different physiological processes, such that ligands that can selectively target one subtype have therapeutic potential for multiple diseases. The present invention is concerned with the β₂ adrenergic receptor, although compounds may interact with one or more other adrenergic receptor (e.g. one or more other β adrenergic receptor).

The term “positively modulates β₂-adrenergic receptor activity” will be understood to mean that the compound is capable of altering the signalling of the receptor.

As used herein, the term “β₂ agonist” is used to mean β₂ adrenergic receptor agonist. In certain embodiments, the term β₂ agonist is understood to include compounds that are primarily β₂ agonists, but may also exhibit some agonism for other adrenergic receptors. In this application, the terms “β₂ adrenergic receptor agonist”, “β₂ AR agonist”, “β₂AR agonist” and “β₂ agonist” may be used interchangeably.

Thus, in certain embodiments, references to β₂ agonists may include both selective and non-selective agonists.

In certain embodiments, references to β₂ agonists may include any ligand that change receptor signalling including but not limited to full and partial agonists. Further, β₂ agonists that may be used in accordance with various aspects and embodiments of the present disclosure may be short-acting, long acting or ultra long-acting.

As used herein, the term “mediated by activation of the β₂ adrenergic receptor” is used to indicate that activation of the receptor regulates or causes a physiological response which will in turn provide a biological effect corresponding to (or leading to) treatment of the disease or disorder.

As used in herein, references to diseases and disorders the treatment of which is “mediated by activation of the β₂ adrenergic receptor” may also refer to diseases and disorders (and in particular the treatment thereof) being, inter alia, “associated with”, “mediated by”, “affected by”, “regulated by”, “modulated by” and “linked to” the β₂ adrenergic receptor.

As described herein, diseases and disorders the treatment of which is mediated by activation of the β₂ adrenergic receptor will be known to those skilled in the art. Thus, the skilled person will understand that in respect of certain of the diseases and disorders described herein the suitability of compounds of the invention for the treatment of such diseases and disorders may be known to those skilled in the art; for example, based on the disclosures referred to herein below (the contents of which are incorporated herein by reference).

In addition to those as may be described herein above, particular diseases and disorders the treatment of which is mediated by activation of the β₂ adrenergic receptor that may be mentioned include:
neurodegenerative diseases, such as MCI (mild cognitive impairment), aMCI (amnestic MCI), vascular dementia, mixed dementia, FTD (front-temporal dementia), HD (Huntington disease), Rett syndrome, PSP (progressive supranuclear palsy), CBD (corticobasal degeneration), SCA (spinocerebellar ataxia), MSA (multiple system atrophy), SDS (Shy-Drager syndrome), olivopontocerebellar atrophy, TBI (traumatic brain injury), CTE (chronic traumatic encephalopathy), stroke, EKS (Wernicke-Korsakoff syndrome), normal pressure hydrocephalus, hypersomnia (narcolepsy), ASD (autistic spectrum disorders), FXS (fragile X syndrome), YSC (tubular sclerosis complex), prion-related disorders, CJD (Creutzfeldt-Jakob disease), depressive disorders, DLC (dementia with Lewy bodies), PD (Parkinson's disease), PDD (PD dementia), ADHD (attention deficit hyperactivity disorder), Alzheimer's disease (AD), early AD and DS (Down syndrome);
muscle dystrophy or a disorder characterised by muscular dystrophy, such as muscle damage, muscle wasting, muscle atrophy, muscle degeneration or sclerosis;
kidney disease, such as CKD (chronic kidney disease), ESRD (end-stage renal disease) and diabetic nephropathy;
inflammation or a disorder characterised by inflammation, such as sepsis, psoriasis, dermatitis, psoriasis-like skin dermatitis, lacerations or HDF (human dermal fibroblasts), and including localised acute inflammation, such as that related to endotoxemia and Acute Lung Injury (ALI), and respiratory conditions associated with inflammation, such as asthma and other pulmonary disorders, such as chronic obstructive pulmonary disease (COPD); and
an autoimmune disease, such as SLE (systemic lupus erythematosus, RA (rheumatoid arthritis), MG (myasthenia gravis) MS and GD (Grave's disease).

The suitability of β₂ adrenergic receptor agonists for treating such conditions may be demonstrated by the data provided herein and by reference to the literature known to those skilled on the art, such as that described herein (the whole contents of which, in particular the experimental results presented, will be understood to be incorporated herein by reference).

In particular, the suitability of β₂ adrenergic receptor agonists for treating certain of the diseases and disorders referred to herein may be identified in and, in some instances, confirmed by the disclosures of WO 2020/198466 A1 and WO 2021/003161 A1 (which, for the avoidance of doubt, are incorporated herein by reference, in particular the examples as provided therein).

In a particular embodiment, there is provided a compound of the first aspect of the invention, as hereinbefore defined, for use in treating neurodegenerative diseases.

In particular embodiments, the neurodegenerative disease is selected from MCI (mild cognitive impairment), aMCI (amnestic MCI), vascular dementia, mixed dementia, FTD (front-temporal dementia), HD (Huntington disease), Rett syndrome, PSP (progressive supranuclear palsy), CBD (corticobasal degeneration), SCA (spinocerebellar ataxia), MSA (multiple system atrophy), SDS (Shy-Drager syndrome), olivopontocerebellar atrophy, TBI (traumatic brain injury), CTE (chronic traumatic encephalopathy), stroke, EKS (Wernicke-Korsakoff syndrome), normal pressure hydrocephalus, hypersomnia (narcolepsy), ASD (autistic spectrum disorders), FXS (fragile X syndrome), YSC (tubular sclerosis complex), prion-related disorders, CJD (Creutzfeldt-Jakob disease), depressive disorders, DLC (dementia with Lewy bodies), PD (Parkinson's disease), PDD (PD dementia), ADHD (attention deficit hyperactivity disorder), Alzheimer's disease (AD), early AD and DS (Down syndrome).

Mittal. S., et al., Science., 357(6354), 891-898 (2017) describes that β₂-adrenergic receptor agonists promote dopamine neuron health by reducing SNCA expression through H2K27 deacetylation and mitochondrial free radicals. This may benefit nigral dopamine neurons, which are prone to mitochondrial bioenergetics dysfunction at early stages of Lewy body neuropathy. β₂-adrenergic receptor agonists are expressed in the substantia nigra and cortex, regions that are progressively affected by Parkinson's disease (PD). Therefore, β₂-adrenergic receptor agonists can be used to reduce the risk and affect of PD.

Hishida. R., The Lancet, 870 (1992) describes that β₂-adrenergic receptor agonists can beneficially affect wearing-off in patients with Parkinson's disease on long-term levodopa.

Uc, E. Y., et al., Clin. Neuropharmacol., 26(4), 207-212 (2003) describes that β₂-adrenergic receptor agonist, albuterol, benefited patients with PD through two mechanisms, an increased response to levodopa and an increase in muscle mass.

O'Neill, et al., Br. J. Pharmacol., 177, 282-297 (2019) describes that β₂-adrenergic receptor agonists restrict microglial activation and protect against the onset and progression of dopamine neuronal cell loss and related motor deficits provoke by central or systemic inflammation. Therefore, targeting β₂-adrenergic receptors with a β₂-adrenergic receptor agonist imbues an intervening prophylactic mechanism to protect against the progression of neurodegeneration and exacerbated decline in motor function associated with systemic and central inflammation. As a result, β₂-adrenergic receptor agonists may be beneficial in the treatment of PD-related neuropathy and motor impairments induced by inflammation.

In alternative embodiments, there is provided a compound of the first aspect of the invention, as hereinbefore defined, for use in treating muscle dystrophy or a disorder characterised by muscular dystrophy.

In particular such embodiments, the muscle dystrophy is muscle damage, muscle wasting, muscle atrophy, muscle degeneration or sclerosis.

Jiang, G., et al., ISRN Pharma., 2011, 1-7 (2011) describes that β₂-AR agonists ameliorate animal wasting in denervation, amyotrophic lateral sclerosis, muscular dystrophy, disuse, aging and myocardial unloading models. Further, in patients with immobilization conditions or muscular dystrophy, β₂-AR agonists increase lean body mass and enhance skeletal muscle functions. Also, β₂-AR agonists were found to promote myocardial recovery in patients with myocardial unloading atrophy resulting from application of left ventricular assist devise.

Bartus, R. T., et al., Neurobiol. Dis., 85, 11-24, 2016 indicates that β₂-adrenergic receptor agonists may enhance muscle bulk and muscle strength in amyotrophic lateral sclerosis (ALS) patients by increasing neurotrophic factors.

In alternative embodiments, there is provided a compound of the first aspect of the invention, as hereinbefore defined, for use in treating kidney disease.

In particular such embodiments, the kidney disease is selected from CKD (chronic kidney disease), ESRD (end-stage renal disease) and diabetic nephropathy.

Cleveland, K., et al., FASEB Journal, 33(1), 514 (2019) describes that β₂-adrenergic receptor agonists have been shown to induce mitochondrial biogenesis (MB) and promote recovery from acute kidney injury, and may find use as a potential therapy for diabetic nephropathy (DN).

Jesinkey, S. R., et al., J. Am. Soc. Nephrol., 25, 1157-1162 (2014) describes the necessity for mitochondrial biogenesis as an adaptive response for meeting the increased metabolic and energy demands during organ recovery after an acute injury. In particular, renal mitochondrial dysfunction has been linked to pathogenesis of acute kidney injury (AKI), a disorder characterised by a rapid decrease in kidney excretory function and subsequent retention of harmful waste products.

In alternative embodiments, there is provided a compound of the first aspect of the invention, as hereinbefore defined, for use in treating inflammation or a disorder characterised by inflammation.

In particular embodiments, the inflammation is (or is characterised by) sepsis, psoriasis, dermatitis, psoriasis-like skin dermatitis, lacerations or HDF (human dermal fibroblasts).

As the skilled person will know, inflammation is a tightly controlled process that ensures proper localization of immune cells, release of pro- and anti-inflammatory mediators, clearance of dead cells, and containment of the pathogen.

The skilled person will know that inflammation may also be a cause of respiratory conditions, such as asthma and other pulmonary disorders, such as chronic obstructive pulmonary disease (COPD).

Grailer, J. J. et al, J Innate Immun, 6, 607-618 (2014) shows that blockade of the β₂ adrenergic receptor reduced survival and enhanced injury in mouse models of endotoxemia and LPS-induced acute lung injury, respectively. These results demonstrate the suitability of β₂AR activation in the treatment of localised acute inflammation, such as that related to endotoxemia and Acute Lung Injury.

Agac, D., et al., Brain, Behaviour and Immunity, 74, 176-185 (2018) describes that there is a unique synergistic pathway that converts acute inflammatory signals into an anti-inflammatory response and likely explains a variety of phenomena known to be involved in β₂-adrenergic receptor agonists mediated immune suppression. In particular, β₂-adrenergic receptor agonists signalling directly controls anti-inflammatory cytokine, IL-10, expression. These results suggest the use of β₂AR agonists in the treatment of inflammatory disorders, such as sepsis.

Liu, F., et al., Cells, 511(9), 1-17 (2020) describes that β₂-adrenergic receptor agonists demonstrated significant anti-psoriasis effects, which may involve regulating the Th17/Tregs axis balances and glycerophospholipid metabolism in response to imiquimod (IMQ) induced psoriasis.

Provost, G. S., et al., J. Investig. Dermatol., 135, 279-288 (2015) describes that β₂-adrenergic receptor agonists reduces human dermal fibroblast (HDF) differentiation, therefore reducing scarring to a patient following a laceration or open wound.

In alternative embodiments, there is provided a compound of the first aspect of the invention, as hereinbefore defined, for use in treating an autoimmune disease.

In particular such embodiments, the autoimmune disease is selected from SLE (systemic lupus erythematosus, RA (rheumatoid arthritis), MG (myasthenia gravis) MS and GD (Grave's disease).

Wu, et al., Front. Pharmacol., 1313(9), 1-9 (2018) describes that β₂-adrenergic receptor agonists may be a target treatment for autoimmune diseases (AD), such as SLE (systemic lupus erythematosus, RA (rheumatoid arthritis), MG (mysasthenia gravis) MS and GD (Grave's disease).

### Pharmaceutical Compositions

As described herein, compounds of the first and, therefore, the second and third aspects of the invention are useful as pharmaceuticals. Such compounds may be administered alone or may be administered by way of known pharmaceutical compositions/formulations.

In a fourth aspect of the invention, there is provided a pharmaceutical composition comprising a compound as defined in the second or third aspect of the invention, and optionally one or more pharmaceutically acceptable adjuvant, diluent and/or carrier.

The skilled person will understand that references herein to compounds of the first aspect of the invention being for particular uses (and, similarly, to uses and methods of use relating to compounds of the invention) may also apply to pharmaceutical compositions comprising compounds of the invention as described herein.

In a fifth aspect of the invention, there is provided a pharmaceutical composition for use in the treatment of hyperglycaemia or a disorder characterized by hyperglycaemia (as defined herein, such as type 2 diabetes) comprising a compound as defined in the first aspect of the invention, and optionally one or more pharmaceutically acceptable adjuvant, diluent and/or carrier.

In an alternative fifth aspect of the invention, there is provided a pharmaceutical composition for use in the treatment or prevention of a non-alcoholic fatty liver disease, as defined herein.

In an alternative fifth aspect of the invention, there is provided a pharmaceutical composition for use in the treatment or prevention of a non-alcoholic fatty liver disease, as defined herein.

The skilled person will understand that compounds of the first (and, therefore, second and third) aspect of the invention may act systemically and/or locally (i.e. at a particular site).

The skilled person will understand that compounds and compositions as described in the first to fifth aspects of the invention will normally be administered orally, intravenously, subcutaneously, buccally, rectally, dermally, nasally, tracheally, bronchially, sublingually, intranasally, topically, by any other parenteral route or via inhalation, in a pharmaceutically acceptable dosage form. Pharmaceutical compositions as described herein will include compositions in the form of tablets, capsules or elixirs for oral administration, suppositories for rectal administration, sterile solutions or suspensions for parenteral or intramuscular administration, and the like. Alternatively, particularly where such compounds of the invention act locally, pharmaceutical compositions may be formulated for topical administration.

Thus, in particular embodiments of the fourth and fifth aspects of the invention, the pharmaceutical formulation is provided in a pharmaceutically acceptable dosage form, including tablets or capsules, liquid forms to be taken orally or by injection, suppositories, creams, gels, foams, inhalants (e.g. to be applied intranasally), or forms suitable for topical administration. For the avoidance of doubt, in such embodiments, compounds of the invention may be present as a solid (e.g. a solid dispersion), liquid (e.g. in solution) or in other forms, such as in the form of micelles.

For example, in the preparation of pharmaceutical formulations for oral administration, the compound may be mixed with solid, powdered ingredients such as lactose, saccharose, sorbitol, mannitol, starch, amylopectin, cellulose derivatives, gelatin, or another suitable ingredient, as well as with disintegrating agents and lubricating agents such as magnesium stearate, calcium stearate, sodium stearyl fumarate and polyethylene glycol waxes. The mixture may then be processed into granules or compressed into tablets.

Soft gelatin capsules may be prepared with capsules containing one or more active compounds (e.g. compounds of the first and, therefore, second and third aspects of the invention, and optionally additional therapeutic agents), together with, for example, vegetable oil, fat, or other suitable vehicle for soft gelatin capsules. Similarly, hard gelatine capsules may contain such compound(s) in combination with solid powdered ingredients such as lactose, saccharose, sorbitol, mannitol, potato starch, corn starch, amylopectin, cellulose derivatives or gelatin.

Dosage units for rectal administration may be prepared (i) in the form of suppositories which contain the compound(s) mixed with a neutral fat base; (ii) in the form of a gelatin rectal capsule which contains the active substance in a mixture with a vegetable oil, paraffin oil, or other suitable vehicle for gelatin rectal capsules; (iii) in the form of a ready-made micro enema; or (iv) in the form of a dry micro enema formulation to be reconstituted in a suitable solvent just prior to administration.

Liquid preparations for oral administration may be prepared in the form of syrups or suspensions, e.g. solutions or suspensions, containing the compound(s) and the remainder of the formulation consisting of sugar or sugar alcohols, and a mixture of ethanol, water, glycerol, propylene glycol and polyethylene glycol. If desired, such liquid preparations may contain colouring agents, flavouring agents, saccharine and carboxymethyl cellulose or other thickening agent. Liquid preparations for oral administration may also be prepared in the form of a dry powder to be reconstituted with a suitable solvent prior to use.

Solutions for parenteral administration may be prepared as a solution of the compound(s) in a pharmaceutically acceptable solvent. These solutions may also contain stabilizing ingredients and/or buffering ingredients and are dispensed into unit doses in the form of ampoules or vials. Solutions for parenteral administration may also be prepared as a dry preparation to be reconstituted with a suitable solvent extemporaneously before use.

The skilled person will understand that compounds of the invention, and pharmaceutically-acceptable salts thereof, may be administered (for example, as formulations as described hereinabove) at varying doses, with suitable doses being readily determined by one of skill in the art. Oral, pulmonary and topical dosages (and subcutaneous dosages, although these dosages may be relatively lower) may range from between about 0.01 μg/kg of body weight per day (μg/kg/day) to about 200 μg/kg/day, preferably about 0.01 to about 10 μg/kg/day, and more preferably about 0.1 to about 5.0 μg/kg/day. For example, when administered orally, treatment with such compounds may comprise administration of a formulations typically containing between about 0.01 μg to about 2000 mg, for example between about 0.1 μg to about 500 mg, or between 1 μg to about 100 mg (e.g. about 20 μg to about 80 mg), of the active ingredient(s). When administered intravenously, the most preferred doses will range from about 0.001 to about 10 μg/kg/hour during constant rate infusion. Advantageously, treatment may comprise administration of such compounds and compositions in a single daily dose, or the total daily dosage may be administered in divided doses of two, three or four times daily (e.g. twice daily with reference to the doses described herein, such as a dose of 10 mg, 20 mg, 30 mg or 40 mg twice daily, or 10 μg, 20 μg, 30 μg or 40 μg twice daily).

In any event, the skilled person (e.g. the physician) will be able to determine the actual dosage which will be most suitable for an individual patient, which is likely to vary with the route of administration, the type and severity of the condition that is to be treated, as well as the species, age, weight, sex, renal function, hepatic function and response of the particular patient to be treated. The above-mentioned dosages are exemplary of the average case; there can, of course, be individual instances where higher or lower dosage ranges are merited, and such are within the scope of this invention.

As described herein above, the skilled person will understand that treatment with compounds of the first aspect of the invention may further comprise (i.e. be combined with) further (i.e. additional/other) treatment(s) for the same condition. In particular, treatment with compounds of the invention may be combined with other means for the treatment of hyperglycaemia or a disorder characterized by hyperglycaemia (as defined herein, such as type 2 diabetes), such as treatment with one or more other therapeutic agent that is useful in the treatment of hyperglycaemia or a disorder characterized by hyperglycaemia (as defined herein, such as type 2 diabetes).

In particular embodiments of the fourth and fifth aspects of the invention, the pharmaceutical composition may further comprise one or more additional (i.e. other) therapeutic agent.

In more particular embodiments, the one or more additional therapeutic agent is an agent for the treatment of type 2 diabetes as known to those skilled in the art, such as metformin, sulfonylureas (e.g. carbutamide, acetohexamide, chlorpropamide, tolbutamide. glipizide (glucotrol), gliclazide, glibenclamide, glyburide (Micronase), glibornuride, gliquidone, glisoxepide, glyclopyramide, glimepiride (Amaryl), glimiprime, JB253 or JB558), thiazolidinediones (e.g. pioglitazone, rosiglitazone (Avandia), lobeglitazone (Duvie) and troglitazone (Rezulin)), dipeptidyl peptidase-4 inhibitors (e.g. sitagliptin, vildagliptin, saxagliptin, linagliptin, anagliptin, teneligliptin, alogliptin, trelagliptin, gemigliptin, dutogliptin and omarigliptin), SGLT2 inhibitors (e.g. dapagliflozin, empagliflozin, canagliflozin, ipragliflozin, tofogliflozin, sergliflozin etabonate, remogliflozin etabonate, and ertugliflozin), and glucagon-like peptide-1 (GLP-1) analogues.

The skilled person will understand that combinations of therapeutic agents may also described as a combination product and/or provided as a kit-of-parts.

In a sixth aspect of the invention, there is provided a combination product comprising:
(A) a compound as defined in the first aspect of the invention; and
(B) one or more additional therapeutic agent,
   wherein each of components (A) and (B) is formulated in admixture, optionally with one or more a pharmaceutically-acceptable adjuvant, diluent or carrier.

In a seventh aspect of the invention, there is provided a kit-of-parts comprising:
(a) a compound as defined in the first (or second and/or third) aspect of the invention, (or a pharmaceutical composition comprising the same) or a pharmaceutical composition as defined in the fourth or fifth aspect of the invention; and
(b) one or more other therapeutic agent, optionally in admixture with one or more pharmaceutically-acceptable adjuvant, diluent or carrier,
   which components (a) and (b) are each provided in a form that is suitable for administration in conjunction with the other.

In particular embodiments (e.g. of the sixth and seventh aspects of the invention), the additional therapeutic agent is a therapeutic agent that is useful for the treatment of hyperglycaemia or a disorder characterized by hyperglycaemia (e.g. type 2 diabetes), as known to those skilled in the art (such as those described herein).

For example, in particular embodiments of the fourth to fifth aspects of the invention, the additional therapeutic agent is an agent that:
(i) is capable of reducing blood sugar levels; and/or
(ii) is an insulin sensitizer; and/or
(iii) is able to enhance insulin release,
   which agents will be readily identified by those skilled in the art and include, in particular, such therapeutic agents that are commercially available (e.g. agents that the subject of a marketing authorization in one or more territory, such as a European or US marketing authorization).

The skilled person will understand that references to therapeutic agents capable of reducing blood glucose levels may refer to compounds capable of reducing levels of blood by at least 10% (such as at least 20%, at least 30% or at least 40%, for example at least 50%, at least 60%, at least 70% or at least 80%, e.g. at least 90%) when compared to the blood glucose levels prior to treatment with the relevant compound.

In alternative embodiments of the sixth and seventh aspects of the invention, the additional therapeutic agent is an agent for the treatment or prevention of a non-alcoholic fatty liver disease (such as NASH), which agents will be readily identified by those skilled in the art and include, in particular, such therapeutic agents that are commercially available (e.g. agents that the subject of a marketing authorization in one or more territory, such as a European or US marketing authorization).

In alternative embodiments of the sixth and seventh aspects of the invention, the additional therapeutic agent is an agent for treating a disease or disorder the treatment of which is mediated by activation of the β₂ adrenergic receptor, which diseases and disorders will include those described herein, and which agents will be readily identified by those skilled in the art and include, in particular, such therapeutic agents that are commercially available (e.g. agents that the subject of a marketing authorization in one or more territory, such as a European or US marketing authorization).

### Preparation of Compounds/Compositions

Pharmaceutical compositions/formulations, combination products and kits as described herein may be prepared in accordance with standard and/or accepted pharmaceutical practice.

Thus, in a further aspect of the invention there is provided a process for the preparation of a pharmaceutical composition/formulation, as hereinbefore defined, which process comprises bringing into association a compound of the invention, as hereinbefore defined, with one or more pharmaceutically-acceptable adjuvant, diluent or carrier.

In further aspects of the invention, there is provided a process for the preparation of a combination product or kit-of-parts as hereinbefore defined, which process comprises bringing into association a compound of the invention, as hereinbefore defined, or a pharmaceutically acceptable salt thereof with the other therapeutic agent that is useful in the treatment of hyperglycaemia or a disorder characterized by hyperglycaemia (e.g. type 2 diabetes), and at least one pharmaceutically-acceptable adjuvant, diluent or carrier.

As used herein, references to bringing into association will mean that the two components are rendered suitable for administration in conjunction with each other.

Thus, in relation to the process for the preparation of a kit of parts as hereinbefore defined, by bringing the two components “into association with” each other, we include that the two components of the kit of parts may be:
(i) provided as separate formulations (i.e. independently of one another), which are subsequently brought together for use in conjunction with each other in combination therapy; or
(ii) packaged and presented together as separate components of a “combination pack” for use in conjunction with each other in combination therapy.

Compounds as defined in the first aspect of the invention (i.e. compounds of the invention) may be prepared in accordance with techniques that are well known to those skilled in the art, such as those described in the examples provided hereinafter.

For example, there is provided a process for the preparation of a compound of formula I, or a pharmaceutically acceptable salt thereof, wherein R¹ represents H and the remaining substituents are as defined in the first aspect of the invention, which process comprises:
(i) reaction of a compound of formula II

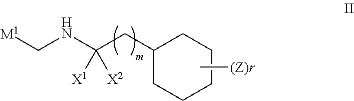

wherein X¹, X², Z, r and m are as defined herein, and wherein M¹ represents a suitable metal or metal halide, with a compound of formula III

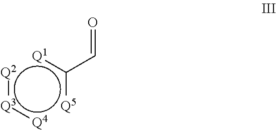

wherein Q¹ to Q⁵ (and, therefore ring Q) are as defined herein, under conditions known to those skilled in the art;
(ii) reaction of a compound of formula IV

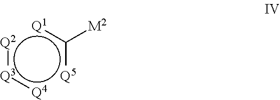

wherein Q¹ to Q⁵ are as defined herein, and wherein M² represents a suitable metal or metal halide, with a compound of formula V

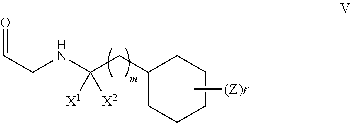

wherein X¹, X², Z, r and m are as defined herein, under conditions known to those skilled in the art.

Compounds of formulae II, III, IV, V are either commercially available, are known in the literature, or may be obtained either by analogy with the processes described herein, or by conventional synthetic procedures, in accordance with standard techniques, from available starting materials (e.g. appropriately substituted benzaldehydes, styrenes or phenacyl bromides (or phenacylchloride, and the like) using appropriate reagents and reaction conditions. In this respect, the skilled person may refer to inter alia “Comprehensive Organic Synthesis” by B. M. Trost and I. Fleming, Pergamon Press, 1991. Further references that may be employed include “Science of Synthesis”, Volumes 9-17 (Hetarenes and Related Ring Systems), Georg Thieme Verlag, 2006.

The substituents X and R¹, as hereinbefore defined, may be modified one or more times, after or during the processes described above for preparation of compounds of formula I by way of methods that are well known to those skilled in the art. Examples of such methods include substitutions, reductions, oxidations, dehydrogenations, alkylations, dealkylations, acylations, hydrolyses, esterifications, etherifications, halogenations and nitrations. The precursor groups can be changed to a different such group, or to the groups defined in formula I, at any time during the reaction sequence. The skilled person may also refer to “Comprehensive Organic Functional Group Transformations” by A. R. Katritzky, O. Meth-Cohn and C. W. Rees, Pergamon Press, 1995 and/or “Comprehensive Organic Transformations” by R. C. Larock, Wiley-VCH, 1999.

Such compounds may be isolated from their reaction mixtures and, if necessary, purified using conventional techniques as known to those skilled in the art. Thus, processes for preparation of compounds of the invention as described herein may include, as a final step, isolation and optionally purification of the compound of the invention (e.g. isolation and optionally purification of the compound of formula I).

The skilled person will understand that compounds of formula I having specific stereochemistry may be provided by reacting suitable starting materials having the required stereochemistry in processes as described herein. Further, the skilled person will understand that suitable starting materials having the required stereochemistry may be prepared by analogy with the processes described herein.

It will be appreciated by those skilled in the art that, in the processes described above and hereinafter, the functional groups of intermediate compounds may need to be protected by protecting groups. The protection and deprotection of functional groups may take place before or after a reaction in the above-mentioned schemes.

Protecting groups may be applied and removed in accordance with techniques that are well known to those skilled in the art and as described hereinafter. For example, protected compounds/intermediates described herein may be converted chemically to unprotected compounds using standard deprotection techniques. The type of chemistry involved will dictate the need, and type, of protecting groups as well as the sequence for accomplishing the synthesis. The use of protecting groups is fully described in “Protective Groups in Organic Synthesis”, 3rd edition, T. W. Greene & P. G. M. Wutz, Wiley-Interscience (1999).

Compounds as described herein (in particular, compounds as defined in the first and, therefore, second and third aspects of the invention) may have the advantage that they may be more efficacious than, be less toxic than, be longer acting than, be more potent than, produce fewer side effects than, be more easily absorbed than, and/or have a better pharmacokinetic profile (e.g. higher oral bioavailability and/or lower clearance) than, and/or have other useful pharmacological, physical, or chemical properties over, compounds known in the prior art, whether for use in the above-stated indications or otherwise. In particular, such compounds may have the advantage that they are more efficacious and/or exhibit advantageous properties in vivo.

Without wishing to be bound by theory, compounds as described herein are thought to be potent agonists of the β₂-adrenergic receptor, which allows for increased glucose uptake in skeletal muscle cells.

In addition, compounds as described herein are thought to be agonists of the β₂-adrenergic receptor without (or with only a minimal effect in) inducing cAMP production. It is thought that this allows for effects such as the increased glucose uptake in skeletal muscle cells with lower levels of side effects than would result from other treatments. Further, combining compounds as described herein with other therapeutic agents, such as those that are able to decrease blood glucose levels, is thought to provide an effective combination therapy.

### EXAMPLES

The present invention is illustrated by way of the following examples.

Chemicals and reagents were obtained from commercial suppliers and were used as received unless otherwise stated. All reactions involving moisture sensitive reagents were performed in oven or flame dried glassware under a positive pressure of nitrogen or argon.

### Abbreviations

Abbreviations as used herein will be known to those skilled in the art. In particular, the following abbreviations may be used herein.
atm atmosphere
aq aqueous
Boc₂O di-tert-butyl dicarbonate
DIPEA N,N-Diisopropylethylamine
DMAP N,N-dimethylaminopyridine
MTBE methyl tert-butyl ether
Pd/C palladium on carbon
rt room temperature
sat saturated
TFA trifluoracetic acid
TMSOTf trimethylsilyl trifluoromethanesulfonate

### Example Compounds

In the event that there is a discrepancy between nomenclature and the structure of compounds as depicted graphically, it is the latter that presides (unless contradicted by any experimental details that may be given and/or unless it is clear from the context).

### Example 1: (R)-1-(3-Fluorophenyl)-2-((((1s,4S)-4-methoxycyclohexyl)-methyl)amino)ethan-1-ol

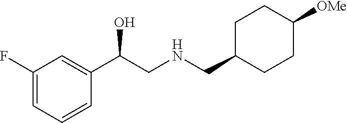 [SMILES: CO[C@H]1CC[C@@H](CNC[C@H](O)c2cccc(F)c2)CC1]

### (a) Methyl (1s,4s)-4-methoxycyclohexane-1-carboxylate

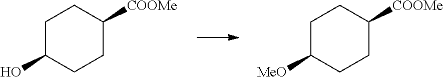 [SMILES: COC(=O)[C@H]1CC[C@@H](O)CC1 ; COC(=O)[C@H]1CC[C@@H](OC)CC1]

N1,N1,N8,N8-Tetramethylnaphthalene-1,8-diamine (1.5 g, 6.95 mmol) was added to an ice-cooled solution of methyl (1s,4s)-4-hydroxycyclohexane-1-carboxylate (1.0 g, 6.32 mmol) in CH₂Cl₂ (60 mL). The mixture was stirred at rt for 30 min. Trimethyl oxonium tetrafluoroborate (1.3 g, 8.53 mmol) was added at 0° C. and the mixture was stirred at rt for 18 h. H₂O and CH₂Cl₂ were added and the phases were separated. The aq phase was extracted with CH₂Cl₂ and the combined extracts were washed with brine, dried (Na₂SO₄) and concentrated. The residue was purified by chromatography to give the sub-title compound (0.86 g, 79%).

### (b) ((1s,4s)-4-Methoxycyclohexyl)methanol

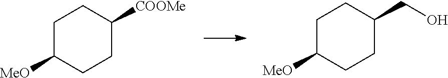 [SMILES: COC(=O)[C@H]1CC[C@@H](OC)CC1 ; CO[C@H]1CC[C@@H](CO)CC1]

Methyl (1s,4s)-4-methoxycyclohexane-1-carboxylate (850 mg g, 4.94 mmol) in THF (7 mL) was added dropwise to a stirred ice-cooled mixture of LiAlH₄ (2.4 M in THF, 2.5 mL, 5.92 mmol) and THF (8 mL). The mixture was stirred at rt for 3 h and cooled in an ice-bath. NaOH (aq, 1 M) was carefully added. H₂O and Et₂O were added and the phases were separated. The aq phase was extracted with Et₂O and the combined extracts were washed with brine, dried (Na₂SO₄) and concentrated to give the sub-title compound (665 mg, 93%), which was used in following step without further purification.

### (c) ((1s,4s)-4-Methoxycyclohexyl)methyl methanesulfonate

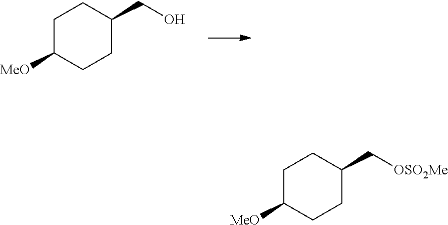

Triethylamine (1.27 mL, 9.12 mmol) was added dropwise to a stirred ice-cooled mixture of ((1s,4s)-4-methoxycyclohexyl)methanol (658 mg, 4.56 mmol), methanesulfonyl chloride (354 μL, 4.56 mmol) and CH₂Cl₂ (17 mL). The mixture was stirred at rt for 1 h and CH₂Cl₂ (20 mL) was added. The mixture was washed with HCl (aq, 0.5 M) and H₂O, dried (MgSO₄) and concentrated to give the sub-title compound (975 mg, 96%), which was used in the next step without further purification.

### (d) N-Benzyl-1-((1s,4s)-4-methoxycyclohexyl)methanamine

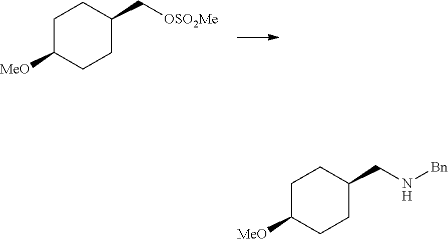

A mixture of ((1s,4s)-4-methoxycyclohexyl)methyl methanesulfonate (300 mg, 1.35 mmol) and benzyl amine (1.00 mL, 9.45 mmol) was heated under microwave irradiation at 100° C. for 2 h. The mixture was concentrated and the residue was partitioned between NaHCO₃ (aq, sat) and EtOAc. The layers were separated and the organic phase was dried (Na₂SO₄) and concentrated to give the sub-title compound (314 mg, 99.7%), which was used in following step without further purification.

### (e) (R)-2-(Benzyl(((1s,4S)-4-methoxycyclohexyl)methyl)amino)-1-(3-fluoro-phenyl)ethan-1-ol

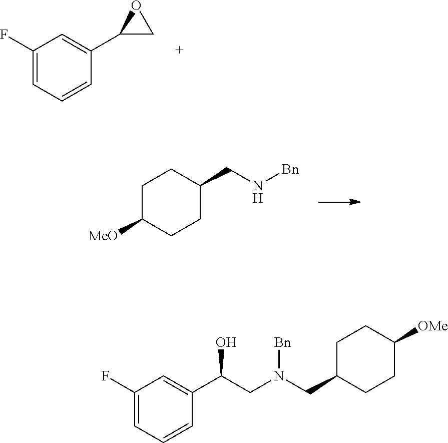 [SMILES: Fc1cccc([C@@H]2CO2)c1 ; CO[C@H]1CC[C@@H](CNCc2ccccc2)CC1 ; CO[C@H]1CC[C@@H](CN(Cc2ccccc2)C[C@H](O)c2cccc(F)c2)CC1]

A mixture of N-benzyl-1-((1s,4s)-4-methoxycyclohexyl)methanamine (70 mg, 0.30 mmol), (R)-2-(3-fluorophenyl)oxirane (62 mg, 0.45 mmol) and iPrOH (0.5 mL) was stirred at 80° C. for 16 h. The mixture was concentrated and the residue purified by chromatography to give the sub-title compound (86 mg, 77%).

### (f) (R)-1-(3-Fluorophenyl)-2-((((1s,4S)-4-methoxycyclohexyl)methyl)amino)ethan-1-ol

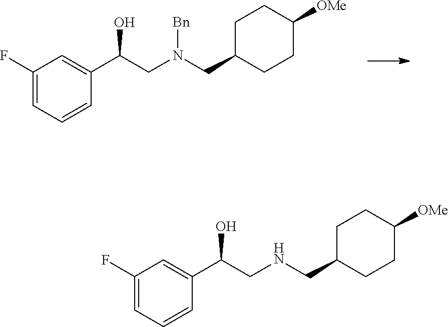 [SMILES: CO[C@H]1CC[C@@H](CN(Cc2ccccc2)C[C@H](O)c2cccc(F)c2)CC1 ; CO[C@H]1CC[C@@H](CNC[C@H](O)c2cccc(F)c2)CC1]

HCl (2.0 M in Et₂O, 110 μL, 0.22 mmol) was added to a solution of (R)-2-(benzyl-(((1s,4S)-4-methoxycyclohexyl)methyl)amino)-1-(3-fluorophenyl)ethan-1-ol (74 mg, 0.20 mmol) in Et₂O. The mixture was concentrated and the residue dissolved in H₂O (5.0 mL). Pd/C (10%, 21 mg, 0.02 mmol) was added and the mixture was hydrogenated at ambient temperature and pressure for 4 h. The mixture was filtered through a pad of Celite, which was washed with H₂O. The pH of the combined filtrates was adjusted to 8 by addition of NaHCO₃(aq, sat) and the mixture was extracted with EtOAc. The combined extracts were dried (Na₂SO₄), concentrated and the residue was purified by chromatography to give the title compound (38 mg, 68%).

¹H NMR (400 MHz, CDCl₃) δ 7.33-7.26 (m, 1H), 7.15-7.08 (m, 2H), 6.95 (tdd, J=8.4, 2.6, 1.1 Hz, 1H), 4.74 (dd, J=9.2, 3.5 Hz, 1H), 3.46-3.38 (m, 1H), 3.37-3.14 (m, 5H), 2.93 (dd, J=12.2, 3.5 Hz, 1H), 2.67 (dd, J=12.2, 9.2 Hz, 1H), 2.63-2.49 (m, 2H), 1.93-1.82 (m, 2H), 1.61-1.47 (m, 2H), 1.47-1.22 (m, 5H).

### Example 2: (R)-1-(5-Fluoropyridin-3-yl)-2-((((1s,4S)-4-methoxycyclohexyl)-methyl)amino)ethan-1-ol

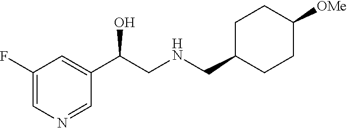 [SMILES: CO[C@H]1CC[C@@H](CNC[C@H](O)c2cncc(F)c2)CC1]

### (a) (R)-2-(Benzyl(((1s,4S)-4-methoxycyclohexyl)methyl)amino)-1-(5-fluoropyridin-3-yl)ethan-1-ol

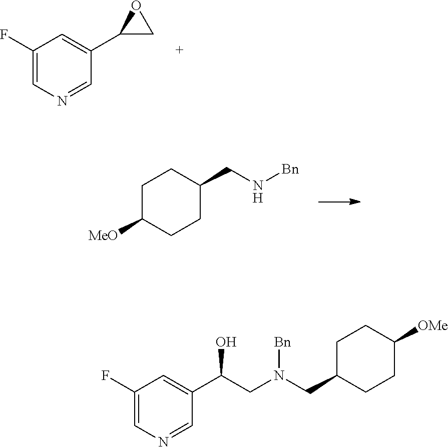 [SMILES: Fc1cncc([C@@H]2CO2)c1 ; CO[C@H]1CC[C@@H](CNCc2ccccc2)CC1 ; CO[C@H]1CC[C@@H](CN(Cc2ccccc2)C[C@H](O)c2cncc(F)c2)CC1]

The sub-title compound was prepared from N-benzyl-1-((1s,4s)-4-methoxycyclo-hexyl)methanamine and (R)-3-fluoro-5-(oxiran-2-yl)pyridine in accordance with the procedure in Example 1, Step (e).

### (b) (R)-1-(5-Fluoropyridin-3-yl)-2-((((1s,4S)-4-methoxycyclohexyl)methyl)-amino)ethan-1-ol

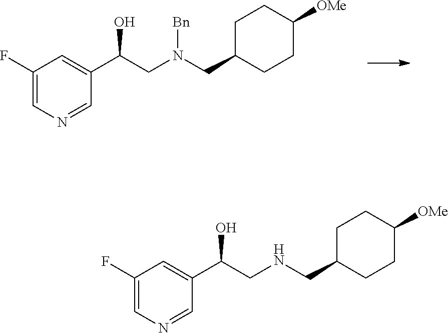 [SMILES: CO[C@H]1CC[C@@H](CN(Cc2ccccc2)C[C@H](O)c2cncc(F)c2)CC1 ; CO[C@H]1CC[C@@H](CNC[C@H](O)c2cncc(F)c2)CC1]

A mixture of (R)-2-(benzyl(((1s,4S)-4-methoxycyclohexyl)methyl)amino)-1-(5-fluoro-pyridin-3-yl)ethan-1-ol (60 mg, 0.16 mmol), Pd/C (10%, 34 mg, 0.03 mmol) and iPrOH (1.0 mL) was hydrogenated at ambient temperature and pressure for 4 h. An additional amount of Pd/C (10%, 17 mg, 0.015 mmol) was added and the hydrogenation was continued for 16 h. The mixture was filtered through a pad of Celite, which was washed with MeOH. The combined filtrates were concentrated and the residue purified by chromatography to give the title compound (20 mg, 44%).

¹H NMR (400 MHz, CDCl₃) δ 8.51-8.48 (m, 1H), 8.42 (d, J=2.8 Hz, 1H), 7.61-7.56 (m, 1H), 5.55 (dd, J=10.4, 2.2 Hz, 1H), 3.42-3.36 (m, 1H), 3.33 (dd, J=12.4, 2.2 Hz, 1H), 3.24 (s, 3H), 3.06 (dd, J=12.4, 10.4 Hz, 1H), 3.01-2.83 (m, 2H), 2.01-1.82 (m, 3H), 1.76-1.63 (m, 2H), 1.53-1.27 (m, 4H).

### Example 3: (R)-1-(3-Fluorophenyl)-2-((((1r,4R)-4-methoxycyclohexyl)methyl)amino)-ethan-1-ol

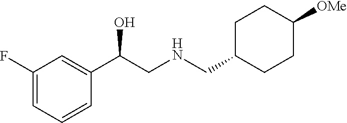 [SMILES: CO[C@H]1CC[C@H](CNC[C@H](O)c2cccc(F)c2)CC1]

The title compound was prepared from methyl (1r,4r)-4-hydroxycyclohexane-1-carboxylate in accordance with the procedures in Example 1, Steps (a) to (f).

¹H NMR (400 MHz, CDCl₃) δ 7.33-7.26 (m, 1H), 7.15-7.08 (m, 2H), 6.99-6.92 (m, 1H), 4.79 (dd, J=9.2, 3.4 Hz, 1H), 3.6-3.3 (br s, 2H, overlapping), 3.34 (s, 3H), 3.12-3.02 (m, 1H), 2.95 (dd, J=12.2, 3.5 Hz, 1H), 2.71 (dd, J=12.2, 9.2 Hz, 1H), 2.63-2.47 (m, 2H), 2.11-2.05 (m, 2H), 1.91-1.81 (m, 2H), 1.55-1.42 (m, 1H), 1.22-1.10 (m, 2H), 1.03-0.90 (m, 2H).

### Example 4: (R)-1-(5-Fluoropyridin-3-yl)-2-((((1r,4R)-4-methoxycyclohexyl)-methyl)amino)ethan-1-ol

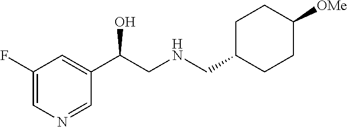 [SMILES: CO[C@H]1CC[C@H](CNC[C@H](O)c2cncc(F)c2)CC1]

The title compound was prepared from methyl (1r,4r)-4-methoxycyclohexane-1-carboxylate and (R)-3-fluoro-5-(oxiran-2-yl)pyridine in accordance with the procedures in Example 1, Steps (a) to (d) and Example 2, Steps (a) and (b).

¹H NMR (400 MHz, CDCl₃) δ 8.54-8.30 (m, 2H), 7.58-7.52 (m, 1H), 6.0-4.0 (br s, 2H, overlapping), 5.36 (d, J=8.2 Hz, 1H), 3.30 (s, 3H), 3.11-3.04 (m, 1H), 3.02-2.93 (m, 1H), 2.90-2.77 (m, 2H), 2.15-1.90 (m, 4H), 1.83-1.70 (m, 1H), 1.28-0.91 (m, 4H).

### Example 5: (R)-1-(3-Fluorophenyl)-2-((2-((1s,4S)-4-methoxycyclohexyl)propan-2-yl)-amino)ethan-1-ol

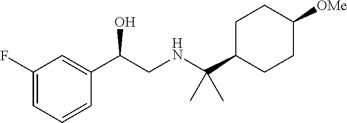 [SMILES: CO[C@H]1CC[C@@H](C(C)(C)NC[C@H](O)c2cccc(F)c2)CC1]

### (a) 2-((1s,4s)-4-Methoxycyclohexyl)propan-2-ol

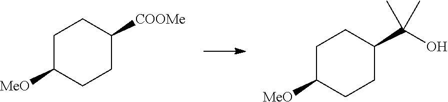

Methylmagnesium bromide (3.0 M in Et₂O, 1.74 mL, 5.23 mmol) was added dropwise to a solution of methyl (1s,4s)-4-methoxycyclohexane-1-carboxylate, see Example 1, Step (a), (300 mg, 1.74 mmol) in THF (25 mL) at −78° C. The mixture was stirred at rt for 2 h and NH₄Cl (aq, sat) was added. The mixture was extracted with EtOAc and the combined extracts were dried (Na₂SO₄) and concentrated. The residue was purified by chromatography to give the sub-title compound (240 mg, 80%)

### (b) 2-Chloro-N-(2-((1s,4s)-4-methoxycyclohexyl)propan-2-yl)acetamide

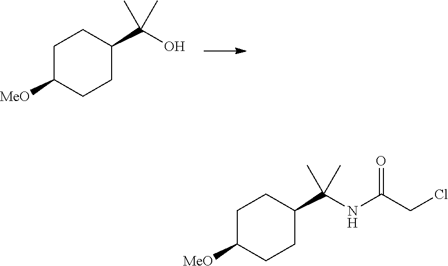 [SMILES: CO[C@H]1CC[C@@H](C(C)(C)O)CC1 ; CO[C@H]1CC[C@@H](C(C)(C)NC(=O)CCl)CC1]

2-Chloroacetonitrile (176 μL, 210 mg, 2.78 mmol) was added to a solution of 2-((1s,4s)-4-methoxycyclohexyl)propan-2-ol (240 mg, 1.39 mmol) in acetic acid (3 mL). The solution was cooled in an ice-bath and sulfuric acid (conc, 149 μL, 273 mg, 2.78 mmol) was slowly added while keeping the temperature below 10° C. The mixture was stirred at rt for 16 h and poured onto ice. The mixture was extracted with CH₂Cl₂ and the combined extracts were washed with NaHCO₃(aq, sat), dried (Na₂SO₄) and concentrated. The residue was purified by chromatography to give the sub-title compound (134 mg, 39%).

### (c) 2-((1s,4s)-4-Methoxycyclohexyl)propan-2-amine

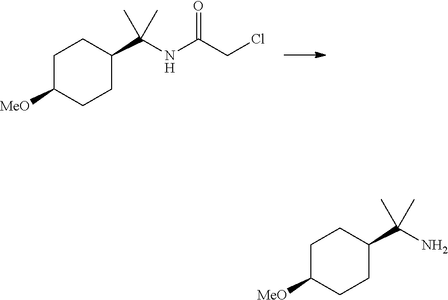 [SMILES: CO[C@H]1CC[C@@H](C(C)(C)NC(=O)CCl)CC1 ; CO[C@H]1CC[C@@H](C(C)(C)N)CC1]

Thiourea (52 mg, 0.68 mmol) and AcOH (300 μL) were added to a solution of 2-chloro-N-(2-((1s,4s)-4-methoxycyclohexyl)propan-2-yl)acetamide (135 mg, 0.54 mmol) in EtOH (1.5 mL). The mixture was heated at 90° C. for 21 h and allowed to cool. H₂O (5 mL) was added and the mixture was filtered. The filtrate was concentrated to ca 5 mL and the pH was adjusted to 12 with NaOH (aq, 10 M) and the mixture was extracted with CH₂Cl₂. The combined extracts were dried (Na₂SO₄) and concentrated to give the sub-title compound (78 mg, 84%).

### (d) (R)-1-(3-Fluorophenyl)-2-((2-((1s,4S)-4-methoxycyclohexyl)propan-2-yl)amino)-ethan-1-ol

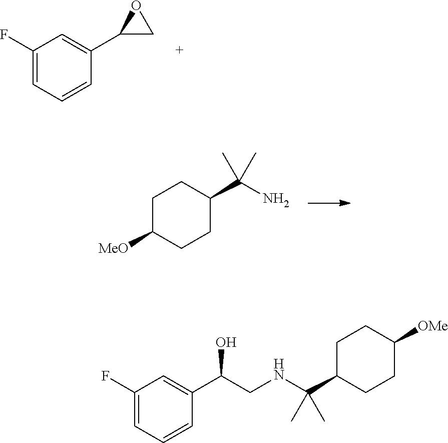 [SMILES: Fc1cccc([C@@H]2CO2)c1 ; CO[C@H]1CC[C@@H](C(C)(C)N)CC1 ; CO[C@H]1CC[C@@H](C(C)(C)NC[C@H](O)c2cccc(F)c2)CC1]

The title compound was prepared from 2-((1s,4s)-4-methoxycyclohexyl)propan-2-amine and (R)-2-(3-fluorophenyl)oxirane in accordance with the procedure in Example 1, Step (e).

¹H NMR (400 MHz, CDCl₃) δ 7.34-7.26 (m, 1H), 7.21-7.13 (m, 2H), 6.99-6.92 (m, 1H), 5.47-4.61 (m, 3H), 3.50-3.36 (m, 1H), 3.27 (s, 3H), 3.04 (dd, J=12.0, 2.8 Hz, 1H), 2.69 (dd, J=12.0, 9.5 Hz, 1H), 2.11-1.95 (m, 2H), 1.64-1.47 (m, 2H), 1.48-1.36 (m, 2H), 1.37-1.25 (m, 3H), 1.23 (s, 3H), 1.21 (s, 3H).

### Example 6: (R)-1-(5-fluoropyridin-3-yl)-2-((2-((1s,4S)-4-methoxycyclohexyl)propan-2-yl)amino)ethan-1-ol

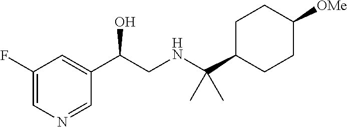 [SMILES: CO[C@H]1CC[C@@H](C(C)(C)NC[C@H](O)c2cncc(F)c2)CC1]

The title compound was prepared from 2-((1s,4s)-4-methoxycyclohexyl)propan-2-amine and (R)-3-fluoro-5-(oxiran-2-yl)pyridine in accordance with the procedure in Example 1, Step (e).

¹H NMR (400 MHz, CDCl₃) δ 8.45-8.41 (m, 1H), 8.38 (d, J=2.8 Hz, 1H), 7.57-7.49 (m, 1H), 4.97-4.88 (m, 1H), 4.60-3.55 (m, 2H), 3.47-3.39 (m, 1H), 3.28 (s, 3H), 3.02 (dd, J=12.0, 3.2 Hz, 1H), 2.63 (dd, J=12.0, 9.4 Hz, 1H), 2.08-1.96 (m, 2H), 1.60-1.48 (m, 2H), 1.47-1.22 (m, 5H), 1.17 (s, 3H), 1.15 (s, 3H).

### Example 7: (R)-1-(5-Fluoropyridin-3-yl)-2-((2-((1r,4R)-4-methoxycyclohexyl)propan-2-yl)amino)ethan-1-ol

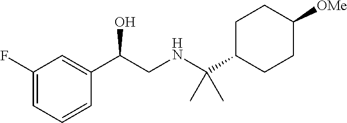 [SMILES: CO[C@H]1CC[C@H](C(C)(C)NC[C@H](O)c2cccc(F)c2)CC1]

The title compound was prepared from 2-((1r,4R)-4-methoxycyclohexyl)propan-2-amine (prepared from methyl (1r,4r)-4-methoxycyclohexane-1-carboxylate in accordance with the procedures in Example 5, Steps (a) to (c)) and (R)-2-(3-fluorophenyl)oxirane in accordance with the procedures in Example 1, Step (e).

¹H NMR (400 MHz, CDCl₃) δ 7.36-7.24 (m, 1H), 7.17-7.09 (m, 2H), 7.01-6.91 (m, 1H), 4.70 (dd, J=8.8, 3.2 Hz, 1H), 3.8-3.0 (br s, 2H, overlapping), 3.34 (s, 3H), 3.11-3.00 (m, 1H), 2.93 (dd, J=11.9, 3.4 Hz, 1H), 2.58 (dd, J=11.9, 8.9 Hz, 1H), 2.21-2.09 (m, 2H), 1.87-1.76 (m, 2H), 1.36-1.02 (m, 11H).

### Example 8: (R)-1-(5-Fluoropyridin-3-yl)-2-((2-((1r,4R)-4-methoxycyclohexyl)propan-2-yl)amino)ethan-1-ol

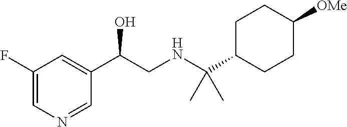 [SMILES: CO[C@H]1CC[C@H](C(C)(C)NC[C@H](O)c2cncc(F)c2)CC1]

The title compound was prepared from 2-((1r,4R)-4-methoxycyclohexyl)propan-2-amine (prepared from methyl (1r,4r)-4-methoxycyclohexane-1-carboxylate in accordance with the procedures in example 5, Steps (a) to (c)) and (R)-3-fluoro-5-(oxiran-2-yl)pyridine in accordance with the procedures in Example 1, Step (e).

¹H NMR (400 MHz, CDCl₃) δ 8.45-8.36 (m, 2H), 7.58-7.47 (m, 1H), 4.92-4.82 (m, 1H), 4.4-3.0 (br s, 2H), 3.33 (s, 3H), 3.11-3.03 (m, 1H), 3.01 (dd, J=12.1, 3.2 Hz, 1H), 2.62 (dd, J=12.0, 9.2 Hz, 1H), 2.22-2.07 (m, 2H), 1.91-1.77 (m, 2H), 1.40-1.03 (m, 11H).

### Example 9: (R)-1-(3-Fluorophenyl)-2-((2-((1s,4S)-4-methoxycyclohexyl)ethyl)amino)-ethan-1-ol

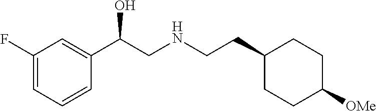 [SMILES: CO[C@H]1CC[C@@H](CCNC[C@H](O)c2cccc(F)c2)CC1]

### (a) 2-((1s,4s)-4-Methoxycyclohexyl)acetonitrile

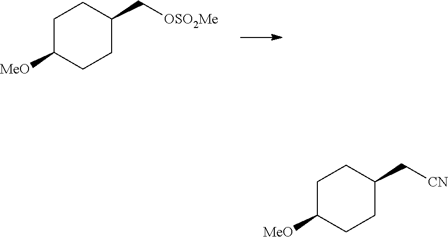

NaCN (312 mg, 6.36 mmol) was added to a solution of ((1s,4s)-4-methoxycyclohexyl)-methyl methanesulfonate, see Example 1, Step (c), (471 mg, 2.12 mmol) in DMF (4.0 mL).

The mixture was stirred at 80° C. for 3 h and cooled to rt. H₂O was added and the mixture was extracted with Et₂O. The combined extracts were washed with H₂O, dried (MgSO₄) and concentrated to give the sub-title compound (306 mg, 94%).

### (b) 2-((1s,4s)-4-Methoxycyclohexyl)ethan-1-amine hydrochloride

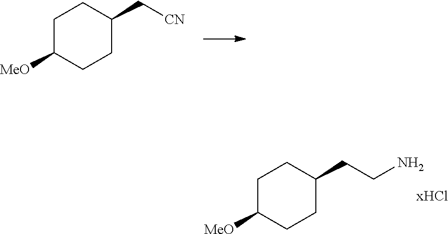 [SMILES: CO[C@H]1CC[C@@H](CC#N)CC1 ; CO[C@H]1CC[C@@H](CCN)CC1]

A mixture of 2-((1s,4s)-4-methoxycyclohexyl)acetonitrile (135 mg, 0.88 mg), Pt₂O·H₂O (22 mg, 0.088 mmol), CHCl₃ (0.2 mL) and EtOH (2.0 mL) was hydrogenated at 4 atm overnight. The mixture was filtered through a pad of Celite, which was washed with EtOH. The combined filtrates were concentrated and the residue was dissolved in EtOH (2.0 mL), and CHCl₃ (0.2 mL) and Pt₂O hydrate (22 mg, 0.088 mmol) was added. The mixture was hydrogenated at 4 atm for 3 h. The mixture was filtered through a pad of Celite, which was washed with EtOH. The combined filtrates were concentrated and the residue dried to give the sub-title compound (121 mg, 71%).

### (c) (R)-1-(3-Fluorophenyl)-2-((2-((1s,4S)-4-methoxycyclohexyl)ethyl)amino)ethan-1-ol

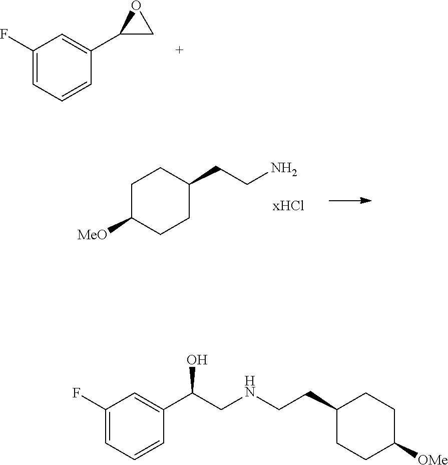 [SMILES: Fc1cccc([C@@H]2CO2)c1 ; CO[C@H]1CC[C@@H](CCN)CC1 ; CO[C@H]1CC[C@@H](CCNC[C@H](O)c2cccc(F)c2)CC1]

Finely ground KOH (38 mg, 0.70 mmol) followed by (R)-2-(3-fluorophenyl)oxirane (96 mg, 0.70 mmol) were added to a stirred mixture of 2-((1s,4s)-4-methoxycyclohexyl)ethan-1-amine hydrochloride (135 mg, 0.70 mmol) and iPrOH (1.1 mL) at rt. The mixture was stirred at 80° C. for 6 h and concentrated. The residue was partitioned between CH₂Cl₂ and H₂O (10 mL) and the layers were separated. The aq phase was extracted with CH₂Cl₂ and the combined extracts were washed with brine, dried (Na₂SO₄) and concentrated. The residue was purified by chromatography to yield the title compound (28 mg, 14%).

¹H NMR (400 MHz, CDCl₃) δ 7.33-7.26 (m, 1H), 7.18-7.10 (m, 2H), 6.99-6.92 (m, 1H), 4.97 (dd, J=9.6, 3.1 Hz, 1H), 4.78-4.39 (m, 2H), 3.42-3.35 (m, 1H), 3.28 (s, 3H), 3.03 (dd, J=12.3, 3.1 Hz, 1H), 2.91-2.74 (m, 3H), 1.90-1.78 (m, 2H), 1.62-1.51 (m, 2H), 1.48-1.20 (m, 7H).

### Example 10: (R)-1-(5-Fluoropyridin-3-yl)-2-((2-((1s,4S)-4-methoxycyclohexyl)ethyl)-amino)ethan-1-ol

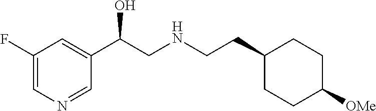 [SMILES: CO[C@H]1CC[C@@H](CCNC[C@H](O)c2cncc(F)c2)CC1]

The title compound was prepared in accordance with the procedures in Example 9, using (R)-3-fluoro-5-(oxiran-2-yl)pyridine in the final step.

¹H NMR (400 MHz, CDCl₃) δ 8.45-8.34 (m, 2H), 7.56-7.48 (m, 1H), 5.05 (dd, J=9.7, 3.1 Hz, 1H), 5.01-4.78 (m, 2H), 3.42-3.34 (m, 1H), 3.27 (s, 3H), 3.06 (dd, J=12.3, 3.1 Hz, 1H), 2.92-2.75 (m, 3H), 1.90-1.79 (m, 2H), 1.62-1.51 (m, 2H), 1.49-1.21 (m, 7H).

### Example 11: (R)-1-(3-Fluorophenyl)-2-((2-((1r,4R)-4-methoxycyclohexyl)ethyl)amino)-ethan-1-ol

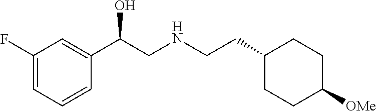 [SMILES: CO[C@H]1CC[C@H](CCNC[C@H](O)c2cccc(F)c2)CC1]

The title compound was prepared in accordance with the procedures in Example 9, Steps (a) to (c) from ((1r,4r)-4-methoxycyclohexyl)methyl methanesulfonate (prepared in accordance with the procedures in Example 1, Steps (a) to (c) from methyl (1r,4r)-4-hydroxycyclohexane-1-carboxylate).

¹H NMR (400 MHz, CDCl₃) δ 7.32-7.26 (m, 1H), 7.14-7.08 (m, 2H), 6.97-6.92 (m, 1H), 4.69 (dd, J=8.9, 3.6 Hz, 1H), 3.34 (s, 3H), 3.12-3.02 (m, 1H), 2.90 (dd, J=12.2, 3.7 Hz, 1H), 2.77-2.57 (m, 3H), 2.6-2.2 (br s, 2H), 2.09-2.01 (m, 2H), 1.84-1.72 (m, 2H), 1.43-1.36 (m, 2H), 1.35-1.25 (m, 1H), 1.23-1.07 (m, 2H), 0.94 (m, 2H).

### Example 12: (R)-1-(5-Fluoropyridin-3-yl)-2-((2-((1r,4R)-4-methoxycyclohexyl)ethyl)-amino)ethan-1-ol

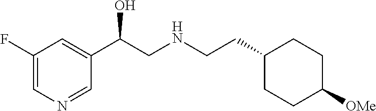 [SMILES: CO[C@H]1CC[C@H](CCNC[C@H](O)c2cncc(F)c2)CC1]

The title compound was prepared in accordance with the procedures in Example 9, Steps (a) to (c) using (R)-3-fluoro-5-(oxiran-2-yl)pyridine in Step (c) and ((1r,4r)-4-methoxycyclohexyl)methyl methanesulfonate (prepared in accordance with the procedures in Example 1, Steps (a) to (c) from methyl (1r,4r)-4-hydroxycyclohexane-1-carboxylate).

¹H NMR (400 MHz, CDCl₃) δ 8.41-8.36 (m, 2H), 7.51-7.47 (m, 1H), 4.75 (dd, J=9.2, 3.6 Hz, 1H), 3.34 (s, 3H), 3.11-3.03 (m, 1H), 2.96 (dd, J=12.3, 3.7 Hz, 1H), 2.78-2.59 (m, 3H), 2.6-2.2 (br s, 2H), 2.11-1.99 (m, 2H), 1.84-1.72 (m, 2H), 1.44-1.36 (m, 2H), 1.36-1.22 (m, 1H), 1.23-1.11 (m, 2H), 1.02-0.89 (m, 2H).

### Example 13: (R)-1-(3-Fluorophenyl)-2-((1-((1s,4S)-4-methoxycyclohexyl)-2-methyl-propan-2-yl)amino)ethan-1-ol

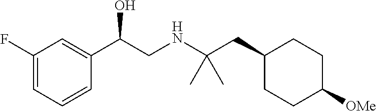 [SMILES: CO[C@H]1CC[C@@H](CC(C)(C)NC[C@H](O)c2cccc(F)c2)CC1]

### (a) 1-((1s,4s)-4-Methoxycyclohexyl)-2-methylpropan-2-amine

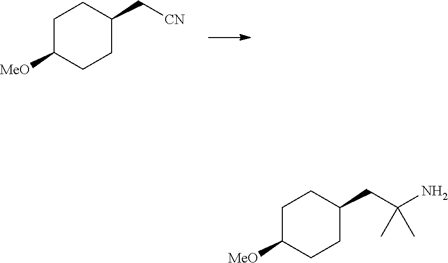

A mixture of CeCl₃ (434 mg, 1.76 mmol) and THF (5 mL) was stirred vigorously for 45 min at rt. The white suspension was cooled to −78° C., and methyllithium (1.6 M in Et₂O, 1.10 mL, 1.76 mmol) was added dropwise. The mixture was stirred at −78° C. for 30 min and a solution of 2-((1s,4s)-4-methoxycyclohexyl)acetonitrile, see Example 9, Step (a), (90 mg, 0.59 mmol) in THF (2 mL) was added dropwise. The mixture was stirred for 2 h at −65° C. NH₄OH (aq, 2 mL) was added dropwise at −65° C. and the mixture was allowed to warm to rt and filtered through a pad of Celite, which was washed with CH₂Cl₂. The combined organic phases were dried (Na₂SO₄) and concentrated to give the sub-title compound (91 mg, 84%), which was used in the next step without further purification.

### (b) (R)-1-(3-Fluorophenyl)-2-((1-((1s,4S)-4-methoxycyclohexyl)-2-methylpropan-2-yl)amino)ethan-1-ol

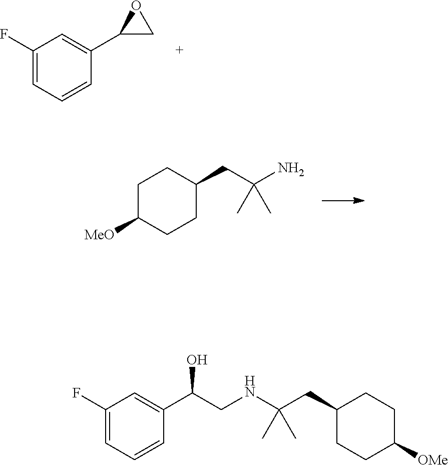 [SMILES: Fc1cccc([C@@H]2CO2)c1 ; CO[C@H]1CC[C@@H](CC(C)(C)N)CC1 ; CO[C@H]1CC[C@@H](CC(C)(C)NC[C@H](O)c2cccc(F)c2)CC1]

The title compound was prepared in accordance with the procedure in Example 1, Step (e) from 1-((1s,4s)-4-methoxycyclohexyl)-2-methylpropan-2-amine and (R)-2-(3-fluoro-phenyl)oxirane.

¹H NMR (400 MHz, CDCl₃) δ 7.33-7.26 (m, 1H), 7.18-7.12 (m, 2H), 6.99-6.93 (m, 1H), 5.45-5.00 (m, 2H), 4.95 (dd, J=9.5, 2.9 Hz, 1H), 3.38-3.31 (m, 1H), 3.28 (s, 3H), 3.03 (dd, J=12.0, 2.9 Hz, 1H), 2.71 (dd, J=12.0, 9.5 Hz, 1H), 1.85-1.73 (m, 2H), 1.57-1.30 (m, 9H), 1.26 (s, 3H), 1.24 (s, 3H).

### Example 14: (R)-1-(5-Fluoropyridin-3-yl)-2-((1-((1s,4S)-4-methoxycyclohexyl)-2-methylpropan-2-yl)amino)ethan-1-ol

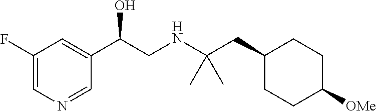 [SMILES: CO[C@H]1CC[C@@H](CC(C)(C)NC[C@H](O)c2cncc(F)c2)CC1]

The title compound was prepared in accordance with the procedure in Example 1, Step (e) from 1-((1s,4s)-4-methoxycyclohexyl)-2-methylpropan-2-amine, see Example 13, step (a), and (R)-3-fluoro-5-(oxiran-2-yl)pyridine.

¹H NMR (400 MHz, CDCl₃) δ 8.51-8.46 (m, 1H), 8.41 (d, J=2.7 Hz, 1H), 7.61-7.55 (m, 1H), 5.49-5.40 (m, 1H), 3.40-3.33 (m, 1H), 3.26 (s, 3H), 3.21 (dd, J=12.1, 2.1 Hz, 1H), 2.93 (dd, J=12.1, 10.3 Hz, 1H), 1.88-1.73 (m, 2H), 1.69-1.28 (m, 17H).

### Example 15: (R)-1-(3-Fluorophenyl)-2-((1-((1r,4R)-4-methoxycyclohexyl)-2-methyl-propan-2-yl)amino)ethan-1-ol

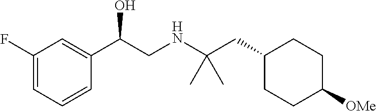 [SMILES: CO[C@H]1CC[C@H](CC(C)(C)NC[C@H](O)c2cccc(F)c2)CC1]

The title compound was prepared in accordance with the procedure in Example 1, Step (e) from 1-((1r,4r)-4-methoxycyclohexyl)-2-methylpropan-2-amine (prepared from methyl (1r,4r)-4-methoxycyclohexane-1-carboxylate in accordance with the procedures in Example 1, Steps (a) to (c), Example 9, Step (a) and Example 13, Step (a)) and (R)-2-(3-fluorophenyl)oxirane.

¹H NMR (400 MHz, CDCl₃) δ 7.34-7.26 (m, 1H), 7.18-7.11 (m, 2H), 7.00-6.93 (m, 1H), 5.18-4.66 (m, 3H), 3.32 (s, 3H), 3.08-2.97 (m, 2H), 2.71 (dd, J=12.0, 9.4 Hz, 1H), 2.06-1.95 (m, 2H), 1.85-1.74 (m, 2H), 1.44 (d, J=5.1 Hz, 2H), 1.41-1.29 (m, 1H), 1.25 (s, 3H), 1.23 (s, 3H), 1.23-1.11 (m, 2H), 1.09-0.94 (m, 2H).

### Example 16: (R)-1-(5-Fluoropyridin-3-yl)-2-((1-((1r,4R)-4-methoxycyclohexyl)-2-methyl-propan-2-yl)amino)ethan-1-ol

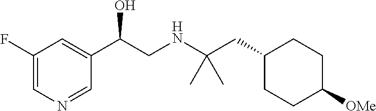 [SMILES: CO[C@H]1CC[C@H](CC(C)(C)NC[C@H](O)c2cncc(F)c2)CC1]

The title compound was prepared in accordance with the procedure in Example 1, Step (e) from 1-((1r,4r)-4-methoxycyclohexyl)-2-methylpropan-2-amine (prepared from methyl (1r,4r)-4-methoxycyclohexane-1-carboxylate in accordance with the procedures in Example 1, Steps (a) to (c), Example 9, Step (a) and Example 13, Step (a)) and (R)-3-fluoro-5-(oxiran-2-yl)pyridine.

¹H NMR (400 MHz, CDCl₃) δ 8.49-8.44 (m, 1H), 8.39 (d, J=2.7 Hz, 1H), 7.60-7.53 (m, 1H), 6.84-5.52 (m, 2H), 5.32-5.24 (m, 1H), 3.31 (s, 3H), 3.15 (dd, J=12.1, 2.4 Hz, 1H), 3.07-2.97 (m, 1H), 2.85 (dd, J=12.1, 10.0 Hz, 1H), 2.08-1.94 (m, 2H), 1.86-1.73 (m, 2H), 1.63-1.49 (m, 2H), 1.46-1.31 (m, 7H), 1.27-1.12 (m, 2H), 1.10-0.95 (m, 2H).

### Example 17: (R)-1-(3-Fluorophenyl)-2-((3-((1r,4S)-4-methoxycyclohexyl)propyl)-amino)ethan-1-ol

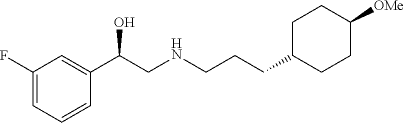

### (a) 4-Methoxycyclohexan-1-one

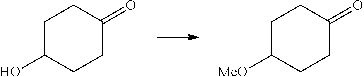 [SMILES: O=C1CCC(O)CC1 ; COC1CCC(=O)CC1]

N¹,N¹,N⁸,N⁸-Tetramethylnaphthalene-1,8-diamine (4.1 g, 19.3 mmol) was added to an ice-cooled solution of 4-hydroxycyclohexan-1-one (2.0 g, 17.5 mmol) in CH₂Cl₂ (100 mL). The mixture was stirred at rt for 30 min and cooled to 0° C. Trimethyl oxonium tetrafluoroborate (3.5 g, 23.7 mmol) was added and the mixture was stirred at rt for 24 h. H₂O and CH₂Cl₂ were added and layers were separated. The aq phase was extracted with CH₂Cl₂ and the combined extracts were washed with brine, dried (Na₂SO₄) and concentrated. The residue was purified by chromatography to give the sub-title compound (1.78 g, 79%).

### (b) Ethyl 2-(4-methoxycyclohexylidene)acetate

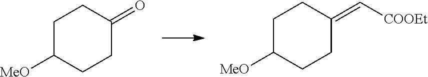 [SMILES: COC1CCC(=O)CC1 ; CCOC(=O)C=C1CCC(OC)CC1]

NaH (60% in mineral oil, 637 mg, 15.9 mmol) was added in one portion to a stirred ice-cooled solution of ethyl 2-diethoxyphosphorylacetate (3.17 mL, 15.9 mmol) in THF (7 mL). The mixture was stirred at 0° C. for 30 min and 4-methoxycyclohexan-1-one (1.7 g, 13.3 mmol) in THF (3 mL) was added drop-wise. The mixture was stirred at 0° C. for 30 min and NH₄Cl (aq, sat) was added. The mixture was extracted with EtOAc and the combined extracts were washed with brine, dried (Na₂SO₄) and concentrated to give the sub-title compound (2.6 g, 99%).

### (c) Ethyl 2-((1r,4r)-4-methoxycyclohexyl)acetate

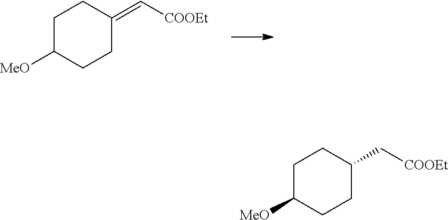 [SMILES: CCOC(=O)C=C1CCC(OC)CC1 ; CCOC(=O)C[C@H]1CC[C@H](OC)CC1]

A mixture of ethyl 2-(4-methoxycyclohexylidene)acetate (1.8 g, 9.08 mmol), Pd/C (10%, 96.6 mg, 0.09 mmol) and MeOH (10 mL) was hydrogenated at ambient temperature and pressure for 1.5 h. The mixture was filtered through a pad of Celite, which was washed with MeOH. The combined filtrates were concentrated and the residue was purified by chromatography to give the sub-title compound (926 mg, 51%) along with the diastereomer ethyl 2-((1s,4s)-4-methoxycyclohexyl)acetate (649 mg, 36%).

### (d) 2-((1r,4r)-4-Methoxycyclohexyl)ethan-1-ol

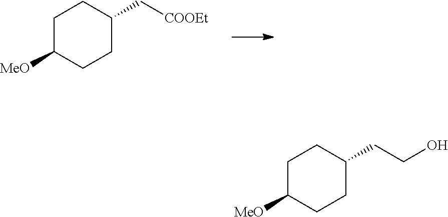 [SMILES: CCOC(=O)C[C@H]1CC[C@H](OC)CC1 ; CO[C@H]1CC[C@H](CCO)CC1]

LiAlH₄ (2.4 M in THF, 3.0 mL, 7.2 mmol) was added dropwise to an ice-cooled solution of ethyl 2-((1r,4r)-4-methoxycyclohexyl)acetate (962 mg, 4.8 mmol) in THF (20 mL). The mixture was stirred at 0° C. for 10 min and at rt for 1 h. The mixture was cooled in an ice-bath and NH₄Cl (aq, sat, 20 mL) was added dropwise. The mixture was extracted with CH₂Cl₂ and the combined extracts were dried (Na₂SO₄) and concentrated to give the sub-title compound (695 mg, 91%), which was used in the next step without further purification.

### (e) 2-((1r,4r)-4-Methoxycyclohexyl)ethyl methanesulfonate

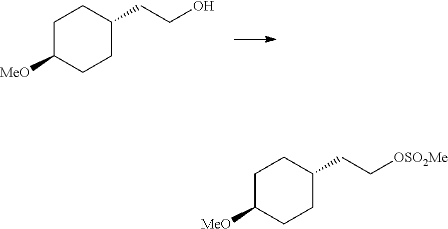

Methanesulfonyl chloride (375 μL, 9.7 mmol) was added dropwise to an ice-cooled mixture of 2-((1r,4r)-4-methoxycyclohexyl)ethan-1-ol (695 mg, 4.4 mmol)), triethylamine (796 μL, 5.7 mmol) and CH₂Cl₂ (10 mL) and the mixture was stirred at 0° C. for 30 min. H₂O was added and the layers were separated. The aq phase was extracted with CH₂Cl₂ and the combined organic phases were washed with H₂O and brine, dried (Na₂SO₄) and concentrated to give the sub-title compound (926 mg, 89%), which was used in next step without further purification.

### (f) 3-((1r,4s)-4-Methoxycyclohexyl)propanenitrile

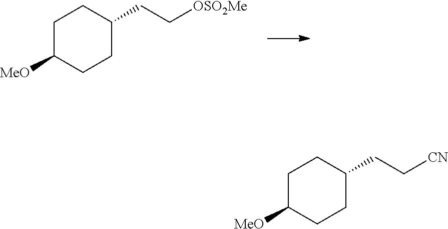

NaCN (415 mg, 8.5 mmol) was added to a solution of 2-((1r,4r)-4-methoxycyclo-hexyl)ethyl methanesulfonate (667 mg, 2.8 mmol) in DMF (3 mL). The mixture was stirred at 80° C. for 20 h and cooled to rt. H₂O was added and the mixture was extracted with Et₂O. The combined extracts were washed with H₂O, dried (Na₂SO₄) and concentrated to give the sub-title compound (460 mg, 98%), which was used in next step without further purification.

### (g) 3-((1r,4s)-4-Methoxycyclohexyl)propan-1-amine

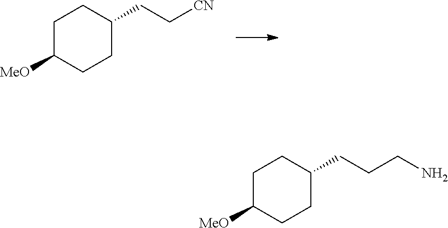 [SMILES: CO[C@H]1CC[C@H](CCC#N)CC1 ; CO[C@H]1CC[C@H](CCCN)CC1]

LiAlH₄ (2.4 M in THF, 2.1 mL, 5.00 mmol) was added dropwise to an ice-cooled solution of 3-((1r,4s)-4-methoxycyclohexyl)propanenitrile (415 mg, 2.48 mmol) in THF (10 mL). The mixture was stirred at 0° C. for 30 min and NH₄Cl (aq, sat, 10 mL) was added dropwise. The mixture was extracted with CH₂Cl₂ and the combined extracts were dried (Na₂SO₄) and concentrated to give the sub-title compound (359 mg, 84%), which was used in the next step without further purification.

### (h) (R)-1-(3-Fluorophenyl)-2-((3-((1s,4S)-4-methoxycyclohexyl)-3-methylbutyl)-amino)ethan-1-ol

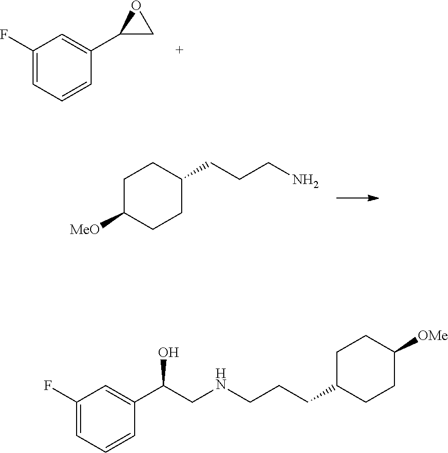

The title compound was prepared in accordance with the procedure in Example 1, Step (e) from 3-((1r,4s)-4-methoxycyclohexyl)propan-1-amine and (R)-2-(3-fluorophenyl)oxirane.

¹H NMR (400 MHz, CDCl₃) δ 7.33-7.26 (m, 1H), 7.14-7.07 (m, 2H), 6.98-6.91 (m, 1H), 4.70 (dd, J=9.0, 3.6 Hz, 1H), 3.34 (s, 3H), 3.06 (tt, J=10.8, 4.1 Hz, 1H), 2.89 (dd, J=12.2, 3.6 Hz, 1H), 2.74-2.41 (m, 5H), 2.09-2.00 (m, 2H), 1.82-1.71 (m, 2H), 1.53-1.42 (m, 2H), 1.27-1.09 (m, 5H), 0.98-0.86 (m, 2H).

### Example 18: (R)-1-(5-Fluoropyridin-3-yl)-2-((3-((1r,4S)-4-methoxycyclohexyl)propyl)-amino)ethan-1-ol

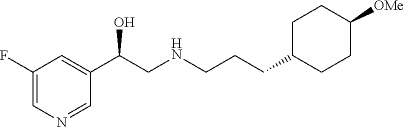

The title compound was prepared in accordance with the procedure in Example 1, Step (e) from 3-((1r,4s)-4-methoxycyclohexyl)propan-1-amine, see Example 17, Step (g), and (R)-3-fluoro-5-(oxiran-2-yl)pyridine.

¹H NMR (400 MHz, CDCl₃) δ 8.41-8.32 (m, 2H), 7.51-7.46 (m, 1H), 4.74 (dd, J=9.2, 3.6 Hz, 1H), 3.33 (s, 3H), 3.10-3.01 (m, 1H), 2.93 (dd, J=12.3, 3.7 Hz, 1H), 2.75-2.35 (m, 5H), 2.11-1.96 (m, 2H), 1.84-1.71 (m, 2H), 1.55-1.43 (m, 2H), 1.28-1.06 (m, 5H), 0.98-0.80 (m, 2H).

### Example 19: (R)-1-(3-Fluorophenyl)-2-((3-((1s,4R)-4-methoxycyclohexyl)propyl)amino)-ethan-1-ol

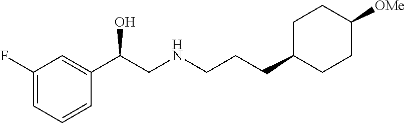 [SMILES: CO[C@H]1CC[C@@H](CCCNC[C@H](O)c2cccc(F)c2)CC1]

The title compound was prepared in accordance with the procedure in Example 1, Step (e) from 3-((1r,4s)-4-methoxycyclohexyl)propan-1-amine (prepared from ethyl 2-((1s,4s)-4-methoxycyclohexyl)acetate (see Example 17, Step (c)) in accordance with the procedures in Example 17, Steps (d) to (g)) and (R)-2-(3-fluorophenyl)oxirane.

¹H NMR (400 MHz, CDCl₃) δ 7.26-7.20 (m, 1H), 7.09-7.01 (m, 2H), 6.92-6.83 (m, 1H), 4.63 (dd, J=9.0, 3.6 Hz, 1H), 3.37-3.29 (m, 1H), 3.23 (s, 3H), 2.84 (dd, J=12.2, 3.6 Hz, 1H), 2.65-2.48 (m, 3H), 2.04 (br s, 2H), 1.82-1.72 (m, 2H), 1.49-1.27 (m, 6H), 1.27-1.06 (m, 5H).

### Example 20: (R)-1-(5-Fluoropyridin-3-yl)-2-((3-((1s,4R)-4-methoxycyclohexyl)propyl)-amino)ethan-1-ol

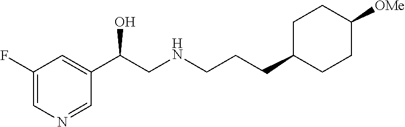 [SMILES: CO[C@H]1CC[C@@H](CCCNC[C@H](O)c2cncc(F)c2)CC1]

The title compound was prepared in accordance with the procedure in Example 1, Step (e) from 3-((1r,4s)-4-methoxycyclohexyl)propan-1-amine (prepared from ethyl 2-((1s,4s)-4-methoxycyclohexyl)acetate (see Example 17, Step (c)) in accordance with the procedures in Example 17, Steps (d) to (g)) and (R)-3-fluoro-5-(oxiran-2-yl)pyridine

¹H NMR (400 MHz, CDCl₃) δ 8.38 (t, J=1.7 Hz, 1H), 8.36 (d, J=2.8 Hz, 1H), 7.52-7.46 (m, 1H), 4.74 (dd, J=9.2, 3.6 Hz, 1H), 3.42-3.37 (m, 1H), 3.29 (s, 3H), 2.94 (dd, J=12.3, 3.7 Hz, 1H), 2.73-2.55 (m, 4H), 2.51 (br s, 1H), 1.93-1.77 (m, 2H), 1.55-1.34 (m, 6H), 1.34-1.20 (m, 5H).

### Example 21: (R)-1-(3-Fluorophenyl)-2-((4-((1r,4S)-4-methoxycyclohexyl)-2-methyl-butan-2-yl)amino)ethan-1-ol hydrochloride

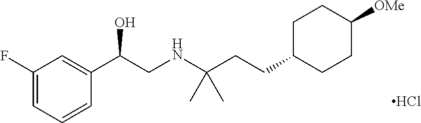 [SMILES: CO[C@H]1CC[C@H](CCC(C)(C)NC[C@H](O)c2cccc(F)c2)CC1 ; Cl]

### (a) 4-((1r,4s)-4-Methoxycyclohexyl)-2-methylbutan-2-amine

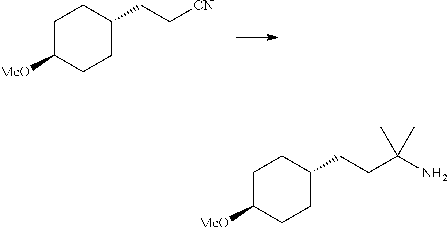

The sub-title compound was prepared in accordance with the procedure in Example 13, Step (a) from 3-((1r,4s)-4-methoxycyclohexyl)propanenitrile (prepared from ethyl 2-((1s,4s)-4-methoxycyclohexyl)acetate (see Example 17, Step (c)) in accordance with the procedures in Example 17, Steps (d) to (f)).

### (b) (R)-1-(3-Fluorophenyl)-2-((4-((1r,4S)-4-methoxycyclohexyl)-2-methylbutan-2-yl)amino)ethan-1-ol hydrochoride

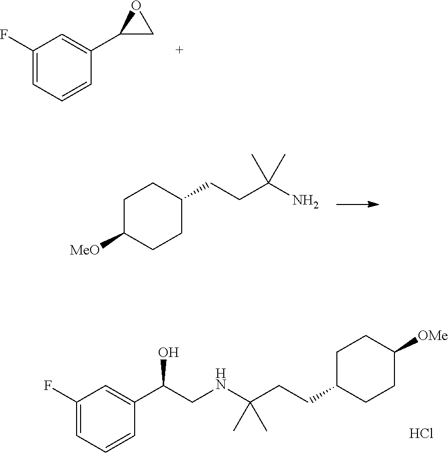 [SMILES: Fc1cccc([C@@H]2CO2)c1 ; CO[C@H]1CC[C@H](CCC(C)(C)N)CC1 ; CO[C@H]1CC[C@H](CCC(C)(C)NC[C@H](O)c2cccc(F)c2)CC1 ; Cl]

(R)-1-(3-Fluorophenyl)-2-((4-((1r,4S)-4-methoxycyclohexyl)-2-methylbutan-2-yl)-amino)ethan-1-ol was prepared from 4-((1r,4s)-4-methoxycyclohexyl)-2-methylbutan-2-amine and (R)-2-(3-fluorophenyl)oxirane in accordance with the procedure in Example 1, Step (e). 42 mg (0.12 mmol) of this material was dissolved in Et₂O (2 mL) and HCl (2 M in Et₂O, 61 μL, 0.12 mmol) was added dropwise at rt. The mixture was stirred at rt for 15 min and the solids were collected, washed with E₂O and dried to give the title compound (46 mg, 99%).

¹H NMR (400 MHz, CDCl₃) δ 10.01 (br s, 1H), 8.00 (t, J=10.4 Hz, 1H), 7.35-7.27 (m, 1H), 7.24-7.17 (m, 2H), 7.08-6.88 (m, 1H), 5.71 (br s, 1H), 5.39 (d, J=10.2 Hz, 1H), 3.32 (s, 3H), 3.25-3.16 (m, 1H), 3.06-2.97 (m, 1H), 2.92 (q, J=10.5 Hz, 1H), 2.04-1.93 (m, 2H), 1.80-1.65 (m, 4H), 1.43 (s, 3H), 1.40 (s, 3H), 1.31-1.23 (m, 2H), 1.16-0.99 (m, 3H), 0.98-0.82 (m, 2H).

### Example 22: (R)-1-(5-Fluoropyridin-3-yl)-2-((4-((1r,4S)-4-methoxycyclohexyl)-2-methylbutan-2-yl)amino)ethan-1-ol dihydrochloride

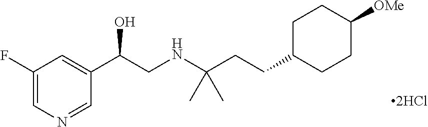

The title compound was prepared from 4-((1r,4s)-4-methoxycyclohexyl)-2-methylbutan-2-amine, see Example 21, step (a), and (R)-3-fluoro-5-(oxiran-2-yl)pyridine in accordance with the procedure in Example 1, Step (e) followed by the procedure in Example 21, Step (b).

¹H NMR (400 MHz, CDCl₃) δ 10.06 (br s, 1H), 8.52 (s, 1H), 8.41 (d, J=2.7 Hz, 1H), 8.12 (t, J=11.8 Hz, 1H), 7.61 (dt, J=8.9, 2.3 Hz, 1H), 5.88 (br s, 1H), 5.53 (d, J=9.9 Hz, 1H), 3.32 (s, 3H), 3.29-3.18 (m, 1H), 3.03 (tt, J=10.7, 4.1 Hz, 1H), 2.94 (q, J=10.4 Hz, 1H), 2.09-1.96 (m, 2H), 1.93 (br s, 1H), 1.81-1.67 (m, 3H), 1.45 (s, 3H), 1.43 (s, 3H), 1.33-1.23 (m, 2H), 1.21-1.02 (m, 3H), 0.99-0.86 (m, 2H).

### Example 23: (R)-1-(3-Fluorophenyl)-2-((4-((1s,4R)-4-methoxycyclohexyl)-2-methylbutan-2-yl)amino)ethan-1-ol hydrochloride

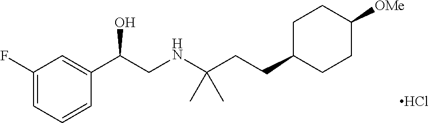 [SMILES: CO[C@H]1CC[C@@H](CCC(C)(C)NC[C@H](O)c2cccc(F)c2)CC1 ; Cl]

The title compound was prepared from 4-((1s,4r)-4-methoxycyclohexyl)-2-methylbutan-2-amine (prepared from ethyl 2-((1s,4s)-4-methoxycyclohexyl)acetate, see Example 17, Step (c) in accordance with the procedures in Example 17, Steps (d) to (f) followed by the procedures in Example 21, Steps (a) and (b)).

¹H NMR (400 MHz, CDCl₃) δ 10.01 (br s, 1H), 8.03 (br s, 1H), 7.33-7.27 (m, 1H), 7.24-7.18 (m, 2H), 7.03-6.90 (m, 1H), 5.39 (dd, J=10.3, 2.0 Hz, 1H), 3.38-3.33 (m, 1H), 3.26 (s, 3H), 3.25-3.14 (m, 1H), 2.92 (q, J=10.5 Hz, 1H), 1.87-1.67 (m, 4H), 1.42 (s, 3H), 1.40 (s, 3H), 1.39 (br s, 1H), 1.35-1.12 (m, 8H).

### Example 24: (R)-1-(5-Fluoropyridin-3-yl)-2-((4-((1s,4R)-4-methoxycyclohexyl)-2-methylbutan-2-yl)amino)ethan-1-ol dihydrochloride

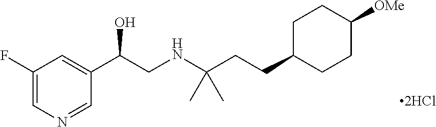 [SMILES: CO[C@H]1CC[C@@H](CCC(C)(C)NC[C@H](O)c2cncc(F)c2)CC1 ; "·2HCl"]

The title compound was prepared from 4-((1s,4r)-4-methoxycyclohexyl)-2-methylbutan-2-amine (prepared from ethyl 2-((1s,4s)-4-methoxycyclohexyl)acetate, see Example 17, Step (c) in accordance with the procedures in Example 17, Steps (d) to (f) followed by the procedure in Example 21, Step (a)) and (R)-3-fluoro-5-(oxiran-2-yl)pyridine in accordance with the procedure in Example 21, Step (b).

¹H NMR (400 MHz, CDCl₃) δ 10.05 (br s, 1H), 8.52 (s, 1H), 8.41 (d, J=2.7 Hz, 1H), 8.21-8.02 (m, 1H), 7.68-7.52 (m, 1H), 5.90 (br s, 1H), 5.53 (d, J=9.7 Hz, 1H), 3.40-3.34 (m, 1H), 3.27 (s, 3H), 3.26-3.17 (m, 1H), 2.94 (q, J=10.4 Hz, 1H), 2.00 (b rs, 1H), 1.87-1.77 (m, 2H), 1.77-1.68 (m, 2H), 1.44 (s, 3H), 1.42 (s, 3H), 1.41 (m, 1H), 1.36-1.15 (m, 7H).

### Example 25: (R)-2-((((1s,4S)-4-(Benzyloxy)cyclohexyl)methyl)amino)-1-(3-fluoro-phenyl)ethan-1-ol

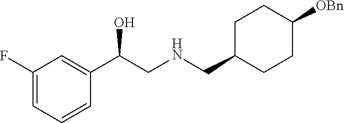 [SMILES: O[C@@H](CNC[C@H]1CC[C@@H](OCc2ccccc2)CC1)c1cccc(F)c1]

### (a) Benzyl 2,2,2-trichloroacetimidate

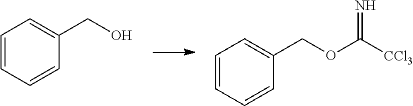

1,8-Diazabicyclo[5.4.0]undec-7-ene (235 μL, 1.57 mmol) was added to a stirred ice-cooled mixture of benzyl alcohol (1.63 mL, 15.72 mmol), trichloroacetonitrile (1.74 mL, 17.29 mmol) and hexane (30 mL). After 40 min at 0° C. the mixture had turned into a solution and hexane (20 mL) and saturated NH₄Cl (aq, sat, 30 mL) were added. The organic phase was collected and washed with NH₄Cl (aq, sat), dried (Na₂SO₄) and concentrated (keeping the temperature below 36° C.) to give the sub-title compound (3.8 g, 97%).

### (b) Methyl (1s,4s)-4-(benzyloxy)cyclohexane-1-carboxylate

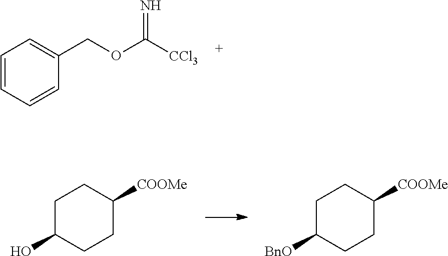

Benzyl trichloroacetimidate (1.41 mL, 7.58 mmol) and trifluoromethanesulfonic acid (85 μL, 0.94 mmol) were added to a solution of methyl (1s,4s)-4-hydroxycyclohexane-1-carboxylate (1.0 g, 6.32 mmol) in cyclohexane/CHCl₃ (2/1 v/v, 18 mL) at rt. The solution was stirred for 18 h at rt, diluted with EtOAc, washed with NaHCO₃(aq, sat), H₂O and brine, dried (Na₂SO₄) and concentrated. The residue was purified by chromatography to give the sub-title compound (1.06 mg, 68%).

### (c) ((1s,4s)-4-(Benzyloxy)cyclohexyl)methyl methanesulfonate

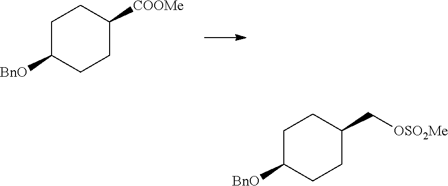

The sub-title compound was prepared from methyl (1s,4s)-4-(benzyloxy)cyclohexane-1-carboxylate in accordance with the procedures in Example 1, Steps (b) and (c).

### (d) N-Benzyl-1-((1s,4s)-4-(benzyloxy)cyclohexyl)methanamine hydrochloride

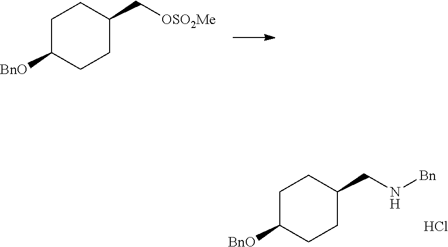

A mixture of ((1r,4r)-4-(benzyloxy)cyclohexyl)methyl methanesulfonate (305 mg, 1.02 mmol) and benzyl amine (1 mL) was heated by microwave irradiation at 100° C. for 2 h. The mixture was concentrated and the residue was partitioned between NaHCO₃(aq, sat) and EtOAc. The layers were separated and the organic phase was dried (Na₂SO₄) and concentrated. The residue was dissolved in CH₂Cl₂ (15 mL) and Boc₂O (325 mg, 1.49 mmol) and triethylamine (332 μL, 241 mg, 2.38 mmol) were added. The mixture was stirred at rt for 22 h and another portion of Boc₂O (65 mg, 0.30 mmol) was added and stirring was continued at rt for 2 h. H₂O was added and the mixture was extracted with CH₂Cl₂. The combined extracts were dried (Na₂SO₄), concentrated and the residue was purified by chromatography. HCl (4.0 M in 1,4-dioxane, 4.0 mL, 16.00 mmol) was added and the mixture was stirred at rt for 2 h and concentrated to give the sub-title product (270 mg, 76%).

### (e) (R)-2-(Benzyl(((1s,4S)-4-(benzyloxy)cyclohexyl)methyl)amino)-1-(3-fluoro-phenyl)ethan-1-ol

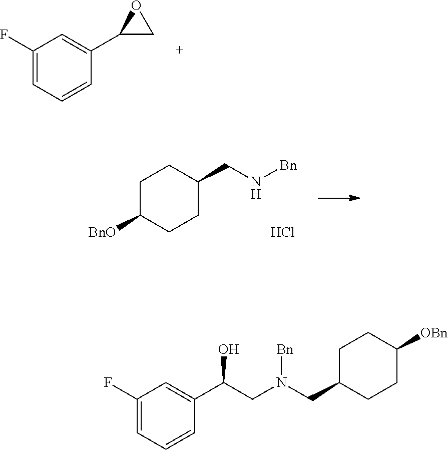 [SMILES: Fc1cccc([C@@H]2CO2)c1 ; c1ccc(CNC[C@H]2CC[C@@H](OCc3ccccc3)CC2)cc1 ; Cl ; O[C@@H](CN(Cc1ccccc1)C[C@H]1CC[C@@H](OCc2ccccc2)CC1)c1cccc(F)c1]

A mixture of N-benzyl-1-((1s,4s)-4-(benzyloxy)cyclohexyl)methanamine hydrochloride (127 mg, 0.37 mmol), (R)-2-(3-fluorophenyl)oxirane (101 mg, 0.73 mmol) KOH (20 mg, 0.37 mmol) and iPrOH (0.56 mL) was stirred at 80° C. for 17 h. The mixture was concentrated and the residue purified by chromatography to give the sub-title compound (117 mg, 71%).

### (f) (R)-2-((((1s,4S)-4-(Benzyloxy)cyclohexyl)methyl)amino)-1-(3-fluorophenyl)-ethan-1-ol

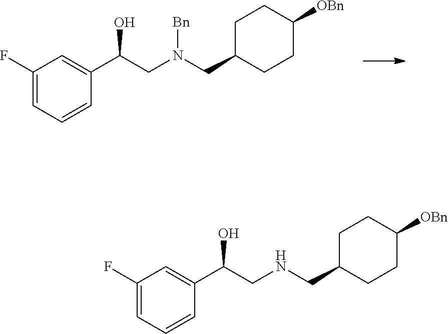 [SMILES: O[C@@H](CN(Cc1ccccc1)C[C@H]1CC[C@@H](OCc2ccccc2)CC1)c1cccc(F)c1 ; O[C@@H](CNC[C@H]1CC[C@@H](OCc2ccccc2)CC1)c1cccc(F)c1]

A mixture of ((R)-2-(benzyl(((1s,4S)-4-(benzyloxy)cyclohexyl)methyl)amino)-1-(3-fluorophenyl)ethan-1-ol (99 mg, 0.22 mmol) Pd/C (10%, 47 mg, 0.044 mmol) and iPrOH (5 mL) was hydrogenated at ambient temperature and pressure for 4 h. The mixture was filtered through a pad of Celite, which was washed with MeOH. The combined filtrates were concentrated and the residue purified by chromatography to give the title compound (60 mg, 76%).

¹H NMR (400 MHz, CDCl₃) δ 7.39-7.23 (m, 6H), 7.17-7.10 (m, 2H), 6.95 (tdd, J=8.4, 2.6, 1.2 Hz, 1H), 4.89 (dd, J=9.5, 3.2 Hz, 1H), 4.48 (s, 2H), 4.2-3.7 (br s, 2H), 3.66-3.58 (m, 1H), 3.01 (dd, J=12.3, 3.2 Hz, 1H), 2.76 (dd, J=12.3, 9.5 Hz, 1H), 2.69 (dd, J=11.9, 6.6 Hz, 1H), 2.61 (dd, J=11.9, 6.6 Hz, 1H), 2.00-1.85 (m, 2H), 1.71-1.51 (m, 3H), 1.52-1.35 (m, 4H).

### Example 26:(1S,4s)-4-((((R)-2-(3-Fluorophenyl)-2-hydroxyethyl)amino)methyl)-cyclohexan-1-ol

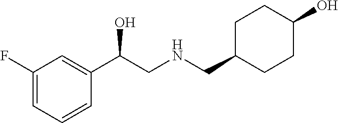 [SMILES: O[C@@H](CNC[C@H]1CC[C@@H](O)CC1)c1cccc(F)c1]

TMSOTf (66 μL, 81 mg, 0.36 mmol) was added dropwise to a solution of (R)-2-((((1s,4S)-4-(benzyloxy)cyclohexyl)methyl)amino)-1-(3-fluorophenyl)ethan-1-ol, see Example 25, Step (f), (52 mg, 0.15 mmol) in CHCl₃ (1 mL) at rt. The solution was stirred at rt for 17 h. NaHCO₃(aq, sat) was added and the aq water layer was collected and extracted with CH₂Cl₂. The combined extracts were dried (Na₂SO₄) and concentrated. The residue was purified by chromatography to give the title compound (21 mg, 54%).

¹H NMR (400 MHz, CD₃OD) δ 7.40-7.31 (m, 1H), 7.21-7.16 (m, 1H), 7.16-7.11 (m, 1H), 7.03-6.95 (m, 1H), 4.83 (dd, J=8.5, 4.7 Hz, 1H), 3.96-3.84 (m, 1H), 2.85-2.71 (m, 2H), 2.63 (dd, J=11.9, 6.8 Hz, 1H), 2.55 (dd, J=11.9, 6.8 Hz, 1H), 1.78-1.66 (m, 2H), 1.66-1.37 (m, 7H).

### Example 27: (R)-2-((((1s,4S)-4-(Benzyloxy)cyclohexyl)methyl)amino)-1-(5-fluoropyridin-3-yl)ethan-1-ol

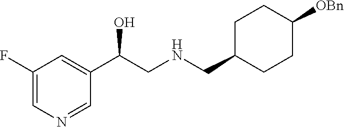 [SMILES: O[C@@H](CNC[C@H]1CC[C@@H](OCc2ccccc2)CC1)c1cncc(F)c1]

### (a) (R)-2-(Benzyl(((1s,4S)-4-(benzyloxy)cyclohexyl)methyl)amino)-1-(5-fluoro-pyridin-3-yl)ethan-1-ol

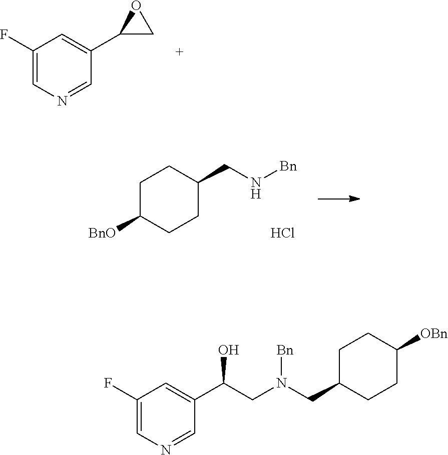 [SMILES: Fc1cncc([C@@H]2CO2)c1 ; c1ccc(CNC[C@H]2CC[C@@H](OCc3ccccc3)CC2)cc1 ; Cl ; O[C@@H](CN(Cc1ccccc1)C[C@H]1CC[C@@H](OCc2ccccc2)CC1)c1cncc(F)c1]

A mixture of N-benzyl-1-((1s,4s)-4-(benzyloxy)cyclohexyl)methanamine hydrochloride (127 mg, 0.37 mmol), (R)-3-fluoro-5-(oxiran-2-yl)pyridine (87 mg, 0.62 mmol), KOH (20 mg, 0.37 mmol) and iPrOH (0.56 mL) was stirred at 80° C. for 24 h. The mixture was allowed to cool to rt and H₂O was added. The mixture was and extracted with CH₂Cl₂ and the combined extracts were dried (Na₂SO₄) and concentrated. The residue was purified by chromatography to give the sub-title compound (90 mg, 55%).

### (b) (R)-2-((((1s,4S)-4-(Benzyloxy)cyclohexyl)methyl)amino)-1-(5-fluoropyridin-3-yl)ethan-1-ol

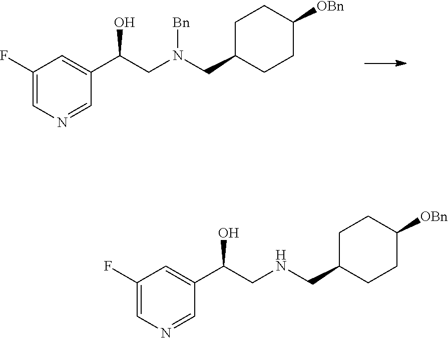 [SMILES: O[C@@H](CN(Cc1ccccc1)C[C@H]1CC[C@@H](OCc2ccccc2)CC1)c1cncc(F)c1 ; O[C@@H](CNC[C@H]1CC[C@@H](OCc2ccccc2)CC1)c1cncc(F)c1]

A mixture of (R)-2-(benzyl(((1s,4S)-4-(benzyloxy)cyclohexyl)methyl)amino)-1-(5-fluoropyridin-3-yl)ethan-1-ol (77 mg, 0.17 mmol) Pd/C (10%, 36 mg, 0.034 mmol) and iPrOH (5 mL) was hydrogenated at ambient temperature and pressure for 18 h. The mixture was filtered through a pad of Celite, which was washed with MeOH. The combined filtrates were concentrated and the residue purified by chromatography to give the title compound (60 mg, 76%).

¹H NMR (400 MHz, CDCl₃) δ 8.40-8.38 (m, 1H), 8.37 (d, J=2.8 Hz, 1H), 7.53-7.46 (m, 1H), 7.39-7.31 (m, 4H), 7.29-7.23 (m, 1H), 4.72 (dd, J=9.3, 3.7 Hz, 1H), 4.51 (s, 2H), 3.68-3.62 (m, 1H), 2.95 (dd, J=12.3, 3.7 Hz, 1H), 2.67-2.57 (m, 2H), 2.53 (dd, J=11.8, 5.4 Hz, 1H), 2.00-1.87 (m, 2H), 1.60-1.36 (m, 7H).

### Example 28:(1S,4s)-4-((((R)-2-(5-Fluoropyridin-3-yl)-2-hydroxyethyl)amino)-methyl)cyclohexan-1-ol

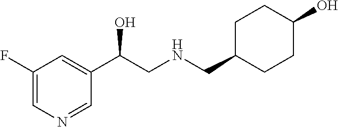 [SMILES: O[C@@H](CNC[C@H]1CC[C@@H](O)CC1)c1cncc(F)c1]

TMSOTf (68 μL, 84 mg, 0.38 mmol) was added dropwise to a solution of (R)-2-((((1s,4S)-4-(benzyloxy)cyclohexyl)methyl)amino)-1-(5-fluoropyridin-3-yl)ethan-1-ol, see Example 27, Step (b), (27 mg, 0.07 mmol) in CHCl₃ (2 mL) at rt. The solution was stirred at rt for 17 h. NaHCO₃(aq, sat) was added and the pH was adjusted to 10 with NaOH (aq, 1 M). The mixture was extracted with CH₂Cl₂ and the combined extracts were dried (Na₂SO₄) and concentrated. The residue was purified by chromatography to give the title compound (5 mg, 25%).

¹H NMR (400 MHz, CDCl₃) δ 8.40-8.38 (m, 1H), 8.37 (d, J=2.8 Hz, 1H), 7.52-7.47 (m, 1H), 4.77-4.69 (m, 1H), 4.04-3.98 (m, 1H), 2.95 (dd, J=12.3, 3.7 Hz, 1H), 2.68-2.57 (m, 2H), 2.53 (dd, J=11.8, 5.7 Hz, 1H), 1.80-1.67 (m, 2H), 1.63-1.35 (m, 7H).

### Example 29: (R)-2-((((1r,4R)-4-(Benzyloxy)cyclohexyl)methyl)amino)-1-(3-fluoro-phenyl)ethan-1-ol

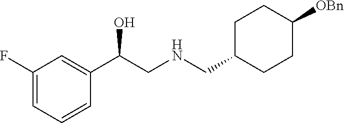 [SMILES: O[C@@H](CNC[C@H]1CC[C@H](OCc2ccccc2)CC1)c1cccc(F)c1]

The title compound was prepared from methyl (1r,4r)-4-hydroxycyclohexane-1-carboxylate in accordance with the procedures in Example 25, Steps (b) to (f).

¹H NMR (400 MHz, CDCl₃) δ 7.29-7.15 (m, 6H), 7.10-6.99 (m, 2H), 6.95-6.83 (m, 1H), 4.72 (dd, J=9.2, 3.2 Hz, 1H), 4.48 (s, 2H), 3.30-3.15 (m, 1H), 2.88 (dd, J=12.2, 3.3 Hz, 1H), 2.78 (br s, 2H), 2.64 (dd, J=12.4, 9.2 Hz, 1H), 2.54-2.42 (m, 2H), 2.12-2.00 (m, 2H), 1.86-1.74 (m, 2H), 1.53-1.36 (m, 1H), 1.29-1.16 (m, 2H), 0.98-0.82 (m, 2H).

### Example 30:(1R,4r)-4-((((R)-2-(3-Fluorophenyl)-2-hydroxyethyl)amino)methyl)-cyclohexan-1-ol

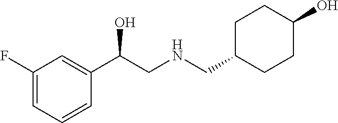 [SMILES: O[C@@H](CNC[C@H]1CC[C@H](O)CC1)c1cccc(F)c1]

The title compound was prepared from (R)-2-((((1r,4R)-4-(benzyloxy)cyclohexyl)-methyl)amino)-1-(3-fluorophenyl)ethan-1-ol, see Example 29, in accordance with the procedure in Example 26.

¹H NMR (400 MHz, CD₃CN) δ 7.41-7.29 (m, 1H), 7.23-7.07 (m, 2H), 7.05-6.90 (m, 1H), 4.70 (dd, J=9.0, 3.8 Hz, 1H), 3.48-3.35 (m, 1H), 2.83 (dd, J=12.2, 3.8 Hz, 1H), 2.63 (dd, J=12.2, 9.0 Hz, 1H), 2.54-2.43 (m, 2H), 1.92-1.83 (m, 2H), 1.84-1.71 (m, 2H), 1.48-1.31 (m, 1H), 1.23-1.08 (m, 2H), 1.03-0.86 (m, 2H).

### Example 31: (R)-2-((((1r,4R)-4-(Benzyloxy)cyclohexyl)methyl)amino)-1-(5-fluoropyridin-3-yl)ethan-1-ol

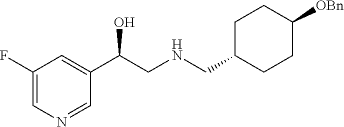 [SMILES: O[C@@H](CNC[C@H]1CC[C@H](OCc2ccccc2)CC1)c1cncc(F)c1]

The title compound was prepared from methyl (1r,4r)-4-hydroxycyclohexane-1-carboxylate in accordance with the procedures in Example 25, Steps (b) to (f), using (R)-3-fluoro-5-(oxiran-2-yl)pyridine in Step (e).

¹H NMR (400 MHz, CDCl₃) δ 8.40-8.38 (m, 1H), 8.37 (d, J=2.8 Hz, 1H), 7.52-7.47 (m, 1H), 7.37-7.30 (m, 4H), 7.30-7.22 (m, 1H), 4.80 (dd, J=9.4, 3.5 Hz, 1H), 4.55 (s, 2H), 3.30 (tt, J=10.8, 4.2 Hz, 1H), 3.19-2.88 (m, 3H), 2.67 (dd, J=12.3, 9.4 Hz, 1H), 2.58 (dd, J=11.8, 6.8 Hz, 1H), 2.51 (dd, J=11.8, 6.5 Hz, 1H), 2.18-2.07 (m, 2H), 1.92-1.79 (m, 2H), 1.55-1.40 (m, 1H), 1.38-1.23 (m, 2H), 1.06-0.90 (m, 2H).

### Example 32:(1R,4r)-4-((((R)-2-(5-Fluoropyridin-3-yl)-2-hydroxyethyl)amino)-methyl)cyclohexan-1-ol

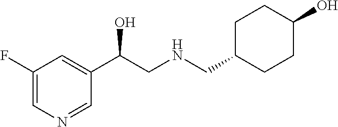 [SMILES: O[C@@H](CNC[C@H]1CC[C@H](O)CC1)c1cncc(F)c1]

The title compound was prepared from (R)-2-((((1r,4R)-4-(benzyloxy)cyclohexyl)-methyl)amino)-1-(5-fluoropyridin-3-yl)ethan-1-ol, see Example 31, in accordance with the procedure in Example 28.

¹H NMR (400 MHz, CDCl₃) δ 8.40-8.34 (m, 2H), 7.52-7.46 (m, 1H), 4.72 (dd, J=9.2, 3.7 Hz, 1H), 3.57 (tt, J=10.9, 4.3 Hz, 1H), 2.94 (dd, J=12.3, 3.6 Hz, 1H), 2.64 (dd, J=12.3, 9.3 Hz, 1H), 2.55 (dd, J=11.8, 6.7 Hz, 1H), 2.48 (dd, J=11.8, 6.5 Hz, 1H), 2.05-1.96 (m, 2H), 1.89-1.78 (m, 2H), 1.47-1.34 (m, 1H), 1.33-1.20 (m, 2H), 1.08-0.94 (m, 2H).

### Example 33: (R)-2-((2-((1s,4S)-4-(Benzyloxy)cyclohexyl)propan-2-yl)amino)-1-(3-fluorophenyl)ethan-1-ol

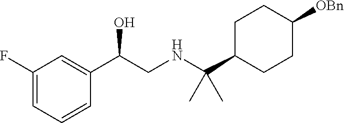 [SMILES: CC(C)(NC[C@H](O)c1cccc(F)c1)[C@H]1CC[C@@H](OCc2ccccc2)CC1]

### (a) 2-((1s,4s)-4-(Benzyloxy)cyclohexyl)propan-2-ol

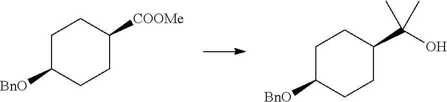 [SMILES: COC(=O)[C@H]1CC[C@@H](OCc2ccccc2)CC1 ; CC(C)(O)[C@H]1CC[C@@H](OCc2ccccc2)CC1]

Methylmagnesium bromide (3.0 M in Et₂O, 1.46 mL, 4.38 mmol) was added dropwise to a solution of methyl (1r,4r)-4-(benzyloxy)cyclohexane-1-carboxylate, see Example 25, Step (b), (363 mg, 1.46 mmol) in THF (10 mL) at −78° C. The mixture was stirred at rt for 16 h and NH₄Cl (aq, sat) was added. The mixture was extracted with EtOAc and the combined extracts dried (Na₂SO₄) and concentrated. The residue was purified by chromatography to give the sub-title compound (240 mg, 80%).

### (b) (R)-2-((2-((1s,4S)-4-(Benzyloxy)cyclohexyl)propan-2-yl)amino)-1-(3-fluoro-phenyl)ethan-1-ol

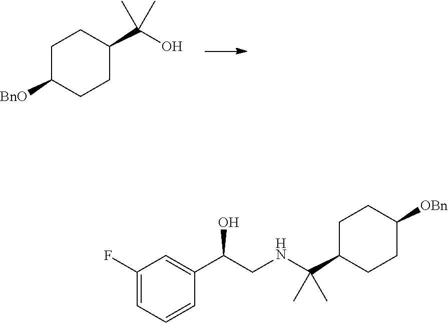 [SMILES: CC(C)(O)[C@H]1CC[C@@H](OCc2ccccc2)CC1 ; CC(C)(NC[C@H](O)c1cccc(F)c1)[C@H]1CC[C@@H](OCc2ccccc2)CC1]

The title compound was prepared from 2-((1s,4s)-4-(benzyloxy)cyclohexyl)propan-2-ol in accordance with the procedures in Example 5, Steps (b) to (d).

¹H NMR (400 MHz, CDCl₃) δ 7.37-7.23 (m, 6H), 7.18-7.11 (m, 2H), 6.99-6.92 (m, 1H), 4.76 (dd, J=9.1, 3.3 Hz, 1H), 4.49 (s, 2H), 3.69-3.61 (m, 1H), 3.3-2.5 (2H, m) 2.96 (dd, J=11.9, 3.4 Hz, 1H), 2.60 (dd, J=11.9, 9.1 Hz, 1H), 2.12-2.01 (m, 2H), 1.58-1.41 (m, 4H), 1.41-1.28 (m, 3H), 1.12 (d, J=5.3 Hz, 6H).

### Example 34:(1S,4s)-4-(2-(((R)-2-(3-Fluorophenyl)-2-hydroxyethyl)amino)propan-2-yl)cyclohexan-1-ol

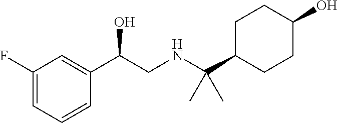 [SMILES: CC(C)(NC[C@H](O)c1cccc(F)c1)[C@H]1CC[C@@H](O)CC1]

A mixture of (R)-2-((2-((1s,4S)-4-(benzyloxy)cyclohexyl)propan-2-yl)amino)-1-(3-fluorophenyl)ethan-1-ol (43 mg, 0.11 mmol), Pd/C (10%, 30 mg, 0.028 mmol), ammonium formate (141 mg, 2.23 mmol) and iPrOH (1 mL) was stirred at 70° C. for 18 h. The mixture was concentrated and the residue was purified by chromatography to give the title compound (5 mg, 15%).

¹H NMR (400 MHz, CDCl₃) δ 7.34-7.27 (m, 1H), 7.16-7.09 (m, 2H), 6.95 (tdd, J=8.5, 2.6, 1.1 Hz, 1H), 4.57 (dd, J=8.8, 3.7 Hz, 1H), 4.08-4.03 (m, 1H), 2.89 (dd, J=11.9, 3.7 Hz, 1H), 2.52 (dd, J=11.9, 8.8 Hz, 1H), 1.91-1.79 (m, 3H), 1.57-1.40 (m, 6H), 1.04 (s, 3H), 1.03 (s, 3H).

### Example 35: (R)-2-((2-((1s,4S)-4-(Benzyloxy)cyclohexyl)propan-2-yl)amino)-1-(5-fluoropyridin-3-yl)ethan-1-ol

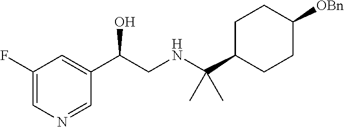 [SMILES: CC(C)(NC[C@H](O)c1cncc(F)c1)[C@H]1CC[C@@H](OCc2ccccc2)CC1]

The title compound was prepared from 2-((1s,4s)-4-(benzyloxy)cyclohexyl)propan-2-amine (prepared from 2-((1s,4s)-4-(benzyloxy)cyclohexyl)propan-2-ol, see Example 33, Step (a), in accordance with the procedures in Example 5, Steps (b) and (c)) and (R)-3-fluoro-5-(oxiran-2-yl)pyridine in accordance with the procedure in Example 1, Step (e).

¹H NMR (400 MHz, CDCl₃) δ 8.49-8.46 (m, 1H), 8.38 (d, J=2.7 Hz, 1H), 7.60-7.53 (m, 1H), 7.37-7.30 (m, 4H), 7.29-7.23 (m, 1H), 5.36-5.26 (m, 1H), 5.23-4.56 (m, 2H), 4.47 (s, 2H), 3.67-3.60 (m, 1H), 3.14 (dd, J=12.1, 2.3 Hz, 1H), 2.81 (dd, J=12.1, 9.9 Hz, 1H), 2.17-1.98 (m, 2H), 1.69-1.43 (m, 5H), 1.41-1.23 (m, 8H).

### Example 36:(1S,4s)-4-(2-(((R)-2-(5-Fluoropyridin-3-yl)-2-hydroxyethyl)amino)propan-2-yl)cyclohexan-1-ol

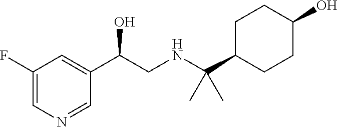 [SMILES: CC(C)(NC[C@H](O)c1cncc(F)c1)[C@H]1CC[C@@H](O)CC1]

A mixture of of (R)-2-((2-((1s,4S)-4-(benzyloxy)cyclohexyl)propan-2-yl)amino)-1-(5-fluoropyridin-3-yl)ethan-1-ol, see Example 35, (55 mg, 0.14 mmol), Pd/C (10%, 76 mg, 0.07 mmol), formic acid (0.8 mL) and MeOH (3.2 mL) was hydrogenated at ambient temperature and pressure for 18 h. The mixture was filtered through a pad of Celite, which was washed with MeOH. The combined filtrates were concentrated and the residue purified by chromatography (amino-functionalized silica gel) to give the title compound (4 mg, 10%).

¹H NMR (400 MHz, CDCl₃) δ 8.40-8.38 (m, 1H), 8.38 (d, J=2.8 Hz, 1H), 7.53-7.47 (m, 1H), 4.63 (dd, J=8.9, 3.7 Hz, 1H), 4.10-4.01 (m, 1H), 2.94 (dd, J=12.1, 3.8 Hz, 1H), 2.51 (dd, J=12.1, 8.9 Hz, 1H), 1.93-1.79 (m, 2H), 1.59-1.39 (m, 6H), 1.34-1.19 (m, 2H), 1.05 (s, 3H), 1.04 (s, 3H).

### Example 37: (R)-2-((2-((1r,4R)-4-(Benzyloxy)cyclohexyl)propan-2-yl)amino)-1-(3-fluoro-phenyl)ethan-1-ol

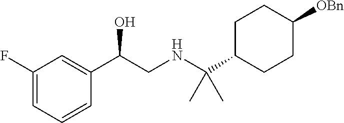 [SMILES: CC(C)(NC[C@H](O)c1cccc(F)c1)[C@H]1CC[C@H](OCc2ccccc2)CC1]

The title compound was prepared from methyl (1r,4r)-4-hydroxycyclohexane-1-carboxylate in accordance with procedures in Example 25, Steps (b) to (f).

¹H NMR (400 MHz, CDCl₃) δ 7.36-7.32 (m, 4H), 7.32-7.24 (m, 2H), 7.17-7.10 (m, 2H), 6.99-6.93 (m, 1H), 4.75-4.66 (m, 1H), 4.55 (s, 2H), 3.26 (tt, J=10.9, 4.2 Hz, 1H), 3.2-2.1 (2H, m) 2.93 (dd, J=11.9, 3.5 Hz, 1H), 2.58 (dd, J=11.9, 9.0 Hz, 1H), 2.23-2.09 (m, 2H), 1.88-1.74 (m, 2H), 1.37-1.18 (m, 3H), 1.16-1.00 (m, 8H).

### Example 38:(1R,4r)-4-(2-(((R)-2-(3-Fluorophenyl)-2-hydroxyethyl)amino)propan-2-yl)cyclohexan-1-ol

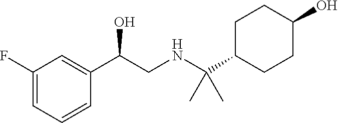 [SMILES: CC(C)(NC[C@H](O)c1cccc(F)c1)[C@H]1CC[C@H](O)CC1]

The title compound was prepared from (R)-2-((2-((1r,4R)-4-(benzyloxy)cyclohexyl)-propan-2-yl)amino)-1-(3-fluorophenyl)ethan-1-ol, see Example 37, in accordance with procedure in Example 26.

¹H NMR (400 MHz, CDCl₃) δ 7.34-7.27 (m, 1H), 7.15-7.08 (m, 2H), 6.96 (tdd, J=8.5, 2.6, 1.1 Hz, 1H), 4.56 (dd, J=8.7, 3.7 Hz, 1H), 3.53 (tt, J=10.8, 4.3 Hz, 1H), 2.88 (dd, J=11.9, 3.7 Hz, 1H), 2.52 (dd, J=11.9, 8.7 Hz, 1H), 2.08-1.97 (m, 2H), 1.85-1.70 (m, 2H), 1.29-1.17 (m, 3H), 1.16-1.06 (m, 2H), 1.02 (s, 3H), 1.01 (s, 3H).

### Example 39: (R)-2-((2-((1r,4R)-4-(Benzyloxy)cyclohexyl)propan-2-yl)amino)-1-(5-fluoropyridin-3-yl)ethan-1-ol

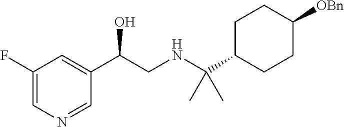 [SMILES: CC(C)(NC[C@H](O)c1cncc(F)c1)[C@H]1CC[C@H](OCc2ccccc2)CC1]

The title compound was prepared from 2-((1r,4r)-4-(benzyloxy)cyclohexyl)propan-2-amine (prepared from methyl (1r,4r)-4-(benzyloxy)cyclohexane-1-carboxylate in accordance with procedure in Example 25, Step (b), followed by the procedures in Example 5, Steps (a) to (d) using (R)-3-fluoro-5-(oxiran-2-yl)pyridine in Step (d).

¹H NMR (400 MHz, CDCl₃) δ 8.46-8.42 (m, 1H), 8.39 (d, J=2.8 Hz, 1H), 7.56-7.50 (m, 1H), 7.38-7.30 (m, 4H), 7.30-7.23 (m, 1H), 5.09-4.99 (m, 1H), 4.54 (s, 2H), 3.26 (tt, J=10.8, 4.2 Hz, 1H), 3.05 (dd, J=12.1, 2.9 Hz, 1H), 2.69 (dd, J=12.1, 9.5 Hz, 1H), 2.23-2.11 (m, 2H), 1.88-1.80 (m, 2H), 1.44-1.33 (m, 1H), 1.32-1.04 (m, 10H).

### Example 40:(1R,4r)-4-(2-(((R)-2-(5-Fluoropyridin-3-yl)-2-hydroxyethyl)amino)propan-2-yl)cyclohexan-1-ol

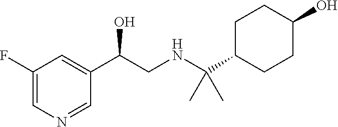 [SMILES: CC(C)(NC[C@H](O)c1cncc(F)c1)[C@H]1CC[C@H](O)CC1]

The title compound was prepared from (R)-2-((2-((1r,4R)-4-(benzyloxy)cyclohexyl)-propan-2-yl)amino)-1-(5-fluoropyridin-3-yl)ethan-1-ol, see Example 39, in accordance with procedure in Example 26.

¹H NMR (400 MHz, CDCl₃) δ 8.43-8.33 (m, 2H), 7.53-7.45 (m, 1H), 4.62 (dd, J=8.8, 3.8 Hz, 1H), 3.54 (tt, J=10.8, 4.3 Hz, 1H), 2.92 (dd, J=12.0, 3.8 Hz, 1H), 2.51 (dd, J=12.0, 8.8 Hz, 1H), 2.13-1.97 (m, 2H), 1.84-1.67 (m, 2H), 1.30-1.17 (m, 3H), 1.16-1.07 (m, 2H), 1.02 (s, 3H), 1.02 (s, 3H).

### Example 41: (R)-2-((2-((1s,4S)-4-(Benzyloxy)cyclohexyl)ethyl)amino)-1-(3-fluoro-phenyl)ethan-1-ol

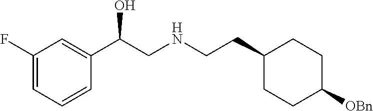 [SMILES: O[C@@H](CNCC[C@H]1CC[C@@H](OCc2ccccc2)CC1)c1cccc(F)c1]

### (a) 2-((1s,4s)-4-(Benzyloxy)cyclohexyl)acetonitrile

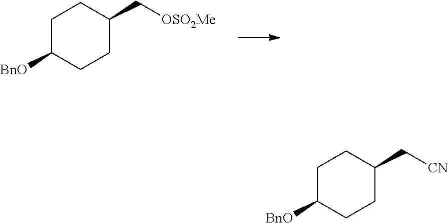

NaCN (326 mg, 6.66 mmol) was added to a solution of ((1r,4r)-4-(benzyloxy)cyclo-hexyl)methyl methanesulfonate, see Example 25, Step (c), (662 mg, 2.22 mmol) in DMF (7 mL). The mixture was stirred at 80° C. for 3 h and cooled to rt. H₂O was added and the mixture was extracted with Et₂O. The combined extracts were washed with H₂O, dried (MgSO₄) and concentrated to give the sub-title compound (480 mg, 94%).

### (b) 2-((1s,4s)-4-(Benzyloxy)cyclohexyl)ethan-1-amine

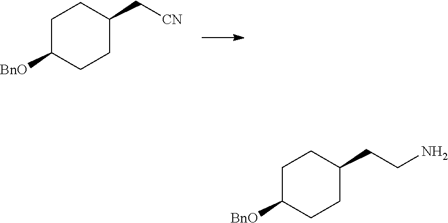 [SMILES: N#CC[C@H]1CC[C@@H](OCc2ccccc2)CC1 ; NCC[C@H]1CC[C@@H](OCc2ccccc2)CC1]

LiAlH₄ (1 M in THF, 3.5 mL, 3.50 mmol) was added dropwise to an ice-cooled solution of 2-((1s,4s)-4-(benzyloxy)cyclohexyl)acetonitrile (400 mg, 1.74 mmol) in THF (10 mL). The mixture was stirred at rt for 1 h. The mixture was cooled in an ice-bath and H₂O (0.13 mL) was added. After stirring at 0° C. for 10 min, NaOH (aq, 4 M, 0.26 mL) was added and the mixture was stirred at 0° C. for 10 min. H₂O (0.40 mL) was added and the mixture was stirred at 0° C. for 10 min and filtered through a pad of Celite, which was washed with CH₂Cl₂. The combined filtrates were dried (Na₂SO₄) and concentrated to give the sub-title compound (353 mg, 87%), which was used in the next step without further purification.

### (c) (R)-2-((2-((1s,4S)-4-(Benzyloxy)cyclohexyl)ethyl)amino)-1-(3-fluorophenyl)-ethan-1-ol

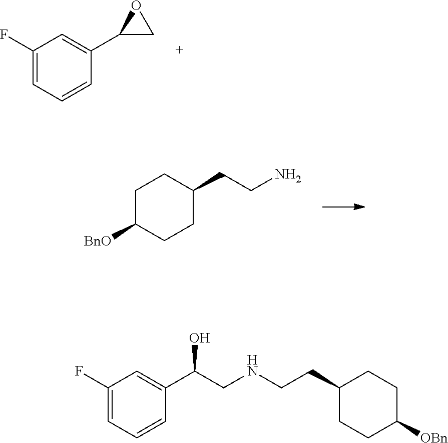 [SMILES: Fc1cccc([C@@H]2CO2)c1 ; NCC[C@H]1CC[C@@H](OCc2ccccc2)CC1 ; O[C@@H](CNCC[C@H]1CC[C@@H](OCc2ccccc2)CC1)c1cccc(F)c1]

The title compound was prepared from (R)-2-(3-fluorophenyl)oxirane and 2-((1s,4s)-4-(benzyloxy)cyclohexyl)ethan-1-amine in accordance with the procedure in Example 1, Step (e).

¹H NMR (400 MHz, CDCl₃) δ 7.40-7.30 (m, 4H), 7.30-7.23 (m, 2H), 7.16-7.07 (m, 2H), 6.99-6.91 (m, 1H), 4.67 (dd, J=8.9, 3.7 Hz, 1H), 4.50 (s, 2H), 3.64-3.57 (m, 1H), 2.90 (dd, J=12.2, 3.7 Hz, 1H), 2.77-2.59 (m, 3H), 1.98-1.81 (m, 2H), 1.56-1.31 (m, 9H).

### Example 42:(1S,4s)-4-(2-(((R)-2-(3-Fluorophenyl)-2-hydroxyethyl)amino)ethyl)-cyclohexan-1-ol

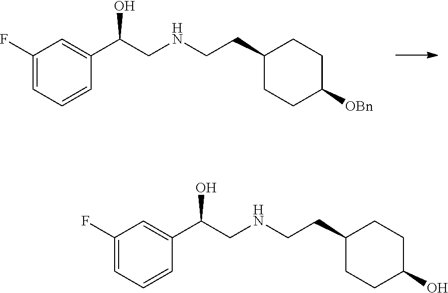 [SMILES: O[C@@H](CNCC[C@H]1CC[C@@H](OCc2ccccc2)CC1)c1cccc(F)c1 ; O[C@@H](CNCC[C@H]1CC[C@@H](O)CC1)c1cccc(F)c1]

The title compound was prepared from (R)-2-((2-((1s,4S)-4-(benzyloxy)cyclohexyl)-ethyl)amino)-1-(3-fluorophenyl)ethan-1-ol in accordance with procedure in Example 36.

¹H NMR (400 MHz, CDCl₃) δ 7.33-7.26 (m, 1H), 7.15-7.07 (m, 2H), 6.95 (tdd, J=8.5, 2.6, 1.1 Hz, 1H), 4.71 (dd, J=9.0, 3.6 Hz, 1H), 4.02-3.85 (m, 1H), 2.90 (dd, J=12.2, 3.6 Hz, 1H), 2.79-2.57 (m, 3H), 1.77-1.62 (m, 2H), 1.61-1.29 (m, 10H).

### Example 43: (R)-2-((2-((1s,4S)-4-(Benzyloxy)cyclohexyl)ethyl)amino)-1-(3-fluoro-phenyl)ethan-1-ol

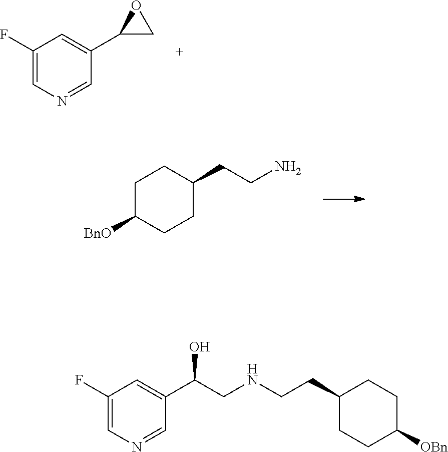 [SMILES: Fc1cncc([C@@H]2CO2)c1 ; NCC[C@H]1CC[C@@H](OCc2ccccc2)CC1 ; O[C@@H](CNCC[C@H]1CC[C@@H](OCc2ccccc2)CC1)c1cncc(F)c1]

The title compound was prepared from (R)-3-fluoro-5-(oxiran-2-yl)pyridine and 2-((1s,4s)-4-(benzyloxy)cyclohexyl)ethan-1-amine in accordance with the procedure in Example 1, Step (e).

¹H NMR (400 MHz, CDCl₃) δ 8.40-8.38 (m, 1H), 8.37 (d, J=2.8 Hz, 1H), 7.52-7.47 (m, 1H), 7.39-7.30 (m, 4H), 7.30-7.23 (m, 1H), 4.73 (dd, J=9.2, 3.7 Hz, 1H), 4.50 (s, 2H), 3.66-3.57 (m, 1H), 2.96 (dd, J=12.3, 3.7 Hz, 1H), 2.78-2.69 (m, 1H), 2.69-2.60 (m, 2H), 1.97-1.82 (m, 2H), 1.57-1.34 (m, 9H).

### Example 44:(1S,4s)-4-(2-(((R)-2-(5-Fluoropyridin-3-yl)-2-hydroxyethyl)amino)-ethyl)cyclohexan-1-ol

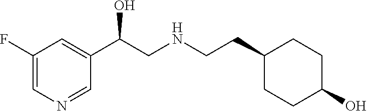 [SMILES: O[C@@H](CNCC[C@H]1CC[C@@H](O)CC1)c1cncc(F)c1]

The title compound was prepared from (R)-2-((2-((1s,4S)-4-(benzyloxy)cyclohexyl)-ethyl)amino)-1-(5-fluoropyridin-3-yl)ethan-1-ol in accordance with the procedure in Example 36.

¹H NMR (400 MHz, CDCl₃) δ 8.39-8.37 (m, 1H), 8.36 (d, J=2.8 Hz, 1H), 7.52-7.46 (m, 1H), 4.76 (dd, J=9.2, 3.6 Hz, 1H), 4.00-3.91 (m, 1H), 2.94 (dd, J=12.3, 3.6 Hz, 1H), 2.77-2.60 (m, 3H), 2.5-2.1 (m, 3H), 1.76-1.64 (m, 2H), 1.60-1.32 (m, 9H).

### Example 45: (R)-2-((2-((1r,4R)-4-(Benzyloxy)cyclohexyl)ethyl)amino)-1-(3-fluoro-phenyl)ethan-1-ol

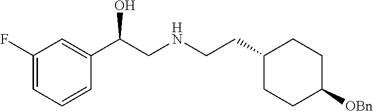 [SMILES: O[C@@H](CNCC[C@H]1CC[C@H](OCc2ccccc2)CC1)c1cccc(F)c1]

The title compound was prepared in accordance with the procedure in Example 1, Step (e) from 2-((1r,4r)-4-(benzyloxy)cyclohexyl)ethan-1-amine (prepared from methyl (1r,4r)-4-hydroxycyclohexane-1-carboxylate in accordance with the procedures in Example 25, Step (b) and Example 1, Steps (b) and (c) and Example 41, Step (a)) and (R)-2-(3-fluorophenyl)oxirane.

¹H NMR (400 MHz, CDCl₃) δ 7.38-7.24 (m, 6H), 7.15-7.07 (m, 2H), 6.99-6.91 (m, 1H), 4.67 (dd, J=8.9, 3.7 Hz, 1H), 4.56 (s, 2H), 3.29 (tt, J=10.8, 4.2 Hz, 1H), 2.90 (dd, J=12.3, 3.7 Hz, 1H), 2.74-2.57 (m, 3H), 2.15-2.04 (m, 2H), 1.83-1.72 (m, 2H), 1.42-1.34 (m, 2H), 1.34-1.22 (m, 3H), 1.01-0.85 (m, 2H).

### Example 46:(1R,4r)-4-(2-(((R)-2-(3-Fluorophenyl)-2-hydroxyethyl)amino)ethyl)-cyclohexan-1-ol

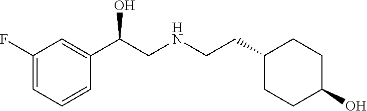 [SMILES: O[C@@H](CNCC[C@H]1CC[C@H](O)CC1)c1cccc(F)c1]

The title compound was prepared from (R)-2-((2-((1r,4R)-4-(benzyloxy)cyclohexyl)-ethyl)amino)-1-(3-fluorophenyl)ethan-1-ol, see Example 45, in accordance with the procedure in Example 36.

¹H NMR (400 MHz, CDCl₃) δ 7.33-7.26 (m, 1H), 7.15-7.07 (m, 2H), 6.95 (tdd, J=8.4, 2.6, 1.1 Hz, 1H), 4.68 (dd, J=8.9, 3.7 Hz, 1H), 3.54 (tt, J=10.9, 4.3 Hz, 1H), 2.89 (dd, J=12.2, 3.7 Hz, 1H), 2.76-2.55 (m, 3H), 2.01-1.91 (m, 2H), 1.83-1.68 (m, 2H), 1.44-1.33 (m, 2H), 1.31-1.16 (m, 3H), 1.05-0.88 (m, 2H).

### Example 47: (R)-2-((2-((1r,4R)-4-(Benzyloxy)cyclohexyl)ethyl)amino)-1-(5-fluoropyridin-3-yl)ethan-1-ol

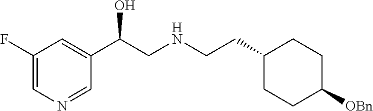 [SMILES: O[C@@H](CNCC[C@H]1CC[C@H](OCc2ccccc2)CC1)c1cncc(F)c1]

The title compound was prepared in accordance with the procedure in Example 1, Step (e) from 2-((1r,4r)-4-(benzyloxy)cyclohexyl)ethan-1-amine (prepared from methyl (1r,4r)-4-hydroxycyclohexane-1-carboxylate in accordance with the procedures in Example 25, Step (b) and Example 1, Steps (b) and (c) and Example 41, Step (a)) and (R)-3-fluoro-5-(oxiran-2-yl)pyridine.

¹H NMR (400 MHz, CDCl₃) δ 8.41-8.38 (m, 1H), 8.36 (d, J=2.8 Hz, 1H), 7.53-7.46 (m, 1H), 7.38-7.29 (m, 4H), 7.29-7.22 (m, 1H), 4.90 (dd, J=9.5, 3.4 Hz, 1H), 4.54 (s, 2H), 4.12-3.85 (m, 2H), 3.28 (tt, J=10.8, 4.1 Hz, 1H), 2.99 (dd, J=12.3, 3.4 Hz, 1H), 2.84-2.64 (m, 3H), 2.15-2.02 (m, 2H), 1.83-1.71 (m, 2H), 1.53-1.40 (m, 2H), 1.38-1.19 (m, 3H), 1.03-0.85 (m, 2H).

### Example 48:(1R,4r)-4-(2-(((R)-2-(5-Fluoropyridin-3-yl)-2-hydroxyethyl)amino)-ethyl)cyclohexan-1-ol

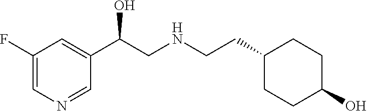 [SMILES: O[C@@H](CNCC[C@H]1CC[C@H](O)CC1)c1cncc(F)c1]

The title compound was prepared from (R)-2-((2-((1r,4R)-4-(benzyloxy)cyclohexyl)-ethyl)amino)-1-(5-fluoropyridin-3-yl)ethan-1-ol in accordance with the procedure in Example 36.

¹H NMR (400 MHz, CDCl₃) δ 8.40-8.38 (m, 1H), 8.38-8.37 (m, 1H), 7.52-7.47 (m, 1H), 4.73 (dd, J=9.2, 3.7 Hz, 1H), 3.55 (tt, J=10.8, 4.3 Hz, 1H), 2.95 (dd, J=12.3, 3.7 Hz, 1H), 2.79-2.55 (m, 3H), 2.04-1.90 (m, 2H), 1.81-1.69 (m, 2H), 1.45-1.34 (m, 2H), 1.33-1.15 (m, 3H), 1.06-0.90 (m, 2H).

### Example 49:(1S,4s)-4-(2-(((R)-2-(3-Fluorophenyl)-2-hydroxyethyl)amino)-2-methylpropyl)cyclohexan-1-ol hydrochloride

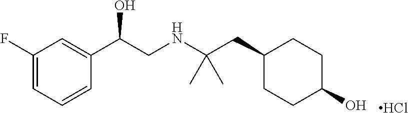 [SMILES: CC(C)(C[C@H]1CC[C@@H](O)CC1)NC[C@H](O)c1cccc(F)c1 ; Cl]

### (a) Methyl (1s,4s)-4-((tert-butyldimethylsilyl)oxy)cyclohexane-1-carboxylate

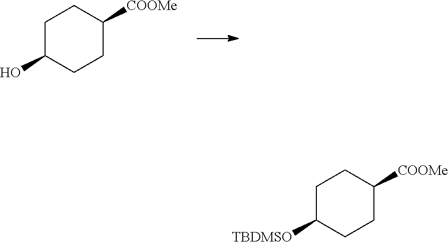 [SMILES: COC(=O)[C@H]1CC[C@@H](O)CC1 ; COC(=O)[C@H]1CC[C@@H](O[Si](C)(C)C(C)(C)C)CC1]

Imidazole (323 mg, 4.74 mmol) and tert-butyldimethylsilyl chloride (572 mg, 3.79 mmol) were added to a solution of methyl (1s,4s)-4-hydroxycyclohexane-1-carboxylate (500 mg, 3.16 mmol) in DMF (10 mL). The mixture was stirred at rt for 20 h and imidazole (43.0 mg, 0.63 mmol) and tert-butyldimethylsilyl chloride (95.3 mg, 0.63 mol) were added. The mixture was stirred at rt for 1 h and Et₂O and H₂O were added. The aq phase was collected and extracted with Et₂O. The combined extracts were washed with H₂O and brine, dried (Na₂SO₄) and concentrated. The residue was purified by chromatography to give the sub-title compound (718 mg, 83%).

### (b) 1-((1s,4s)-4-((tert-Butyldimethylsilyl)oxy)cyclohexyl)-2-methylpropan-2-amine

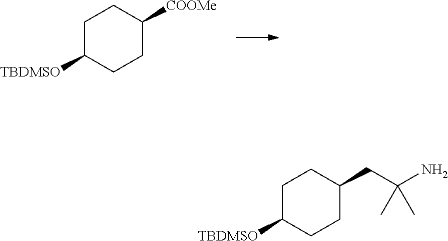 [SMILES: COC(=O)[C@H]1CC[C@@H](O[Si](C)(C)C(C)(C)C)CC1 ; CC(C)(N)C[C@H]1CC[C@@H](O[Si](C)(C)C(C)(C)C)CC1]

The sub-title compound was prepared from methyl (1s,4s)-4-((tert-butyldimethylsilyl)-oxy)cyclohexane-1-carboxylate in accordance with the procedures in Example 41, Step (b), Example 17, Step (e), Example 9, Step (a) and Example 13, Step (a).

### (c) (R)-2-((1-((1s,4S)-4-((tert-Butyldimethylsilyl)oxy)cyclohexyl)-2-methylpropan-2-yl)amino)-1-(3-fluorophenyl)ethan-1-ol

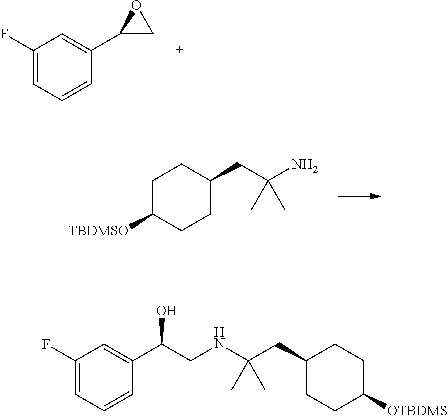 [SMILES: Fc1cccc([C@@H]2CO2)c1 ; CC(C)(N)C[C@H]1CC[C@@H](O[Si](C)(C)C(C)(C)C)CC1 ; CC(C)(C[C@H]1CC[C@@H](O[Si](C)(C)C(C)(C)C)CC1)NC[C@H](O)c1cccc(F)c1]

A mixture of 1-((1s,4s)-4-((tert-butyldimethylsilyl)oxy)cyclohexyl)-2-methylpropan-2-amine (269 mg, 0.94 mmol), (R)-2-(3-fluorophenyl)oxirane (100 mg, 0.72 mmol) and iPrOH (1.1 mL) was stirred at 80° C. for 20 h. The mixture was allowed to cool and MeCN (1 mL) was added. The solids were collected and washed with a small amount of MeCN and dried to give the sub-title compound (160 mg, 52%)

### (d) (1S,4s)-4-(2-(((R)-2-(3-fluorophenyl)-2-hydroxyethyl)amino)-2-methyl-propyl)cyclohexan-1-ol hydrochloride

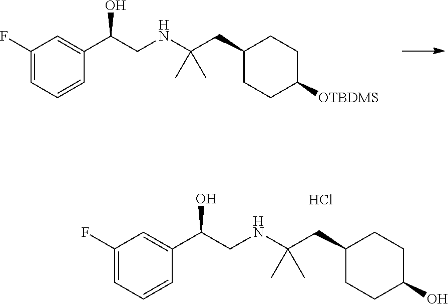 [SMILES: CC(C)(C[C@H]1CC[C@@H](O[Si](C)(C)C(C)(C)C)CC1)NC[C@H](O)c1cccc(F)c1 ; CC(C)(C[C@H]1CC[C@@H](O)CC1)NC[C@H](O)c1cccc(F)c1 ; Cl]

NH₄F (262 mg, 7.08 mmol) was added to a solution of (R)-2-((1-((1s,4S)-4-((tert-butyldimethylsilyl)oxy)cyclohexyl)-2-methylpropan-2-yl)amino)-1-(3-fluorophenyl)ethan-1-ol (100 mg, 0.24 mmol) in MeOH (4 mL). The mixture was stirred at 60° C. for 3 days and concentrated. The residue was extracted with CH₂Cl₂ and the combined extracts were concentrated. The residue was purified by chromatography on amino-functionalized silica gel to give (1S,4s)-4-(2-(((R)-2-(3-fluorophenyl)-2-hydroxyethyl)amino)-2-methylpropyl)cyclohexan-1-ol (39 mg, 53%). (32 mg, 0.10 mmol) of this material was dissolved in Et₂O (3.0 mL) and HCl (2.0 M in Et₂O, 54 μL, 0.11 mmol) was added. The solids were collected and washed with Et₂O to give the title compound (31 mg, 46%).

¹H NMR (400 MHz, CDCl₃) δ 7.45-7.38 (m, 1H), 7.30-7.21 (m, 2H), 7.11-7.03 (m, 1H), 4.94 (dd, J=10.3, 3.0 Hz, 1H), 3.90-3.80 (m, 1H), 3.19 (dd, J=12.5, 3.0 Hz, 1H), 3.04 (dd, J=12.5, 10.3 Hz, 1H), 1.77-1.45 (m, 11H), 1.40 (s, 3H), 1.39 (s, 3H).

### Example 50:(1S,4s)-4-(2-(((R)-2-(5-Fluoropyridin-3-yl)-2-hydroxyethyl)amino)-2-methylpropyl)cyclohexan-1-ol hydrochloride

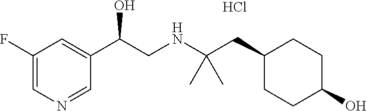 [SMILES: CC(C)(C[C@H]1CC[C@@H](O)CC1)NC[C@H](O)c1cncc(F)c1 ; Cl]

The title compound was prepared from methyl (1s,4s)-4-hydroxycyclohexane-1-carboxylate in accordance with the procedures in Example 49 using (R)-3-fluoro-5-(oxiran-2-yl)pyridine in Step (c).

¹H NMR (400 MHz, CD₃OD) δ 8.54-8.51 (m, 1H), 8.47-8.44 (m, 1H), 7.83-7.77 (m, 1H), 5.13-5.03 (m, 1H), 3.90-3.82 (m, 1H), 3.30-3.26 (m, 1H), 3.14 (dd, J=12.6, 10.2 Hz, 1H), 1.74-1.47 (m, 11H), 1.42 (s, 3H), 1.40 (s, 3H).

### Example 51:(1R,4r)-4-(2-(((R)-2-(3-Fluorophenyl)-2-hydroxyethyl)amino)-2-methylpropyl)cyclohexan-1-ol hydrochloride

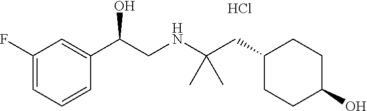 [SMILES: CC(C)(C[C@H]1CC[C@H](O)CC1)NC[C@H](O)c1cccc(F)c1 ; Cl]

The title compound was prepared from methyl (1r,4r)-4-hydroxycyclohexane-1-carboxylate in accordance with the procedures in Example 49.

¹H NMR (400 MHz, CD₃OD) δ 7.46-7.37 (m, 1H), 7.30-7.20 (m, 2H), 7.11-7.02 (m, 1H), 5.01-4.89 (m, 1H), 3.54-3.42 (m, 1H), 3.21-3.09 (m, 1H), 3.07-2.96 (m, 1H), 1.98-1.89 (m, 2H), 1.88-1.78 (m, 2H), 1.64-1.53 (m, 2H), 1.49-1.33 (m, 7H), 1.34-1.22 (m, 2H), 1.22-1.06 (m, 2H).

### Example 52:(1R,4r)-4-(2-(((R)-2-(5-Fluoropyridin-3-yl)-2-hydroxyethyl)amino)-2-methylpropyl)cyclohexan-1-ol

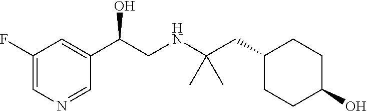 [SMILES: CC(C)(C[C@H]1CC[C@H](O)CC1)NC[C@H](O)c1cncc(F)c1]

The title compound was prepared from methyl (1r,4r)-4-hydroxycyclohexane-1-carboxylate in accordance with the procedures in Example 49 using (R)-3-fluoro-5-(oxiran-2-yl)pyridine in Step (c) but without the formation of a salt in the last step.

¹H NMR (400 MHz, CDCl₃) δ 8.42-8.34 (m, 2H), 7.53-7.46 (m, 1H), 4.75-4.62 (m, 1H), 3.53 (tt, J=10.8, 4.3 Hz, 1H), 3.01-2.88 (m, 1H), 2.59-2.48 (m, 1H), 2.01-1.89 (m, 2H), 1.83-1.73 (m, 2H), 1.42-1.18 (m, 6H), 1.15-0.97 (m, 8H).

### Example 53: (R)-2-((3-((1r,4S)-4-(Benzyloxy)cyclohexyl)propyl)amino)-1-(3-fluoro-phenyl)ethan-1-ol

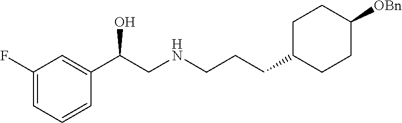

### (a) Ethyl 2-((1r,4r)-4-(benzyloxy)cyclohexyl)acetate

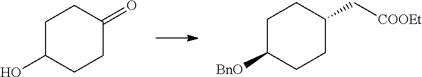 [SMILES: O=C1CCC(O)CC1 ; CCOC(=O)C[C@H]1CC[C@H](OCc2ccccc2)CC1]

The sub-title compound and the isomeric ethyl 2-((1s,4s)-4-(benzyloxy)cyclohexyl)acetate was prepared from 4-hydroxycyclohexan-1-one in accordance with the procedures in Example 25, Step (b), followed by the procedures in Example 17, Steps (b) and (c).

### (b) 3-((1r,4s)-4-(Benzyloxy)cyclohexyl)propan-1-amine

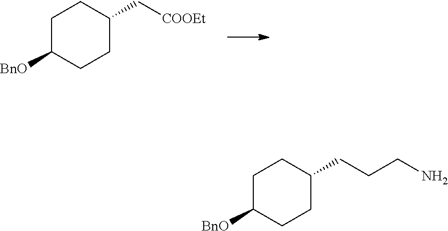 [SMILES: CCOC(=O)C[C@H]1CC[C@H](OCc2ccccc2)CC1 ; NCCC[C@H]1CC[C@H](OCc2ccccc2)CC1]

The sub-title compound was prepared from ethyl 2-((1r,4r)-4-(benzyloxy)cyclohexyl)-acetate in accordance with the procedures in Example 17, Steps (d) to (g).

### (c) (R)-2-((3-((1r,4S)-4-(Benzyloxy)cyclohexyl)propyl)amino)-1-(3-fluorophenyl)-ethan-1-ol

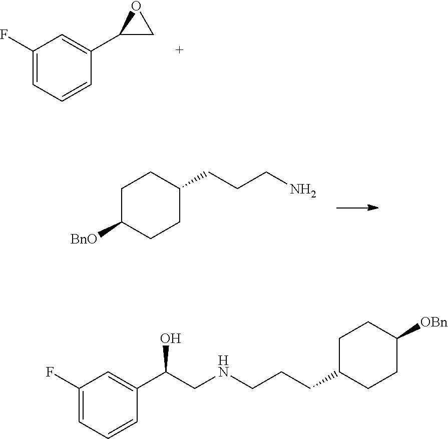

The title compound was prepared from 3-((1r,4s)-4-(benzyloxy)cyclohexyl)propan-1-amine and (R)-2-(3-fluorophenyl)oxirane in accordance with the procedure in Example 1, Step (e).

¹H NMR (400 MHz, CDCl₃) δ 7.37-7.30 (m, 4H), 7.30-7.23 (m, 2H), 7.16-7.07 (m, 2H), 6.99-6.92 (m, 1H), 4.71 (dd, J=9.0, 3.6 Hz, 1H), 4.55 (s, 2H), 3.28 (tt, J=10.9, 4.2 Hz, 1H), 2.92 (dd, J=12.2, 3.6 Hz, 1H), 2.74-2.56 (m, 3H), 2.33 (br s, 2H), 2.15-2.03 (m, 2H), 1.83-1.72 (m, 2H), 1.56-1.44 (m, 2H), 1.36-1.13 (m, 5H), 0.97-0.82 (m, 2H).

### Example 54: (R)-2-((3-((1r,4S)-4-(Benzyloxy)cyclohexyl)propyl)amino)-1-(5-fluoro-pyridin-3-yl)ethan-1-ol

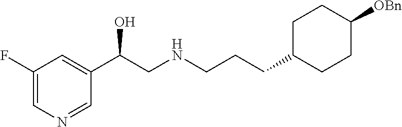

The title compound was prepared from 3-((1r,4s)-4-(benzyloxy)cyclohexyl)propan-1-amine, see Example 53, Step (b), and (R)-3-fluoro-5-(oxiran-2-yl)pyridine in accordance with the procedure in Example 1, Step (e).

¹H NMR (400 MHz, CDCl₃) δ 8.43 (t, J=1.7 Hz, 1H), 8.38 (d, J=2.7 Hz, 1H), 7.55-7.49 (m, 1H), 7.37-7.30 (m, 4H), 7.29-7.23 (m, 1H), 5.02 (dd, J=9.7, 3.2 Hz, 1H), 4.55 (s, 2H), 3.81 (br s, 2H), 3.27 (tt, J=10.8, 4.2 Hz, 1H), 3.07 (dd, J=12.3, 3.2 Hz, 1H), 2.87-2.68 (m, 3H), 2.14-2.02 (m, 2H), 1.83-1.71 (m, 2H), 1.69-1.56 (m, 2H), 1.34-1.13 (m, 5H), 1.00-0.79 (m, 2H).

### Example 55: (R)-2-((3-((1s,4R)-4-(Benzyloxy)cyclohexyl)propyl)amino)-1-(3-fluoro-phenyl)ethan-1-ol

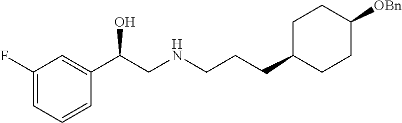 [SMILES: O[C@@H](CNCCC[C@H]1CC[C@@H](OCc2ccccc2)CC1)c1cccc(F)c1]

The title compound was prepared from ethyl 2-((1s,4s)-4-(benzyloxy)cyclohexyl)acetate (see Example 53, Step (a)) in accordance with the procedures in Example 53, Steps (b) and (c).

¹H NMR (400 MHz, CDCl₃) δ 7.39-7.30 (m, 4H), 7.30-7.23 (m, 2H), 7.16-7.08 (m, 2H), 6.99-6.92 (m, 1H), 4.74 (dd, J=9.1, 3.5 Hz, 1H), 4.50 (s, 2H), 3.63-3.57 (m, 1H), 2.93 (dd, J=12.3, 3.6 Hz, 1H), 2.77-2.56 (m, 3H), 2.39 (br s, 2H), 1.94-1.85 (m, 2H), 1.60-1.34 (m, 8H), 1.35-1.20 (m, 3H).

### Example 56: (R)-2-((3-((1s,4R)-4-(Benzyloxy)cyclohexyl)propyl)amino)-1-(5-fluoro-pyridin-3-yl)ethan-1-ol

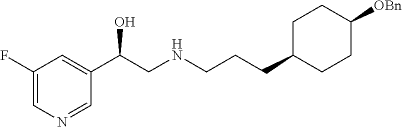 [SMILES: O[C@@H](CNCCC[C@H]1CC[C@@H](OCc2ccccc2)CC1)c1cncc(F)c1]

The title compound was prepared from ethyl 2-((1s,4s)-4-(benzyloxy)cyclohexyl)acetate (see Example 53, Step (a)) in accordance with the procedures in Example 17, Steps (d) to (g).

¹H NMR (400 MHz, CDCl₃) δ 8.40 (t, J=1.7 Hz, 1H), 8.37 (d, J=2.8 Hz, 1H), 7.53-7.47 (m, 1H), 7.39-7.30 (m, 4H), 7.29-7.23 (m, 1H), 4.78 (dd, J=9.3, 3.6 Hz, 1H), 4.50 (s, 2H), 3.63-3.58 (m, 1H), 2.97 (dd, J=12.3, 3.6 Hz, 1H), 2.77-2.57 (m, 3H), 2.35 (br s, 2H), 1.95-1.85 (m, 2H), 1.60-1.36 (m, 8H), 1.36-1.19 (m, 3H).

### Example 57:(1S,4r)-4-(3-(((R)-2-(3-Fluorophenyl)-2-hydroxyethyl)amino)propyl)-cyclohexan-1-ol

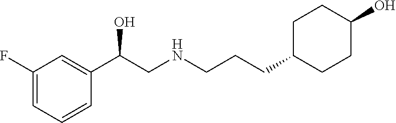 [SMILES: O[C@@H](CNCCC[C@H]1CC[C@H](O)CC1)c1cccc(F)c1]

### (a) Ethyl 2-(4-((tert-butyldimethylsilyl)oxy)cyclohexyl)acetate

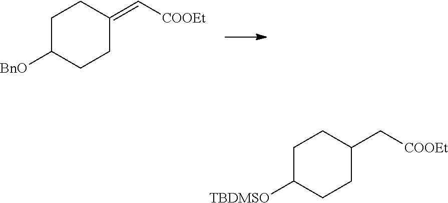 [SMILES: CCOC(=O)C=C1CCC(OCc2ccccc2)CC1 ; CCOC(=O)CC1CCC(O[Si](C)(C)C(C)(C)C)CC1]

A mixture of ethyl 2-(4-(benzyloxy)cyclohexylidene)acetate, prepared from in 4-hydroxycyclohexan-1-one in accordance with the procedure in Example 25, Step (b) followed by the procedure in Example 17, Step (b), (2.8 g, 10.21 mmol), Pd/C (10%, 163 mg, 0.15 mmol) and MeOH (20 mL) was hydrogenated at ambient pressure and temperature for 2 h. The mixture was filtered through Celite, which was washed with MeOH. The combined filtrates were concentrated and the residue was dissolved in DMF (10 mL). Imidazole (932 mg, 13.69 mmol) and tert-butyldimethylsilyl chloride (1.7 g, 10.95 mmol) were added and the mixture was stirred at rt for 17 h. Et₂O and H₂O were added and the layers were separated. The aq phase was extracted with Et₂O and the combined extracts were washed with H₂O and brine, dried (Na₂SO₄) and concentrated to give the sub-title compound (2.2 g, 81%) as a 0.8:1 cis:trans mixture.

### (b) 3-((1r,4s)-4-((tert-Butyldimethylsilyl)oxy)cyclohexyl)propanenitrile

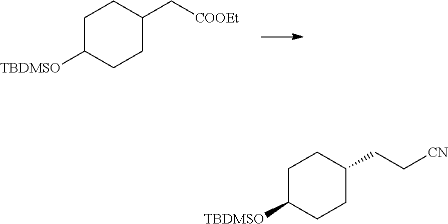 [SMILES: CCOC(=O)CC1CCC(O[Si](C)(C)C(C)(C)C)CC1 ; CC(C)(C)[Si](C)(C)O[C@H]1CC[C@H](CCC#N)CC1]

The sub-title compound along with the isomeric 3-((1s,4r)-4-((tert-butyldimethylsilyl)-oxy)cyclohexyl)propanenitrile was prepared from ethyl 2-(4-((tert-butyldimethylsilyl)-oxy)cyclohexyl)acetate in accordance with the procedures in Example 17, Steps (d) to (f)

### (c) 3-((1r,4s)-4-((tert-Butyldimethylsilyl)oxy)cyclohexyl)propan-1-amine

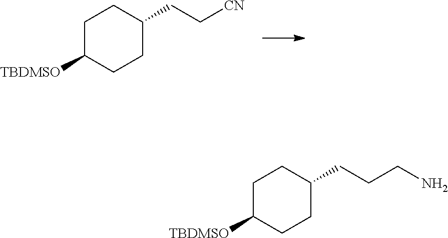 [SMILES: CC(C)(C)[Si](C)(C)O[C@H]1CC[C@H](CCC#N)CC1 ; CC(C)(C)[Si](C)(C)O[C@H]1CC[C@H](CCCN)CC1]

The sub-title compound was prepared from 3-((1r,4s)-4-((tert-butyldimethylsilyl)-oxy)cyclohexyl)propanenitrile in accordance with the procedure in Example 17, Step (g).

### (d) (R)-2-((3-((1r,4S)-4-((tert-Butyldimethylsilyl)oxy)cyclohexyl)propyl)amino)-1-(3-fluorophenyl)ethan-1-ol

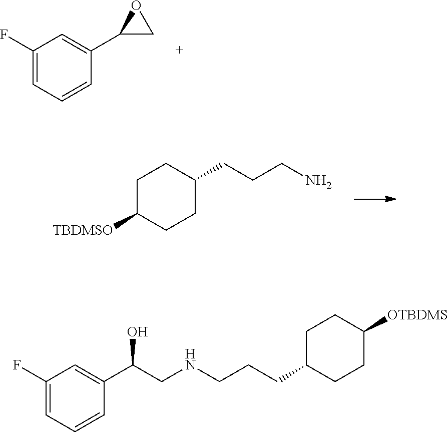 [SMILES: Fc1cccc([C@@H]2CO2)c1 ; CC(C)(C)[Si](C)(C)O[C@H]1CC[C@H](CCCN)CC1 ; CC(C)(C)[Si](C)(C)O[C@H]1CC[C@H](CCCNC[C@H](O)c2cccc(F)c2)CC1]

The sub-title compound was prepared from 3-((1r,4s)-4-((tert-butyldimethylsilyl)-oxy)cyclohexyl)propan-1-amine and (R)-3-(3-fluorophenyl)oxiran in accordance with the procedure in Example 1, Step (e).

### (e) (1S,4r)-4-(3-(((R)-2-(3-Fluorophenyl)-2-hydroxyethyl)amino)propyl)cyclohexan-1-ol

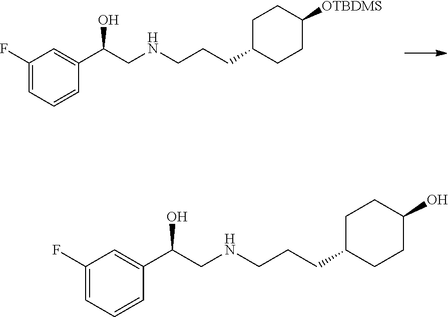 [SMILES: CC(C)(C)[Si](C)(C)O[C@H]1CC[C@H](CCCNC[C@H](O)c2cccc(F)c2)CC1 ; O[C@@H](CNCCC[C@H]1CC[C@H](O)CC1)c1cccc(F)c1]

NH₄F (41 mg, 1.10 mmol) was added to a solution of (R)-2-((3-((1r,4S)-4-((tert-butyldimethylsilyl)oxy)cyclohexyl)propyl)amino)-1-(3-fluorophenyl)ethan-1-ol (15 mg, 0.04 mmo) in MeOH (1 mL). The mixture was stirred at 60° C. for 48 h and concentrated. The residue was extracted with acetone and the combined extracts were concentrated to give the title compound (4 mg, 37%).

¹H NMR (400 MHz, CD₃OD) δ 7.39-7.31 (m, 1H), 7.21-7.16 (m, 1H), 7.16-7.11 (m, 1H), 7.03-6.95 (m, 1H), 4.80 (dd, J=8.5, 4.4 Hz, 1H), 3.51-3.42 (m, 1H), 2.85-2.73 (m, 2H), 2.73-2.59 (m, 2H), 2.00-1.88 (m, 3H), 1.84-1.74 (m, 2H), 1.61-1.50 (m, 2H), 1.36-1.14 (m, 7H), 1.04-0.93 (m, 2H).

### Example 58:(1S,4r)-4-(3-(((R)-2-(5-Fluoropyridin-3-yl)-2-hydroxyethyl)amino)-propyl)cyclohexan-1-ol

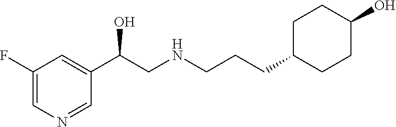 [SMILES: O[C@@H](CNCCC[C@H]1CC[C@H](O)CC1)c1cncc(F)c1]

The title compound was prepared from 3-((1r,4s)-4-((tert-butyldimethylsilyl)-oxy)cyclohexyl)propan-1-amine (see Example 57, Step (c)) and (R)-3-fluoro-5-(oxiran-2-yl)pyridine in accordance with the procedure in Example 1, Step (e) followed by the procedure in Example 57, Step (e).

¹H NMR (400 MHz, CD₃OD) δ 8.42 (t, J=1.7 Hz, 1H), 8.36 (d, J=2.8 Hz, 1H), 7.74-7.61 (m, 1H), 4.91-4.87 (m, 1H), 3.53-3.40 (m, 1H), 2.87-2.70 (m, 2H), 2.70-2.50 (m, 2H), 2.00-1.87 (m, 2H), 1.85-1.68 (m, 2H), 1.54 (p, J=7.5 Hz, 2H), 1.33-1.10 (m, 5H), 1.04-0.84 (m, 2H).

### Example 59:(1R,4s)-4-(3-(((R)-2-(3-Fluorophenyl)-2-hydroxyethyl)amino)propyl)-cyclohexan-1-ol

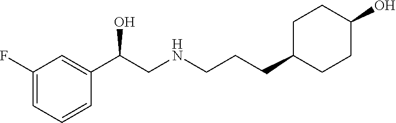 [SMILES: O[C@@H](CNCCC[C@H]1CC[C@@H](O)CC1)c1cccc(F)c1]

The title compound was prepared from 3-((1s,4r)-4-((tert-butyldimethylsilyl)-oxy)cyclohexyl)propanenitrile (see Example 57, Step (b)) in accordance with the procedures in Example 57, Steps (c) to (e).

¹H NMR (400 MHz, CD₃OD) δ 7.35 (td, J=8.0, 5.8 Hz, 1H), 7.20-7.16 (m, 1H), 7.16-7.11 (m, 1H), 7.03-6.94 (m, 1H), 4.80 (dd, J=8.0, 4.8 Hz, 1H), 3.94-3.82 (m, 1H), 2.81-2.71 (m, 2H), 2.71-2.57 (m, 2H), 1.75-1.64 (m, 2H), 1.61-1.46 (m, 6H), 1.46-1.38 (m, 2H), 1.38-1.23 (m, 3H).

### Example 60:(1R,4s)-4-(3-(((R)-2-(5-Fluoropyridin-3-yl)-2-hydroxyethyl)amino)propyl)-cyclohexan-1-ol

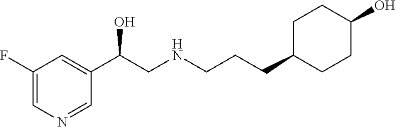 [SMILES: O[C@@H](CNCCC[C@H]1CC[C@@H](O)CC1)c1cncc(F)c1]

The title compound was prepared from 3-((1s,4r)-4-((tert-butyldimethylsilyl)-oxy)cyclohexyl)propanenitrile (see Example 57, Step (b)) in accordance with the procedures in Example 57, Steps (c) to (e) but using (R)-3-fluoro-5-(oxiran-2-yl)pyridine instead of (R)-3-(3-fluorophenyl)oxiran in Step (d).

¹H NMR (400 MHz, CD₃OD) δ 8.42 (t, J=1.7 Hz, 1H), 8.37 (d, J=2.7 Hz, 1H), 7.72-7.62 (m, 1H), 4.91-4.88 (m, 1H), 3.91-3.83 (m, 1H), 2.86-2.74 (m, 2H), 2.71-2.56 (m, 2H), 1.75-1.64 (m, 3H), 1.61-1.46 (m, 7H), 1.46-1.37 (m, 2H), 1.37-1.24 (m, 4H).

### Example 61:(1S,4r)-4-(3-(((R)-2-(3-Fluorophenyl)-2-hydroxyethyl)amino)-3-methyl-butyl)cyclohexan-1-ol

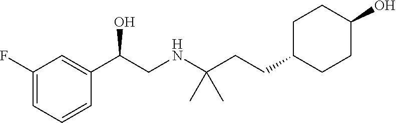 [SMILES: CC(C)(CC[C@H]1CC[C@H](O)CC1)NC[C@H](O)c1cccc(F)c1]

### (a) 4-((1r,4s)-4-((tert-Butyldimethylsilyl)oxy)cyclohexyl)-2-methylbutan-2-amine

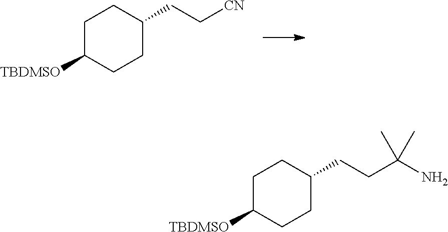 [SMILES: CC(C)(C)[Si](C)(C)O[C@H]1CC[C@H](CCC#N)CC1 ; CC(C)(N)CC[C@H]1CC[C@H](O[Si](C)(C)C(C)(C)C)CC1]

The sub-title compound was prepared from 3-((1r,4s)-4-((tert-butyldimethylsilyl)oxy)-cyclohexyl)propanenitrile (see Example 57, Step (b)) in accordance with the procedures in Example 13, Step (a).

### (b) (R)-2-((4-((1r,4S)-4-((tert-Butyldimethylsilyl)oxy)cyclohexyl)-2-methylbutan-2-yl)amino)-1-(3-fluorophenyl)ethan-1-ol

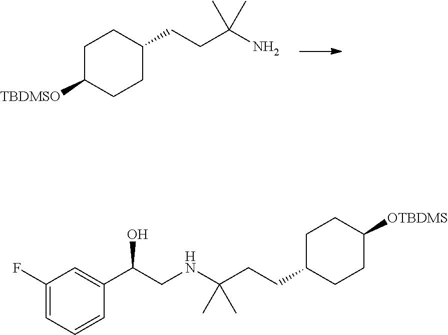 [SMILES: CC(C)(N)CC[C@H]1CC[C@H](O[Si](C)(C)C(C)(C)C)CC1 ; CC(C)(CC[C@H]1CC[C@H](O[Si](C)(C)C(C)(C)C)CC1)NC[C@H](O)c1cccc(F)c1]

The sub-title compound was prepared from 4-((1r,4s)-4-((tert-butyldimethylsilyl-oxy)cyclohexyl)-2-methylbutan-2-amine and (R)-2-(3-fluorophenyl)oxirane in accordance with the procedure in Example 1, Step (e).

### (c) (1S,4r)-4-(3-(((R)-2-(3-Fluorophenyl)-2-hydroxyethyl)amino)-3-methylbutyl)-cyclohexan-1-ol

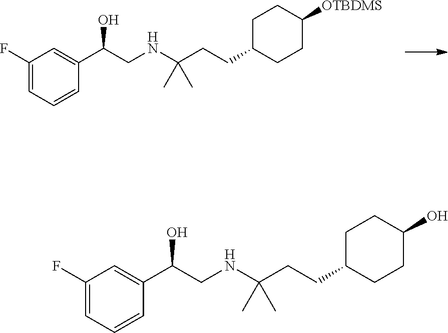 [SMILES: CC(C)(CC[C@H]1CC[C@H](O[Si](C)(C)C(C)(C)C)CC1)NC[C@H](O)c1cccc(F)c1 ; CC(C)(CC[C@H]1CC[C@H](O)CC1)NC[C@H](O)c1cccc(F)c1]

The title compound was prepared from (R)-2-((4-((1r,4S)-4-((tert-butyldimethylsilyl)-oxy)cyclohexyl)-2-methylbutan-2-yl)amino)-1-(3-fluorophenyl)ethan-1-ol in accordance with the procedure in Example 57, Step (e).

¹H NMR (400 MHz, CDCl₃) δ 7.33-7.26 (m, 1H), 7.18-7.05 (m, 2H), 6.98-6.92 (m, 1H), 4.67 (dd, J=8.8, 3.5 Hz, 1H), 3.53 (tt, J=10.8, 4.3 Hz, 1H), 2.89 (dd, J=11.9, 3.6 Hz, 1H), 2.79 (br s, 3H), 2.55 (dd, J=11.9, 8.9 Hz, 1H), 2.00-1.90 (m, 2H), 1.81-1.70 (m, 2H), 1.43-1.33 (m, 2H), 1.28-1.09 (m, 5H), 1.08 (s, 3H), 1.07 (s, 3H), 1.01-0.86 (m, 2H).

### Example 62:(1S,4r)-4-(3-(((R)-2-(2-Fluorophenyl)-2-hydroxyethyl)amino)-3-methyl-butyl)cyclohexan-1-ol

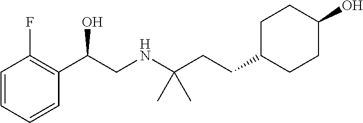

The title compound was prepared in accordance with the procedures in Example 61, using (R)-2-(2-fluorophenyl)oxirane in Step (b).

¹H NMR (400 MHz, CDCl₃) δ 7.58-7.51 (m, 1H), 7.31-7.20 (m, 1H), 7.15 (td, J=7.5, 1.3 Hz, 1H), 7.00 (ddd, J=10.6, 8.1, 1.2 Hz, 1H), 4.91 (dd, J=8.6, 3.6 Hz, 1H), 3.54 (tt, J=10.8, 4.3 Hz, 1H), 2.93 (ddd, J=11.9, 3.7, 1.0 Hz, 1H), 2.52 (ddd, J=11.9, 8.6, 0.7 Hz, 1H), 2.28 (br s, 2H), 2.02-1.91 (m, 2H), 1.82-1.70 (m, 2H), 1.43-1.29 (m, 2H), 1.29-1.06 (m, 6H), 1.04 (s, 6H), 1.01-0.86 (m, 2H).

### Example 63:(1S,4r)-4-(3-(((R)-2-(5-Fluoropyridin-3-yl)-2-hydroxyethyl)amino)-3-methylbutyl)cyclohexan-1-ol

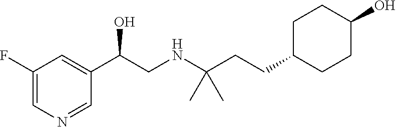 [SMILES: CC(C)(CC[C@H]1CC[C@H](O)CC1)NC[C@H](O)c1cncc(F)c1]

The title compound was prepared in accordance with the procedures in Example 61, using (R)-3-fluoro-5-(oxiran-2-yl)pyridine in Step (b).

¹H NMR (400 MHz, CDCl₃) δ 8.42-8.32 (m, 2H), 7.51-7.46 (m, 1H), 4.64 (dd, 3=8.8, 3.7 Hz, 1H), 3.54 (tt, 3=10.9, 4.3 Hz, 1H), 2.91 (dd, 3=12.1, 3.8 Hz, 1H), 2.50 (dd, J=12.1, 8.8 Hz, 1H), 2.11 (br s, 3H), 2.03-1.91 (m, 2H), 1.83-1.70 (m, 2H), 1.41-1.30 (m, 2H), 1.29-1.06 (m, 5H), 1.05 (s, 6H), 1.02-0.84 (m, 2H).

### Example 64:(1R,4s)-4-(3-(((R)-2-(3-Fluorophenyl)-2-hydroxyethyl)amino)-3-methyl-butyl)cyclohexan-1-ol

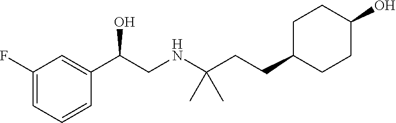 [SMILES: CC(C)(CC[C@H]1CC[C@@H](O)CC1)NC[C@H](O)c1cccc(F)c1]

The title compound was prepared from 3-((1s,4r)-4-((tert-butyldimethylsilyl)oxy)-cyclohexyl)propanenitrile (see Example 57, Step (b)) in accordance with the procedures in Example 61.

¹H NMR (400 MHz, CDCl₃) δ 7.33-7.26 (m, 1H), 7.17-7.06 (m, 2H), 6.98-6.91 (m, 1H), 4.60 (dd, 3=8.7, 3.6 Hz, 1H), 3.98-3.92 (m, 1H), 2.87 (dd, 3=11.9, 3.7 Hz, 1H), 2.52 (dd, 3=11.9, 8.7 Hz, 1H), 2.32 (br s, 3H), 1.75-1.63 (m, 2H), 1.60-1.44 (m, 4H), 1.43-1.29 (m, 4H), 1.28-1.13 (m, 3H), 1.05 (s, 6H).

### Example 65:(1R,4s)-4-(3-(((R)-2-(2-Fluorophenyl)-2-hydroxyethyl)amino)-3-methyl-butyl)cyclohexan-1-ol

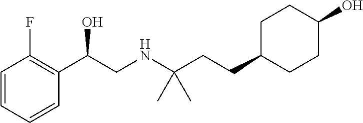 [SMILES: CC(C)(CC[C@H]1CC[C@@H](O)CC1)NC[C@H](O)c1ccccc1F]

The title compound was prepared from 3-((1s,4r)-4-((tert-butyldimethylsilyl)oxy)-cyclohexyl)propanenitrile (see Example 57, Step (b)) in accordance with the procedures in Example 61, using (R)-2-(2-fluorophenyl)oxirane in Step (b).

¹H NMR (400 MHz, CDCl₃) δ 7.61-7.52 (m, 1H), 7.28-7.20 (m, 1H), 7.16 (td, 3=7.5, 1.3 Hz, 1H), 7.00 (ddd, 3=10.6, 8.2, 1.3 Hz, 1H), 4.98 (dd, 3=8.8, 3.4 Hz, 1H), 3.98-3.91 (m, 1H), 2.98 (ddd, J=12.0, 3.5, 1.0 Hz, 1H), 2.89 (br s, 2H), 2.58 (dd, J=12.0, 8.8 Hz, 1H), 1.75-1.63 (m, 2H), 1.60-1.45 (m, 4H), 1.38 (dtd, J=17.1, 10.0, 3.4 Hz, 4H), 1.28-1.16 (m, 4H), 1.09 (s, 6H).

### Example 66:(1R,4s)-4-(3-(((R)-2-(5-Fluoropyridin-3-yl)-2-hydroxyethyl)amino)-3-methylbutyl)cyclohexan-1-ol dihydrochloride

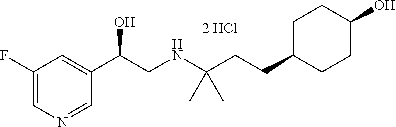 [SMILES: CC(C)(CC[C@H]1CC[C@@H](O)CC1)NC[C@H](O)c1cncc(F)c1]

(1R,4s)-4-(3-(((R)-2-(5-Fluoropyridin-3-yl)-2-hydroxyethyl)amino)-3-methylbutyl)-cyclohexan-1-ol was prepared from 3-((1s,4r)-4-((tert-butyldimethylsilyl)oxy)-cyclohexyl)propanenitrile (see Example 57(b)) in accordance with the procedures in Example 61, using (R)-3-fluoro-5-(oxiran-2-yl)pyridine in Step (b). Its dihydrochloride—the title compound—was prepared in accordance with the procedure in Example 49, Step (c).

¹H NMR (400 MHz, CD₃OD) δ 9.02-8.97 (m, 1H), 8.96-8.90 (m, 1H), 8.65-8.60 (m, 1H), 5.35 (dd, J=9.7, 2.8 Hz, 1H), 3.92-3.86 (m, 1H), 3.44 (dd, J=12.7, 3.0 Hz, 1H), 3.22 (dd, J=12.7, 9.7 Hz, 1H), 1.81-1.65 (m, 4H), 1.60-1.49 (m, 4H), 1.49-1.42 (m, 2H), 1.40 (s, 6H), 1.38-1.25 (m, 3H).

### Example 67: N-((1R,4r)-4-((((R)-2-(3-Fluorophenyl)-2-hydroxyethyl)amino)methyl)-cyclohexyl)acetamide

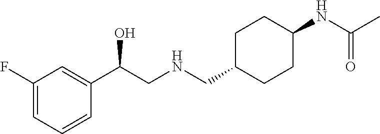 [SMILES: CC(=O)N[C@H]1CC[C@H](CNC[C@H](O)c2cccc(F)c2)CC1]

### (a) tert-Butyl ((1r,4r)-4-(hydroxymethyl)cyclohexyl)carbamate

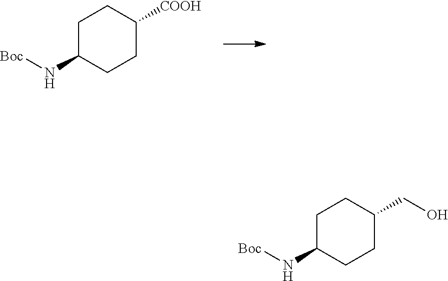 [SMILES: CC(C)(C)OC(=O)N[C@H]1CC[C@H](C(=O)O)CC1 ; CC(C)(C)OC(=O)N[C@H]1CC[C@H](CO)CC1]

Borane dimethyl sulphide complex (1.21 mL, 12.74 mmol) was slowly added dropwise to a an ice-cooled solution of (1r,4r)-4-((tert-butoxycarbonyl)amino)cyclohexane-1-carboxylic acid (1 g, 4.11 mmol) in THF (14 mL). The cooling bath was removed and the mixture was stirred at rt for 3 h. MeOH (0.7 mL) was added and the mixture was concentrated. MeOH (1 mL) was added to the residue and the mixture was concentrated. This procedure was repeated two more times. CH₂Cl₂ was added to the residue and the mixture was washed with brine, dried (Na₂SO₄) and concentrated to give the sub-title compound (925 mg, 98%), which was used in the next step without further purification.

### (b) ((1r,4r)-4-((tert-Butoxycarbonyl)amino)cyclohexyl)methyl methanesulfonate

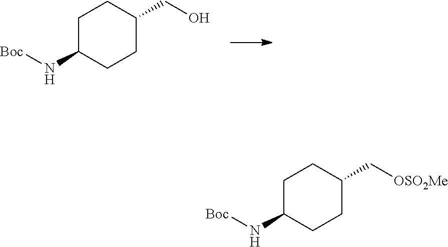

Methanesulfonyl chloride (0.33 mL, 4.32 mmol) was added dropwise to an ice-cooled mixture of tert-butyl ((1r,4r)-4-(hydroxymethyl)cyclohexyl)carbamate (900 mg, 3.92 mmol), triethylamine (0.71 mL, 5.10 mmol) and CH₂Cl₂ (25 mL). The mixture was stirred at 0° C. for 30 min and H₂O was added. The layers were separated and the aq phase was extracted with CH₂Cl₂. The combined organic phases were washed with H₂O and brine, dried (Na₂SO₄) and concentrated to give the sub-title compound (1190 mg, 99%), which was used in the next step without further purification.

### (c) tert-Butyl ((1r,4r)-4-((benzylamino)methyl)cyclohexyl)carbamate

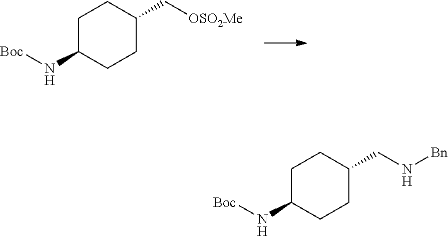

A mixture of ((1r,4r)-4-((tert-butoxycarbonyl)amino)cyclohexyl)methyl methanesulfonate (1080 mg, 3.51 mmol) and benzyl amine (3.45 mL, 31.62 mmol) was heated at 100° C. for 18 h. The mixture was concentrated and the residue was partitioned between NaHCO₃ (aq, sat) and EtOAc. The layers were separated and the organic phase dried (Na₂SO₄) and concentrated. The residue was crystallized from Et₂O/hexane (1/5, 10 mL). The solid was filtered off and washed with hexane. The filtrate was concentrated and the residue was purified by chromatography. The total yield of the sub-title compound was (950 mg, 85%).

### (d) tert-Butyl ((1R,4r)-4-((benzyl((R)-2-(3-fluorophenyl)-2-hydroxyethyl)amino)-methyl)cyclohexyl)carbamate

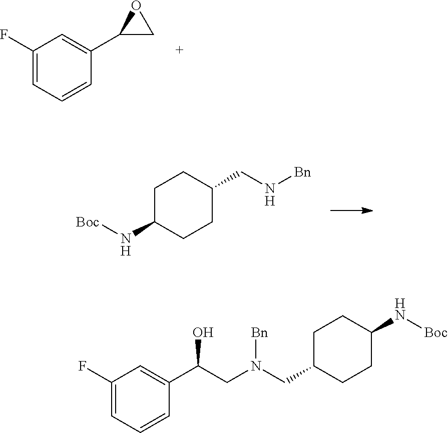 [SMILES: Fc1cccc([C@@H]2CO2)c1 ; CC(C)(C)OC(=O)N[C@H]1CC[C@H](CNCc2ccccc2)CC1 ; CC(C)(C)OC(=O)N[C@H]1CC[C@H](CN(Cc2ccccc2)C[C@H](O)c2cccc(F)c2)CC1]

The sub-title compound was prepared from tert-butyl ((1r,4r)-4-((benzylamino)-methyl)cyclohexyl)carbamate and (R)-2-(3-fluorophenyl)oxirane in accordance with the procedure in Example 1, Step (e).

### (e) (R)-2-((((1r,4R)-4-Aminocyclohexyl)methyl)(benzyl)amino)-1-(3-fluorophenyl)-ethan-1-ol

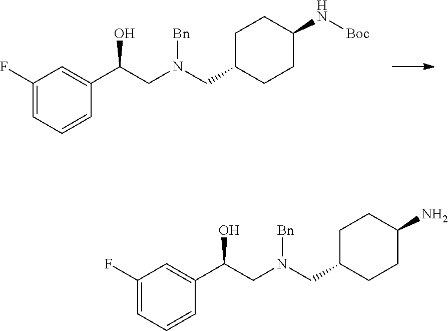 [SMILES: CC(C)(C)OC(=O)N[C@H]1CC[C@H](CN(Cc2ccccc2)C[C@H](O)c2cccc(F)c2)CC1 ; N[C@H]1CC[C@H](CN(Cc2ccccc2)C[C@H](O)c2cccc(F)c2)CC1]

Trifluoroacetic acid (0.82 mL, 10.73 mmol) was added to a solution of tert-butyl ((1R,4r)-4-((benzyl((R)-2-(3-fluorophenyl)-2-hydroxyethyl)amino)methyl)cyclohexyl)carbamate (490 mg, 1.07 mmol) in CH₂Cl₂(2.5 mL) at rt. The mixture was stirred at rt for 1 h and concentrated. The residue was treated with CH₂Cl₂, and NaHCO₃(aq, sat) was added. The mixture was stirred at rt for 15 min and the layers were separated. The aq phase was extracted with CH₂Cl₂ and the combined organic phases were dried (Na₂SO₄) and concentrated. The residue was purified by chromatography to give the sub-title compound (315 mg, 82%).

### (f) N-((1R,4r)-4-((Benzyl((R)-2-(3-fluorophenyl)-2-hydroxyethyl)amino)methyl)-cyclohexyl)acetamide

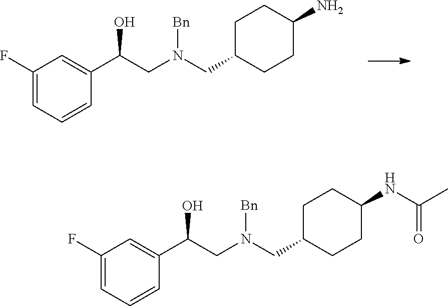 [SMILES: N[C@H]1CC[C@H](CN(Cc2ccccc2)C[C@H](O)c2cccc(F)c2)CC1 ; CC(=O)N[C@H]1CC[C@H](CN(Cc2ccccc2)C[C@H](O)c2cccc(F)c2)CC1]

Triethylamine (19 mL, 0.14 mmol) and acetyl chloride (10 μL, 0.14 mmol) were added to a stirred ice-cooled solution of (R)-2-((((1r,4R)-4-aminocyclohexyl)methyl)(benzyl)-amino)-1-(3-fluorophenyl)ethan-1-ol (50 mg, 0.14 mmol) in CH₂Cl₂ (0.4 mL). The cooling bath was removed and the mixture was stirred at rt for 1 h. NaHCO₃(aq, sat) was added and the mixture was extracted with CH₂Cl₂. The combined extracts were dried (Na₂SO₄) and concentrated. The residue was purified by chromatography to give the sub-title compound (45 mg, 81%).

### (g) N-((1R,4r)-4-((((R)-2-(3-fluorophenyl)-2-hydroxyethyl)amino)methyl)cyclohexyl)-acetamide

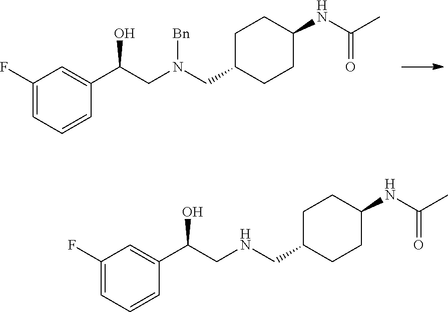 [SMILES: CC(=O)N[C@H]1CC[C@H](CN(Cc2ccccc2)C[C@H](O)c2cccc(F)c2)CC1 ; CC(=O)N[C@H]1CC[C@H](CNC[C@H](O)c2cccc(F)c2)CC1]

Triethylsilane (0.16 mL, 1.00 mmol) was added dropwise to a stirred mixture of N-((1R,4r)-4-((benzyl((R)-2-(3-fluorophenyl)-2-hydroxyethyl)amino)methyl)cyclo-hexyl)acetamide (40 mg, 0.10 mmol), Pd/C (10%, 21 mg, 0.020 mmol) and MeOH (0.3 mL) at rt. The mixture was stirred at rt for 1 h and filtered through a pad of Celite, which was washed with MeOH. The combined filtrates were concentrated and the residue was purified by chromatography using amino functionalized silica gel to give the title compound (22 mg, 71%).

¹H NMR (400 MHz, CD₃OD) δ 7.45-7.36 (m, 1H), 7.30-7.18 (m, 2H), 7.11-7.01 (m, 1H), 5.05 (dd, J=10.4, 3.2 Hz, 1H), 3.72-3.59 (m, 1H), 3.25 (dd, J=12.8, 2.8 Hz, 1H), 3.15-3.04 (m, 1H), 3.00-2.95 (m, 2H), 2.02-1.87 (m, 7H), 1.85-1.72 (m, 1H), 1.40-1.25 (m, 2H), 1.25-1.10 (m, 2H).

### Example 68: N-((1R,4r)-4-((((R)-2-(5-Fluoropyridin-3-yl)-2-hydroxyethyl)amino)-methyl)cyclohexyl)acetamide

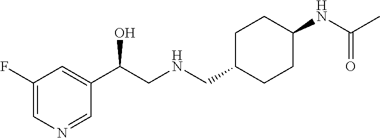 [SMILES: CC(=O)N[C@H]1CC[C@H](CNC[C@H](O)c2cncc(F)c2)CC1]

The title compound was prepared in accordance with the procedures in Example 67, using (R)-3-fluoro-5-(oxiran-2-yl)pyridine in Step (d).

¹H NMR (400 MHz, CD₃OD) δ 8.63-8.43 (m, 2H), 7.88-7.70 (m, 1H), 5.15 (dd, J=10.4, 3.2 Hz, 1H), 3.69-3.55 (m, 1H), 3.34-3.33 (m, 1H), 3.24-3.13 (m, 1H), 3.02-2.97 (m, 2H), 2.03-1.86 (m, 7H), 1.83-1.72 (m, 1H), 1.36-1.13 (m, 4H).

### Example 69: N-((1R,4r)-4-((((R)-2-(3-Fluorophenyl)-2-hydroxyethyl)amino)-methyl)cyclohexyl)benzamide

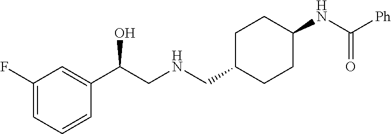 [SMILES: O=C(N[C@H]1CC[C@H](CNC[C@H](O)c2cccc(F)c2)CC1)c1ccccc1]

### (a) N-((1R,4r)-4-((Benzyl((R)-2-(3-fluorophenyl)-2-hydroxyethyl)amino)methyl)-cyclohexyl)benzamide

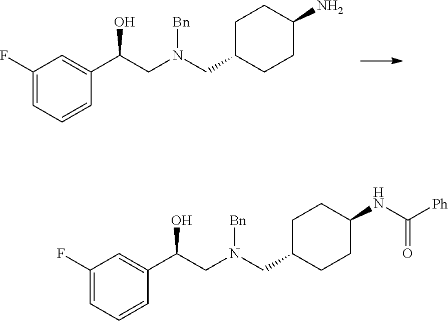 [SMILES: N[C@H]1CC[C@H](CN(Cc2ccccc2)C[C@H](O)c2cccc(F)c2)CC1 ; O=C(N[C@H]1CC[C@H](CN(Cc2ccccc2)C[C@H](O)c2cccc(F)c2)CC1)c1ccccc1]

Triethylamine (25 μL, 0.18 mmol) and benzoyl chloride (18 μL, 0.15 mmol) were added to an ice-cooled solution of (R)-2-((((1r,4R)-4-aminocyclohexyl)methyl)(benzyl)amino)-1-(3-fluorophenyl)ethan-1-ol, see Example 67, Step (e), (50 mg, 0.14 mmol) in CH₂Cl₂ (0.4 mL). The cooling bath was removed and the mixture was stirred at rt for 1 h. NaHCO₃(aq, sat) was added and the mixture was extracted with CH₂Cl₂. The combined extracts were dried (Na₂SO₄) and concentrated. The residue was purified by chromatography to give the sub-title compound (51 mg, 79%).

### (b) N-((1R,4r)-4-((((R)-2-(3-Fluorophenyl)-2-hydroxyethyl)amino)methyl)cyclo-hexyl)benzamide

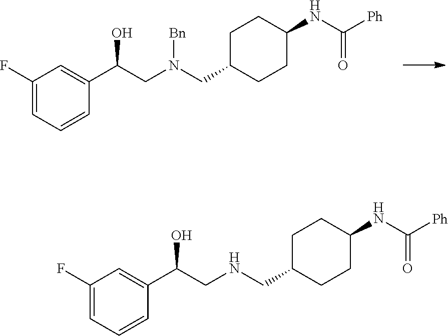 [SMILES: O=C(N[C@H]1CC[C@H](CN(Cc2ccccc2)C[C@H](O)c2cccc(F)c2)CC1)c1ccccc1 ; O=C(N[C@H]1CC[C@H](CNC[C@H](O)c2cccc(F)c2)CC1)c1ccccc1]

A mixture of N-((1R,4r)-4-((benzyl((R)-2-(3-fluorophenyl)-2-hydroxyethyl)amino)-methyl)cyclohexyl)benzamide (41 mg, 0.089 mmol), Pd/C (10%, 9 mg, 0.01 mmol) and iPrOH (0.4 mL) was hydrogenated at ambient pressure an temperature for 24 h. The mixture was filtered through a pad of Celite pad, which was washed with iPrOH. The combined filtrates were concentrated and the residue was purified by chromatography to give the title compound (26 mg, 79%).

¹H NMR (400 MHz, CD₃OD) δ 7.83-7.76 (m, 2H), 7.56-7.47 (m, 1H), 7.48-7.40 (m, 2H), 7.39-7.31 (m, 1H), 7.20-7.10 (m, 2H), 7.04-6.95 (m, 1H), 4.83-4.78 (m, 1H), 3.92-3.79 (m, 1H), 2.79-2.69 (m, 2H), 2.61-2.43 (m, 2H), 2.06-1.98 (m, 2H), 1.93-1.84 (m, 2H), 1.64-1.33 (m, 3H), 1.20-1.00 (m, 2H).

### Example 70: N-((1R,4r)-4-((((R)-2-(5-Fluoropyridin-3-yl)-2-hydroxyethyl)amino)methyl)-cyclohexyl)benzamide

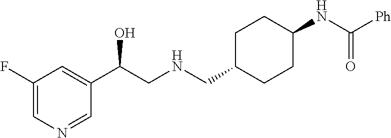 [SMILES: O=C(N[C@H]1CC[C@H](CNC[C@H](O)c2cncc(F)c2)CC1)c1ccccc1]

The title compound was prepared in accordance with the procedures in Example 67, Steps (a) to (e) using (R)-3-fluoro-5-(oxiran-2-yl)pyridine in Step (d), followed by the procedures in Example 69.

¹H NMR (400 MHz, CD₃OD) δ 8.50-8.47 (m, 1H), 8.46-8.41 (m, 1H), 7.85-7.77 (m, 2H), 7.78-7.69 (m, 1H), 7.59-7.47 (m, 1H), 7.49-7.40 (m, 2H), 5.09 (dd, J=9.8, 3.4 Hz, 1H), 3.94-3.77 (m, 1H), 3.18 (dd, J=12.6, 3.4 Hz, 1H), 3.13-3.00 (m, 1H), 2.88 (d, J=7.2 Hz, 2H), 2.11-2.03 (m, 2H), 2.02-1.88 (m, 2H), 1.83-1.63 (m, 1H), 1.59-1.33 (m, 2H), 1.29-1.17 (m, 2H).

### Example 71: N-((1R,4r)-4-((((R)-2-(3-Fluorophenyl)-2-hydroxyethyl)amino)methyl)-cyclohexyl)methanesulfonamide

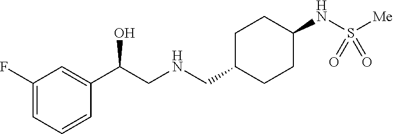 [SMILES: CS(=O)(=O)N[C@H]1CC[C@H](CNC[C@H](O)c2cccc(F)c2)CC1]

### (a) N-((1R,4r)-4-((Benzyl((R)-2-(3-fluorophenyl)-2-hydroxyethyl)amino)methyl)-cyclohexyl)methanesulfonamide

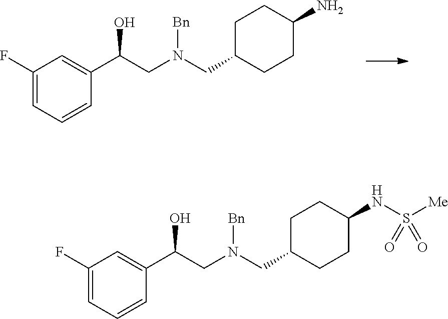 [SMILES: N[C@H]1CC[C@H](CN(Cc2ccccc2)C[C@H](O)c2cccc(F)c2)CC1 ; CS(=O)(=O)N[C@H]1CC[C@H](CN(Cc2ccccc2)C[C@H](O)c2cccc(F)c2)CC1]

Triethylamine (25 μL, 0.18 mmol) and methanesulfonyl chloride (12 μL, 0.15 mmol)) were added to an ice-cooled solution of (R)-2-((((1r,4R)-4-aminocyclohexyl)methyl)-(benzyl)amino)-1-(3-fluorophenyl)ethan-1-ol, see Example 67, Step (e), (50 mg, 0.14 mmol) in CH₂Cl₂ (1.0 mL). The cooling bath was removed and the mixture was stirred at rt for 1 h. NaHCO₃(aq, sat) was added and the mixture was extracted with CH₂Cl₂. The combined extracts were dried (Na₂SO₄) and concentrated. The residue was purified by chromatography to give the sub-title compound (39 mg, 64%).

### (b) N-((1R,4r)-4-((((R)-2-(3-Fluorophenyl)-2-hydroxyethyl)amino)methyl)cyclo-hexyl)methanesulfonamide

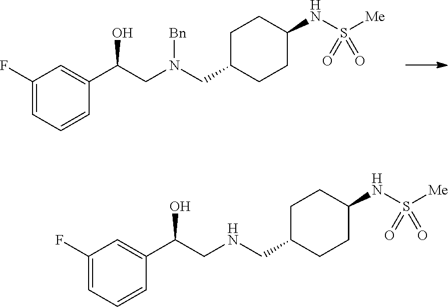 [SMILES: CS(=O)(=O)N[C@H]1CC[C@H](CN(Cc2ccccc2)C[C@H](O)c2cccc(F)c2)CC1 ; CS(=O)(=O)N[C@H]1CC[C@H](CNC[C@H](O)c2cccc(F)c2)CC1]

The title compound was prepared from N-((1R,4r)-4-((benzyl((R)-2-(3-fluorophenyl)-2-hydroxyethyl)amino)methyl)cyclohexyl)methanesulfonamide in accordance with the procedure in Example 67, Step (g).

¹H NMR (400 MHz, CD₃OD) δ 7.42-7.29 (m, 1H), 7.22-7.06 (m, 2H), 7.05-6.92 (m, 1H), 4.82-4.73 (m, 1H), 3.22-3.08 (m, 1H), 2.94 (s, 3H), 2.78-2.67 (m, 2H), 2.58-2.40 (m, 2H), 2.13-1.97 (m, 2H), 1.91-1.75 (m, 2H), 1.55-1.38 (m, 1H), 1.39-1.22 (m, 2H), 1.12-0.94 (m, 2H).

### Example 72: N-((1R,4r)-4-((((R)-2-(5-Fluoropyridin-3-yl)-2-hydroxyethyl)amino)methyl)-cyclohexyl)methanesulfonamide

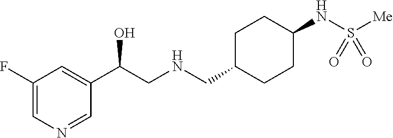 [SMILES: CS(=O)(=O)N[C@H]1CC[C@H](CNC[C@H](O)c2cncc(F)c2)CC1]

The title compound was prepared in accordance with the procedures in Example 67, Steps (a) to (e) using (R)-3-fluoro-5-(oxiran-2-yl)pyridine in Step (d), followed by the procedures in Example 71.

¹H NMR (400 MHz, CD₃OD) δ 8.43-8.41 (m, 1H), 8.36 (d, J=2.7 Hz, 1H), 7.74-7.58 (m, 1H), 4.93-4.85 (m, 1H), 3.25-3.08 (m, 1H), 2.94 (s, 3H), 2.84-2.72 (m, 2H), 2.58-2.43 (m, 2H), 2.10-1.99 (m, 2H), 1.92-1.76 (m, 2H), 1.53-1.38 (m, 1H), 1.38-1.21 (m, 2H), 1.15-0.97 (m, 2H).

Examples 73 to 78: The title compounds were prepared from (1s,4s)-4-((tert-butoxycarbonyl)amino)cyclohexane-1-carboxylic acid in accordance with the procedures in Example 67 to 72.

### Example 73: N-((1S,4s)-4-((((R)-2-(3-Fluorophenyl)-2-hydroxyethyl)amino)-methyl)cyclohexyl)acetamide

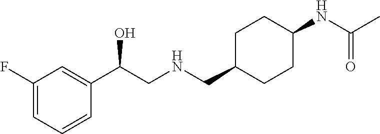 [SMILES: CC(=O)N[C@H]1CC[C@@H](CNC[C@H](O)c2cccc(F)c2)CC1]

¹H NMR (400 MHz, CD₃OD) δ 7.48-7.38 (m, 1H), 7.32-7.18 (m, 2H), 7.14-6.99 (m, 1H), 5.04 (dd, J=10.6, 3.0 Hz, 1H), 3.97-3.88 (m, 1H), 3.26 (dd, J=12.6, 3.0 Hz, 1H), 3.16-3.01 (m, 3H), 1.98 (s, 3H), 1.95-1.86 (m, 1H), 1.79-1.60 (m, 6H), 1.58-1.40 (m, 2H).

### Example 74: N-((1S,4s)-4-((((R)-2-(5-Fluoropyridin-3-yl)-2-hydroxyethyl)amino)-methyl)cyclohexyl)acetamide

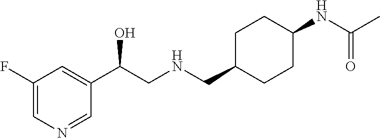 [SMILES: CC(=O)N[C@H]1CC[C@@H](CNC[C@H](O)c2cncc(F)c2)CC1]

¹H NMR (400 MHz, CD₃OD) δ 8.51-8.49 (m, 1H), 8.47-8.44 (m, 1H), 7.81-7.71 (m, 1H), 5.14 (dd, J=10.2, 3.0 Hz, 1H), 3.95-3.89 (m, 1H), 3.27 (d, J=3.2 Hz, 1H), 3.22-3.09 (m, 1H), 3.05-3.00 (m, 2H), 1.96 (s, 3H), 1.93-1.86 (m, 1H), 1.79-1.59 (m, 6H), 1.59-1.41 (m, 2H).

### Example 75: N-((1S,4s)-4-((((R)-2-(3-Fluorophenyl)-2-hydroxyethyl)amino)methyl)-cyclohexyl)benzamide

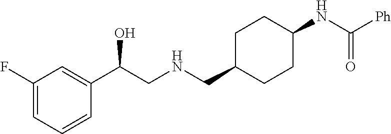 [SMILES: O=C(N[C@H]1CC[C@@H](CNC[C@H](O)c2cccc(F)c2)CC1)c1ccccc1]

¹H NMR (400 MHz, CD₃OD) δ 7.82-7.74 (m, 2H), 7.56-7.47 (m, 1H), 7.49-7.39 (m, 2H), 7.40-7.28 (m, 1H), 7.21-7.10 (m, 2H), 7.05-6.91 (m, 1H), 4.81 (dd, J=7.0, 5.8 Hz, 1H), 4.14-3.98 (m, 1H), 2.79-2.72 (m, 2H), 2.72-2.58 (m, 2H), 1.83-1.61 (m, 7H), 1.61-1.50 (m, 2H).

### Example 76: N-((1S,4s)-4-((((R)-2-(5-Fluoropyridin-3-yl)-2-hydroxyethyl)amino)methyl)-cyclohexyl)benzamide

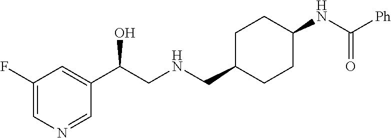 [SMILES: O=C(N[C@H]1CC[C@@H](CNC[C@H](O)c2cncc(F)c2)CC1)c1ccccc1]

¹H NMR (400 MHz, CD₃OD) δ 8.50-8.47 (m, 1H), 8.43 (d, J=2.8 Hz, 1H), 7.84-7.77 (m, 2H), 7.78-7.71 (m, 1H), 7.57-7.48 (m, 1H), 7.50-7.41 (m, 2H), 5.10 (dd, J=10.0, 3.2 Hz, 1H), 4.14-4.01 (m, 1H), 3.19 (dd, J=12.6, 3.4 Hz, 1H), 3.14-2.92 (m, 3H), 2.01-1.89 (m, 1H), 1.87-1.70 (m, 6H), 1.68-1.57 (m, 2H).

### Example 77: N-((1S,4s)-4-((((R)-2-(5-Fluoropyridin-3-yl)-2-hydroxyethyl)amino)methyl)-cyclohexyl)benzamide

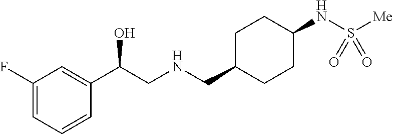 [SMILES: CS(=O)(=O)N[C@H]1CC[C@@H](CNC[C@H](O)c2cccc(F)c2)CC1]

¹H NMR (400 MHz, CD₃OD) δ 7.42-7.29 (m, 1H), 7.24-7.08 (m, 2H), 7.06-6.93 (m, 1H), 4.83-4.75 (m, 1H), 3.58-3.49 (m, 1H), 2.94 (s, 3H), 2.80-2.69 (m, 2H), 2.64-2.47 (m, 2H), 1.81-1.71 (m, 2H), 1.71-1.56 (m, 5H), 1.49-1.32 (m, 2H).

### Example 78: N-((1S,4s)-4-((((R)-2-(5-Fluoropyridin-3-yl)-2-hydroxyethyl)amino)methyl)-cyclohexyl)methanesulfonamide

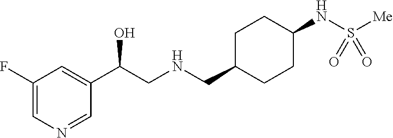 [SMILES: CS(=O)(=O)N[C@H]1CC[C@@H](CNC[C@H](O)c2cncc(F)c2)CC1]

¹H NMR (400 MHz, CD₃OD) δ 8.43-8.41 (m, 1H), 8.37 (d, J=2.7 Hz, 1H), 7.72-7.63 (m, 1H), 4.89 (dd, J=7.8, 5.0 Hz, 1H), 3.61-3.48 (m, 1H), 2.94 (s, 3H), 2.88-2.75 (m, 2H), 2.66-2.47 (m, 2H), 1.81-1.69 (m, 2H), 1.68-1.56 (m, 5H), 1.48-1.33 (m, 2H).

### Example 79: (R)-2-((2-((1r,4R)-4-Aminocyclohexyl)propan-2-yl)amino)-1-(3-fluoro-phenyl)ethan-1-ol

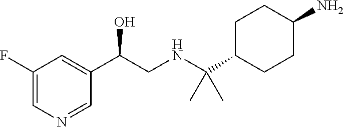 [SMILES: CC(C)(NC[C@H](O)c1cncc(F)c1)[C@H]1CC[C@H](N)CC1]

### (a) tert-Butyl ((1r,4r)-4-carbamoylcyclohexyl)carbamate

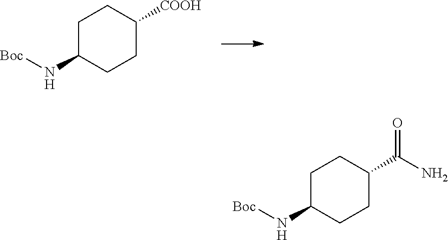 [SMILES: CC(C)(C)OC(=O)N[C@H]1CC[C@H](C(=O)O)CC1 ; CC(C)(C)OC(=O)N[C@H]1CC[C@H](C(N)=O)CC1]

Triethylamine (1.15 mL, 8.22 mmol) and pivaloyl chloride (607 μL, 4.93 mmol) were added to an ice-cooled solution of (1r,4r)-4-((tert-butoxycarbonyl)amino)cyclohexane-1-carboxylic acid (1 g, 4.11 mmol) in CHCl₃ (13 mL). The mixture was stirred at 0° C. for 1 h and NH₄OH (aq, 25%, 3.08 mL, 41.10 mmol) was added. The mixture was stirred at rt for 2 h and concentrated. The residue was suspended in MeCN. The mixture was filtered and the solids were washed with H₂O and petroleum ether and dried to give the sub-title compound (617 mg, 62%).

### (b) tert-Butyl ((1r,4r)-4-cyanocyclohexyl)carbamate

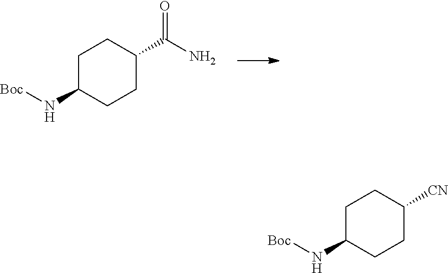 [SMILES: CC(C)(C)OC(=O)N[C@H]1CC[C@H](C(N)=O)CC1 ; CC(C)(C)OC(=O)N[C@H]1CC[C@H](C#N)CC1]

POCl₃ (1.15 mL, 12.38 mmol) was added dropwise to a stirred ice cooled solution of tert-butyl ((1r,4r)-4-carbamoylcyclohexyl)carbamate (600 mg, 2.48 mmol) in pyridine (10 mL). The mixture was stirred at 0° C. for 30 min and poured onto ice/H₂O. The mixture was extracted with EtOAc and the combined extracts were washed with H₂O and brine, dried (Na₂SO₄) and concentrated to give the sub-title compound (440 mg, 79%).

### (c) tert-Butyl ((1r,4r)-4-(2-aminopropan-2-yl)cyclohexyl)carbamate

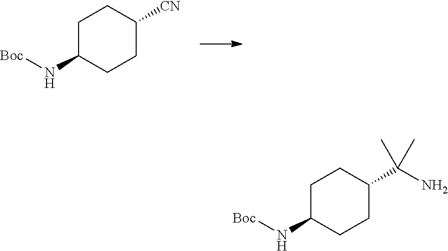 [SMILES: CC(C)(C)OC(=O)N[C@H]1CC[C@H](C#N)CC1 ; CC(C)(C)OC(=O)N[C@H]1CC[C@H](C(C)(C)N)CC1]

The sub-title compound was prepared from tert-butyl ((1r,4r)-4-cyanocyclohexyl)-carbamate in accordance with the procedures in Example 13, Step (a).

### (d) tert-Butyl ((1R,4r)-4-(2-(((R)-2-(5-fluoropyridin-3-yl)-2-hydroxyethyl)amino)-propan-2-yl)cyclohexyl)carbamate

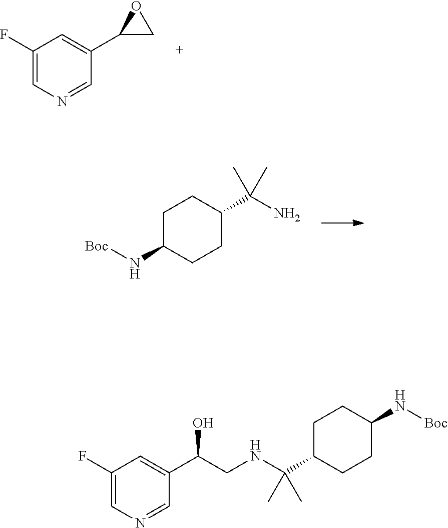 [SMILES: Fc1cncc([C@@H]2CO2)c1 ; CC(C)(C)OC(=O)N[C@H]1CC[C@H](C(C)(C)N)CC1 ; CC(C)(C)OC(=O)N[C@H]1CC[C@H](C(C)(C)NC[C@H](O)c2cncc(F)c2)CC1]

The sub-title compound was prepared from tert-butyl ((1r,4r)-4-(2-aminopropan-2-yl)cyclohexyl)carbamate and (R)-3-fluoro-5-(oxiran-2-yl)pyridine in accordance with the procedure in Example 1, Step (e).

### (e) (R)-2-((2-((1r,4R)-4-Aminocyclohexyl)propan-2-yl)amino)-1-(3-fluorophenyl)-ethan-1-ol

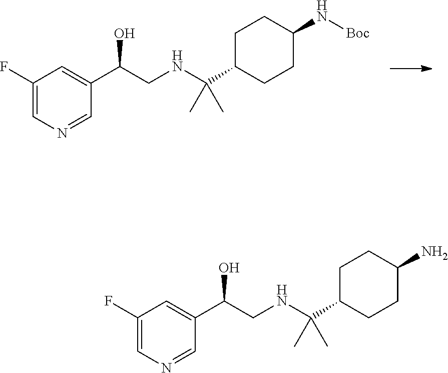 [SMILES: CC(C)(C)OC(=O)N[C@H]1CC[C@H](C(C)(C)NC[C@H](O)c2cncc(F)c2)CC1 ; CC(C)(NC[C@H](O)c1cncc(F)c1)[C@H]1CC[C@H](N)CC1]

The title compound was prepared from tert-butyl ((1R,4r)-4-(2-(((R)-2-(5-fluoropyridin-3-yl)-2-hydroxyethyl)amino)propan-2-yl)cyclohexyl)carbamate in accordance with the procedure in Example 67, Step (e).

¹H NMR (400 MHz, CDCl₃) δ 8.39-8.35 (2H, m) 7.51-7.46 (1H, m) 4.62 (1H, dd, J=3.5, 8.8 Hz) 4.41-4.31 (1H, m) 3.41-3.26 (1H, m) 2.91 (1H, dd, J=3.76, 12 Hz) 2.50 (1, dd, J=8.9, 12 Hz) 2.10-2.02 (2H, m) 1.81-1.70 (2H, m) 1.43 (9H, s) 1.27-1.01 (2H, m) 1.01 (6, d, J=3.4 Hz).

### Example 80: (R)-2-((2-((1r,4R)-4-Aminocyclohexyl)propan-2-yl)amino)-1-(3-fluoro-phenyl)ethan-1-ol

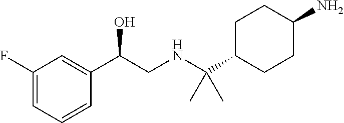 [SMILES: CC(C)(NC[C@H](O)c1cccc(F)c1)[C@H]1CC[C@H](N)CC1]

The title compound was prepared in accordance with the procedure in Example 79 using (R)-2-(3-fluorophenyl)oxirane in Step (d).

¹H NMR (400 MHz, CDCl₃) δ 7.33-7.27 (1H, m) 7.14-7.08 (2H, m) 6.98-6.92 (1H, m) 4.56 (1H, dd, J=3.7, 8.7 Hz) 2.87 (1H, dd, J=3.8, 11.9 Hz) 2.62-2.54 (1H, m) 2.51 (1H, dd, J=8.7, 11.9 Hz) 2.01-1.87 (2H, m) 1.78-1.70 (2H, m) 1.25-1.04 (5H, m) 1.01 (6H, d, J=3.3 Hz).

### Example 81: N-((1R,4r)-4-(2-(((R)-2-(5-Fluoropyridin-3-yl)-2-hydroxyethyl)amino)-propan-2-yl)cyclohexyl)acetamide

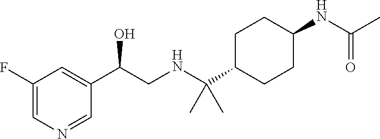 [SMILES: CC(=O)N[C@H]1CC[C@H](C(C)(C)NC[C@H](O)c2cncc(F)c2)CC1]

The title compound was prepared from (R)-2-((2-((1r,4R)-4-aminocyclohexyl)propan-2-yl)amino)-1-(3-fluorophenyl)ethan-1-ol, see Example 79, in accordance with the procedure in Example 67, Step (f).

¹H NMR (400 MHz, CDCl₃) δ 8.40-8.37 (2H, m) 7.52-7.47 (1H, m) 5.23 (1H, d, J=8.0 Hz) 4.62 (1H, dd, J=3.8, 8.0 Hz) 3.75-3.64 (1H, m) 2.92 (1H, dd, 3.7, 12.,0 Hz) 2.51 (1H, dd, J=9.0, 12.0) 2.10-2.02 (2H, m) 1.83-1.74 (2H, m) 1.26-1.04 (5H, m) 1.02 (6H, d, J=3.5 Hz).

### Example 82: N-((1R,4r)-4-(2-(((R)-2-(3-Fluorophenyl)-2-hydroxyethyl)amino)propan-2-yl)cyclohexyl)acetamide

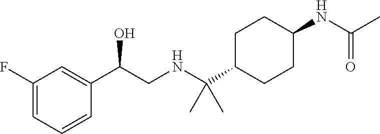 [SMILES: CC(=O)N[C@H]1CC[C@H](C(C)(C)NC[C@H](O)c2cccc(F)c2)CC1]

The title compound was prepared from (R)-2-((2-((1r,4R)-4-aminocyclohexyl)propan-2-yl)amino)-1-(5-fluoropyridin-3-yl)ethan-1-ol, see Example 80, in accordance with the procedure in Example 67, Step (f).

¹H NMR (400 MHz, CDCl₃) δ 7.33-7.27 (1H, m) 7.15-7.09 (2H, m) 6.99-6.92 (1H, m) 5.30 (1H, d, J=7.7 Hz) 4.70 (1H, dd, J=3.3, 8.8 Hz) 3.73-3.62 (1H, m) 2.92 (1H, dd, J=3.5, 11.9 Hz) 2.9-2.7 (2H, br s) 2.57 (1H, dd, J=8.8, 11.9 Hz) 2.08-2.00 (2H, m) 1.94 (3H, s) 1.83-1.75 (2H, m) 1.33-1.01 (5H, m) 1.06 (6H, d, J=5.2 Hz).

### Example 83: N-((1R,4r)-4-(2-(((R)-2-(5-Fluoropyridin-3-yl)-2-hydroxyethyl)amino)-propan-2-yl)cyclohexyl)methanesulfonamide

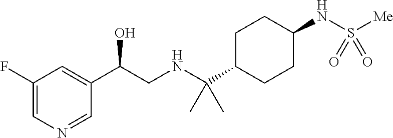 [SMILES: CC(C)(NC[C@H](O)c1cncc(F)c1)[C@H]1CC[C@H](NS(C)(=O)=O)CC1]

The title compound was prepared from (R)-2-((2-((1r,4R)-4-aminocyclohexyl)propan-2-yl)amino)-1-(5-fluoropyridin-3-yl)ethan-1-ol, see Example 79, in accordance with the procedure in Example 71, Step (a).

¹H NMR (400 MHz, CDCl₃) δ 8.41-8.37 (2H, m) 7.53-7.48 (1H, m) 4.74 (1H, dd, J=3, 6, 9 Hz) 4.17 (1H, d, J=6.7 Hz) 3.42-3.17 (3H, m) 2.98 (3H, s) 2.95 (1H, dd, J=3.7, 12 Hz) 2.56 (1H, dd, J=9, 12 Hz) 2.19-2.11 (1H, m) 1.87-1.77 (1H, m) 1.31-1.11 (6H, m) 1.07 (6H, d, J=4.2 Hz).

### Example 84: N-((1R,4r)-4-(2-(((R)-2-(3-Fluorophenyl)-2-hydroxyethyl)amino)propan-2-yl)cyclohexyl)benzamide

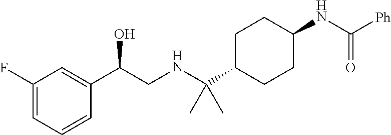 [SMILES: CC(C)(NC[C@H](O)c1cccc(F)c1)[C@H]1CC[C@H](NC(=O)c2ccccc2)CC1]

The title compound was prepared from (R)-2-((2-((1r,4R)-4-aminocyclohexyl)propan-2-yl)amino)-1-(5-fluoropyridin-3-yl)ethan-1-ol, see Example 80, in accordance with the procedure in Example 69, Step (a).

¹H NMR (400 MHz, CDCl₃) δ 7.74-7.69 (2H, m) 7.51-7.26 (4H, m) 7.22-7.17 (2H, m) 6.98-6.92 (1H, m) 5.96-5.88 (1H, m) 5.23 (1H, d, J=8.9 Hz) 3.95-3.83 (1H, m) 3.16-3.06 (3H, m) 2.81 (1H, dd, J=9.9, 11.9 Hz) 2.21-2.13 (2H, m) 2.0-1.90 (2H, m) 1.41-1.12 (13H, m).

### Example 85: (R)-2-((1-((1r,4R)-4-Aminocyclohexyl)-2-methylpropan-2-yl)amino)-1-(5-fluoropyridin-3-yl)ethan-1-ol

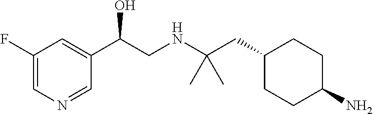 [SMILES: CC(C)(C[C@H]1CC[C@H](N)CC1)NC[C@H](O)c1cncc(F)c1]

### (a) tert-Butyl ((1r,4r)-4-(cyanomethyl)cyclohexyl)carbamate

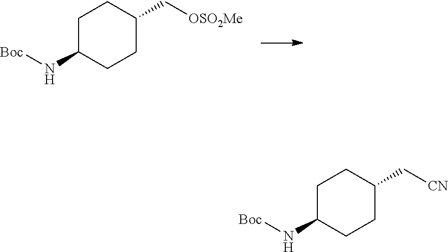

NaCN (576 mg, 11.75 mmol) was added to a solution of ((1r,4r)-4-((tert-butoxy-carbonyl)amino)cyclohexyl)methyl methanesulfonate, see Example 67, Step (c), (1.20 g, 3.92 mmol) in DMSO (14.5 mL). The mixture was stirred at 90° C. for 4 h and cooled to rt. Brine was added and the mixture was extracted with EtOAc. The combined extracts were washed with brine, dried (Na₂SO₄) and concentrated. The residue was purified by chromatography to give the sub-title compound (871 mg, 93%).

### (b) tert-Butyl ((1r,4r)-4-(2-amino-2-methylpropyl)cyclohexyl)carbamate

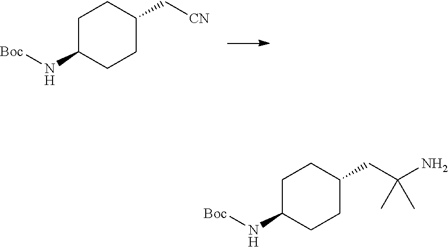 [SMILES: CC(C)(C)OC(=O)N[C@H]1CC[C@H](CC#N)CC1 ; CC(C)(N)C[C@H]1CC[C@H](NC(=O)OC(C)(C)C)CC1]

The sub-title compound was prepared from tert-butyl ((1r,4r)-4-(cyanomethyl)-cyclohexyl)carbamate in accordance with the procedures in Example 13, Step (a).

### (c) tert-butyl ((1R,4r)-4-(2-(((R)-2-(3-Fluorophenyl)-2-hydroxyethyl)amino)-2-methylpropyl)cyclohexyl)carbamate

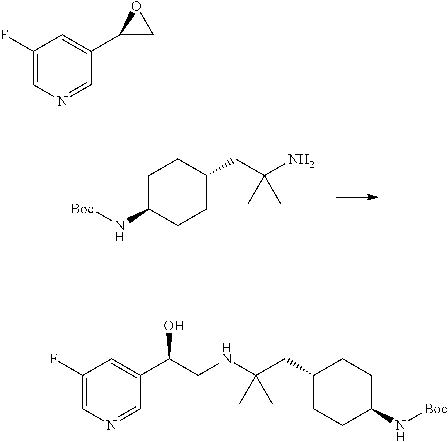 [SMILES: Fc1cncc([C@@H]2CO2)c1 ; CC(C)(N)C[C@H]1CC[C@H](NC(=O)OC(C)(C)C)CC1 ; CC(C)(C[C@H]1CC[C@H](NC(=O)OC(C)(C)C)CC1)NC[C@H](O)c1cncc(F)c1]

The sub-title compound was prepared from tert-butyl ((1r,4r)-4-(2-amino-2-methyl-propyl)cyclohexyl)carbamate and (R)-3-fluoro-5-(oxiran-2-yl)pyridine in accordance with the procedure in Example 1, Step (e).

### (d) (R)-2-((1-((1r,4R)-4-Aminocyclohexyl)-2-methylpropan-2-yl)amino)-1-(5-fluoro-pyridin-3-yl)ethan-1-ol

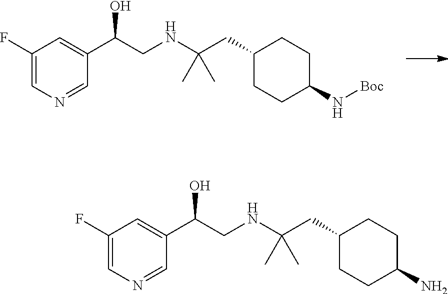 [SMILES: CC(C)(C[C@H]1CC[C@H](NC(=O)OC(C)(C)C)CC1)NC[C@H](O)c1cncc(F)c1 ; CC(C)(C[C@H]1CC[C@H](N)CC1)NC[C@H](O)c1cncc(F)c1]

The title compound was prepared from tert-butyl ((1R,4r)-4-(2-(((R)-2-(5-fluoropyridin-3-yl)-2-hydroxyethyl)amino)-2-methylpropyl)cyclohexyl)carbamate in accordance with the procedure in Example 67, Step (e).

¹H NMR (400 MHz, CDCl₃) δ 8.38-8.35 (2H, m) 7.50-7.46 (1H, m) 4.62 (1H, dd, J=3.8, 8.8 Hz) 2.89 (1H, dd, J=3.8, 12.0 Hz) 2.58-2.51 (1H, m) 2.52 (1H, dd, J=8.8, 12.0 Hz) 1.85-1.78 (2H, m) 1.77-1.71 (2H, m) 1.30-1.21 (3H, m) 1.12-0.98 (4H, m) 1.06 (6H, s).

### Example 86: (R)-2-((1-((1r,4R)-4-Aminocyclohexyl)-2-methylpropan-2-yl)amino)-1-(3-fluorophenyl)ethan-1-ol

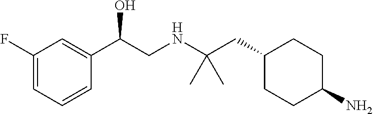 [SMILES: CC(C)(C[C@H]1CC[C@H](N)CC1)NC[C@H](O)c1cccc(F)c1]

The title compound was prepared in accordance with the procedure in Example 85 using (R)-2-(3-fluorophenyl)oxirane in Step (c).

¹H NMR (400 MHz, CDCl₃) δ 7.32-7.26 (1H, m) 7.13-7.07 (2H, m) 6.97-6.91 (1H, m) 4.57 (1H, dd, J=3.7, 8.5 Hz) 2.86 (1H, dd, J=3.7, 11.9 Hz) 2.59-2.50 (1H, m) 2.53 (1H, dd, J=8.7, 11.9 Hz) 2.0-1.9 (2H, br s) 1.84-1.77 (2H, m) 1.77-1.69 (2H, m) 1.32-1.19 (3H, m) 1.12-0.96 (4H, m) 1.05 (6H, s).

### Example 87: N-((1R,4r)-4-(2-(((R)-2-(5-Fluoropyridin-3-yl)-2-hydroxyethyl)amino)-2-methylpropyl)cyclohexyl)acetamide

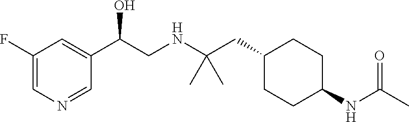 [SMILES: CC(=O)N[C@H]1CC[C@H](CC(C)(C)NC[C@H](O)c2cncc(F)c2)CC1]

The title compound was prepared from (R)-2-((1-((1r,4R)-4-aminocyclohexyl)-2-methylpropan-2-yl)amino)-1-(5-fluoropyridin-3-yl)ethan-1-ol in accordance with the procedure in Example 67, Step (f).

¹H NMR (400 MHz, CDCl₃) δ 8.39-8.35 (2H, m) 7.50-7.46 (1H, m) 5.33 (1H, d, J=8.0 Hz) 4.66 (3.8, 8.9 Hz) 3.72-3.61 (1H, m) 3.1-2.9 (1H, br s) 2.93 (1H, dd, J=3.8, 12 Hz) 2.54 (1H, dd, J=8.8, 12.0 Hz) 1.97-1.91 (2H, m) 1.94 (3H, s) 1.80-1.73 (2H, m) 1.34-1.22 (3H, m) 1.15-1.09 (3H, m) 1.08 (6H, d, J=2.0 Hz).

### Example 88: N-((1R,4r)-4-(2-(((R)-2-(3-Fluorophenyl)-2-hydroxyethyl)amino)-2-methyl-propyl)cyclohexyl)acetamide

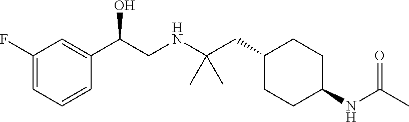 [SMILES: CC(=O)N[C@H]1CC[C@H](CC(C)(C)NC[C@H](O)c2cccc(F)c2)CC1]

The title compound was prepared from (R)-2-((1-((1r,4R)-4-aminocyclohexyl)-2-methylpropan-2-yl)amino)-1-(3-fluorophenyl)ethan-1-ol in accordance with the procedure in Example 67, Step (f).

¹H NMR (400 MHz, CDCl₃) δ 7.34-7.27 (1H, m) 7.14-7.08 (2H, m) 6.99-6.93 (1H, m) 5.22 (1H, d, J=7.8 Hz) 4.65 (1H, dd, J=3.8, 8.8 Hz) 3.73-3.62 (1H, m) 2.92 (1H, dd, J=3.8, 11.9 Hz) 1.98-1.92 (2H, m) 1.95 (3H, s) 1.82-1.74 (2H, m) 1.36-1.34 (3H, m) 1.18-1.04 (4H, m) 1.10 (6H, d, J=3.1 Hz).

### Example 89: N-((1R,4r)-4-(2-(((R)-2-(5-Fluoropyridin-3-yl)-2-hydroxyethyl)amino)-2-methylpropyl)cyclohexyl)methanesulfonamide

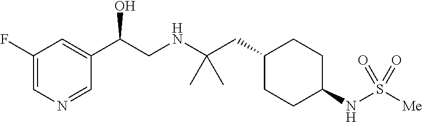 [SMILES: CC(C)(C[C@H]1CC[C@H](NS(C)(=O)=O)CC1)NC[C@H](O)c1cncc(F)c1]

The title compound was prepared from (R)-2-((1-((1r,4R)-4-aminocyclohexyl)-2-methylpropan-2-yl)amino)-1-(5-fluoropyridin-3-yl)ethan-1-ol in accordance with the procedure in Example 71, Step (a).

¹H NMR (400 MHz, CDCl₃) δ 8.39-8.36 (2H, m) 7.51-7.46 (1H, m) 4.70 (1H, dd, J=3.6, 8.9 Hz) 4.5-4.3 (1H, br s) 3.26-3.17 (1H, m) 2.96 (3H, s) 2.94 (2H, dd, J=3.8, 12.0 Hz) 2.55 (2H, dd, J=8.9, 12.0 Hz) 2.06-1.99 (2H, m) 1.84-1.77 (2H, m) 1.36-1.20 (5H, m) 1.16-1.03 (2H, m) 1.09 (6H, d, J=3.2 Hz).

### Example 90: N-((1R,4r)-4-(2-(((R)-2-(3-Fluorophenyl)-2-hydroxyethyl)amino)-2-methylpropyl)cyclohexyl)methanesulfonamide

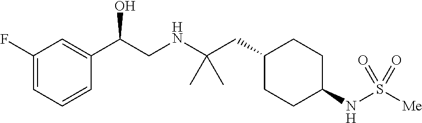 [SMILES: CC(C)(C[C@H]1CC[C@H](NS(C)(=O)=O)CC1)NC[C@H](O)c1cccc(F)c1]

The title compound was prepared from (R)-2-((1-((1r,4R)-4-aminocyclohexyl)-2-methylpropan-2-yl)amino)-1-(3-fluorophenyl)ethan-1-ol, see Example 86, in accordance with the procedure in Example 71, Step (a).

¹H NMR (400 MHz, CDCl₃) δ 7.34-7.27 (1H, m) 7.13-7.08 (2H, m) 6.99-6.93 (1H, m) 4.64 (1H, dd, J=3.7, 8.5 Hz) 4.07 (1H, d, J=6.0 Hz) 3.28-3.18 (1H, m) 2.97 (3H, s) 2.90 (1H, dd, J=3.7, 11.9 Hz) 2.56 (1H, dd, J=8.6, 11.9 Hz) 2.08-2.01 (2H, m) 1.85-1.78 (2H, m) 1.35-1.20 (5H, m) 1.16-1.04 (2H, m) 1.09 (6H, d, J=3.7 Hz).

### Example 91: N-((1R,4r)-4-(2-(((R)-2-(3-Fluorophenyl)-2-hydroxyethyl)amino)-2-methylpropyl)cyclohexyl)benzamide

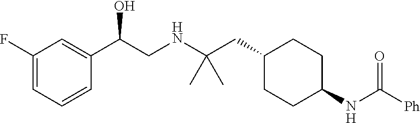 [SMILES: CC(C)(C[C@H]1CC[C@H](NC(=O)c2ccccc2)CC1)NC[C@H](O)c1cccc(F)c1]

The title compound was prepared from (R)-2-((1-((1r,4R)-4-aminocyclohexyl)-2-methylpropan-2-yl)amino)-1-(3-fluorophenyl)ethan-1-ol, see Example 86, in accordance with the procedure in Example 69, Step (a).

¹H NMR (400 MHz, CDCl₃) δ 7.73-7.72 (2H, m) 7.52-7.40 (3H, m) 7.34-7.28 (2H, m) 7.14-7.10 (2H, m) 6.99-6.93 (1H, m) 5.87 (1H, d, J=8.2 Hz) 4.65 (1H, dd, J=3.8 Hz) 3.96-3.85 (1H, m) 2.93 (1H, dd, J=3.8, 11.9 Hz) 2.57 (1H, dd, J=8.7, 11.9 Hz) 2.13-2.05 (2H, m) 1.88-1.78 (2H, m) 1.44-1.12 (7H, m) 1.11 (6H, d, J=3.1 Hz).

### Example 92: (R)-2-((4-((1r,4S)-4-Aminocyclohexyl)-2-methylbutan-2-yl)amino)-1-(5-fluoropyridin-3-yl)ethan-1-ol

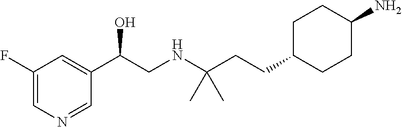 [SMILES: CC(C)(CC[C@H]1CC[C@H](N)CC1)NC[C@H](O)c1cncc(F)c1]

### (a) tert-Butyl ((1r,4r)-4-formylcyclohexyl)carbamate

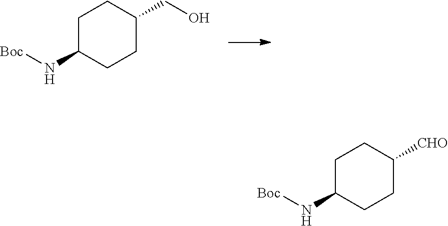 [SMILES: CC(C)(C)OC(=O)N[C@H]1CC[C@H](CO)CC1 ; CC(C)(C)OC(=O)N[C@H]1CC[C@H](C=O)CC1]

DIPEA (1.88 mL, 10.89 mmol) followed by a solution of sulfur trioxide pyridine complex (1.73, 10.89 mmol) dissolved in DMSO (7.3 mL) were added dropwise to a stirred ice-cooled solution of tert-butyl ((1r,4r)-4-(hydroxymethyl)cyclohexyl)carbamate, see Example 67, Step (a), (640 mg, 2.79 mmol) in CH₂Cl₂ (4.8 mL). The mixture was stirred at 0° C. for 2 h and HCl (aq, 1 M) was added at 0° C. The mixture was extracted with CH₂Cl₂ and the combined extracts were washed with brine and dried (Na₂SO₄). The residue was purified by chromatography to give the sub-title compound (634 mg, 99%).

### (b) tert-Butyl ((1r,4r)-4-(2-cyanovinyl)cyclohexyl)carbamate

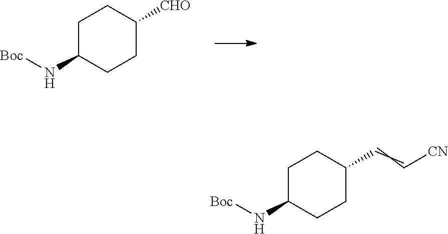 [SMILES: CC(C)(C)OC(=O)N[C@H]1CC[C@H](C=O)CC1 ; CC(C)(C)OC(=O)N[C@H]1CC[C@H](C=CC#N)CC1]

Diethyl cyanomethyl phosphonate (542 μL, 3.35 mmol) was added dropwise to an ice-cooled stirred mixture of potassium tert-butoxide (376 mg, 3.35 mmol) and THF (19 mL). The mixture was stirred at 0° C. for 1 h and a solution of tert-butyl ((1r,4r)-4-formylcyclohexyl)carbamate (634 mg, 2.79 mmol) in THF (19 mL) was slowly added. The cooling bath was removed and the mixture was stirred at rt for 1 h. EtOAc and H₂O were added and the phases were separated. The aq phase was extracted with EtOAc and the combined extracts were washed with citric acid (aq, 10%), NaHCO₃(aq, sat) and brine, dried (Na₂SO₄) and concentrated to give the sub-title compound (607 mg, 87%) as a 1:2 mixture of E- and Z-isomers.

### (c) tert-Butyl ((1r,4r)-4-(2-cyanoethyl)cyclohexyl)carbamate

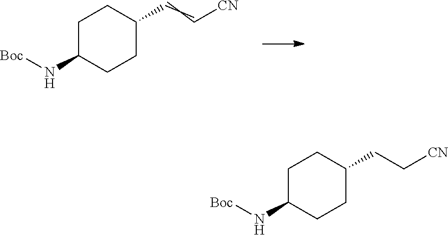 [SMILES: CC(C)(C)OC(=O)N[C@H]1CC[C@H](C=CC#N)CC1 ; CC(C)(C)OC(=O)N[C@H]1CC[C@H](CCC#N)CC1]

A mixture of tert-butyl ((1r,4r)-4-(2-cyanovinyl)cyclohexyl)carbamate (568 mg, 2.27 mmol) Pd/C (10%, 48 mg, 0.45 mmol) and EtOH (50 mL) was hydrogenated at ambient temperature and pressure for 18 h. The mixture was filtered through Celite and concentrated to give the sub-title compound (546 mg, 95%).

### (d) tert-Butyl ((1s,4r)-4-(3-amino-3-methylbutyl)cyclohexyl)carbamate

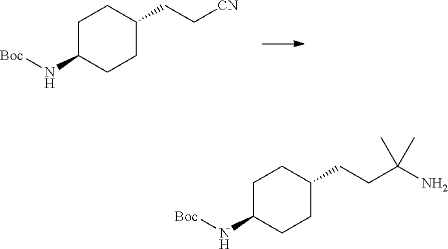 [SMILES: CC(C)(C)OC(=O)N[C@H]1CC[C@H](CCC#N)CC1 ; CC(C)(N)CC[C@H]1CC[C@H](NC(=O)OC(C)(C)C)CC1]

The title compound was prepared from tert-butyl ((1r,4r)-4-(2-cyanoethyl)cyclo-hexyl)carbamate in accordance with the procedure in Example 13, Step (a).

### (e) tert-Butyl ((1S,4r)-4-(3-(((R)-2-(5-fluoropyridin-3-yl)-2-hydroxyethyl)amino)-3-methylbutyl)cyclohexyl)carbamate

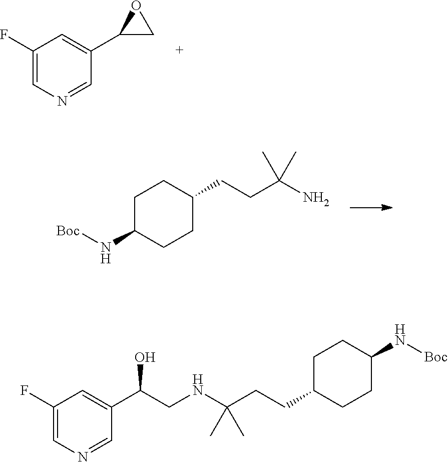 [SMILES: Fc1cncc([C@@H]2CO2)c1 ; CC(C)(N)CC[C@H]1CC[C@H](NC(=O)OC(C)(C)C)CC1 ; CC(C)(CC[C@H]1CC[C@H](NC(=O)OC(C)(C)C)CC1)NC[C@H](O)c1cncc(F)c1]

The sub-title compound was prepared from tert-butyl ((1s,4r)-4-(3-amino-3-methyl-butyl)cyclohexyl)carbamate and (R)-3-fluoro-5-(oxiran-2-yl)pyridine in accordance with the procedure in Example 1, Step (e).

### (f) (R)-2-((4-((1r,4S)-4-Aminocyclohexyl)-2-methylbutan-2-yl)amino)-1-(5-fluoropyridin-3-yl)ethan-1-ol

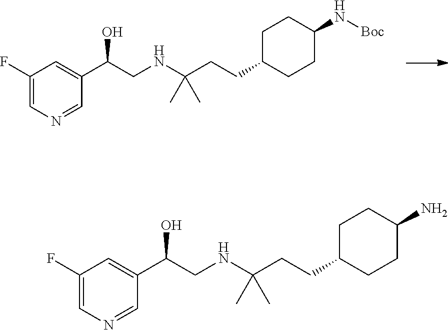 [SMILES: CC(C)(CC[C@H]1CC[C@H](NC(=O)OC(C)(C)C)CC1)NC[C@H](O)c1cncc(F)c1 ; CC(C)(CC[C@H]1CC[C@H](N)CC1)NC[C@H](O)c1cncc(F)c1]

The title compound was prepared from tert-butyl (4-(3-(((R)-2-(3-fluorophenyl)-2-hydroxyethyl)amino)-3-methylbutyl)cyclohexyl)carbamate in accordance with the procedure in Example 67, Step (e).

¹H NMR (400 MHz, CDCl₃) δ 8.33-8.29 (2H, m) 7.45-7.40 (1H, m) 4.56 (1H, dd, J=3.7, 8.8 Hz) 2.83 (1H, dd, J=3.8, 12.0 Hz) 2.56-2.47 (1H, m) 2.44 (1H, dd, J=8.9, 12.0 Hz) 1.81-1.73 (2H, m) 1.70-1.64 (2H, m) 1.32-1.25 (2H, m) 1.12-0.98 (4H, m) 0.97 (6H, s) 0.95-0.80 (2H, m).

### Example 93: N-((1S,4r)-4-(3-(((R)-2-(5-Fluoropyridin-3-yl)-2-hydroxyethyl)amino)-3-methylbutyl)cyclohexyl)acetamide

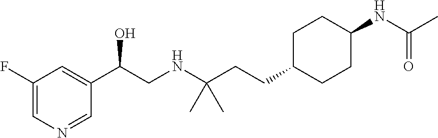 [SMILES: CC(=O)N[C@H]1CC[C@H](CCC(C)(C)NC[C@H](O)c2cncc(F)c2)CC1]

The title compound was prepared from (R)-2-((4-((1r,4S)-4-aminocyclohexyl)-2-methylbutan-2-yl)amino)-1-(5-fluoropyridin-3-yl)ethan-1-ol, see example 92, in accordance with the procedure in Example 67, Step (f).

¹H NMR (400 MHz, CDCl₃) δ 8.40-8.35 (2H, m) 7.52-7.46 (1H, m) 5.30 (1H, d, J=8.0 Hz) 4.66 (1H, dd, J=3.6, 9.0 Hz) 3.74-3.63 (1H, m) 2.92 (1H, dd, J=3.8, 12.0 Hz) 2.8-2.6 (1H, br s) 2.52 (1H, dd, J=9.0, 12.0 Hz) 2.01-1.94 (2H, m) 1.94 (3H, s) 1.78-1.71 (2H, m) 1.39-1.32 (2H, m) 1.20-0.97 (7H, m) 1.05 (6H, s).

### Example 94: N-((1S,4r)-4-(3-(((R)-2-(5-Fluoropyridin-3-yl)-2-hydroxyethyl)amino)-3-methylbutyl)cyclohexyl)methanesulfonamide

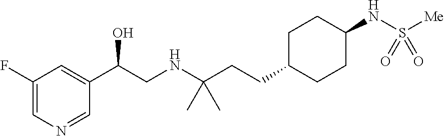 [SMILES: CC(C)(CC[C@H]1CC[C@H](NS(C)(=O)=O)CC1)NC[C@H](O)c1cncc(F)c1]

The title compound was prepared from (R)-2-((4-((1r,4S)-4-aminocyclohexyl)-2-methylbutan-2-yl)amino)-1-(5-fluoropyridin-3-yl)ethan-1-ol, see example 92, in accordance with the procedure in Example 71, Step (a).

¹H NMR (400 MHz, CDCl₃) δ 8.40-8.36 (2H, m) 7.52-7.48 (1H, m) 4.75 (1H, dd, J=3.8, 9.0 Hz) 4.3-4.2 (1H, br s) 3.28-3.18 (1H, br s) 2.99-2.93 (1H, m) 2.97 (3H, s) 2.56 (1H, dd, J=8.9, 12.0 Hz) 2.09-2.01 (2H, m) 1.82-1.75 (2H, m) 1.42-1.36 (2H, m) 1.29-0.94 (7H, m) 1.09 (6H, s).

### Example 95: Methyl (1S,4s)-4-(2-(((R)-2-(3-Fluorophenyl)-2-hydroxyethyl)amino)-propan-2-yl)cyclohexane-1-carboxylate

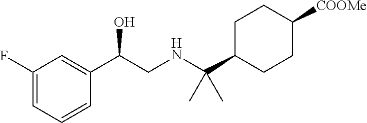 [SMILES: COC(=O)[C@H]1CC[C@@H](C(C)(C)NC[C@H](O)c2cccc(F)c2)CC1]

### (a) 2-((1r,4r)-4-(Benzyloxy)cyclohexyl)propan-2-amine

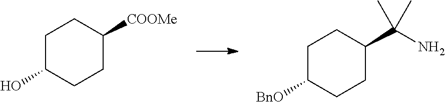 [SMILES: COC(=O)[C@H]1CC[C@H](O)CC1 ; CC(C)(N)[C@H]1CC[C@H](OCc2ccccc2)CC1]

The sub-title compound was prepared from methyl (1r,4r)-4-hydroxycyclohexane-1-carboxylate in accordance with the procedure in Example 25, Step (b), followed by the procedures in Example 5, Steps (a) to (c).

### (b) tert-Butyl (2-((1r,4r)-4-(benzyloxy)cyclohexyl)propan-2-yl)carbamate

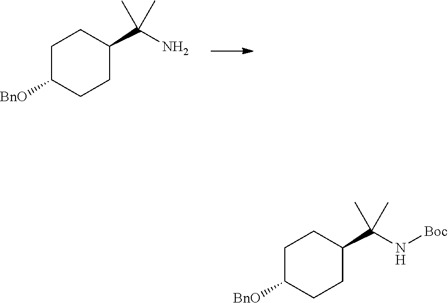 [SMILES: CC(C)(N)[C@H]1CC[C@H](OCc2ccccc2)CC1 ; CC(C)(C)OC(=O)NC(C)(C)[C@H]1CC[C@H](OCc2ccccc2)CC1]

A mixture of 2-((1r,4r)-4-(benzyloxy)cyclohexyl)propan-2-amine (414 mg, 1.67 mmol) and Boc₂O (1.83 g, 8.37 mmol) was stirred at 60° C. for 18 h. The mixture was dissolved in EtOH (7 mL) and imidazole (627 mg, 9.20 mmol) was carefully added (gas evolution). The mixture was stirred at rt for 30 min and concentrated. The residue was dissolved in CHCl₃ and the ice cooled mixture was washed with HCl (aq, 1%). The organic layer was collected, washed with brine, dried (Na₂SO₄) and concentrated. The residue was purified by chromatography to give the sub-title compound (510 mg, 88%).

### (c) tert-Butyl (2-((1r,4r)-4-hydroxycyclohexyl)propan-2-yl)carbamate

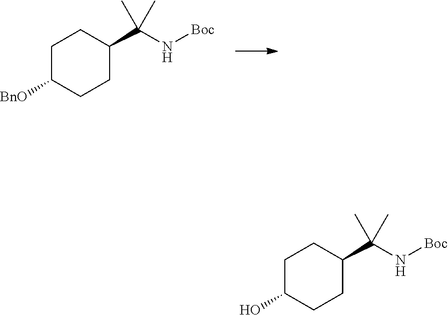 [SMILES: CC(C)(C)OC(=O)NC(C)(C)[C@H]1CC[C@H](OCc2ccccc2)CC1 ; CC(C)(C)OC(=O)NC(C)(C)[C@H]1CC[C@H](O)CC1]

A mixture of tert-butyl (2-((1r,4r)-4-(benzyloxy)cyclohexyl)propan-2-yl)carbamate (745 mg, 2.14 mmol), Pd/C (10%, 228 mg, 0.21 mmol) and MeOH (7 mL) was hydrogenated at ambient temperature and pressure for 1 h. The mixture was filtered through a pad of Celite, which was washed with MeOH. The combined filtrates were concentrated to give the sub-title compound (532 mg, 96%).

### (d) (1r,4r)-4-(2-((tert-Butoxycarbonyl)amino)propan-2-yl)cyclohexyl methanesulfonate

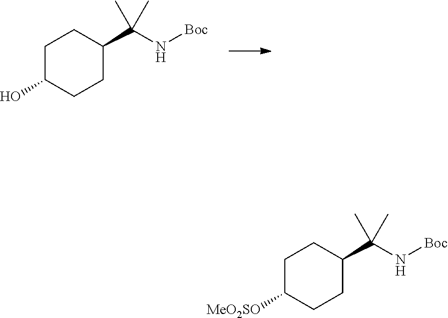

The sub-title compound was prepared from tert-butyl (2-((1r,4r)-4-hydroxycyclo-hexyl)propan-2-yl)carbamate in accordance with the procedure in Example 1, Step (c).

### (e) tert-Butyl (2-((1s,4s)-4-cyanocyclohexyl)propan-2-yl)carbamate

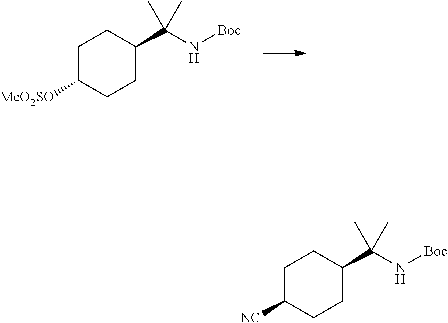

NaCN (685 mg, 14.00 mmol) was added to a solution of (1r,4r)-4-(2-((tert-butoxy-carbonyl)amino)propan-2-yl)cyclohexyl methanesulfonate (670 mg, 2.00 mmol) in DMSO (8 mL). The mixture was stirred at 85° C. for 18 h and cooled to rt. H₂O was added and the mixture was extracted with CH₂Cl₂. The combined extracts were dried (Na₂SO₄) and concentrated, and the residue was purified by chromatography to give the sub-title compound (307 mg, 58%).

### (f) Methyl (1s,4s)-4-(2-aminopropan-2-yl)cyclohexane-1-carboxylate

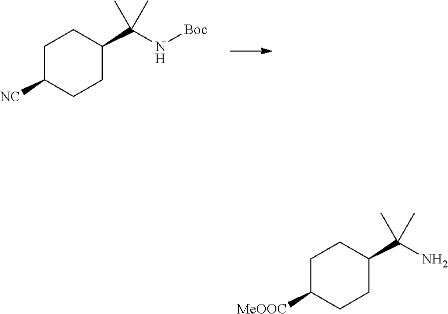 [SMILES: CC(C)(C)OC(=O)NC(C)(C)[C@H]1CC[C@@H](C#N)CC1 ; COC(=O)[C@H]1CC[C@@H](C(C)(C)N)CC1]

A mixture of tert-butyl (2-((1s,4s)-4-cyanocyclohexyl)propan-2-yl)carbamate (300 mg, 1.13 mmol), HCl (aq, 35%, 1.99 mL, 22.52 mmol) and MeOH (4 mL) was stirred at 80° C. for 84 h. HCl (aq, 35%, 1.00 mL, 11.26 mmol) was added and stirring was continued for 16 h. The mixture was concentrated and MeOH (5 mL) and HCl (aq, 35%, 3.00 mL, 33.78 mmol) were added. The mixture was stirred at 80° C. for 20 h and concentrated. The residue was dried over P₂O₅ and dissolved in MeOH (4 mL). The stirred solution was cooled in an ice-bath and SOCl₂ (817 μL, 11.26 mmol) was added dropwise. The mixture was heated at 60° C. for 16 h and concentrated. The residue was dissolved in H₂O and the pH was adjusted to 12 by NaOH (aq, 4 M). The mixture was extracted with CH₂Cl₂ and the combined extract were dried (Na₂SO₄) and concentrated to give the sub-title compound (177 mg, 79%).

### (g) Methyl (1S,4s)-4-(2-(((R)-2-(3-fluorophenyl)-2-hydroxyethyl)amino)propan-2-yl)cyclohexane-1-carboxylate

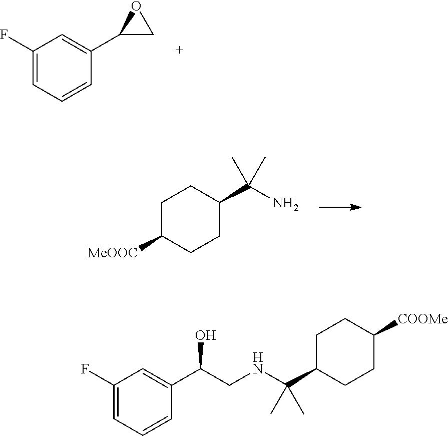 [SMILES: Fc1cccc([C@@H]2CO2)c1 ; COC(=O)[C@H]1CC[C@@H](C(C)(C)N)CC1 ; COC(=O)[C@H]1CC[C@@H](C(C)(C)NC[C@H](O)c2cccc(F)c2)CC1]

The title compound was prepared from methyl (1s,4s)-4-(2-aminopropan-2-yl)cyclo-hexane-1-carboxylate and (R)-2-(3-fluorophenyl)oxirane in accordance with the procedure in Example 1, Step (e).

¹H NMR (400 MHz, CDCl₃) δ 7.33-7.26 (m, 1H), 7.14-7.07 (m, 2H), 6.95 (tdd, J=8.5, 2.6, 1.1 Hz, 1H), 4.54 (dd, J=8.6, 3.7 Hz, 1H), 3.69 (s, 3H), 2.86 (dd, J=11.9, 3.7 Hz, 1H), 2.68-2.61 (m, 1H), 2.50 (dd, J=11.9, 8.6 Hz, 1H), 2.28-2.17 (m, 2H), 1.69-1.55 (m, 2H), 1.51-1.37 (m, 2H), 1.30-1.09 (m, 3H), 0.99 (s, 3H), 0.98 (s, 3H).

### Example 96: Methyl (1S,4s)-4-(2-(((R)-2-(5-Fluoropyridin-3-yl)-2-hydroxyethyl)amino)-propan-2-yl)cyclohexane-1-carboxylate

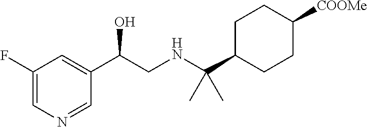 [SMILES: COC(=O)[C@H]1CC[C@@H](C(C)(C)NC[C@H](O)c2cncc(F)c2)CC1]

The title compound was prepared from methyl (1s,4s)-4-(2-aminopropan-2-yl)cyclo-hexane-1-carboxylate, see Example 95, Step (f), and (R)-3-fluoro-5-(oxiran-2-yl)pyridine in accordance with the procedure in Example 1, Step (e).

¹H NMR (400 MHz, CDCl₃) δ 8.41-8.39 (m, 1H), 8.38 (d, J=2.8 Hz, 1H), 7.53-7.47 (m, 1H), 4.72 (dd, J=9.0, 3.5 Hz, 1H), 3.69 (s, 3H), 2.95 (dd, J=12.1, 3.5 Hz, 1H), 2.69-2.62 (m, 1H), 2.54 (dd, J=12.1, 9.0 Hz, 1H), 2.29-2.18 (m, 2H), 1.69-1.58 (m, 2H), 1.52-1.39 (m, 2H), 1.32-1.14 (m, 3H), 1.05 (s, 3H), 1.04 (s, 3H).

### Example 97:(1S,4s)-4-(2-(((R)-2-(5-Fluoropyridin-3-yl)-2-hydroxyethyl)amino)propan-2-yl)cyclohexane-1-carboxylic acid

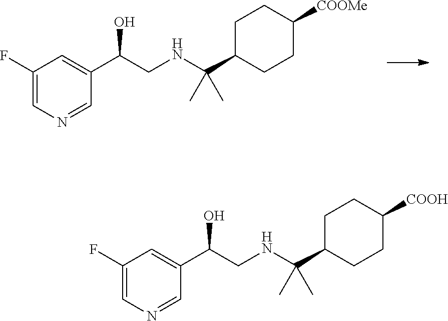 [SMILES: COC(=O)[C@H]1CC[C@@H](C(C)(C)NC[C@H](O)c2cncc(F)c2)CC1 ; CC(C)(NC[C@H](O)c1cncc(F)c1)[C@H]1CC[C@@H](C(=O)O)CC1]

A mixture of methyl (1S,4s)-4-(2-(((R)-2-(5-fluoropyridin-3-yl)-2-hydroxyethyl)-amino)propan-2-yl)cyclohexane-1-carboxylate (27 mg, 0.08 mmol), NaOH (aq, 1 M, 480 μL, 0.48 mmol) and dioxane (1 mL) was stirred at 60° C. for 20 h. NaOH (aq, 1 M, 240 μL) was added and the mixture was stirred at 60° C. for 2 h. The mixture was concentrated and the residue was dissolved in H₂O. The pH was adjusted to 7 by KHSO₄ (aq, 1 M) and the mixture was concentrated and purified by reverse phase chromatography to give the title compound (10 mg, 39%).

H NMR (400 MHz, D₂O) δ 7.80-7.73 (m, 1H), 5.13 (dd, J=10.1, 3.0 Hz, 1H), 3.34 (dd, J=13.0, 3.1 Hz, 1H), 3.24 (dd, J=13.0, 10.2 Hz, 1H), 2.67-2.57 (m, 1H), 2.23-2.12 (m, 2H), 1.79-1.67 (m, 1H), 1.65-1.46 (m, 4H), 1.39-1.19 (m, 8H).

### Example 98: (1S,4s)-4-(2-(((R)-2-(3-Fluorophenyl)-2-hydroxyethyl)amino)propan-2-yl)-cyclohexane-1-carboxylic acid

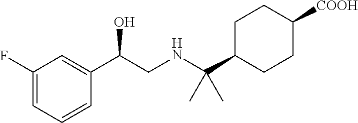 [SMILES: CC(C)(NC[C@H](O)c1cccc(F)c1)[C@H]1CC[C@@H](C(=O)O)CC1]

A solution of LiOH·H₂O (30 mg, 0.71 mmol) in H₂O (4 mL) was added to a stirred ice-cooled solution of methyl (1S,4s)-4-(2-(((R)-2-(3-fluorophenyl)-2-hydroxyethyl)-amino)propan-2-yl)cyclohexane-1-carboxylate (40 mg, 0.12 mol) in THF (4 mL). The mixture was allowed to slowly warm to rt and was stirred at rt for 48 h. AcOH (47 μL, 0.83 mmol) was added and the mixture was stirred at rt for 15 min and filtered through a plug of cotton, which was rinsed with H₂O/MeCN; 1:1). The filtrate was concentrated and the residue was purified by reverse phase chromatography (eluent 0.1% AcOH in H₂O:MeCN=90:10 to 10:90) to give the title compound (37 mg, 97%).

¹H NMR (400 MHz, D₂O) δ 7.47 (td, J=8.1, 5.9 Hz, 1H), 7.28-7.20 (m, 2H), 7.20-7.12 (m, 1H), 5.01 (dd, J=9.8, 3.0 Hz, 1H), 3.30 (dd, J=13.0, 3.2 Hz, 1H), 3.26-3.16 (m, 1H), 2.57-2.46 (m, 1H), 2.22-2.09 (m, 2H), 1.79-1.65 (m, 1H), 1.62-1.44 (m, 4H), 1.33 (s, 3H), 1.32 (s, 3H), 1.29-1.20 (m, 2H).

### Example 99: Methyl (1R,4r)-4-(2-(((R)-2-(3-fluorophenyl)-2-hydroxyethyl)amino)propan-2-yl)cyclohexane-1-carboxylate

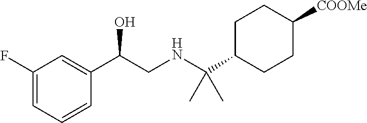 [SMILES: COC(=O)[C@H]1CC[C@H](C(C)(C)NC[C@H](O)c2cccc(F)c2)CC1]

### (a) tert-Butyl (2-(4-hydroxycyclohexyl)propan-2-yl)carbamate

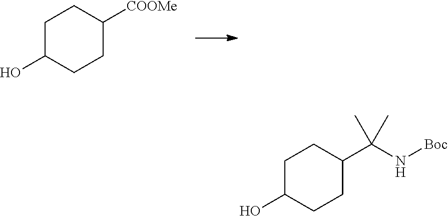 [SMILES: COC(=O)C1CCC(O)CC1 ; CC(C)(C)OC(=O)NC(C)(C)C1CCC(O)CC1]

The sub-title compound was prepared from methyl 4-hydroxycyclohexane-1-carboxylate in accordance with the procedures in Example 95, Steps (a) to (c).

### (b) tert-Butyl (2-(4-oxocyclohexyl)propan-2-yl)carbamate

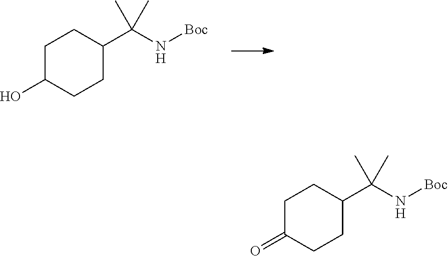 [SMILES: CC(C)(C)OC(=O)NC(C)(C)C1CCC(O)CC1 ; CC(C)(C)OC(=O)NC(C)(C)C1CCC(=O)CC1]

NaHCO₃ (653 mg, 7.77 mmol) followed by Dess-Martin periodinane (1.58 g, 3.73 mmol) were added in single portions to a solution of butyl (2-(4-hydroxycyclohexyl)propan-2-yl)carbamate (800 mg, 3.11 mmol) in CH₂Cl₂ (30 mL). The mixture was stirred at rt for 1.5 h. NaOH (aq, 1 M) was added and the mixture was stirred for 30 min. The layers were separated, and the aq phase was extracted with CH₂Cl₂. The combined organic phases were washed with H₂O and brine, dried (Na₂SO₄) and concentrated. The residue was purified by filtration trough a pad of silica gel to give the sub-title compound (790 mg, 99%).

### (c) tert-Butyl (2-((1r,4r)-4-cyanocyclohexyl)propan-2-yl)carbamate

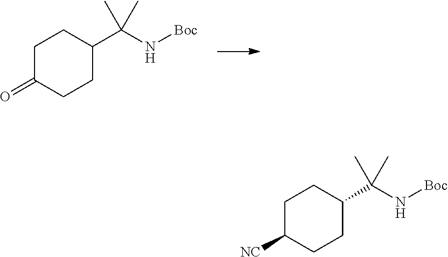 [SMILES: CC(C)(C)OC(=O)NC(C)(C)C1CCC(=O)CC1 ; CC(C)(C)OC(=O)NC(C)(C)[C@H]1CC[C@H](C#N)CC1]

Toluenesulfonylmethyl isocyanide (549 mg, 2.81 mmol) and t-BuOH (308 μl, 3.24 mmol) were added to a stirred solution of tert-butyl (2-(4-oxocyclohexyl)propan-2-yl)carbamate (552 mg, 2.16 mmol) in THF (40 mL) at 42° C. t-BuOK (183 mg, 1.63 mmol) was added in one portion and the mixture was stirred at 42° C. for 50 min. The mixture was filtered through a pad of Celite and concentrated. The residue was purified by chromatography followed by chiral chromatography (Chiralpak IH (30 mm×250 mm, 5 μm, eluent system 10% iPrOH in heptane, flow rate 40 mL/min) to give the sub-title compound (121 mg, 21%).

### (d) Methyl (1r,4r)-4-(2-aminopropan-2-yl)cyclohexane-1-carboxylate

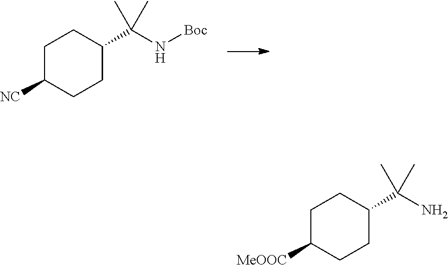 [SMILES: CC(C)(C)OC(=O)NC(C)(C)[C@H]1CC[C@H](C#N)CC1 ; COC(=O)[C@H]1CC[C@H](C(C)(C)N)CC1]

The sub-title compound was prepared from tert-butyl (2-((1r,4r)-4-cyanocyclohexyl)-propan-2-yl)carbamate in accordance with the procedure in Example 95, Step (f).

### (d) Methyl (1R,4r)-4-(2-(((R)-2-(3-fluorophenyl)-2-hydroxyethyl)amino)propan-2-yl)cyclohexane-1-carboxylate

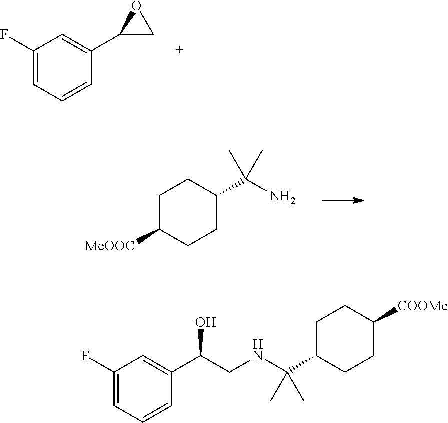 [SMILES: Fc1cccc([C@@H]2CO2)c1 ; COC(=O)[C@H]1CC[C@H](C(C)(C)N)CC1 ; COC(=O)[C@H]1CC[C@H](C(C)(C)NC[C@H](O)c2cccc(F)c2)CC1]

The title compound was prepared from methyl (1r,4r)-4-(2-aminopropan-2-yl)cyclo-hexane-1-carboxylate and (R)-2-(3-fluorophenyl)oxirane in accordance with the procedure in Example 1, Step (e).

¹H NMR (400 MHz, CDCl₃) δ 7.30 (td, J=8.1, 5.9 Hz, 1H), 7.18-7.05 (m, 2H), 6.95 (tdd, J=8.4, 2.6, 1.1 Hz, 1H), 4.56 (dd, J=8.6, 3.8 Hz, 1H), 3.66 (s, 3H), 2.87 (dd, J=11.9, 3.7 Hz, 1H), 2.56-2.46 (m, 1H), 2.22 (tt, J=12.3, 3.6 Hz, 1H), 2.10-1.98 (m, 2H), 1.90-1.75 (m, 2H), 1.48-1.32 (m, 2H), 1.26 (tt, J=12.1, 3.0 Hz, 1H), 1.12-1.02 (m, 2H), 1.01 (s, 3H), 1.01 (s, 3H).

### Example 100: Methyl (1R,4r)-4-(2-(((R)-2-(5-fluoropyridin-3-yl)-2-hydroxyethyl)amino)-propan-2-yl)cyclohexane-1-carboxylate

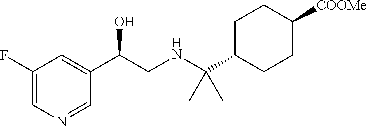 [SMILES: COC(=O)[C@H]1CC[C@H](C(C)(C)NC[C@H](O)c2cncc(F)c2)CC1]

The title compound was prepared from methyl (1r,4r)-4-(2-aminopropan-2-yl)cyclo-hexane-1-carboxylate, see Example 99, Step (d), and (R)-3-fluoro-5-(oxiran-2-yl)pyridine in accordance with the procedure in Example 1, Step (e).

¹H NMR (400 MHz, CDCl₃) δ 8.41-8.34 (m, 2H), 7.52-7.46 (m, 1H), 4.63 (dd, J=8.9, 3.7 Hz, 1H), 2.92 (dd, J=12.0, 3.8 Hz, 1H), 2.57-2.44 (m, 1H), 2.22 (tt, J=12.3, 3.6 Hz, 1H), 2.09-2.00 (m, 2H), 1.89-1.76 (m, 1H), 1.40 (qd, J=13.1, 3.5 Hz, 2H), 1.26 (tt, J=12.0, 2.9 Hz, 2H), 1.13-0.99 (m, 8H; including 1.03 (s, 3H), 1.02 (s, 3H))

### Example 101:(1R,4r)-4-(2-(((R)-2-(3-Fluorophenyl)-2-hydroxyethyl)amino)propan-2-yl)cyclohexane-1-carboxylic acid

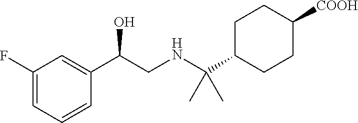 [SMILES: CC(C)(NC[C@H](O)c1cccc(F)c1)[C@H]1CC[C@H](C(=O)O)CC1]

The title compound was prepared from methyl (1S,4s)-4-(2-(((R)-2-(3-fluorophenyl)-2-hydroxyethyl)amino)propan-2-yl)cyclohexane-1-carboxylate, see Example 99, in accordance with the procedure in Example 98.

¹H NMR (400 MHz, D₂O) δ 7.47 (td, J=8.0, 5.8 Hz, 1H), 7.31-7.20 (m, 2H), 7.16 (m, 1H), 5.02 (dd, J=9.9, 3.1 Hz, 1H), 3.37-3.15 (m, 2H), 2.21-2.08 (m, 1H), 2.07-1.91 (m, 2H), 1.84-1.64 (m, 3H), 1.44-1.30 (m, 8H; including 1.35 (s, 6H)), 1.30-1.16 (m, 2H).

### Example 102:(1R,4r)-4-(2-(((R)-2-(5-fluoropyridin-3-yl)-2-hydroxyethyl)amino)propan-2-yl)cyclohexane-1-carboxylic acid

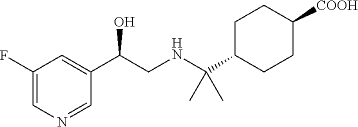 [SMILES: CC(C)(NC[C@H](O)c1cncc(F)c1)[C@H]1CC[C@H](C(=O)O)CC1]

The title compound was prepared from methyl (1S,4s)-4-(2-(((R)-2-(5-fluoropyridin-3-yl)-2-hydroxyethyl)amino)propan-2-yl)cyclohexane-1-carboxylate, see Example 100, in accordance with the procedure in Example 98.

¹H NMR (400 MHz, D₂O) δ 8.63-8.18 (m, 2H), 7.68 (d, J=8.9 Hz, 1H), 5.02 (dd, J=10.1, 2.9 Hz, 1H), 3.27 (dd, J=13.0, 3.0 Hz, 1H), 3.17 (dd, J=13.0, 10.2 Hz, 1H), 2.13-2.00 (m, 1H), 1.98-1.85 (m, 2H), 1.77-1.55 (m, 3H), 1.35-1.23 (m, 8H; including 1.28 (s, 3H), 1.27 (s, 3H)), 1.23-1.10 (m, 2H).

### Example 103: Methyl (1S,4s)-4-(2-(((R)-2-(3-fluorophenyl)-2-hydroxyethyl)amino)-2-methylpropyl)cyclohexane-1-carboxylate

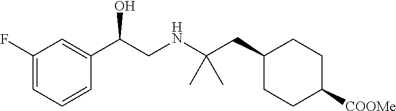 [SMILES: COC(=O)[C@H]1CC[C@@H](CC(C)(C)NC[C@H](O)c2cccc(F)c2)CC1]

The title compound was prepared from methyl (1r,4r)-4-hydroxycyclohexane-1-carboxylate in accordance with the procedures in Example 49, Step (a); Example 41, Step (c); Example 67, Step (b); Example 17, Step (f); Example 13, Step (a); Example 95, Step (b); Example 49, Step (c); Example 1, Step (b); Example 17, Step (f); Example 95, Step (f) and finally Example 1, Step (e).

¹H NMR (400 MHz, CDCl₃) δ 7.34-7.26 (m, 1H), 7.14-7.07 (m, 2H), 6.95 (tdd, J=8.5, 2.6, 1.0 Hz, 1H), 4.56 (dd, J=8.5, 3.8 Hz, 1H), 3.67 (s, 3H), 2.88 (dd, J=11.9, 3.8 Hz, 1H), 2.52 (dd, J=11.9, 8.5 Hz, 1H), 2.49-2.43 (m, 1H), 1.95-1.81 (m, 2H), 1.65-1.50 (m, 5H), 1.40-1.21 (m, 4H), 1.05 (s, 6H).

### Example 104: Methyl (1S,4s)-4-(2-(((R)-2-(5-fluoropyridin-3-yl)-2-hydroxyethyl)amino)-2-methylpropyl)cyclohexane-1-carboxylate

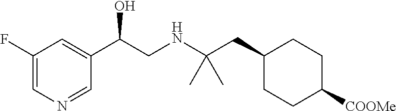 [SMILES: COC(=O)[C@H]1CC[C@@H](CC(C)(C)NC[C@H](O)c2cncc(F)c2)CC1]

The title compound was prepared in accordance with the procedures in Example 103, using (R)-3-fluoro-5-(oxiran-2-yl)pyridine in the final step.

¹H NMR (400 MHz, CDCl₃) δ 8.34-8.32 (m, 1H), 8.32 (d, J=2.8 Hz, 1H), 7.46-7.41 (m, 1H), 4.68 (dd, J=9.0, 3.5 Hz, 1H), 3.61 (s, 3H), 2.91 (dd, J=12.1, 3.5 Hz, 1H), 2.50 (dd, J=12.1, 9.0 Hz, 1H), 2.46-2.37 (m, 1H), 1.90-1.77 (m, 2H), 1.58-1.45 (m, 5H), 1.35-1.16 (m, 4H), 1.06 (s, 3H), 1.05 (s, 3H).

### Example 105:(1S,4s)-4-(2-(((R)-2-(3-Fluorophenyl)-2-hydroxyethyl)amino)-2-methylpropyl)cyclohexane-1-carboxylic acid

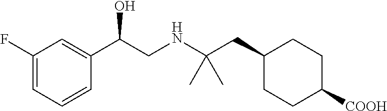 [SMILES: CC(C)(C[C@H]1CC[C@@H](C(=O)O)CC1)NC[C@H](O)c1cccc(F)c1]

NaOH (aq, 1 M, 905 μL) was added to a stirred solution of methyl (1S,4s)-4-(2-(((R)-2-(3-fluorophenyl)-2-hydroxyethyl)amino)-2-methylpropyl)cyclohexane-1-carboxylate (53 mg, 0.15 mmol) in dioxane (3 mL) at rt. The solution was stirred at 60° C. for 20 h, allowed to cool and concentrated. The residue was dissolved in H₂O and the pH was adjusted to 7 by KHSO₄ (aq, 1 M). The precipitate was collected and suspended in H₂O. The H₂O was decanted and this procedure was repeated three times. The solid was dried (P₂O₅) for 48 h to give the title compound (35 mg, 69%).

¹H NMR (400 MHz, AcOH-d₄) δ 7.40-7.32 (m, 1H), 7.25-7.21 (m, 1H), 7.21-7.16 (m, 1H), 7.06-7.00 (m, 1H), 5.20 (dd, J=10.7, 2.8 Hz, 1H), 3.42 (dd, J=12.3, 2.8 Hz, 1H), 3.13 (dd, J=12.3, 10.7 Hz, 1H), 2.64-2.51 (m, 1H), 2.01-1.89 (m, 2H), 1.78-1.58 (m, 7H), 1.51-1.33 (m, 8H).

### Example 106:(1S,4s)-4-(2-(((R)-2-(5-Fluoropyridin-3-yl)-2-hydroxyethyl)amino)-2-methylpropyl)cyclohexane-1-carboxylic acid

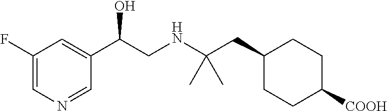 [SMILES: CC(C)(C[C@H]1CC[C@@H](C(=O)O)CC1)NC[C@H](O)c1cncc(F)c1]

NaOH (aq, 1 M, 460 μL, 0.46 mmol) was added to a solution of methyl (1S,4s)-4-(2-(((R)-2-(5-fluoropyridin-3-yl)-2-hydroxyethyl)amino)propan-2-yl)cyclohexane-1-carboxylate (27 mg, 0.08 mmol) in dioxane (1 mL) at rt. The solution was stirred at 60° C. for 18 h and NaOH (aq, 1 M, 230 μL, 0.23 mmol) was added. The solution was stirred at 60° C. for 5 h, allowed to cool and concentrated. The residue was purified by reverse phase chromatography to give the title compound (6 mg, 23%).

¹H NMR (400 MHz, D₂O) δ 8.52-8.38 (m, 2H), 7.79-7.73 (m, 1H), 5.13 (dd, J=10.1, 3.0 Hz, 1H), 3.37 (dd, J=12.9, 3.0 Hz, 1H), 3.26 (dd, J=12.9, 10.1 Hz, 1H), 2.38-2.25 (m, 1H), 1.80-1.53 (m, 9H), 1.48-1.34 (m, 8H).

### Example 107: Methyl (1R,4r)-4-(2-(((R)-2-(3-Fluorophenyl)-2-hydroxyethyl)amino)-2-methylpropyl)cyclohexane-1-carboxylate

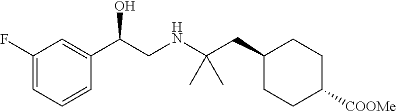 [SMILES: COC(=O)[C@H]1CC[C@H](CC(C)(C)NC[C@H](O)c2cccc(F)c2)CC1]

### (a) Methyl 4-((tert-butyldimethylsilyl)oxy)cyclohexane-1-carboxylate

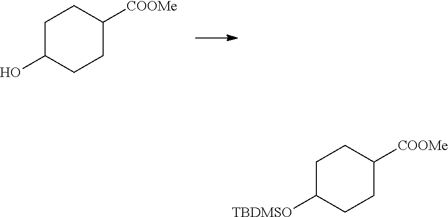 [SMILES: COC(=O)C1CCC(O)CC1 ; COC(=O)C1CCC(O[Si](C)(C)C(C)(C)C)CC1]

Imidazole (1.29 g, 18.96 mmol) and tert-butyldimethylsilyl chloride (2.29 g, 15.17 mmol) were added to a stirred solution of methyl 4-hydroxycyclohexane-1-carboxylate (2.00 g, 12.64 mmol) in DMF (20 mL) at rt. The mixture was stirred at rt for 18 h and Et₂O and H₂O were added. The aq phase was collected and extracted with Et₂O. The combined organic phases were washed with H₂O and brine, dried (Na₂SO₄) and concentrated. The residue was purified by chromatography to give the sub-title compound (3.39 g, 98%).

### (b) (4-((tert-Butyldimethylsilyl)oxy)cyclohexyl)methyl methanesulfonate

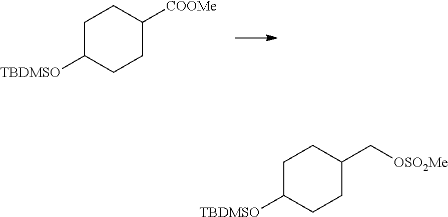

The sub-title compound was prepared from methyl 4-((tert-butyldimethylsilyl)oxy)-cyclohexane-1-carboxylate in accordance with the procedures in Example 41, Step (b) and Example 17, Step (e).

### (c) 2-(4-((tert-Butyldimethylsilyl)oxy)cyclohexyl)acetonitrile

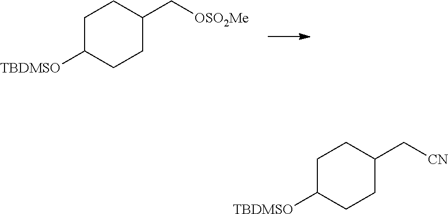

NaCN (1.58 g, 32.29 mmo) was added to a solution of (4-((tert-butyldimethylsilyl)-oxy)cyclohexyl)methyl methanesulfonate (2.60 g, 8.07 mmol) in DMSO (10 mL). The mixture was stirred at 80° C. for 12 h and cooled to rt. H₂O was added and the mixture was extracted with Et₂O. The combined extracts were washed with H₂O, dried (Na₂SO₄) and concentrated to give the sub-title compound (1.85 g, 90%).

### (d) tert-Butyl (1-(4-((tert-butyldimethylsilyl)oxy)cyclohexyl)-2-methylpropan-2-yl)-carbamate

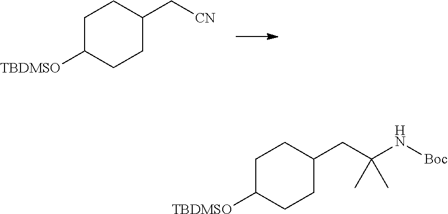 [SMILES: CC(C)(C)[Si](C)(C)OC1CCC(CC#N)CC1 ; CC(C)(CC1CCC(O[Si](C)(C)C(C)(C)C)CC1)NC(=O)OC(C)(C)C]

The sub-title compound was prepared from 2-(4-((tert-butyldimethylsilyl)oxy)cyclo-hexyl)acetonitrile in accordance with the procedures in Example 13, Step (a) and Example 95, Step (b).

### (e) tert-Butyl (1-(4-hydroxycyclohexyl)-2-methylpropan-2-yl)carbamate

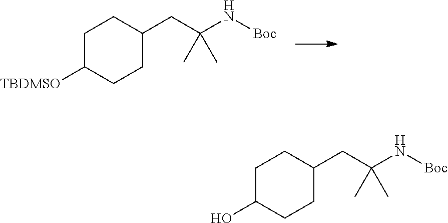 [SMILES: CC(C)(CC1CCC(O[Si](C)(C)C(C)(C)C)CC1)NC(=O)OC(C)(C)C ; CC(C)(CC1CCC(O)CC1)NC(=O)OC(C)(C)C]

NH₄F (5.59 g, 150.91 mmol) was added to a solution of tert-butyl (1-(4-((tert-butyl-dimethylsilyl)oxy)cyclohexyl)-2-methylpropan-2-yl)carbamate (1.94 g, 5.03 mmol) in MeOH (34 mL). The mixture was stirred at 60° C. for 48 h and at 80° C. for 7 days and concentrated. The residue was extracted with CH₂Cl₂ and the combined extracts were filtered through a plug of cotton and concentrated. The residue was purified by chromatography to give the sub-title compound (1.18 g, 87%).

### (f) Methyl (1R,4r)-4-(2-(((R)-2-(3-fluorophenyl)-2-hydroxyethyl)amino)-2-methyl-propyl)cyclohexane-1-carboxylate

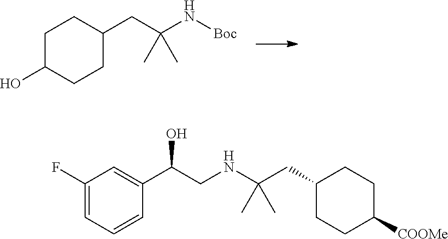 [SMILES: CC(C)(CC1CCC(O)CC1)NC(=O)OC(C)(C)C ; COC(=O)[C@H]1CC[C@H](CC(C)(C)NC[C@H](O)c2cccc(F)c2)CC1]

The title compound was prepared from tert-butyl (1-(4-hydroxycyclohexyl)-2-methyl-propan-2-yl)carbamate in accordance with the procedures in Example 99, Steps (b) and (c); Example 95, Step (f) and Example 1, Step (e).

¹H NMR (400 MHz, CDCl₃) δ 7.33-7.26 (m, 1H), 7.14-7.07 (m, 2H), 6.95 (tdd, J=8.4, 2.6, 1.0 Hz, 1H), 4.58 (dd, J=8.6, 3.7 Hz, 1H), 3.66 (s, 3H), 2.89 (dd, J=11.9, 3.8 Hz, 1H), 2.53 (dd, J=11.9, 8.5 Hz, 1H), 2.20 (tt, J=12.2, 3.6 Hz, 1H), 1.94 (dd, J=13.5, 3.5 Hz, 2H), 1.82 (dd, J=13.4, 3.3 Hz, 2H), 1.44 (qd, J=13.1, 3.4 Hz, 2H), 1.36-1.20 (m, 4H), 1.07 (s, 6H), 1.05-0.92 (m, 2H).

### Example 108: Methyl (1R,4r)-4-(2-(((R)-2-(5-fluoropyridin-3-yl)-2-hydroxyethyl)amino)-2-methylpropyl)cyclohexane-1-carboxylate

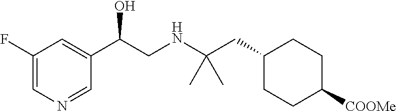 [SMILES: COC(=O)[C@H]1CC[C@H](CC(C)(C)NC[C@H](O)c2cncc(F)c2)CC1]

The title compound was prepared in accordance with the procedures in Example 107 using (R)-3-fluoro-5-(oxiran-2-yl)pyridine in the final step.

¹H NMR (400 MHz, CDCl₃) δ 8.40-8.35 (m, 2H), 7.49 (ddd, J=9.3, 2.6, 1.7 Hz, 1H), 4.64 (dd, J=8.8, 3.7 Hz, 1H), 3.66 (s, 3H), 2.93 (dd, J=12.0, 3.7 Hz, 1H), 2.52 (dd, J=12.0, 8.8 Hz, 1H), 2.21 (tt, J=12.2, 3.6 Hz, 1H), 1.94 (dd, J=14.1, 3.2 Hz, 2H), 1.82 (dd, J=13.1, 3.5 Hz, 2H), 1.44 (qd, J=13.0, 3.3 Hz, 2H), 1.38-1.21 (m, 4H), 1.08 (s, 6H), 1.05-0.95 (m, 2H).

### Example 109:(1R,4r)-4-(2-(((R)-2-(3-fluorophenyl)-2-hydroxyethyl)amino)-2-methyl-propyl)cyclohexane-1-carboxylic acid acetate

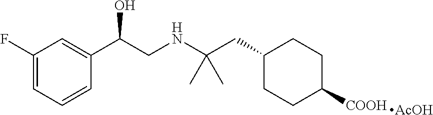 [SMILES: "·AcOH" ; CC(C)(C[C@H]1CC[C@H](C(=O)O)CC1)NC[C@H](O)c1cccc(F)c1]

A solution of LiOH·H₂O (28 mg, 0.67 mmol) in H₂O (4 mL) was added at to an ice-cooled solution of methyl (1R,4r)-4-(2-(((R)-2-(3-fluorophenyl)-2-hydroxyethyl)amino)-2-methylpropyl)cyclohexane-1-carboxylate (39 mg, 0.11 mmol) in THF (4 mL). The mixture was allowed to slowly warm to and was stirred at rt for 16 h. AcOH (45 μL, 0.78 mmol) was added and the mixture was stirred at rt for 15 min and filtered through a plug of cotton, which was rinsed with water/MeCN; 1:1). The filtrate was concentrated and the residue was purified by reverse phase chromatography (eluent 0.1% AcOH in H₂O:MeCN=90:10 to 10:90) to give the title compound (35 mg, 79%).

¹H NMR (400 MHz, DMSO-d₆) δ 7.38-7.30 (m, 1H), 7.20-7.10 (m, 2H), 7.08-6.98 (m, 1H), 4.56 (dd, J=7.7, 4.6 Hz, 1H), 2.65-2.54 (m, 2H), 2.00-1.87 (m, 1H), 1.71 (dd, J=13.3, 3.4 Hz, 2H), 1.38-1.12 (m, 7H), 0.98 (s, 6H), 0.94-0.81 (m, 2H).

### Example 110: (1R,4r)-4-(2-(((R)-2-(5-fluoropyridin-3-yl)-2-hydroxyethyl)amino)-2-methylpropyl)cyclohexane-1-carboxylic acid acetate

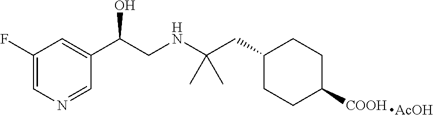 [SMILES: "·AcOH" ; CC(C)(C[C@H]1CC[C@H](C(=O)O)CC1)NC[C@H](O)c1cncc(F)c1]

The title compound was prepared from methyl (1R,4r)-4-(2-(((R)-2-(5-fluoropyridin-3-yl)-2-hydroxyethyl)amino)-2-methylpropyl)cyclohexane-1-carboxylate, see Example 108, in accordance with the procedure in Example 98.

¹H NMR (300 MHz, D₂O) δ 8.45 (s, 2H), 7.75 (d, J=9.3, 1H), 5.05 (dd, J=8.8, 4.2 Hz, 1H), 3.24-3.06 (m, 1H), 2.09 (tt, J=12.2, 3.6 Hz, 1H), 1.90-1.76 (m, 4H), 1.52 (d, J=5.0 Hz, 2H), 1.45-1.33 (m, 3H), 1.31 (s, 6H), 1.18-0.99 (m, 2H).

### Example 111: Methyl (1S,4s)-4-(3-(((R)-2-(3-fluorophenyl)-2-hydroxyethyl)amino)-3-methylbutyl)cyclohexane-1-carboxylate

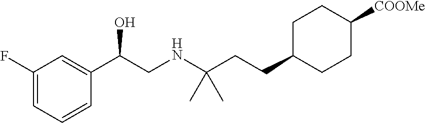 [SMILES: COC(=O)[C@H]1CC[C@@H](CCC(C)(C)NC[C@H](O)c2cccc(F)c2)CC1]

### (a) tert-Butyl (4-((1s,4r)-4-((tert-butyldimethylsilyl)oxy)cyclohexyl)-2-methylbutan-2-yl)carbamate

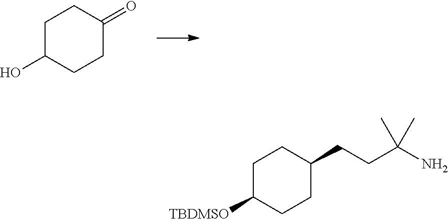 [SMILES: O=C1CCC(O)CC1 ; CC(C)(N)CC[C@H]1CC[C@@H](O[Si](C)(C)C(C)(C)C)CC1]

The sub-title compound was prepared from 4-hydroxycyclohexan-1-one in accordance with the procedures in Example 107, Step (a); Example 17, Steps (b) to (f) and finally Example 13, Step (a).

### (b) tert-Butyl (4-((1s,4r)-4-((tert-butyldimethylsilyl)oxy)cyclohexyl)-2-methylbutan-2-yl)carbamate

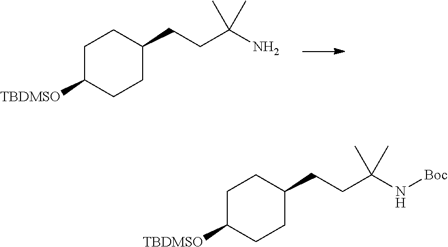 [SMILES: CC(C)(N)CC[C@H]1CC[C@@H](O[Si](C)(C)C(C)(C)C)CC1 ; CC(C)(CC[C@H]1CC[C@@H](O[Si](C)(C)C(C)(C)C)CC1)NC(=O)OC(C)(C)C]

Boc₂O (612 mg, 2.80 mmol) was added to a stirred ice-cooled solution of 4-((1s,4r)-4-((tert-butyldimethylsilyl)oxy)cyclohexyl)-2-methylbutan-2-amine (800 mg, 2.67 mmol) in CH₂Cl₂ (5.5 mL). The mixture was stirred at rt for 18 h and concentrated. The residue was purified by chromatography to give the sub-title compound (580 mg, 54%).

### (c) tert-Butyl (4-((1s,4r)-4-hydroxycyclohexyl)-2-methylbutan-2-yl)carbamate

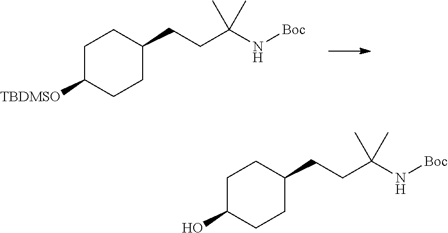 [SMILES: CC(C)(CC[C@H]1CC[C@@H](O[Si](C)(C)C(C)(C)C)CC1)NC(=O)OC(C)(C)C ; CC(C)(CC[C@H]1CC[C@@H](O)CC1)NC(=O)OC(C)(C)C]

NH₄F (1.61 g, 43.53 mmol) was added to a solution of tert-butyl (4-((1s,4r)-4-((tert-butyldimethylsilyl)oxy)cyclohexyl)-2-methylbutan-2-yl)carbamate (580 mg, 1.45 mmol) in MeOH (5 mL) at rt. The mixture was stirred at 60° C. for 70 h and allowed to cool to rt. H₂O was added and the mixture was extracted with CH₂Cl₂. The combined extracts were dried (Na₂SO₄) and concentrated. The residue was dissolved in THF (2 mL) and tetrabutylammonium fluoride (1 M in THF, 2.2 mL, 2.18 mmol) was added. The mixture was stirred at 60° C. for 2 h and concentrated. The residue was suspended in CH₂Cl₂ and filtered through a cotton wool pad. The filtrate was concentrated and the residue dissolved in MeOH (5 mL). NH₄F (0.81 g, 21.77 mmol) was added and the mixture was stirred at 60° C. for 18 h and concentrated. The residue was suspended in CH₂Cl₂ and filtered through a cotton wool pad. The residue was concentrated to give the sub-title compound (367 mg, 89%).

### (d) tert-Butyl (2-methyl-4-(4-oxocyclohexyl)butan-2-yl)carbamate

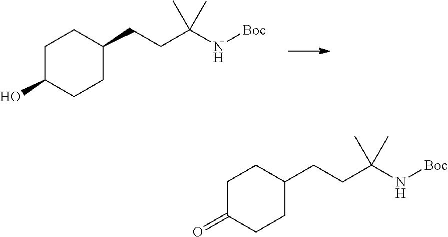 [SMILES: CC(C)(CC[C@H]1CC[C@@H](O)CC1)NC(=O)OC(C)(C)C ; CC(C)(CCC1CCC(=O)CC1)NC(=O)OC(C)(C)C]

Dess-Martin periodinane (1.13 g, 2.65 mmol) was added in one portion to a solution of tert-butyl (4-((1s,4r)-4-hydroxycyclohexyl)-2-methylbutan-2-yl)carbamate (631 mg, 2.21 mmol) in CH₂Cl₂ (20 mL) at rt. The mixture was stirred at rt for 2 h. H₂O (300 μL) was added and the mixture was stirred at rt for 20 h. Dess-Martin periodinane (0.94 g, 2.21 mmol) and H₂O (300 μL) were added and the mixture was stirred at rt for 24 h. Na₂S₂O₃ (aq, sat, 3 mL) and NaHCO₃(aq, sat, 2 mL) were added and the mixture was stirred vigorously for 10 min. The layers were separated, and the aq phase was extracted with CH₂Cl₂. The combined organic phases were washed with brine, dried (Na₂SO₄) and concentrated. The residue was purified by chromatography to give the sub-title compound (610 mg, 97%).

### (e) tert-Butyl (4-(4-cyanocyclohexyl)-2-methylbutan-2-yl)carbamate

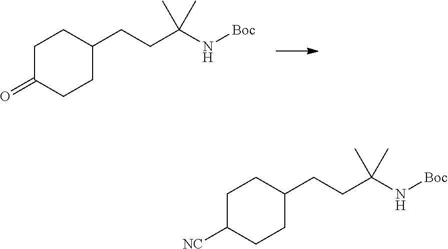 [SMILES: CC(C)(CCC1CCC(=O)CC1)NC(=O)OC(C)(C)C ; CC(C)(CCC1CCC(C#N)CC1)NC(=O)OC(C)(C)C]

A solution of tert-butyl (2-methyl-4-(4-oxocyclohexyl)butan-2-yl)carbamate (600 mg, 2.12 mmol) in THF (35 mL) was added to a stirred mixture of toluenesulfonylmethyl isocyanide (537 mg, 2.75 mmol) and t-BuOH (301 μL, 3.18 mmol) at rt. t-BuOK (475 mg, 4.23 mmol) was added in one portion at 21° C. and the mixture was quickly heated to 45° C. and stirred at that temperature for 30 min. AcOH (two drops), brine (1 mL) and CH₂Cl₂ (5 mL) were added and the layers were separated. The aq phase was extracted with CH₂Cl₂ and the combined organic phases were washed with NaHCO₃(aq, sat) and brine, dried (Na₂SO₄) and concentrated. The residue was purified by chromatography to give the sub-title compound (380 mg, 61%).

### (f) (1s,4s)-4-(3-Amino-3-methylbutyl)cyclohexane-1-carboxylic acid and (1r,4r)-4-(3-Amino-3-methylbutyl)cyclohexane-1-carboxylic acid

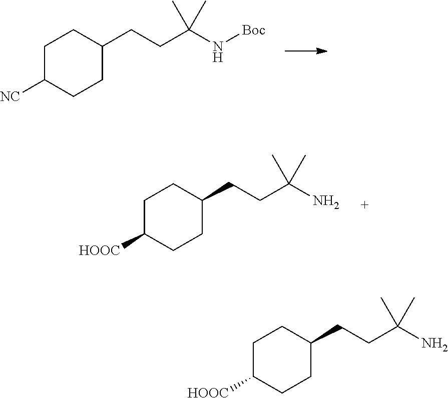 [SMILES: CC(C)(CCC1CCC(C#N)CC1)NC(=O)OC(C)(C)C ; CC(C)(N)CC[C@H]1CC[C@@H](C(=O)O)CC1 ; CC(C)(N)CC[C@H]1CC[C@H](C(=O)O)CC1]

HCl (35%, 2.85 mL, 32.27 mmol) was added to tert-butyl (4-(4-cyanocyclohexyl)-2-methylbutan-2-yl)carbamate (380 mg, 1.29 mmol) and the mixture was stirred at 80° C. for 16 h and concentrated. The residue purified with preparative HPLC (XBridge, 30×100 mm, 5 μm, 0.1% AcOH/MeCN=90:10 to 10:90) to give (1s,4s)-4-(3-amino-3-methylbutyl)cyclohexane-1-carboxylic acid (62 mg, 23%) and (1r,4r)-4-(3-amino-3-methylbutyl)cyclohexane-1-carboxylic acid (104 mg, 38%).

### (g) Methyl (1s,4s)-4-(3-amino-3-methylbutyl)cyclohexane-1-carboxylate

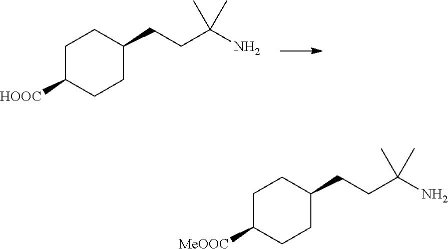 [SMILES: CC(C)(N)CC[C@H]1CC[C@@H](C(=O)O)CC1 ; COC(=O)[C@H]1CC[C@@H](CCC(C)(C)N)CC1]

SOCl₂ (476 μL, 6.56 mmol) was added drop-wise to an ice-cooled solution of (1s,4s)-4-(3-amino-3-methylbutyl)cyclohexane-1-carboxylic acid (140 mg, 0.66 mmol) in MeOH (532 μL, 13.13 mmol). The mixture was stirred at rt for 20 h and concentrated. The residue was dissolved in H₂O and the pH was adjusted to 12 by the addition of NH₃ (aq, sat). The mixture was extracted with CH₂Cl₂ and the combined extracts were washed with brine, dried (Na₂SO₄) and concentrated to give the sub-title compound (152 mg, 99%).

### (h) Methyl (1S,4s)-4-(3-(((R)-2-(3-fluorophenyl)-2-hydroxyethyl)amino)-3-methyl-butyl)cyclohexane-1-carboxylate

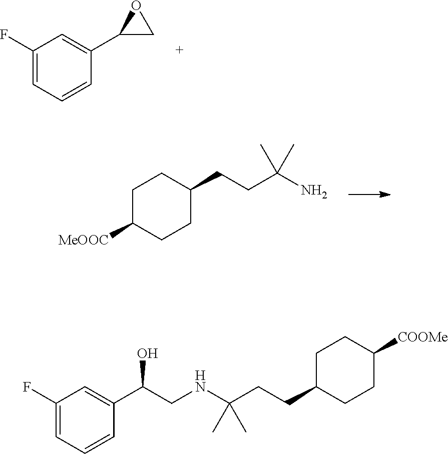 [SMILES: Fc1cccc([C@@H]2CO2)c1 ; COC(=O)[C@H]1CC[C@@H](CCC(C)(C)N)CC1 ; COC(=O)[C@H]1CC[C@@H](CCC(C)(C)NC[C@H](O)c2cccc(F)c2)CC1]

The title compound was prepared from methyl (1s,4s)-4-(3-amino-3-methylbutyl)-cyclohexane-1-carboxylate and (R)-2-(3-fluorophenyl)oxirane in accordance with the procedure in Example 1, Step (e).

¹H NMR (400 MHz, CDCl₃) δ 7.37 (td, J=8.0, 5.8 Hz, 1H), 7.24 (d, J=7.8 Hz, 1H), 7.22-7.17 (m, 1H), 7.07-6.99 (m, 1H), 5.22 (dd, J=10.6, 2.8 Hz, 1H), 3.42 (dd, J=12.4, 2.8 Hz, 1H), 3.13 (dd, J=12.4, 10.6 Hz, 1H), 2.30 (tt, J=12.2, 3.6 Hz, 1H), 2.02-1.98 (m, 1H), 1.90-1.82 (m, 2H), 1.82-1.72 (m, 2H), 1.45 (td, J=13.0, 3.2 Hz, 2H), 1.41 (s, 6H), 1.37-1.19 (m, 4H), 1.07-0.93 (m, 2H).

### Example 112: Methyl (1S,4s)-4-(3-(((R)-2-(5-fluoropyridin-3-yl)-2-hydroxyethyl)amino)-3-methylbutyl)cyclohexane-1-carboxylate

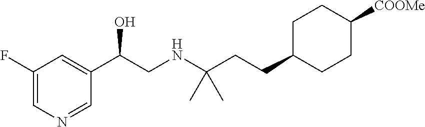 [SMILES: COC(=O)[C@H]1CC[C@@H](CCC(C)(C)NC[C@H](O)c2cncc(F)c2)CC1]

The title compound was prepared in accordance with the procedures in Example 111 using (R)-3-fluoro-5-(oxiran-2-yl)pyridine in the final step.

¹H NMR (400 MHz, CDCl₃) δ 8.42-8.40 (m, 1H), 8.39 (d, J=2.8 Hz, 1H), 7.60-7.55 (m, 1H), 7.30-7.15 (br s, 3H), 5.07 (dd, J=10.2, 2.6 Hz, 1H), 3.67 (s, 3H), 3.01 (dd, J=12.0, 3.0 Hz, 1H), 2.79 (dd, J=11.8, 10.2 Hz, 1H), 2.56-2.47 (m, 1H), 2.00 (s, 3H), 1.98-1.89 (m, 2H), 1.59-1.43 (m, 6H), 1.32-1.20 (m, 9H).

### Example 113: (1S,4s)-4-(3-(((R)-2-(3-Fluorophenyl)-2-hydroxyethyl)amino)-3-methyl-butyl)cyclohexane-1-carboxylic acid

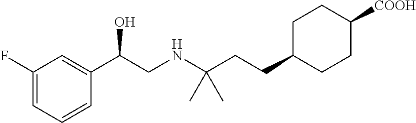 [SMILES: CC(C)(CC[C@H]1CC[C@@H](C(=O)O)CC1)NC[C@H](O)c1cccc(F)c1]

A solution of LiOH·H₂O (21 mg, 0.49 mmol) in H₂O (3 mL) was added to a stirred ice-cooled solution of methyl (1S,4s)-4-(3-(((R)-2-(3-fluorophenyl)-2-hydroxyethyl)amino)-3-methylbutyl)cyclohexane-1-carboxylate (30 mg, 0.08 mmol) in THF (3 mL). The mixture was allowed to slowly warm to rt and was stirred at rt for 16 h. AcOH (35 μL, 0.61 mmol) was added and the mixture was stirred at rt for 15 min and filtered through a plug of cotton, which was rinsed with water/MeCN; 1:1). The filtrate was concentrated and the residue was purified by preparative HPLC (Atlantis T3, 30×100 mm, 5 μm, 0.1% AcOH water:MeCN=90:10 to 10:90) to give the title compound (21 mg, 74%).

¹H NMR (400 MHz, AcOH-d₄) δ 7.36 (td, J=8.0, 5.8 Hz, 1H), 7.23 (d, J=7.6 Hz, 1H), 7.22-7.17 (m, 1H), 7.07-6.99 (m, 1H), 5.20 (dd, J=10.6, 2.8 Hz, 1H), 3.42 (dd, J=12.4, 2.8 Hz, 1H), 3.12 (dd, J=12.4, 10.6 Hz, 1H), 2.63 (p, J=4.8 Hz, 1H), 2.07-2.05 (d, J=2.8 Hz, 1H), 2.02-1.98 (m, 1H), 1.82-1.69 (m, 2H), 1.68-1.53 (m, 4H), 1.41 (s, 6H), 1.38-1.23 (m, 5H).

### Example 114:(1S,4s)-4-(3-(((R)-2-(5-Fluoropyridin-3-yl)-2-hydroxyethyl)amino)-3-methylbutyl)cyclohexane-1-carboxylic acid

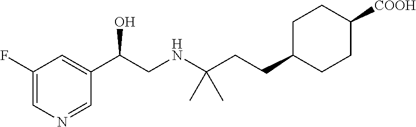 [SMILES: CC(C)(CC[C@H]1CC[C@@H](C(=O)O)CC1)NC[C@H](O)c1cncc(F)c1]

The title compound was prepared from methyl (1S,4s)-4-(3-(((R)-2-(5-fluoropyridin-3-yl)-2-hydroxyethyl)amino)-3-methylbutyl)cyclohexane-1-carboxylate in accordance with the procedure in Example 113 (reaction time 48 h).

¹H NMR (400 MHz, AcOH-d₄) δ 7.37 (td, J=8.0, 5.8 Hz, 1H), 7.24 (d, J=7.8 Hz, 1H), 7.22-7.17 (m, 1H), 7.07-6.99 (m, 1H), 5.22 (dd, J=10.6, 2.8 Hz, 1H), 3.42 (dd, J=12.4, 2.8 Hz, 1H), 3.13 (dd, J=12.4, 10.6 Hz, 1H), 2.30 (tt, J=12.2, 3.6 Hz, 1H), 2.02-1.98 (m, 1H), 1.90-1.82 (m, 2H), 1.82-1.72 (m, 2H), 1.45 (td, J=13.0, 3.2 Hz, 2H), 1.41 (s, 6H), 1.37-1.19 (m, 4H), 1.07-0.93 (m, 2H).

Examples 115 to 118: The title compounds were prepared from (1r,4r)-4-(3-amino-3-methylbutyl)cyclohexane-1-carboxylic acid, see Example 111, Step (f), in accordance with the procedure in Example 111 to 114.

### Example 115: Methyl (1R,4r)-4-(3-(((R)-2-(3-fluorophenyl)-2-hydroxyethyl)amino)-3-methylbutyl)cyclohexane-1-carboxylate

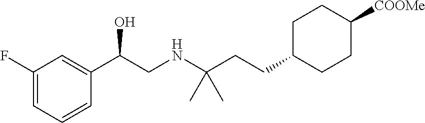 [SMILES: COC(=O)[C@H]1CC[C@H](CCC(C)(C)NC[C@H](O)c2cccc(F)c2)CC1]

¹H NMR (400 MHz, CDCl₃) δ 7.30 (td, 3=8.0, 5.8 Hz, 1H), 7.15-7.09 (m, 2H), 6.96 (tdd, 3=8.4, 2.6, 1.0 Hz, 1H), 4.66 (dd, 3=8.8, 3.4 Hz, 1H), 3.66 (s, 3H), 2.91 (dd, 3=12.0, 3.6 Hz, 1H), 2.55 (dd, J=12.0, 8.8 Hz, 1H), 2.22 (tt, J=12.2, 3.6 Hz, 1H), 2.01-1.91 (m, 2H), 1.85-1.75 (m, 2H), 1.46-1.32 (m, 4H), 1.21-1.11 (m, 3H), 1.08 (s, 6H), 0.98-0.85 (m, 2H).

### Example 116: Methyl (1R,4r)-4-(3-(((R)-2-(5-fluoropyridin-3-yl)-2-hydroxyethyl)amino)-3-methylbutyl)cyclohexane-1-carboxylate

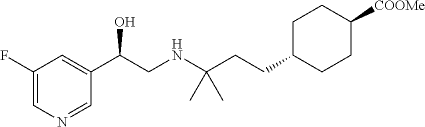 [SMILES: COC(=O)[C@H]1CC[C@H](CCC(C)(C)NC[C@H](O)c2cncc(F)c2)CC1]

¹H NMR (400 MHz, CDCl₃) δ 8.41 (s, 1H), 8.38 (d, J=2.6 Hz, 1H), 7.61-7.54 (m, 1H), 7.11-6.80 (br s, 3H), 5.07 (d, J=9.2 Hz, 1H), 3.65 (s, 3H), 3.01 (dd, J=12.0, 2.4 Hz, 1H), 2.83-2.74 (m, 1H), 2.20 (tt, J=12.2, 3.6 Hz, 1H), 2.05-1.89 (m, 5H), 1.84-1.72 (m, 2H), 1.61-1.49 (m, 2H), 1.36 (qd, J=13.0, 3.4 Hz, 2H), 1.23 (s, 6H).

### Example 117:(1R,4r)-4-(3-(((R)-2-(3-Fluorophenyl)-2-hydroxyethyl)amino)-3-methyl-butyl)cyclohexane-1-carboxylic acid

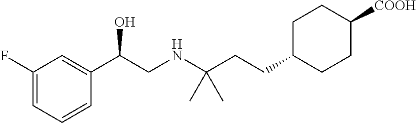 [SMILES: CC(C)(CC[C@H]1CC[C@H](C(=O)O)CC1)NC[C@H](O)c1cccc(F)c1]

¹H NMR (400 MHz, AcOH-d₄) δ 7.37 (td, J=8.0, 5.8 Hz, 1H), 7.24 (d, J=7.8 Hz, 1H), 7.22-7.17 (m, 1H), 7.07-6.99 (m, 1H), 5.22 (dd, J=10.6, 2.8 Hz, 1H), 3.42 (dd, J=12.4, 2.8 Hz, 1H), 3.13 (dd, J=12.4, 10.6 Hz, 1H), 2.30 (tt, J=12.2, 3.6 Hz, 1H), 2.02-1.98 (m, 1H), 1.90-1.82 (m, 2H), 1.82-1.72 (m, 2H), 1.45 (td, J=13.0, 3.2 Hz, 2H), 1.41 (s, 6H), 1.37-1.19 (m, 4H), 1.07-0.93 (m, 2H).

### Example 118:(1R,4r)-4-(3-(((R)-2-(5-Fluoropyridin-3-yl)-2-hydroxyethyl)amino)-3-methylbutyl)cyclohexane-1-carboxylic acid

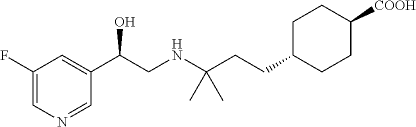 [SMILES: CC(C)(CC[C@H]1CC[C@H](C(=O)O)CC1)NC[C@H](O)c1cncc(F)c1]

¹H NMR (400 MHz, DMSO-d₆+1 drop of TFA) δ 8.59 (d, J=2.8 Hz, 1H), 8.57-8.55 (m, 1H), 8.50 (t, J=9.8 Hz, 1H), 8.30 (t, J=9.8 Hz, 1H), 7.88-7.83 (m, 1H), 4.97 (dd, J=9.8, 2.8 Hz, 1H), 3.27-3.15 (m, 1H), 3.13-3.00 (m, 1H), 2.11 (tt, J=12.2, 3.4 Hz, 1H), 1.92-1.82 (m, 2H), 1.80-1.71 (m, 2H), 1.65-1.56 (m, 2H), 1.33-1.26 (m, 2H), 1.25 (s, 6H), 1.20-1.06 (m, 3H), 0.98-0.81 (m, 2H).

### Example 119: (R)-1-(3-Fluorophenyl)-2-((2-((1S,3R)-3-methoxycyclohexyl)propan-2-yl)amino)ethan-1-ol

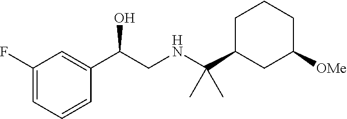 [SMILES: CO[C@@H]1CCC[C@H](C(C)(C)NC[C@H](O)c2cccc(F)c2)C1]

### (a) 2-(3-Methoxyphenyl)propan-2-amine hydrochloride

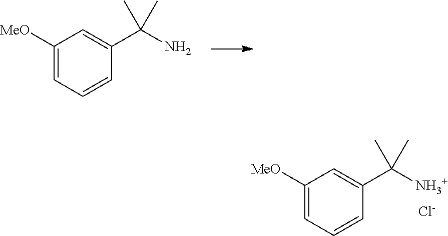

HCl (2 M in Et₂O) was added dropwise to a stirred solution of 2-(3-methoxyphenyl)propan-2-amine (505 mg, 3.06 mmol) in Et₂O (20 mL) at rt. The solution was cooled in an ice-bath and the walls of the flask was scratched with a spatula. The precipitate was collected to give the sub-title compound (415 mg, 67%).

### (b) 2-(3-Methoxycyclohexyl)propan-2-amine hydrochloride

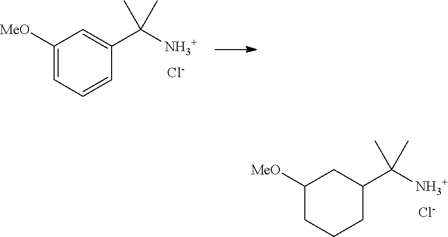

PtO₂·H₂O (48.8 mg, 0.20 mol) was added to a solution of 2-(3-methoxyphenyl)propan-2-amine hydrochloride (200 mg, 0.99 mmol) in H₂O (6 mL). The mixture was hydrogenated at 10 atm and 60° C. for a total time of 112 h (during this period the mixture was filtered five times through Celite and fresh PtO₂·H₂O (48.8 mg, 0.20 mmol) was added before hydrogenation continued). The mixture was filtered through Celite and the filtrate was concentrated. Toluene was added and the mixture was concentrated. This procedure was repeated to give the sub-title compound (176 mg, 85%) as a mixture of cis-isomers.

### (c) (R)-1-(3-Fluorophenyl)-2-((2-((1S,3R)-3-methoxycyclohexyl)propan-2-yl)amino)-ethan-1-ol

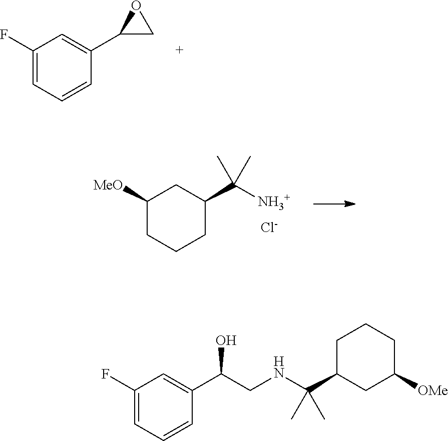 [SMILES: Fc1cccc([C@@H]2CO2)c1 ; CO[C@@H]1CCC[C@H](C(C)(C)[NH3+])C1 ; [Cl-] ; CO[C@@H]1CCC[C@H](C(C)(C)NC[C@H](O)c2cccc(F)c2)C1]

The title compound was prepared from 2-(3-methoxycyclohexyl)propan-2-amine hydrochloride and (R)-2-(3-fluorophenyl)oxirane in accordance with the procedure in Example 1, Step (e) followed, by purification by chiral chromatography (Chiralpak ID, (250×30 mm, 5 μm), eluent n-heptane/iPrOH (93/7), Et₃N 0.2%, flow rate 40 mL/min). The stereochemistry was arbitrarily assigned to be 1S,3R.

[α]_{D}²⁵ −39.3 (c 0.57, CHCl₃).

¹H NMR (400 MHz, CDCl₃) δ 7.34-7.27 (m, 1H), 7.16-7.07 (m, 2H), 6.95 (tdd, J=8.5, 2.6, 1.1 Hz, 1H), 4.55 (dd, J=8.7, 3.7 Hz, 1H), 3.36 (s, 3H), 3.09 (tt, J=10.8, 4.1 Hz, 1H), 2.88 (dd, J=11.9, 3.7 Hz, 1H), 2.50 (dd, J=11.9, 8.7 Hz, 1H), 2.13-2.01 (m, 2H), 1.90-1.79 (m, 1H), 1.71-1.59 (m, 1H), 1.36-1.22 (m, 1H), 1.17 (tt, J=13.2, 3.4 Hz, 1H), 1.09-0.99 (m, 7H), 0.99-0.85 (m, 2H).

### Example 120: (R)-1-(3-Fluorophenyl)-2-((2-((1R,3S)-3-methoxycyclohexyl)propan-2-yl)amino)ethan-1-ol

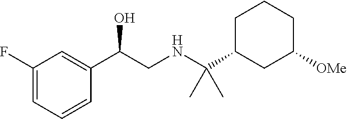 [SMILES: CO[C@H]1CCC[C@@H](C(C)(C)NC[C@H](O)c2cccc(F)c2)C1]

The title compound was isolated in the purification procedure in Example 119, Step (c) and was arbitrarily assigned to be the 1R,3S isomer.

[α]_{D}²⁵ −50.8 (c 0.64, CHCl₃).

¹H NMR (400 MHz, CDCl₃) δ 7.33-7.27 (m, 1H), 7.16-7.06 (m, 2H), 6.99-6.91 (m, 1H), 4.56 (dd, J=8.6, 3.7 Hz, 1H), 3.35 (s, 3H), 3.08 (tt, J=10.8, 4.1 Hz, 1H), 2.87 (dd, J=11.9, 3.7 Hz, 1H), 2.52 (dd, J=11.9, 8.6 Hz, 1H), 2.13-2.01 (m, 2H), 1.91-1.80 (m, 1H), 1.74-1.63 (m, 1H), 1.35-1.23 (m, 1H), 1.18 (tt, J=13.2, 3.4 Hz, 1H), 1.10-1.00 (m, 7H), 1.00-0.83 (m, 2H).

### Example 121: (R)-1-(5-Fluoropyridin-3-yl)-2-((2-((1S,3R)-3-methoxycyclohexyl)propan-2-yl)amino)ethan-1-ol

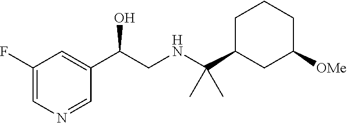 [SMILES: CO[C@@H]1CCC[C@H](C(C)(C)NC[C@H](O)c2cncc(F)c2)C1]

The title compound was prepared from 2-(3-methoxycyclohexyl)propan-2-amine hydrochloride, see Example 119, Step (b), and (R)-3-fluoro-5-(oxiran-2-yl)pyridine in accordance with the procedure in Example 1, Step (e), and purification by chromatography on silica gel, followed by chromatography on amino-functionalized silica-gel and chiral chromatography (Chiralpak IA, (250×30 mm, 5 μm), eluent n-heptane/iPrOH (93/7), Et₃N 0.2%, flow rate 40 mL/min). The stereochemistry was arbitrarily assigned to be 1S,3R.

[α]_{D}²⁵ −42.1 (c 1.05, CHCl₃).

¹H NMR (400 MHz, CDCl₃) δ 8.40-8.38 (m, 1H), 8.37 (d, J=2.8 Hz, 1H), 7.61-7.42 (m, 1H), 4.62 (dd, J=8.9, 3.7 Hz, 1H), 3.36 (s, 3H), 3.09 (tt, J=10.8, 4.1 Hz, 1H), 2.92 (dd, J=12.0, 3.7 Hz, 1H), 2.50 (dd, J=12.0, 8.9 Hz, 1H), 2.14-2.02 (m, 2H), 1.91-1.81 (m, 1H), 1.71-1.61 (m, 1H), 1.36-1.22 (m, 1H), 1.17 (tt, J=13.2, 3.4 Hz, 1H), 1.10-0.97 (m, 7H), 0.97-0.83 (m, 2H).

### Example 122: (R)-1-(5-Fluoropyridin-3-yl)-2-((2-((1R,3S)-3-methoxycyclohexyl)propan-2-yl)amino)ethan-1-ol

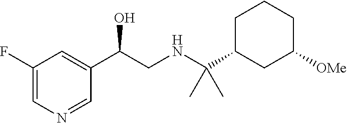 [SMILES: CO[C@H]1CCC[C@@H](C(C)(C)NC[C@H](O)c2cncc(F)c2)C1]

The title compound was isolated in the purification procedure in Example 121 and was arbitrarily assigned to be the 1R,3S isomer.

[α]_{D}²⁵ −52.2 (c 1.00, CHCl₃).

¹H NMR (400 MHz, CDCl₃) δ 8.39-8.36 (m, 2H), 7.53-7.45 (m, 1H), 4.62 (dd, J=8.9, 3.8 Hz, 1H), 3.35 (s, 3H), 3.09 (tt, J=10.8, 4.1 Hz, 1H), 2.91 (dd, J=12.0, 3.8 Hz, 1H), 2.51 (dd, J=12.0, 8.9 Hz, 1H), 2.13-2.02 (m, 2H), 1.92-1.81 (m, 1H), 1.74-1.64 (m, 1H), 1.34-1.23 (m, 1H), 1.18 (tt, J=13.2, 3.4 Hz, 1H), 1.09-0.98 (m, 7H), 0.98-0.86 (m, 2H).

### Example 123: Ethyl 2-(((1R,4r)-4-(2-(((R)-2-(3-fluorophenyl)-2-hydroxyethyl)amino)-propan-2-yl)cyclohexyl)oxy)acetate

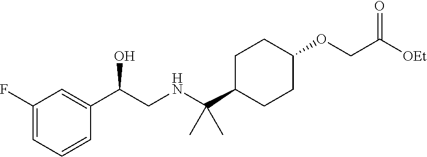 [SMILES: CCOC(=O)CO[C@H]1CC[C@H](C(C)(C)NC[C@H](O)c2cccc(F)c2)CC1]

### (a) tert-Butyl (2-(4-hydroxycyclohexyl)propan-2-yl)carbamate

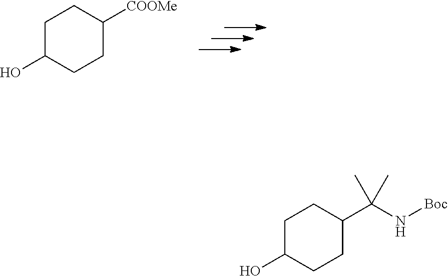 [SMILES: COC(=O)C1CCC(O)CC1 ; CC(C)(C)OC(=O)NC(C)(C)C1CCC(O)CC1]

The sub-title compound was prepared from methyl 4-hydroxycyclohexane-1-carboxylate in accordance with the procedures in Example 25, Step (b), Example 5, Steps (a) to (c), and Example 95, Steps (b) and (c).

### (b) Ethyl 2-(((1r,4r)-4-(2-((tert-butoxycarbonyl)amino)propan-2-yl)cyclohexyl)oxy)-acetate and Ethyl 2-(((1s,4s)-4-(2-((tert-butoxycarbonyl)amino)propan-2-yl)cyclohexyl)-oxy)acetate

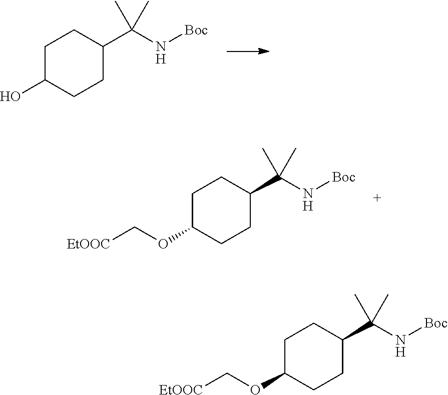 [SMILES: CC(C)(C)OC(=O)NC(C)(C)C1CCC(O)CC1 ; CCOC(=O)CO[C@H]1CC[C@H](C(C)(C)NC(=O)OC(C)(C)C)CC1 ; CCOC(=O)CO[C@H]1CC[C@@H](C(C)(C)NC(=O)OC(C)(C)C)CC1]

Rhodium (II) acetate dimer (0.021 g, 0.047 mmol) followed by ethyl diazoacetate (0.58 mL, 4.66 mmol) were added to a solution of tert-butyl (2-(4-hydroxycyclohexyl)propan-2-yl)carbamate (0.40 g, 1.55 mmol) in CH₂Cl₂(4 mL) at rt. The mixture was stirred for 15 min at rt and concentrated. The residue was purified by chromatography to give the sub-title compounds (0.22 g, 41%) for the cis diastereomer and (0.17 g, 31%) for the trans diastereomer.

### (c) Ethyl 2-(((1r,4r)-4-(2-aminopropan-2-yl)cyclohexyl)oxy)acetate

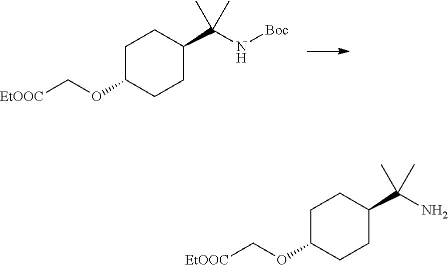 [SMILES: CCOC(=O)CO[C@H]1CC[C@H](C(C)(C)NC(=O)OC(C)(C)C)CC1 ; CCOC(=O)CO[C@H]1CC[C@H](C(C)(C)N)CC1]

TFA (0.470 mL, 6.12 mmol) was added to a solution of ethyl 2-(((1r,4r)-4-(2-((tert-butoxycarbonyl)amino)propan-2-yl)cyclohexyl)oxy) acetate (70 mg, 0.20 mmol) in CH₂Cl₂ (2 mL) at rt. The mixture was stirred at rt for 20 min and concentrated. NaHCO₃(aq, sat) was added to the residue and the mixture was extracted with CH₂Cl₂. The combined extracts were washed with H₂O and brine, dried (Na₂SO₄) and concentrated to give the sub-title compound (47 mg, 95%).

### (d) Ethyl 2-(((1R,4r)-4-(2-(((R)-2-(3-fluorophenyl)-2-hydroxyethyl)amino)propan-2-yl)cyclohexyl)oxy)acetate

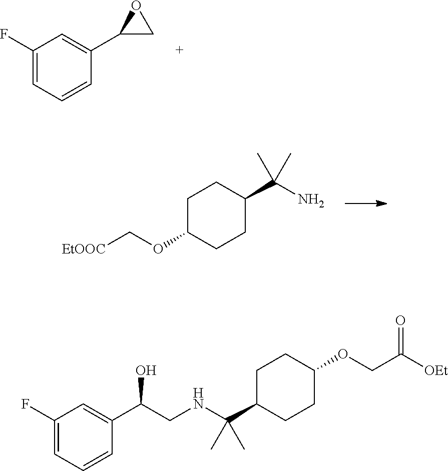 [SMILES: Fc1cccc([C@@H]2CO2)c1 ; CCOC(=O)CO[C@H]1CC[C@H](C(C)(C)N)CC1 ; CCOC(=O)CO[C@H]1CC[C@H](C(C)(C)NC[C@H](O)c2cccc(F)c2)CC1]

The title compound was prepared from ethyl 2-(((1r,4r)-4-(2-aminopropan-2-yl)cyclo-hexyl)oxy)acetate and (R)-2-(3-fluorophenyl)oxirane in accordance with the procedure in Example 1, Step (e).

NMR (400 MHz, CDCl₃) δ: 7.34-7.27 (m, 1H), 7.15-7.06 (m, 2H), 6.95 (tdd, J=8.4, 2.6, 1.0 Hz, 1H), 4.56 (dd, J=8.6, 3.7 Hz, 1H), 4.21 (q, J=7.1 Hz, 2H), 4.11 (s, 2H), 3.27 (tt, J=10.9, 4.2 Hz, 1H), 2.88 (dd, J=11.9, 3.8 Hz, 1H), 2.52 (dd, J=11.9, 8.7 Hz, 1H), 2.18-2.08 (m, 2H), 1.87-1.74 (m, 2H), 1.32-1.20 (m, 6H), 1.14-1.01 (m, 2H), 1.01 (s, 3H), 1.00 (s, 3H).

### Example 124:Ethyl 2-(((1S,4s)-4-(2-(((R)-2-(3-fluorophenyl)-2-hydroxyethyl)amino-)propan-2-yl)cyclohexyl)oxy)acetate

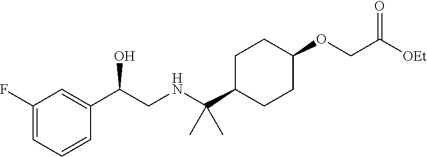 [SMILES: CCOC(=O)CO[C@H]1CC[C@@H](C(C)(C)NC[C@H](O)c2cccc(F)c2)CC1]

The title compound was prepared in accordance with the procedures in Example 123 using ethyl 2-(((1s,4s)-4-(2-((tert-butoxycarbonyl)amino)propan-2-yl)cyclohexyl)oxy)acetate in Step (c).

¹H NMR (400 MHz, CDCl₃) δ: 7.33-7.26 (m, 1H), 7.15-7.07 (m, 2H), 6.95 (tdd, J=8.5, 2.6, 1.1 Hz, 1H), 4.55 (dd, J=8.8, 3.7 Hz, 1H), 4.21 (q, J=7.1 Hz, 2H), 4.06 (s, 2H), 3.66-3.60 (m, 1H), 2.88 (dd, J=11.9, 3.7 Hz, 1H), 2.50 (dd, J=11.9, 8.8 Hz, 1H), 2.08-1.98 (m, 2H), 1.54-1.43 (m, 3H), 1.42-1.32 (m, 3H), 1.31-1.26 (m, 4H), 1.02 (s, 3H), 1.01 (s, 3H).

### Example 125:Ethyl 2-(((1R,4r)-4-(2-(((R)-2-(5-fluoropyridin-3-yl)-2-hydroxyethyl)-amino)propan-2-yl)cyclohexyl)oxy)acetate

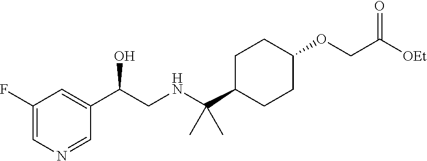 [SMILES: CCOC(=O)CO[C@H]1CC[C@H](C(C)(C)NC[C@H](O)c2cncc(F)c2)CC1]

The title compound was prepared in accordance with the procedures in Example 123 using (R)-3-fluoro-5-(oxiran-2-yl)pyridine in Step (d).

¹H NMR (400 MHz, CDCl₃) δ: 8.42-8.34 (m, 2H), 7.52-7.46 (m, 1H), 4.64 (dd, J=8.8, 3.7 Hz, 1H), 4.21 (q, J=7.2 Hz, 2H), 4.10 (s, 2H), 3.27 (tt, J=10.9, 4.2 Hz, 1H), 2.93 (dd, J=12.0, 3.8 Hz, 1H), 2.52 (dd, J=12.0, 8.9 Hz, 1H), 2.16-2.09 (m, 2H), 1.87-1.74 (m, 2H), 1.31-1.20 (m, 7H), 1.09-1.07 (m, 1H), 1.03 (s, 3H), 1.02 (s, 3H).

### Example 126:2-(((1R,4r)-4-(2-(((R)-2-(3-Fluorophenyl)-2-hydroxyethyl)amino)propan-2-yl)cyclohexyl)oxy)acetic acid

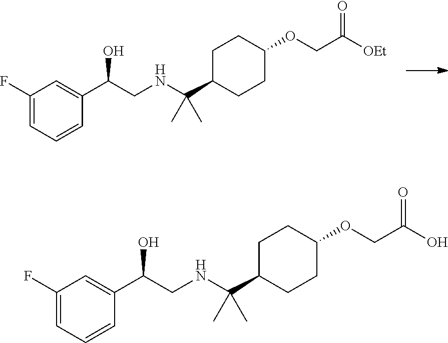 [SMILES: CCOC(=O)CO[C@H]1CC[C@H](C(C)(C)NC[C@H](O)c2cccc(F)c2)CC1 ; CC(C)(NC[C@H](O)c1cccc(F)c1)[C@H]1CC[C@H](OCC(=O)O)CC1]

A solution of LiOH·H₂O (15 mg, 0.378 mmol) in H₂O (3.5 mL) was added to an ice-cooled solution of ethyl 2-(((1R,4r)-4-(2-(((R)-2-(3-fluorophenyl)-2-hydroxyethyl)amino)propan-2-yl)cyclohexyl)oxy)acetate (36 mg, 94 μmol) in THF (3.5 mL). The mixture was slowly allowed to come to rt and was stirred at rt for 16 h. AcOH (27 μl, 0.47 mmol) was added and the mixture was stirred at rt for 15 min and filtered through a cotton plug, which was rinsed with H₂O/MeCN (1:1). The pooled filtrates were concentrated and MeCN:AcOH (v/v=1:1) was added until a solution was formed. Purification using reverse phase chromatography (5% to 95% MeCN/H₂O+0.2% AcOH) gave the title compound (19 mg, 86%).

¹H NMR (400 Hz, D₂O) δ: 7.52-7.43 (m, 1H), 7.29-7.20 (m, 2H), 7.16 (tdd, J=9.1, 2.6, 1.0 Hz, 1H), 5.01 (dd, J=9.8, 3.0 Hz, 1H), 3.97 (s, 2H), 3.46-3.35 (m, 1H), 3.31 (dd, J=12.9, 3.2 Hz, 1H), 3.22 (dd, J=12.9, 9.9 Hz, 1H), 2.25-2.09 (m, 2H), 1.86-1.63 (m, 3H), 1.35 (s, 3H), 1.34 (s, 3H), 1.32-1.20 (m, 4H).

### Example 127:2-(((1R,4r)-4-(2-(((R)-2-(5-Fluoropyridin-3-yl)-2-hydroxyethyl)amino)-propan-2-yl)cyclohexyl)oxy)acetic acid

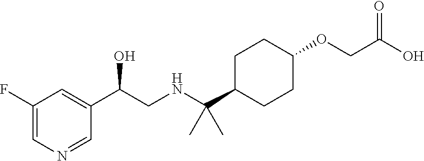 [SMILES: CC(C)(NC[C@H](O)c1cncc(F)c1)[C@H]1CC[C@H](OCC(=O)O)CC1]

The title compound was prepared from ethyl 2-(((1R,4r)-4-(2-(((R)-2-(5-fluoropyridin-3-yl)-2-hydroxyethyl)amino)propan-2-yl)cyclohexyl)oxy)acetate (see Example 125) in accordance with the procedure in Example 126.

¹H NMR (600 Hz, D₂O) δ 8.56 (s, 2H), 7.77 (d, J=8.9 Hz, 1H), 5.14 (dd, J=10.3, 2.8 Hz, 1H), 3.95 (s, 2H), 3.42 (s, 1H), 3.36 (dd, J=13.0, 2.9 Hz, 1H), 3.25 (dd, J=12.9, 10.2 Hz, 1H), 2.23-2.15 (m, 2H), 1.84-1.75 (m, 2H), 1.74-1.65 (m, 1H), 1.36 (s, 3H), 1.35 (s, 3H), 1.32-1.22 (m, 4H).

### Example 128:2-(((1S,4s)-4-(2-(((R)-2-(3-Fluorophenyl)-2-hydroxyethyl)amino)propan-2-yl)cyclohexyl)oxy)acetic acid

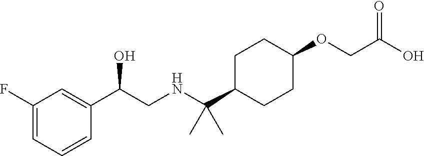 [SMILES: CC(C)(NC[C@H](O)c1cccc(F)c1)[C@H]1CC[C@@H](OCC(=O)O)CC1]

The title compound was prepared from ethyl 2-(((1S,4s)-4-(2-(((R)-2-((3-fluorophenyl)-2-hydroxyethyl)amino)propan-2-yl)cyclohexyl)oxy)acetate in accordance with the procedure in Example 126.

¹H NMR (600 Hz, D₂O) δ: 7.42-7.35 (m, 1H), 7.22-7.11 (m, 2H), 7.07 (tdd, J=9.3, 2.7, 1.0 Hz, 1H), 4.93 (dd, J=9.9, 3.0 Hz, 1H), 3.82 (d, J=1.0 Hz, 2H), 3.63 (s, 1H), 3.22 (dd, J=13.0, 3.1 Hz, 1H), 3.13 (dd, J=12.9, 9.9 Hz, 1H), 2.04-1.91 (m, 2H), 1.70-1.61 (m, 1H), 1.45-1.34 (m, 6H), 1.28 (s, 6H).

### Example 129:(1S,4s)-4-(2-(((R)-2-(3-Fluorophenyl)-2-hydroxyethyl)amino)-2-methylpropyl)cyclohexane-1-carbonitrile

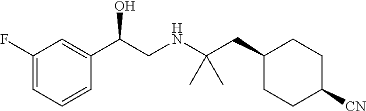 [SMILES: CC(C)(C[C@H]1CC[C@@H](C#N)CC1)NC[C@H](O)c1cccc(F)c1]

### (a) (1s,4s)-4-(2-Amino-2-methylpropyl)cyclohexane-1-carbonitrile

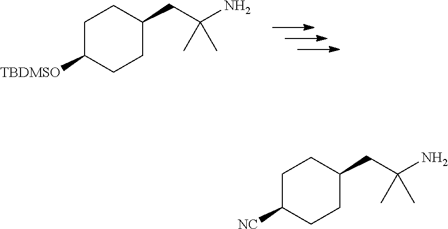 [SMILES: CC(C)(N)C[C@H]1CC[C@@H](O[Si](C)(C)C(C)(C)C)CC1 ; CC(C)(N)C[C@H]1CC[C@@H](C#N)CC1]

The sub-title compound was prepared from 1-((1s,4s)-4-((tert-butyldimethylsilyl)-oxy)cyclohexyl)-2-methylpropan-2-amine (see Example 48, Step (b), in accordance with the procedures in Example 95, Step (b), Example 49, Step (d), Example 99, Steps (b) and (c) and finally Example 123, Step (c).

### (b) (1S,4s)-4-(2-(((R)-2-(3-Fluorophenyl)-2-hydroxyethyl)amino)-2-methyl-propyl)cyclohexane-1-carbonitrile

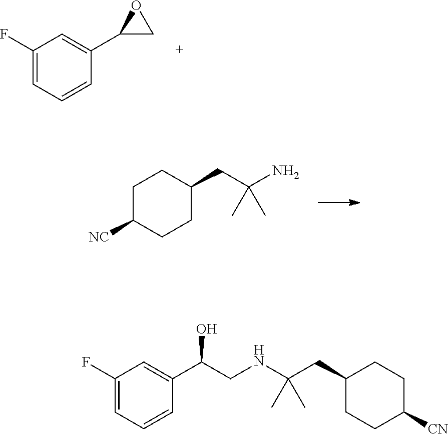 [SMILES: Fc1cccc([C@@H]2CO2)c1 ; CC(C)(N)C[C@H]1CC[C@@H](C#N)CC1 ; CC(C)(C[C@H]1CC[C@@H](C#N)CC1)NC[C@H](O)c1cccc(F)c1]

The title compound was prepared from (R)-2-(3-fluorophenyl)oxirane and (1s,4s)-4-(2-amino-2-methylpropyl)cyclohexane-1-carbonitrile in accordance with the procedure in Example 1, Step (e).

¹H NMR (400 MHz, CDCl₃) δ: 7.34-7.27 (m, 1H), 7.16-7.06 (m, 2H), 6.96 (tdd, J=8.4, 2.6, 1.0 Hz, 1H), 4.61 (dd, J=8.5, 3.7 Hz, 1H), 2.93-2.83 (m, 2H), 2.55 (dd, J=11.9, 8.5 Hz, 1H), 1.99-1.88 (m, 2H), 1.79-1.65 (m, 2H), 1.65-1.50 (m, 2H), 1.47-1.31 (m, 5H), 1.09 (s, 3H), 1.08 (s, 3H).

### Example 130: 6-((R)-2-((1-((1r,4R)-4-Aminocyclohexyl)-2-methylpropan-2-yl)amino)-1-hydroxyethyl)picolinonitrile trihydrochloride

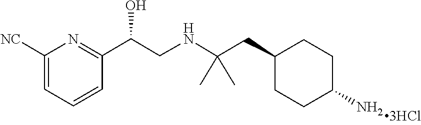 [SMILES: CC(C)(C[C@H]1CC[C@H](N)CC1)NC[C@@H](O)c1cccc(C#N)n1]

### (a) 6-Vinylpicolinonitrile

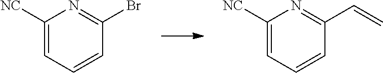

4,4,5,5-Tetramethyl-2-vinyl-1,3,2-dioxaborolane (0.45 m L, 2.60 mmol), tetrakis-(triphenylphosphoranyl)palladium (125 mg, 0.11 mmol) and Na₂CO₃ (344 mg, 3.25 mmol) where added to a mixture of 2-chloro-6-cyanopyridine (300 mg, 2.17 mmol) in dioxane/H₂O (2 mL/0.5 mL) at rt. The mixture was stirred at 90° C. for 7 h, cooled to rt, diluted with H₂O and extracted with EtOAc. The combined extracts were dried (Na₂SO₄) and concentrated. The residue was purified by chromatography to give the sub-title compound (282 mg, 99%), which was used directly in the next step.

### (b) (S)-6-(Oxiran-2-yl)picolinonitrile and (R)-6-(oxiran-2-yl)picolinonitrile

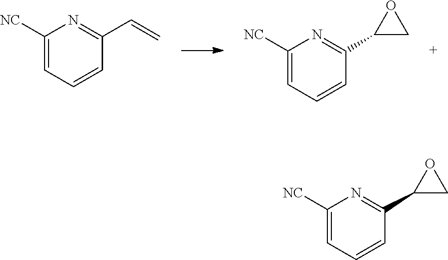

mCPBA (2.67 g, 10.83 mmol) was added in portions to an ice-cooled solution of 6-vinylpicolinonitrile (282 mg, 2.17 mmol) in CH₂Cl₂ (10 mL). The mixture was stirred at rt for 16 h, diluted with CH₂Cl₂ and cooled in an ice-bath. NaHCO₃(aq, 10%) and Na₂SO₃ (aq, 10%) were added and the mixture was stirred for 10 min and the layers separated. The aq layer was extracted with CH₂Cl₂ and the combined organics phases were washed with brine, dried (Na₂SO₄) and concentrated. The residue was purified by chromatography and the enantiomers were separated by preparative HPLC (Chiralpak IC column (30 mm×250 mm, 5 μm), eluent system 20% iPrOH, 80% Heptane, flow rate 40 mL/min) to give the sub-title compounds; (S)-6-(oxiran-2-yl)picolinonitrile (63 mg, 20%) and (R)-6-(oxiran-2-yl)picolinonitrile (74 mg, 23%).

### (c) tert-Butyl ((1R,4r)-4-(2-(((R)-2-(6-cyanopyridin-2-yl)-2-hydroxyethyl)amino)-2-methylpropyl)cyclohexyl)carbamate

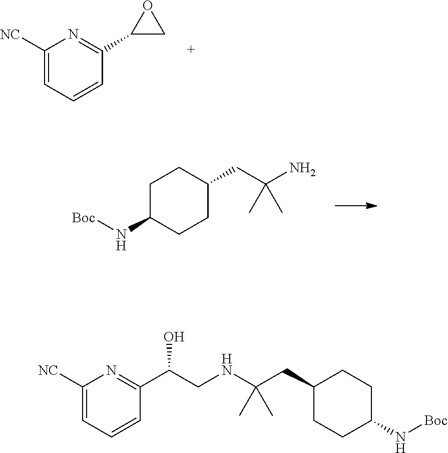 [SMILES: N#Cc1cccc([C@H]2CO2)n1 ; CC(C)(N)C[C@H]1CC[C@H](NC(=O)OC(C)(C)C)CC1 ; CC(C)(C[C@H]1CC[C@H](NC(=O)OC(C)(C)C)CC1)NC[C@@H](O)c1cccc(C#N)n1]

The sub-title compound was prepared from (S)-6-(oxiran-2-yl)picolinonitrile and tert-butyl ((1r,4r)-4-(2-amino-2-methylpropyl)cyclohexyl)carbamate (see Example 85, Step (b)) in accordance with the procedure in Example 1, Step (e).

### (d) 6-((R)-2-((1-((1r,4R)-4-Aminocyclohexyl)-2-methylpropan-2-yl)amino)-1-hydroxyethyl)picolinonitrile trihydrochloride

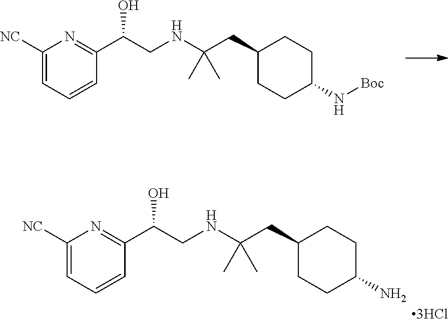 [SMILES: CC(C)(C[C@H]1CC[C@H](NC(=O)OC(C)(C)C)CC1)NC[C@@H](O)c1cccc(C#N)n1 ; CC(C)(C[C@H]1CC[C@H](N)CC1)NC[C@@H](O)c1cccc(C#N)n1]

TFA (0.68 mL, 8.88 mmol) was added dropwise to a solution of tert-butyl ((1R,4r)-4-(2-(((R)-2-(6-cyanopyridin-2-yl)-2-hydroxyethyl)amino)-2-methylpropyl)cyclohexyl)-carbamate (74 mg, 0.18 mmol) in CH₂Cl₂ (2 mL) at rt. The mixture was stirred at rt for 2 h and concentrated. The residue was purified with preparative HPLC (Atlantis T3, 30×100 mm, 5 μm, 0.02% TFA in H₂O:MeCN, 95:5 to 5:95). The product containing fractions were pooled and concentrated. The residue was partitioned between CH₂Cl₂ and NaHCO₃ (aq, sat) and the layers were separated. The aq layer was extracted with CH₂Cl₂ and the combined organic phases were washed with brine, dried (Na₂SO₄) and concentrated. The residue was dissolved in dry CH₂Cl₂/heptane (2 mL, v/v=1:2). HCl (4 M in dioxane, 146 μL, 0.59 mmol) was added. The mixture was sonicated and the solids collected by centrifugation and washed twice with CH₂Cl₂/heptane (2 mL, v/v=1:2) and dried to give the title compound (38 mg, 50%).

¹H NMR (400 MHz, CD₃OD, ppm) δ 8.09 (t, J=7.9 Hz, 1H), 7.99-7.94 (m, 1H), 7.85 (dd, J=7.6, 1.1 Hz, 1H), 5.09 (dd, J=9.0, 3.4 Hz, 1H), 3.49 (dd, J=12.6, 3.4 Hz, 1H), 3.25 (dd, J=12.5, 9.0 Hz, 1H), 3.06 (tt, J=11.8, 4.0 Hz, 1H), 2.11-1.90 (m, 5H), 1.67 (dd, J=4.6, 1.6 Hz, 2H), 1.59-1.46 (m, 4H), 1.45 (s, 3H), 1.44 (s, 3H), 1.31-1.21 (m, 2H).

### Example 131: 6-((R)-2-((1-((1s,4S)-4-Aminocyclohexyl)-2-methylpropan-2-yl)amino)-1-hydroxyethyl)picolinonitrile trihydrochloride

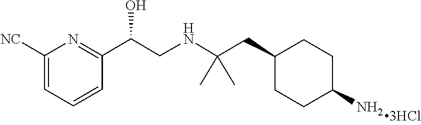 [SMILES: CC(C)(C[C@H]1CC[C@@H](N)CC1)NC[C@@H](O)c1cccc(C#N)n1]

### (a) tert-Butyl ((1s,4s)-4-(2-amino-2-methylpropyl)cyclohexyl)carbamate

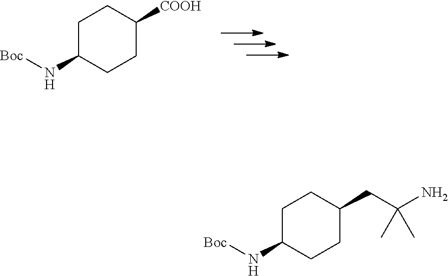 [SMILES: CC(C)(C)OC(=O)N[C@H]1CC[C@@H](C(=O)O)CC1 ; CC(C)(N)C[C@H]1CC[C@@H](NC(=O)OC(C)(C)C)CC1]

The sub-title compound was prepared from (1s,4s)-4-((tert-butoxycarbonyl)amino)-cyclohexane-1-carboxylic acid in accordance with the procedures in Example 67, Steps (a) and (b) and Example 85, Steps (a) and (b).

### (b) 6-((R)-2-((1-((1s,4S)-4-Aminocyclohexyl)-2-methylpropan-2-yl)amino)-1-hydroxyethyl)picolinonitrile

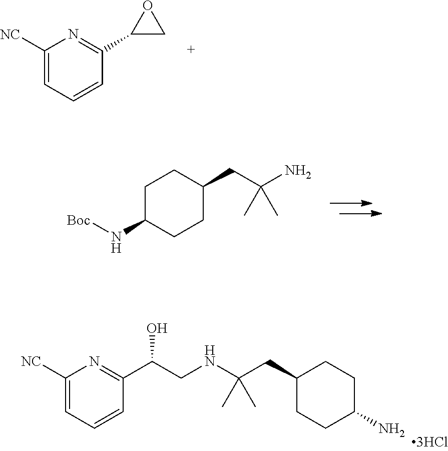 [SMILES: N#Cc1cccc([C@H]2CO2)n1 ; CC(C)(N)C[C@H]1CC[C@@H](NC(=O)OC(C)(C)C)CC1 ; CC(C)(C[C@H]1CC[C@H](N)CC1)NC[C@@H](O)c1cccc(C#N)n1]

The title compound was prepared from (S)-6-(oxiran-2-yl)picolinonitrile and tert-butyl ((1s,4s)-4-(2-amino-2-methylpropyl)cyclohexyl)carbamate in accordance with the procedures in Example 130, Steps (c) and (d).

¹H NMR (400 MHz, CD₃OD) δ 8.09 (t, J=7.8 Hz, 1H), 7.97 (d, J=8.0 Hz, 1H), 7.85 (dd, J=7.6, 0.6 Hz, 1H), 5.09 (dd, J=9.0, 3.4 Hz, 1H), 3.51 (dd, J=12.5, 3.5 Hz, 1H), 3.30-3.23 (m, 2H), 1.93-1.80 (m, 3H), 1.79-1.67 (m, 6H), 1.65-1.55 (m, 2H), 1.44 (s, 6H).

### Example 132: 6-((S)-2-((1-((1r,4S)-4-Aminocyclohexyl)-2-methylpropan-2-yl)amino)-1-hydroxyethyl)picolinonitrile trihydrochloride

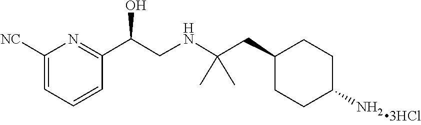 [SMILES: CC(C)(C[C@H]1CC[C@H](N)CC1)NC[C@H](O)c1cccc(C#N)n1]

The title compound was prepared from (R)-6-(oxiran-2-yl)picolinonitrile in accordance with the procedures in Example 130.

¹H NMR (600 MHz, CD₃OD) δ 8.09 (t, J=8.0 Hz, 1H), 7.96 (d, J=8.0 Hz, 1H), 7.85 (dd, J=7.6, 0.6 Hz, 1H), 5.08 (dd, J=9.0, 3.4 Hz, 1H), 3.49 (dd, J=12.6, 3.4 Hz, 1H), 3.25 (dd, J=12.6, 9.0 Hz, 1H), 3.06 (tt, J=12.0, 4.0 Hz, 1H), 2.09-1.92 (m, 4H), 1.71-1.63 (m, 2H), 1.59-1.47 (m, 3H), 1.45 (s, 3H), 1.44 (s, 3H), 1.30-1.18 (m, 2H).

### Example 133: tert-Butyl ((1R,4s)-4-(2-(((5)-2-(6-Cyanopyridin-2-yl)-2-hydroxyethyl)-amino)-2-methylpropyl)cyclohexyl)carbamate

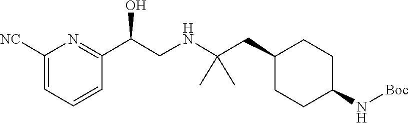 [SMILES: CC(C)(C[C@H]1CC[C@@H](NC(=O)OC(C)(C)C)CC1)NC[C@H](O)c1cccc(C#N)n1]

The title compound was prepared from (R)-6-(oxiran-2-yl)picolinonitrile and tert-butyl ((1s,4s)-4-(2-amino-2-methylpropyl)cyclohexyl)carbamate (see Example 131, Step (a)) in accordance with the procedures in Example 130, Step (c).

¹H NMR (400 MHz, CDCl₃, ppm) δ 7.84 (dd, J=8.0, 7.6 Hz, 1H), 7.78 (dd, J=8.0, 1.2 Hz, 1H), 7.59 (dd, J=7.6, 1.2 Hz, 1H), 4.73 (dd, J=7.7, 4.1 Hz, 1H), 4.66-4.50 (m, 1H), 3.69-3.53 (m, 1H), 3.32 (br s, 1H), 3.11 (dd, J=11.9, 4.2 Hz, 1H), 2.71 (dd, J=11.9, 7.7 Hz, 1H), 1.66-1.48 (m, 7H), 1.43 (s, 9H), 1.35-1.30 (m, 2H), 1.29-1.19 (m, 3H), 1.08 (s, 6H).

### Example 134: 6-((S)-2-((1-((1s,4R)-4-Aminocyclohexyl)-2-methylpropan-2-yl)amino)-1-hydroxyethyl)picolinonitrile trihydrochloride

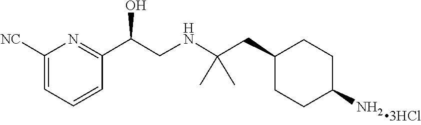 [SMILES: CC(C)(C[C@H]1CC[C@@H](N)CC1)NC[C@H](O)c1cccc(C#N)n1]

The title compound was prepared from tert-butyl ((1R,4s)-4-(2-(((S)-2-(6-cyanopyridin-2-yl)-2-hydroxyethyl)amino)-2-methylpropyl)cyclohexyl)carbamate in accordance with the procedures in Example 130, Step (d).

¹H NMR (400 MHz, CD₃OD) δ 8.09 (t, J=7.9 Hz, 1H), 7.97 (d, J=7.9 Hz, 1H), 7.85 (dd, J=7.6, 0.6 Hz, 1H), 5.10 (dd, J=9.0, 3.4 Hz, 1H), 3.51 (dd, J=12.5, 3.4 Hz, 1H), 3.30-3.24 (m, 2H), 1.97-1.54 (m, 12H), 1.44 (s, 6H).

Examples 135 to 142 were prepared from the appropriate amine in Examples 130, 131, 132 and 134, using acetyl chloride in accordance with the procedure in Example 67, Step (f), or methanesulfonyl chloride, in accordance with the procedure in Example 71, Step (a).

### Example 135: N-((1R,4r)-4-(2-(((R)-2-(6-Cyanopyridin-2-yl)-2-hydroxyethyl)amino)-2-methylpropyl)cyclohexyl)acetamide

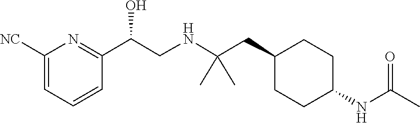 [SMILES: CC(=O)N[C@H]1CC[C@H](CC(C)(C)NC[C@@H](O)c2cccc(C#N)n2)CC1]

¹H NMR (300 MHz, CDCl₃) δ 7.94-7.84 (m, 2H), 7.66-7.58 (m, 1H), 5.43 (d, J=8.2 Hz, 1H), 5.19 (dd, J=8.6, 3.5 Hz, 1H), 3.68-3.58 (m, 1H), 3.48 (dd, J=12.1, 3.5 Hz, 1H), 2.99 (dd, J=12.1, 8.6 Hz, 1H), 1.95 (s, 3H), 1.94-1.90 (m, 2H), 1.85-1.75 (m, 2H), 1.59-1.52 (m, 2H), 1.51-1.40 (m, 1H), 1.36 (s, 3H), 1.35 (s, 3H), 1.21-1.10 (m, 4H).

### Example 136: N-((1R,4r)-4-(2-(((R)-2-(6-Cyanopyridin-2-yl)-2-hydroxyethyl)amino)-2-methylpropyl)cyclohexyl)methanesulfonamide

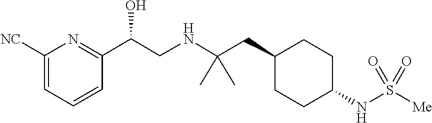 [SMILES: CC(C)(C[C@H]1CC[C@H](NS(C)(=O)=O)CC1)NC[C@@H](O)c1cccc(C#N)n1]

¹H NMR (300 MHz, CDCl₃) δ 7.90-7.82 (m, 1H), 7.79 (dd, J=8.1, 1.2 Hz, 1H), 7.61 (dd, J=7.3, 1.2 Hz, 1H), 4.76 (dd, J=7.5, 4.1 Hz, 1H), 4.28 (br s, 1H), 3.30-3.15 (m, 2H), 3.12 (dd, J=11.9, 4.1 Hz, 1H), 2.97 (s, 3H), 2.75 (dd, J=11.9, 7.5 Hz, 1H), 2.10-1.95 (m, 2H), 1.81 (d, J=13.0 Hz, 2H), 1.37-1.27 (m, 4H), 1.28-1.23 (m, 2H), 1.11-1.07 (m, 6H).

### Example 137: N-((1S,4s)-4-(2-(((R)-2-(6-Cyanopyridin-2-yl)-2-hydroxyethyl)amino)-2-methylpropyl)cyclohexyl)acetamide

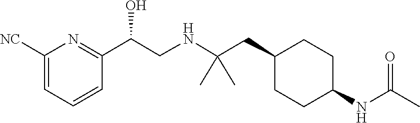 [SMILES: CC(=O)N[C@H]1CC[C@@H](CC(C)(C)NC[C@@H](O)c2cccc(C#N)n2)CC1]

¹H NMR (300 MHz, CDCl₃) δ 7.85 (dd, J=8.0, 7.4 Hz, 1H), 7.80-7.75 (m, 1H), 7.60 (dd, J=7.4, 1.2 Hz, 1H), 5.55 (d, J=7.4 Hz, 1H), 4.72 (dd, J=7.4, 4.1 Hz, 1H), 3.93 (br s, 1H), 3.10 (dd, J=11.9, 4.2 Hz, 1H), 3.07-2.80 (m, 1H), 2.72 (dd, J=11.9, 7.6 Hz, 1H), 1.97 (s, 3H), 1.69-1.46 (m, 7H), 1.37-1.16 (m, 4H), 1.07 (s, 6H).

### Example 138: N-((1S,4s)-4-(2-(((R)-2-(6-Cyanopyridin-2-yl)-2-hydroxyethyl)amino)-2-methylpropyl)cyclohexyl)methanesulfonamide

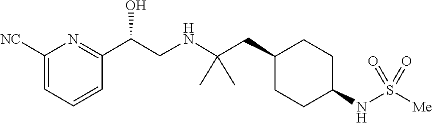 [SMILES: CC(C)(C[C@H]1CC[C@@H](NS(C)(=O)=O)CC1)NC[C@@H](O)c1cccc(C#N)n1]

¹H NMR (400 MHz, CDCl₃) δ 7.86 (t, J=7.8 Hz, 1H), 7.78 (dd, J=8.1, 1.2 Hz, 1H), 7.61 (dd, J=7.5, 1.2 Hz, 1H), 4.75 (dd, J=7.5, 4.1 Hz, 1H), 4.40 (s, 1H), 3.57 (s, 1H), 3.12 (dd, J=11.9, 4.1 Hz, 1H), 2.97 (s, 3H), 2.74 (dd, J=11.9, 7.5 Hz, 1H), 1.75-1.48 (m, 7H), 1.38-1.22 (m, 5H), 1.09 (d, J=1.8 Hz, 6H).

### Example 139: N-((1S,4r)-4-(2-(((S)-2-(6-Cyanopyridin-2-yl)-2-hydroxyethyl)amino)-2-methylpropyl)cyclohexyl)acetamide

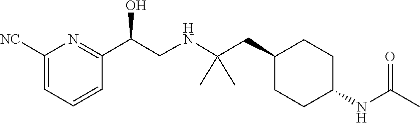 [SMILES: CC(=O)N[C@H]1CC[C@H](CC(C)(C)NC[C@H](O)c2cccc(C#N)n2)CC1]

¹H NMR (400 MHz, CDCl₃) δ 7.88-7.82 (m, 1H), 7.79 (dd, J=8.2, 1.2 Hz, 1H), 7.60 (dd, J=7.4, 1.2 Hz, 1H), 5.29 (d, J=8.2 Hz, 1H), 4.75 (dd, J=7.4, 4.1 Hz, 1H), 3.74-3.61 (m, 1H), 3.11 (dd, J=11.9, 4.1 Hz, 1H), 2.92 (br s, 1H), 2.75 (dd, J=11.9, 7.4 Hz, 1H), 1.99-1.90 (m, 2H), 1.95 (s, 3H), 1.81-1.74 (m, 2H), 1.35-1.27 (m, 4H), 1.17-1.10 (m, 4H), 1.10 (s, 3H), 1.09 (s, 3H).

### Example 140: N-((1S,4r)-4-(2-(((S)-2-(6-Cyanopyridin-2-yl)-2-hydroxyethyl)amino)-2-methylpropyl)cyclohexyl)methanesulfonamide

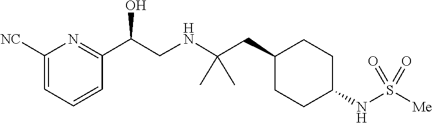 [SMILES: CC(C)(C[C@H]1CC[C@H](NS(C)(=O)=O)CC1)NC[C@H](O)c1cccc(C#N)n1]

¹H NMR (400 MHz, CDCl₃) δ 7.89-7.82 (m, 1H), 7.80-7.75 (m, 1H), 7.60 (dd, J=7.5, 1.1 Hz, 1H), 4.71 (dd, 3=7.5, 4.1 Hz, 1H), 4.32 (br s, 1H), 3.21 (tt, 3=11.4, 4.1 Hz, 1H), 3.07 (dd, J=11.8, 4.1 Hz, 1H), 2.97 (s, 3H), 2.72 (dd, 3=11.8, 7.5 Hz, 1H), 2.08-1.98 (m, 2H), 1.86-1.76 (m, 2H), 1.37-1.19 (m, 6H), 1.16-1.07 (m, 2H), 1.06 (s, 3H), 1.06 (s, 3H).

### Example 141: N-((1R,4s)-4-(2-(((S)-2-(6-Cyanopyridin-2-yl)-2-hydroxyethyl)amino)-2-methylpropyl)cyclohexyl)acetamide

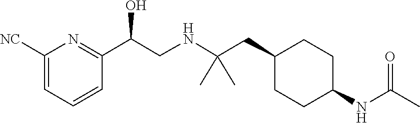 [SMILES: CC(=O)N[C@H]1CC[C@@H](CC(C)(C)NC[C@H](O)c2cccc(C#N)n2)CC1]

¹H NMR (400 MHz, CDCl₃) δ δ 7.87-7.81 (m, 1H), 7.79-7.75 (m, 1H), 7.62-7.57 (m, 1H), 5.52 (d, J=6.6 Hz, 1H), 4.70 (dd, J=7.5, 4.2 Hz, 1H), 4.00-3.89 (m, 1H), 3.09 (dd, J=11.9, 4.2 Hz, 1H), 2.71 (dd, J=11.9, 7.5 Hz, 1H), 2.70-2.30 (br s, 1H), 1.97 (s, 3H), 1.70-1.49 (m, 7H), 1.34-1.19 (m, 5H), 1.07 (s, 6H).

### Example 142: N-((1R,4s)-4-(2-(((S)-2-(6-Cyanopyridin-2-yl)-2-hydroxyethyl)amino)-2-methylpropyl)cyclohexyl)methanesulfonamide

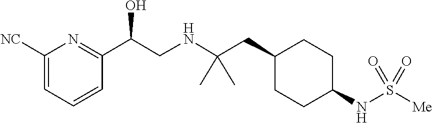 [SMILES: CC(C)(C[C@H]1CC[C@@H](NS(C)(=O)=O)CC1)NC[C@H](O)c1cccc(C#N)n1]

¹H NMR (400 MHz, CDCl₃) δ 7.88-7.82 (m, 1H), 7.79-7.75 (m, 1H), 7.60 (dd, J=7.6, 1.2 Hz, 1H), 4.70 (dd, J=7.5, 4.1 Hz, 1H), 4.42 (br s, 1H), 3.57 (br s, 1H), 3.08 (dd, J=11.9, 4.2 Hz, 1H), 2.97 (s, 3H), 2.72 (dd, J=11.9, 7.5 Hz, 1H), 1.75-1.46 (m, 8H), 1.37-1.19 (m, 5H), 1.07 (s, 6H).

### Example 143: N-((1S,4s)-4-(2-(((R)—H-hydroxy-2-(2-methylpyridin-3-yl)ethyl)amino)-2-methylpropyl)cyclohexyl)acetamide dihydrochloride

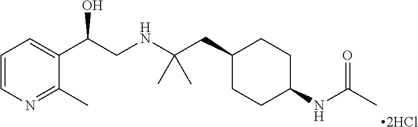 [SMILES: CC(=O)N[C@H]1CC[C@@H](CC(C)(C)NC[C@H](O)c2cccnc2C)CC1 ; "·2HCl"]

### (a) 2-Bromo-1-(2-methylpyridin-3-yl)ethan-1-one hydrobromide

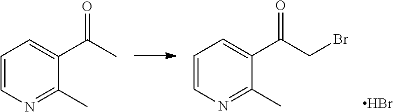

HBr (33% in AcOH, 0.49 mL, 2.85 mmol) followed by pyridinium bromide perbromide (573 mg, 1.79 mmol) were added to a solution of 1-(2-methylpyridin-3-yl)ethan-1-one (220 mg, 1.63 mmol) in AcOH (0.88 mL) at rt. The mixture was stirred at rt for 1 h and cooled in an ice bath. Ice-cold Et₂O (5 mL) was added and the solids were collected by decantation and washed twice with ice-cold Et₂O and dried to give the sub-title compound (480 mg, 99%) which was used directly in the next step without further purification.

### (b) 2-Chloro-1-(2-methylpyridin-3-yl)ethan-1-one

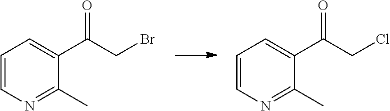

NaCl (aq, sat, 4.8 mL, 32.55 mmol) was added to a stirred mixture of 2-bromo-1-(2-methylpyridin-3-yl)ethan-1-one hydrobromide (480 mg, 0.36 mmol) in THF (8 mL) at rt. The mixture was stirred vigorously at 80° C. for 4 h and cooled to rt. NaHCO₃(aq, sat) was added until pH ˜6, and the mixture was extracted with EtOAc. The combined extracts were dried (Na₂SO₄) and concentrated. The residue was purified by chromatography to give the sub-title compound (263 mg, 69%) which was used in the next step without further purification.

### (c) (R)-2-Chloro-1-(2-methylpyridin-3-yl)ethan-1-ol

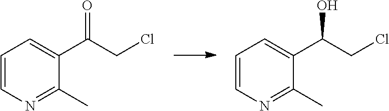

A solution of 2-chloro-1-(2-methylpyridin-3-yl)ethan-1-one (237 mg, 1.40 mmol) in DMF (6 mL) was added to (S,S)—N-(p-toluenesulfonyl)-1,2-diphenylethanediamine(chloro)(p-cymene)ruthenium(II) (7 mg, 12 μmol). HCOOH/Et₃N (5:2, 0.60 mL) was added and the mixture was stirred at rt for 1 h. MeOH (5 ml) was added and the mixture was stirred for 5 min and concentrated. EtOAc/Hexane (9:1; 150 mL) was added to the residue and the mixture was washed with NaHCO₃(aq, sat), brine, dried (Na₂SO₄) and concentrated. The residue was purified by chromatography to give the sub-title compound (166 mg, 69%).

¹H NMR (400 MHz, CDCl₃) δ 8.41 (dd, J=4.8, 1.8 Hz, 1H), 7.86 (dd, J=7.8, 1.8 Hz, 1H), 7.18 (dd, J=7.8, 4.8 Hz, 1H), 5.11 (dd, J=8.7, 3.2 Hz, 1H), 3.73 (dd, J=11.4, 3.2 Hz, 1H), 3.57 (dd, J=11.4, 8.8 Hz, 1H), 2.90-2.80 (br s, 1H), 2.55 (s, 3H).

### (d) (R)-2-Methyl-3-(oxiran-2-yl)pyridine

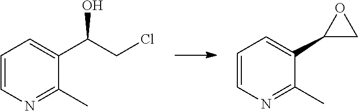

NaOH (aq, 4 M, 0.24 mL, 0.96 mmol) was added to a mixture of of (R)-2-chloro-1-(2-methylpyridin-3-yl)ethan-1-ol (150 mg, 0.87 mmol) in iPrOH (0.87 mL, 11.36 mmol) at rt. The mixture was stirred at rt for 1 h and diluted with EtOAc. H₂O was added and the layers separated were separated. The aq layer was extracted with EtOAc and the combined organic phases were washed with H₂O, brine, dried (Na₂SO₄) and concentrated to give the sub-title compound (118 mg, 99%) which was used immediately in the next step without further purification.

### (e) N-((1S,4s)-4-(2-(((R)-2-Hydroxy-2-(2-methylpyridin-3-yl)ethyl)amino)-2-methylpropyl)cyclohexyl)acetamide dihydrochloride

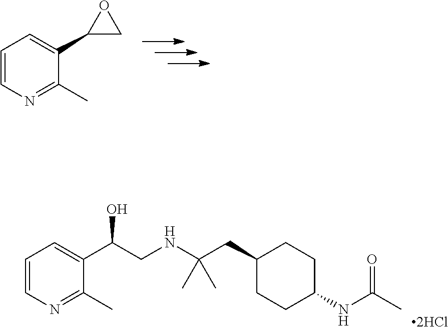

The title compound was prepared from (R)-2-methyl-3-(oxiran-2-yl)pyridine and tert-butyl ((1s,4s)-4-(2-amino-2-methylpropyl)cyclohexyl)carbamate in accordance with the procedures in Example 130, Steps (c) and (d) and Example 67, Step (f).

¹H NMR (400 MHz, D₂O) δ 8.71 (d, J=8.1 Hz, 1H), 8.62 (d, J=5.8 Hz, 1H), 7.97 (dd, J=8.1, 5.8 Hz, 1H), 5.46 (s, 1H), 5.35 (dd, J=10.2, 2.4 Hz, 1H), 3.83-3.77 (m, 1H), 3.31 (dd, J=13.0, 2.4 Hz, 1H), 3.22 (dd, J=13.0, 10.2 Hz, 1H), 2.81 (s, 3H), 1.99 (s, 3H), 1.74-1.55 (m, 9H), 1.42 (d, J=5.8 Hz, 8H).

### Example 144: N-((1S,4s)-4-(2-(((R)-2-Hydroxy-2-(2-methylpyridin-3-yl)ethyl)amino)-2-methylpropyl)cyclohexyl)methanesulfonamide dihydrochloride

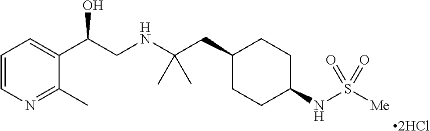 [SMILES: Cc1ncccc1[C@@H](O)CNC(C)(C)C[C@H]1CC[C@@H](NS(C)(=O)=O)CC1 ; "·2HCl"]

The title compound was prepared from (R)-2-methyl-3-(oxiran-2-yl)pyridine and tert-butyl ((1s,4s)-4-(2-amino-2-methylpropyl)cyclohexyl)carbamate in accordance with the procedures in Example 130, Steps (c) and (d) and Example 71, Step (a).

¹H NMR (400 MHz, D₂O) δ 8.71 (dd, J=8.0, 1.0 Hz, 1H), 8.62 (dd, J=6.0, 1.2 Hz, 1H), 7.97 (dd, J=8.0, 6.0 Hz, 1H), 5.46 (s, 1H), 5.36 (dd, J=10.2, 2.6 Hz, 1H), 3.60-3.49 (m, 1H), 3.32 (dd, J=13.2, 2.6 Hz, 1H), 3.22 (dd, J=13.2, 10.2 Hz, 1H), 3.10 (s, 3H), 2.81 (s, 3H), 1.81-1.59 (m, 9H), 1.42 (d, J=5.4 Hz, 8H).

### Example 145: N-((1R,4r)-4-(2-(((R)-2-Hydroxy-2-(2-methylpyridin-3-yl)ethyl)amino)-2-methylpropyl)cyclohexyl)acetamide dihydrochloride

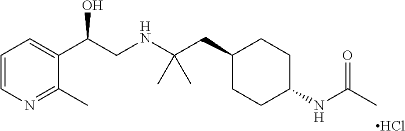 [SMILES: CC(=O)N[C@H]1CC[C@H](CC(C)(C)NC[C@H](O)c2cccnc2C)CC1 ; Cl]

The title compound was prepared from (R)-2-methyl-3-(oxiran-2-yl)pyridine and tert-butyl ((1r,4r)-4-(2-amino-2-methylpropyl)cyclohexyl)carbamate (see Example 85, Step (b)) in accordance with the procedures in Example 130, Steps (c) and (d) and Example 67, Step (f).

¹H NMR (400 MHz, D₂O) δ 8.71 (dd, J=8.2, 1.4 Hz, 1H), 8.62 (dd, J=6.0, 1.6 Hz, 1H), 7.97 (dd, J=8.2, 6.0 Hz, 1H), 5.46 (s, 1H), 5.35 (dd, J=10.3, 2.6 Hz, 1H), 3.58-3.47 (m, 1H), 3.30 (dd, J=13.2, 2.8 Hz, 1H), 3.21 (dd, J=13.2, 10.3 Hz, 1H), 2.81 (s, 3H), 1.95 (s, 3H), 1.91-1.80 (m, 4H), 1.63 (d, J=5.1 Hz, 2H), 1.53-1.44 (m, 1H), 1.42 (d, J=4.6 Hz, 6H), 1.34-1.11 (m, 5H).

### Example 146: N-((1R,4r)-4-(2-(((R)-2Hydroxy-2-(2-methylpyridin-3-yl)ethyl)amino)-2-methylpropyl)cyclohexyl)methanesulfonamide dihydrochloride

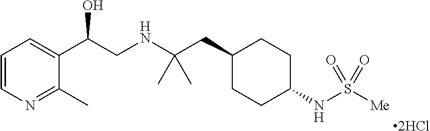 [SMILES: Cc1ncccc1[C@@H](O)CNC(C)(C)C[C@H]1CC[C@H](NS(C)(=O)=O)CC1 ; "·2HCl"]

The title compound was prepared from (R)-2-methyl-3-(oxiran-2-yl)pyridine and tert-butyl ((1r,4r)-4-(2-amino-2-methylpropyl)cyclohexyl)carbamate (see Example 85, Step (b)) in accordance with the procedures in Example 130, Steps (c) and (d) and Example 71, Step (a).

¹H NMR (400 MHz, D₂O) δ 8.55 (d, J=5.4 Hz, 1H), 8.48 (d, J=7.4 Hz, 1H), 7.79 (t, J=6.3 Hz, 1H), 5.46 (s, 1H), 5.31 (d, J=10.0 Hz, 1H), 3.33-3.15 (m, 3H), 3.10 (s, 3H), 2.73 (s, 3H), 2.00 (d, J=11.4 Hz, 2H), 1.86 (d, J=12.4 Hz, 2H), 1.62 (t, J=5.0 Hz, 2H), 1.51-1.45 (m, 1H), 1.41 (d, J=3.8 Hz, 6H), 1.40-1.30 (m, 2H), 1.27-1.13 (m, 2H).

### Example 147: tert-Butyl ((1R,4s)-4-(2-(((S)-2-hydroxy-2-(6-(trifluoromethyl)pyridin-2-yl)ethyl)amino)-2-methylpropyl)cyclohexyl)carbamate

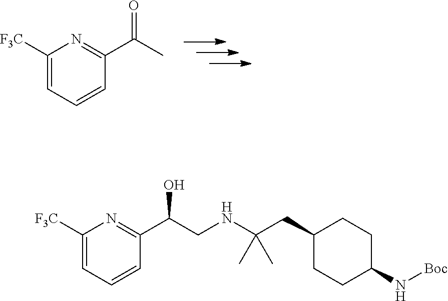 [SMILES: CC(=O)c1cccc(C(F)(F)F)n1 ; CC(C)(C[C@H]1CC[C@@H](NC(=O)OC(C)(C)C)CC1)NC[C@H](O)c1cccc(C(F)(F)F)n1]

The title compound was prepared from 1-(6-(trifluoromethyl)pyridin-2-yl)ethan-1-one in accordance with the procedures in Example 143, Steps (a) to (d), followed by the reaction with tert-butyl ((1s,4s)-4-(2-amino-2-methylpropyl)cyclohexyl)carbamate (see Example 131, Step (a)) in accordance with the procedure in Example 1, Step (e).

¹H NMR (400 MHz, CDCl₃) δ 7.88 (t, J=7.8 Hz, 1H), 7.74 (d, J=7.8 Hz, 1H), 7.58 (d, J=7.8 Hz, 1H), 4.82 (dd, J=7.2, 4.2 Hz, 1H), 4.58 (br s, 1H), 3.64 (br s, 1H), 3.12 (dd, J=11.8, 4.2 Hz, 1H), 2.80 (dd, J=11.8, 7.2 Hz, 1H), 1.65-1.48 (m, 7H), 1.44 (s, 9H), 1.37-1.32 (m, 2H), 1.30-1.19 (m, 2H), 1.10 (s, 6H).

### Example 148: tert-Butyl ((1S,4r)-4-(2-(((S)-2-hydroxy-2-(6-(trifluoromethyl)pyridin-2-yl)ethyl)amino)-2-methylpropyl)cyclohexyl)carbamate

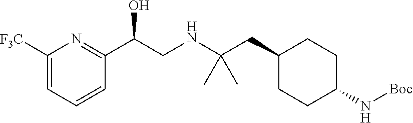 [SMILES: CC(C)(C[C@H]1CC[C@H](NC(=O)OC(C)(C)C)CC1)NC[C@H](O)c1cccc(C(F)(F)F)n1]

The title compound was prepared from 1-(6-(trifluoromethyl)pyridin-2-yl)ethan-1-one in accordance with the procedures in Example 143, Steps (a) to (d), followed by the reaction with tert-butyl ((1r,4r)-4-(2-amino-2-methylpropyl)cyclohexyl)carbamate (see Example 85, Step (b)) in accordance with the procedure in Example 1, Step (e).

¹H NMR (400 MHz, CDCl₃, ppm) δ 7.88 (t, J=7.8 Hz, 1H), 7.72 (d, J=7.8 Hz, 1H), 7.58 (d, J=7.8 Hz, 1H), 4.76 (dd, J=7.2, 4.2 Hz, 1H), 4.43-4.26 (m, 1H), 3.33 (br s, 1H), 3.07 (dd, J=11.8, 4.2 Hz, 1H), 2.75 (dd, J=11.8, 7.2 Hz, 1H), 2.02-1.89 (m, 2H), 1.82-1.70 (m, 2H), 1.44 (s, 9H), 1.31-1.24 (m, 3H), 1.12-1.03 (m, 10H).

### Example 149: N-((1R,4s)-4-(2-(((S)-2-Hydroxy-2-(6-(trifluoromethyl)pyridin-2-yl)-ethyl)amino)-2-methylpropyl)cyclohexyl)acetamide dihydrochloride

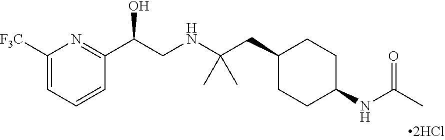 [SMILES: CC(=O)N[C@H]1CC[C@@H](CC(C)(C)NC[C@H](O)c2cccc(C(F)(F)F)n2)CC1]

### (a) (S)-2-((1-((1s,4R)-4-Aminocyclohexyl)-2-methylpropan-2-yl)amino)-1-(6-(trifluoromethyl)pyridin-2-yl)ethan-1-ol tris-trifluoroacetate

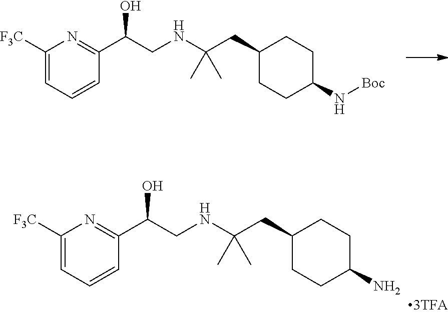 [SMILES: CC(C)(C[C@H]1CC[C@@H](NC(=O)OC(C)(C)C)CC1)NC[C@H](O)c1cccc(C(F)(F)F)n1 ; CC(C)(C[C@H]1CC[C@@H](N)CC1)NC[C@H](O)c1cccc(C(F)(F)F)n1]

TFA (1.0 mL, 13.6 mmol) was added dropwise to a solution of tert-butyl ((1R,4s)-4-(2-(((S)-2-hydroxy-2-(6-(trifluoromethyl)pyridin-2-yl)ethyl)amino)-2-methylpropyl)cyclo-hexyl)carbamate (125 mg, 0.27 mmol) in CH₂Cl₂ (5 mL) at rt. The mixture was stirred at rt for 2 h and concentrated to give the sub-title product (192 mg, 99%).

### (b) N-((1R,4s)-4-(2-(((S)-2-Hydroxy-2-(6-(trifluoromethyl)pyridin-2-yl)ethyl)amino)-2-methylpropyl)cyclohexyl)acetamide dihydrochloride

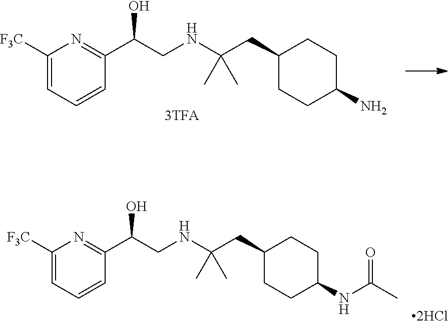 [SMILES: CC(C)(C[C@H]1CC[C@@H](N)CC1)NC[C@H](O)c1cccc(C(F)(F)F)n1 ; CC(=O)N[C@H]1CC[C@@H](CC(C)(C)NC[C@H](O)c2cccc(C(F)(F)F)n2)CC1]

A solution of acetyl chloride (0.14 mmol, 10 μL) in CH₂Cl₂ (1 mL) was added to (S)-2-((1-((1r,4S)-4-aminocyclohexyl)-2-methylpropan-2-yl)amino)-1-(6-(trifluoromethyl)pyridin-2-in CH₂Cl₂ (3 mL) at −10° C. followed by Et₃N (0.14 mL, 1.01 mmol). The mixture was allowed to warm to rt and was stirred at rt for 30 min. NaHCO₃(aq, sat) was added and the mixture was extracted with CH₂Cl₂. The combined extracts were dried (Na₂SO₄) and concentrated. The residue was purified by chromatography and the obtained material was dissolved in CH₂Cl₂/heptane (2 mL, v/v=1:2) and mixed with HCl (4 M in dioxane (0.18 mL, 0.72 mmol) and sonicated. The solids were collected by centrifugation and were washed twice with CH₂Cl₂/heptane (v/v=1:2), and dried to give the title compound (56 mg, 49%).

¹H NMR (400 MHz, D₂O) δ 8.15 (t, J=8.0 Hz, 1H), 7.90-7.83 (m, 2H), 5.14 (dd, J=8.7, 3.6 Hz, 1H), 3.81-3.71 (m, 1H), 3.49 (dd, J=12.8, 3.6 Hz, 1H), 3.34 (dd, J=12.8, 8.8 Hz, 1H), 1.98 (s, 3H), 1.72-1.53 (m, 9H), 1.41 (s, 3H), 1.40 (s, 3H), 1.40-1.34 (m, 2H).

### Example 150: N-((1R,4s)-4-(2-(((S)-2-Hydroxy-2-(6-(trifluoromethyl)pyridin-2-yl)ethyl)-amino)-2-methylpropyl)cyclohexyl)methanesulfonamide dihydrochloride

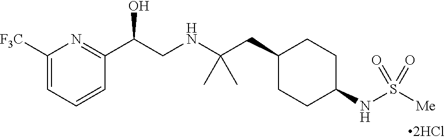 [SMILES: CC(C)(C[C@H]1CC[C@@H](NS(C)(=O)=O)CC1)NC[C@H](O)c1cccc(C(F)(F)F)n1]

The title compound was prepared in accordance with Example 149, using methanesulfonyl chloride instead of acetyl chloride.

¹H NMR (400 MHz, D₂O) δ 8.19-8.13 (m, 1H), 7.91-7.84 (m, 2H), 5.15 (dd, J=8.7, 3.6 Hz, 1H), 3.56-3.46 (m, 2H), 3.35 (dd, J=12.8, 8.8 Hz, 1H), 3.09 (s, 3H), 1.80-1.59 (m, 9H), 1.42 (s, 3H), 1.41 (s, 3H), 1.41-1.33 (2H).

### Example 151: N-((1S,4r)-4-(2-(((S)-2-Hydroxy-2-(6-(trifluoromethyl)pyridin-2-yl)-ethyl)amino)-2-methylpropyl)cyclohexyl)acetamide dihydrochloride

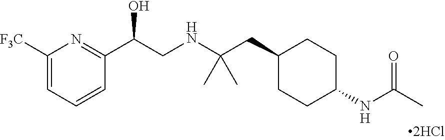 [SMILES: CC(=O)N[C@H]1CC[C@H](CC(C)(C)NC[C@H](O)c2cccc(C(F)(F)F)n2)CC1]

The title compound was prepared from tert-butyl ((1S,4r)-4-(2-(((S)-2-hydroxy-2-(6-(trifluoromethyl)pyridin-2-yl)ethyl)amino)-2-methylpropyl)cyclohexyl)carbamate (see Example 148) in accordance with the procedures in Example 149.

¹H NMR (400 MHz, D₂O) δ 8.16 (t, J=7.9 Hz, 1H), 7.90-7.84 (m, 2H), 5.14 (dd, J=8.8, 3.5 Hz, 1H), 3.76 (s, 1H), 3.58-3.42 (m, 2H), 3.34 (dd, J=12.8, 8.8 Hz, 1H), 1.96 (s, 3H), 1.92-1.78 (m, 4H), 1.50-1.44 (m, 1H), 1.42 (s, 3H), 1.41 (s, 3H), 1.37-1.11 (m, 5H).

### Example 152: N-((1S,4r)-4-(2-(((S)-2-Hydroxy-2-(6-(trifluoromethyl)pyridin-2-yl)-ethyl)amino)-2-methylpropyl)cyclohexyl)methanesulfonamide dihydrochloride

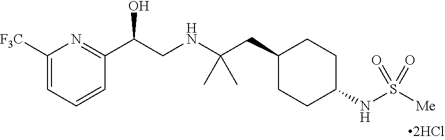 [SMILES: CC(C)(C[C@H]1CC[C@H](NS(C)(=O)=O)CC1)NC[C@H](O)c1cccc(C(F)(F)F)n1]

The title compound was prepared from tert-butyl ((1S,4r)-4-(2-(((S)-2-hydroxy-2-(6-(trifluoromethyl)pyridin-2-yl)ethyl)amino)-2-methylpropyl)cyclohexyl)carbamate (see Example 148) in accordance with the procedures in Example 150.

### Example 153: tert-Butyl ((1R,4r)-4-(2-(((R)-2-(3-fluorophenyl)-2-hydroxyethyl)amino)-2-methylpropyl)cyclohexyl)carbamate

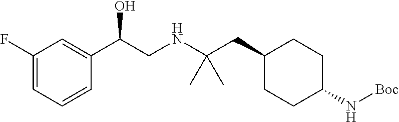 [SMILES: CC(C)(C[C@H]1CC[C@H](NC(=O)OC(C)(C)C)CC1)NC[C@H](O)c1cccc(F)c1]

The title compound was prepared from (R)-2-(3-fluorophenyl)oxirane and tert-butyl ((1r,4r)-4-(2-amino-2-methylpropyl)cyclohexyl)carbamate (see Example 85, Step (b)) in accordance with the procedure in Example 1, Step (e).

¹H NMR (400 MHz, CDCl₃) δ 7.33-7.27 (1H, m) 7.13-7.07 (2H, m) 6.98-6.92 (1H, m) 4.57 (1H, dd, J=3.8, 8.50 Hz) 4.3-4.2 (1H, br s) 3.4-3.3 (1H, br s) 2.88 (1H, dd, J=3.8, 11.9 Hz) 2.52 (1H, dd, J=8.5, 11.9 Hz) 2.01-1.92 (2H, m) 1.80-1.72 (2H, m) 1.44 (9H, s) 1.31-1.25 (3H, m) 1.14-1.02 (4H, m) 1.06 (6H, d, J=0.9 Hz).

Example 154 to 159: The title compounds were prepared from (R)-2-((1-((1r,4R)-4-aminocyclohexyl)-2-methylpropan-2-yl)amino)-1-(3-fluorophenyl)ethan-1-ol, See Example 86, in accordance with the procedure in Example 67, Step (c), using pivaloyl chloride, cyclobutanecarbonyl chloride, cyclopropanecarbonyl chloride, trifluoroacetic anhydride, cyclopentylacetyl chloride or dimethylcarbamoyl chloride, respectively, instead of acetyl chloride.

The hydrochlorides were prepared by dissolving the free base in CH₂Cl₂ and adding 2 equivalents of HCl (2 M in Et₂O) at 0° C., and stirring the mixture at rt for 30 min, followed by addition of Et₂O to precipitate the salt, which was collected, washed with Et₂O and dried.

### Example 154: N-((1R,4r)-4-(2-(((R)-2-(3-Fluorophenyl)-2-hydroxyethyl)amino)-2-methylpropyl)cyclohexyl)pivalamide hydrochloride

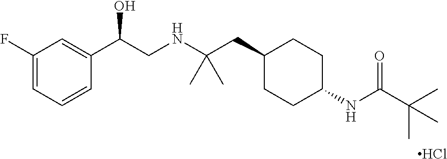 [SMILES: CC(C)(C[C@H]1CC[C@H](NC(=O)C(C)(C)C)CC1)NC[C@H](O)c1cccc(F)c1 ; Cl]

¹H NMR (400 MHz, CDCl₃) δ: 10.11-9.82 (1H, bs), 8.15-7.92 (1H, bs), 7.37-7.27 (1H, m), 7.23-7.13 (2H, m), 7.02-6.94 (1H, m), 5.86-5.56 (1H, m), 5.53-5.19 (2H, m), 3.74-3.57 (1H, m), 3.28-3.08 (1H, m), 3.03-2.84 (1H, m), 1.99-1.73 (4H, m), 1.67-1.59 (2H, m), 1.58-1.52 (1H, m), 1.50 (3H, s), 1.44 (3H, s), 1.15 (9H, s), 1.28-1.03 (4H, m).

### Example 155: N-((1R,4r)-4-(2-(((R)-2-(3-Fluorophenyl)-2-hydroxyethyl)amino)-2-methylpropyl)cyclohexyl)cyclobutanecarboxamide hydrochloride

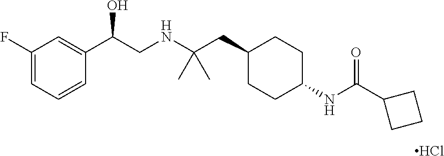 [SMILES: CC(C)(C[C@H]1CC[C@H](NC(=O)C2CCC2)CC1)NC[C@H](O)c1cccc(F)c1 ; Cl]

NMR (400 MHz, CD₃OD) δ 7.45-7.38 (m, 1H), 7.29-7.22 (m, 2H), 7.10-7.03 (m, 1H), 4.95 (dd, J=10.3, 2.9 Hz, 1H), 3.62-3.53 (m, 1H), 3.19 (dd, J=12.4, 3.0 Hz, 1H), 3.12-2.98 (m, 2H), 2.28-2.17 (m, 2H), 2.17-2.05 (m, 2H), 2.03-1.93 (m, 1H), 1.92-1.76 (m, 5H), 1.62 (d, J=5.1 Hz, 2H), 1.50-1.42 (m, 1H), 1.40 (d, J=6.3 Hz, 6H), 1.35-1.14 (m, 4H).

### Example 156: N-((1R,4r)-4-(2-(((R)-2-(3-Fluorophenyl)-2-hydroxyethyl)amino)-2-methylpropyl)cyclohexyl)cyclopropanecarboxamide hydrochloride

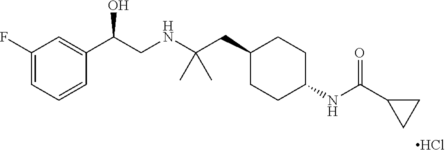 [SMILES: CC(C)(C[C@H]1CC[C@H](NC(=O)C2CC2)CC1)NC[C@H](O)c1cccc(F)c1 ; Cl]

¹H NMR (400 MHz, CD₃OD) δ 7.42 (td, J=8.1, 5.8 Hz, 1H), 7.31-7.20 (m, 2H), 7.07 (td, J=8.1, 2.7 Hz, 1H), 4.95 (dd, J=10.3, 2.9 Hz, 1H), 3.64-3.53 (m, 1H), 3.19 (dd, J=12.5, 3.0 Hz, 1H), 3.03 (dd, J=12.5, 10.4 Hz, 1H), 1.97-1.80 (m, 4H), 1.62 (d, J=5.1 Hz, 2H), 1.54 (ddd, J=12.6, 7.9, 4.6 Hz, 1H), 1.49-1.43 (m, 1H), 1.40 (d, J=6.3 Hz, 6H), 1.36-1.20 (m, 4H), 0.88-0.78 (m, 2H), 0.77-0.66 (m, 2H).

### Example 157: 2,2,2-Trifluoro-N-((1R,4r)-4-(2-(((R)-2-(3-fluorophenyl)-2-hydroxyethyl)-amino)-2-methylpropyl)cyclohexyl)acetamide hydrochloride

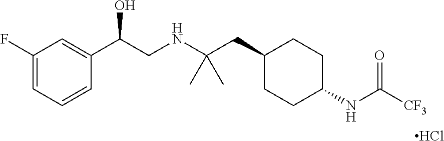 [SMILES: CC(C)(C[C@H]1CC[C@H](NC(=O)C(F)(F)F)CC1)NC[C@H](O)c1cccc(F)c1 ; Cl]

¹H NMR (400 MHz, CDCl₃), δ: 10.20-9.93 (bs, 1H), 8.20-7.93 (bs, 1H), 7.37-7.29 (m, 1H), 7.24-7.14 (m, 2H), 7.03-6.97 (m, 1H), 6.07 (d, J=8.2 Hz, 1H), 5.79-5.61 (bs, 1H), 5.47-5.31 (m, 1H), 3.77-3.66 (m, 1H), 3.27-3.14 (m, 1H), 3.00-2.87 (m, 1H), 2.06-1.93 (m, 2H), 1.92-1.79 (m, 2H), 1.70-1.58 (m, 3H), 1.51 (s, 3H), 1.44 (s, 3H), 1.38-1.23 (m, 2H), 1.23-1.09 (m, 2H).

### Example 158 3-((1R,4r)-4-(2-(((R)-2-(3-Fluorophenyl)-2-hydroxyethyl)amino)-2-methyl-propyl)cyclohexyl)-1,1-dimethylurea hydrochloride

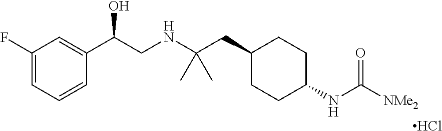 [SMILES: CN(C)C(=O)N[C@H]1CC[C@H](CC(C)(C)NC[C@H](O)c2cccc(F)c2)CC1 ; Cl]

¹H NMR (400 MHz, CD₃OD), δ: 7.45-7.39 (1H, m), 7.29-7.21 (2H, m), 7.11-7.04 (1H, m), 4.92 (1H, dd, J=10.3, 3.0 Hz), 3.52-3.43 (1H, m), 3.19 (1H, dd, J=12.5, 3.0 Hz), 3.03 (1H, dd, J=12.5, 10.3 Hz), 2.88 (6H, s), 1.94-1.81 (4H, m), 1.61 (2H, d, J=5.1 Hz), 1.48-1.41 (1H, m), 1.39 (6H, d, J=5.6 Hz), 1.36-1.26 (2H, m), 1.26-1.13 (2H, m).

### Example 159: N-((1R,4r)-4-(2-(((R)-2-(3-Fluorophenyl)-2-hydroxyethyl)amino)-2-methylpropyl)cyclohexyl)propane-1-sulfonamide hydrochloride

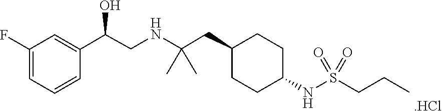 [SMILES: CCCS(=O)(=O)N[C@H]1CC[C@H](CC(C)(C)NC[C@H](O)c2cccc(F)c2)CC1 ; ".HCl"]

A mixture of (R)-2-((1-((1r,4R)-4-aminocyclohexyl)-2-methylpropan-2-yl)amino)-1-(3-fluorophenyl)ethan-1-ol, see Example 86, (25 mg, 81 μmol), propane-1-sulfonyl fluoride (12.3 mg, 97 μmol), 1-hydroxybenzotriazole hydrate (120 μg, 0.8 μmol), 1,1,3,3-tetramethyldisiloxane (21.8 mg, 162 μmol), DIPEA (28 μL, 162 μmol) and DMSO (3 mL) was stirred at rt for 18 h and diluted with ethyl acetate, washed with H₂O, and HCl (aq, 1 M). The organic layer was collected, washed with brine, dried (Na₂SO₄) and concentrated. The residue was purified by reversed-phase chromatography (HCl (aq, 0.01 M)/MeOH) to give the title compound (18 mg, 49%).

¹H NMR (400 MHz, CD₃OD), δ, ppm: 7.46-7.37 (m, 1H), 7.31-7.20 (m, 2H), 7.12-7.03 (m, 1H), 4.94 (dd, J=10.4, 3.0 Hz, 1H), 3.18 (dd, J=12.5, 3.0 Hz, 1H), 3.15-3.07 (m, 1H), 3.06-2.94 (m, 3H), 2.04-1.95 (m, 2H), 1.90-1.74 (m, 4H), 1.60 (d, J=5.1 Hz, 2H), 1.46-1.29 (m, 3H), 1.39 (d, J=6.4 Hz, 6H), 1.26-1.14 (m, 2H), 1.05 (t, J=7.5 Hz, 3H).

### Example 160: 1,1,1-Trifluoro-N-((1R,4r)-4-(2-(((R)-2-(3-fluorophenyl)-2-hydroxyethyl)-amino)-2-methylpropyl)cyclohexyl)methanesulfonamide hydrochloride

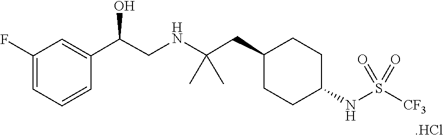 [SMILES: CC(C)(C[C@H]1CC[C@H](NS(=O)(=O)C(F)(F)F)CC1)NC[C@H](O)c1cccc(F)c1 ; ".HCl"]

The title compound was prepared from (R)-2-((1-((1r,4R)-4-aminocyclohexyl)-2-methylpropan-2-yl)amino)-1-(3-fluorophenyl)ethan-1-ol, See Example 86, in accordance with the procedure in Example 71, Step (a), using trifluoromethanesulfonic anhydride instead of methanesulfonyl chloride. The hydrochloride was obtained by purification using reverse phase chromatography as described in Example 159.

NMR (400 MHz, CD₃OD) δ: 7.46-7.38 (m, 1H), 7.30-7.20 (m, 2H), 7.11-7.03 (m, 1H), 4.93 (dd, J=10.4, 2.9 Hz, 1H), 3.30-3.24 (m, 1H), 3.18 (dd, J=12.5, 3.0 Hz, 1H), 3.02 (dd, J=12.5, 10.3 Hz, 1H), 2.02-1.94 (m, 2H), 1.92-1.84 (m, 2H), 1.60 (d, J=5.1 Hz, 2H), 1.50-1.36 (m, 3H), 1.39 (d, J=6.4 Hz, 6H), 1.27-1.13 (m, 2H).

### Example 161: N-((1R,4r)-4-(2-(((R)-2-(3-Fluorophenyl)-2-hydroxyethyl)amino)-2-methypropyl)cyclohexyl)cyclopropanesulfonamide hydrochloride

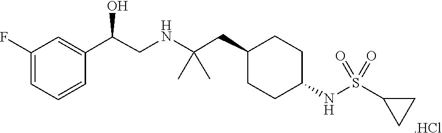 [SMILES: CC(C)(C[C@H]1CC[C@H](NS(=O)(=O)C2CC2)CC1)NC[C@H](O)c1cccc(F)c1 ; ".HCl"]

A solution of cyclopropane sulfonyl chloride (12.4 μL, 102 μmol) in MeCN (1.0 mL) was added dropwise to a mixture of (R)-2-((1-((1r,4R)-4-aminocyclohexyl)-2-methylpropan-2-yl)amino)-1-(3-fluorophenyl)ethan-1-ol, see Example 86, (30 mg, 97 μmol), Et₃N (16.3 μL, 117 μmol), DMAP (5.94 mg, 49 μmol) and MeCN (2.0 mL) at −18° C. The mixture was stirred for 18 h allowing the temperature to reach rt and quenched with NaOH (aq, 4 M, 5 mL). The mixture was extracted with EtOAc and the combined extracts were washed with HCl (aq, 2 M), dried (Na₂SO₄) and concentrated. The residue was purified by chromatography and the relevant fractions were pooled and concentrated. The hydrochloride salt was obtained using the general procedure described for Example 154 to 158.

¹H NMR (400 MHz, CD₃OD) δ: 7.46-7.38 (m, 1H), 7.30-7.21 (m, 2H), 7.11-7.03 (m, 1H), 4.94 (dd, J=10.3, 2.9 Hz, 1H), 3.22-3.13 (m, 2H), 3.07-2.99 (m, 1H), 2.58-2.45 (m, 1H), 2.10-2.00 (m, 2H), 1.90-1.82 (m, 2H), 1.61 (d, J=5.1 Hz, 2H), 1.48-1.31 (m, 3H), 1.39 (d, J=6.4 Hz, 6H), 1.26-1.14 (m, 2H), 1.07-0.93 (m, 4H).

### Example 162: tert-Butyl ((1S,4s)-4-(2-(((R)-2-(3-fluorophenyl)-2-hydroxyethyl)amino)-2-methylpropyl)cyclohexyl)carbamate

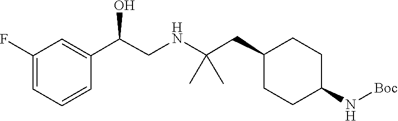 [SMILES: CC(C)(C[C@H]1CC[C@@H](NC(=O)OC(C)(C)C)CC1)NC[C@H](O)c1cccc(F)c1]

A mixture of tert-butyl ((1s,4s)-4-(2-amino-2-methylpropyl)cyclohexyl)carbamate, see Example 131, Step (a), (0.479 g, 1.77 mmol), (R)-2-(3-fluorophenyl)oxirane (294 mg, 2.12 mmol) and iPrOH (4 mL) was heated at 80° C. for 18 h. The mixture was cooled to rt and concentrated. The residue was purified by reverse phase chromatography (H₂O/MeCN/0.1% AcOH) to give the sub-title compound as an acetate salt (389 mg, 47%), which was converted to the free base by treatment with NaHCO₃ and extractive workup using CH₂Cl₂.

¹H NMR (400 MHz, CDCl₃) δ 7.35-7.25 (m, 1H), 7.16-7.05 (m, 2H), 6.99-6.89 (m, 1H), 4.57 (dd, J=8.5, 3.8 Hz, 2H), 3.64 (s, 1H), 2.88 (dd, J=11.8, 3.8 Hz, 1H), 2.53 (dd, J=11.9, 8.5 Hz, 1H), 1.64-1.52 (m, 7H), 1.44 (s, 9H), 1.34-1.17 (m, 5H), 1.06 (s, 6H).

### Example 163: (R)-2-((1-((1s,4S)-4-Aminocyclohexyl)-2-methylpropan-2-yl)amino)-1-(3-fluorophenyl)ethan-1-ol

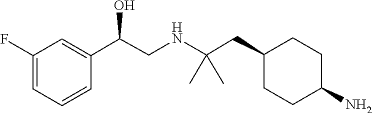 [SMILES: CC(C)(C[C@H]1CC[C@@H](N)CC1)NC[C@H](O)c1cccc(F)c1]

The title compound was prepared from tert-butyl ((1S,4s)-4-(2-(((R)-2-(3-fluorophenyl)-2-hydroxyethyl)amino)-2-methylpropyl)cyclohexyl)carbamate acetate in accordance with the procedure in Example 123, Step (c).

¹H NMR (400 MHz, CD₃OD) δ 7.39-7.31 (m, 1H), 7.20-7.11 (m, 2H), 6.92-7.03 (m, 1H), 4.70 (m, 2H), 2.83 (s, 1H), 2.71-2.64 (m, 2H), 1.70-1.38 (m, 11H), 1.09 (s, 6H).

### Example 164: N-((1S,4s)-4-(2-(((R)-2-(3-Fluorophenyl)-2-hydroxyethyl)amino)-2-methylpropyl)cyclohexyl)acetamide

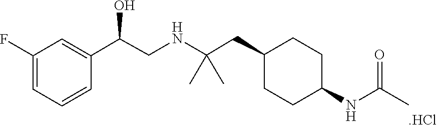 [SMILES: CC(=O)N[C@H]1CC[C@@H](CC(C)(C)NC[C@H](O)c2cccc(F)c2)CC1 ; ".HCl"]

The title compound was prepared from (R)-2-((1-((1s,4S)-4-aminocyclohexyl)-2-methyl-propan-2-yl)amino)-1-(3-fluorophenyl)ethan-1-ol in accordance with the procedure in Example 67, Step (f). The hydrochloride salt was obtained using the general procedure described for Example 154 to 158.

H NMR (400 MHz, CD₃OD) δ 7.42 (td, J=8.2, 6.0 Hz, 1H), 7.32-7.19 (m, 2H), 7.13-7.02 (m, 1H), 4.95 (dd, J=10.3, 2.9 Hz, 1H), 3.87-3.78 (m, 1H), 3.20 (dd, J=12.5, 2.9 Hz, 1H), 3.04 (dd, J=12.5, 10.3 Hz, 1H), 1.96 (s, 3H), 1.71-1.60 (m, 8H), 1.54-1.45 (m, 2H), 1.40 (d, J=6.8 Hz, 6H), 1.33-1.27 (m, 1H).

Examples 165 to 169: The title compounds were prepared from (R)-2-((1-((1s,4S)-4-aminocyclohexyl)-2-methylpropan-2-yl)amino)-1-(3-fluorophenyl)ethan-1-ol in accordance with the procedure in Examples 154 to 158.

### Example 165: N-((1S,4s)-4-(2-(((R)-2-(3-Fluorophenyl)-2-hydroxyethyl)amino)-2-methylpropyl)cyclohexyl)pivalamide hydrochloride

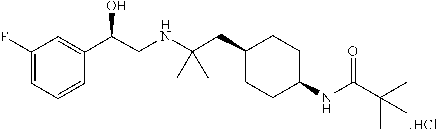 [SMILES: CC(C)(C[C@H]1CC[C@@H](NC(=O)C(C)(C)C)CC1)NC[C@H](O)c1cccc(F)c1 ; ".HCl"]

¹H NMR (400 MHz, CD₃OD) δ 7.32 (td, J=7.7, 5.7 Hz, 1H), 7.22-7.11 (m, 2H), 7.03-6.92 (m, 1H), 4.86 (dd, J=10.4, 2.9 Hz, 1H), 3.73-3.60 (m, 1H), 3.10 (dd, J=12.5, 3.0 Hz, 1H), 2.94 (dd, J=12.4, 10.4 Hz, 1H), 1.68-1.60 (m, 3H), 1.59-1.47 (m, 6H), 1.46-1.36 (m, 2H), 1.30 (d, J=5.8 Hz, 6H), 1.08 (s, 9H).

### Example 166: N-((1S,4s)-4-(2-(((R)-2-(3-Fluorophenyl)-2-hydroxyethyl)amino)-2-methylpropyl)cyclohexyl)cyclopropanecarboxamide hydrochloride

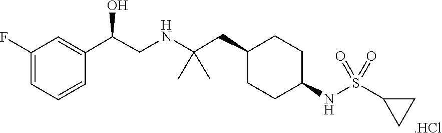 [SMILES: CC(C)(C[C@H]1CC[C@@H](NS(=O)(=O)C2CC2)CC1)NC[C@H](O)c1cccc(F)c1 ; ".HCl"]

¹H NMR (400 MHz, CDCl₃) δ 9.94 (s, 1H), 8.05 (s, 1H), 7.31-7.22 (m, 1H), 7.20-7.07 (m, 2H), 7.00-6.84 (m, 1H), 5.67 (s, 2H), 5.48-5.22 (m, 1H), 3.84 (s, 1H), 3.23-3.05 (m, 1H), 2.93 (s, 1H), 1.94-1.65 (m, 3H), 1.62-1.48 (m, 5H), 1.38 (d, J=16.3 Hz, 6H), 1.30-1.21 (m, 2H), 0.85 (s, 2H), 0.71-0.50 (m, 2H).

### Example 167: N-((1S,4s)-4-(2-(((R)-2-(3-Fluorophenyl)-2-hydroxyethyl)amino)-2-methylpropyl)cyclohexyl)cyclobutanecarboxamide hydrochloride

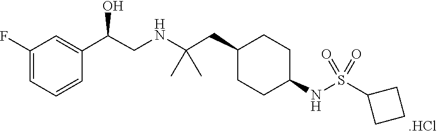 [SMILES: CC(C)(C[C@H]1CC[C@@H](NS(=O)(=O)C2CCC2)CC1)NC[C@H](O)c1cccc(F)c1 ; ".HCl"]

¹H NMR (400 MHz, CDCl₃) δ 10.02 (s, 1H), 8.11 (s, 1H), 7.37-7.28 (m, 1H), 7.26-7.14 (m, 2H), 7.00 (t, J=8.3 Hz, 1H), 5.69 (s, 1H), 5.44 (s, 1H), 5.32 (s, 1H), 3.89 (s, 1H), 3.32-3.10 (m, 1H), 3.08-2.81 (m, 2H), 2.33-2.18 (m, 2H), 2.17-2.04 (m, 2H), 2.03-1.90 (m, 1H), 1.90-1.83 (m, 1H), 1.82-1.69 (m, 3H), 1.68-1.54 (m, 5H), 1.45 (d, J=14.9 Hz, 7H), 1.35-1.21 (m, 2H).

### Example 189: 2,2,2-Trifluoro-N-((1S,4s)-4-(2-(((R)-2-(3-Fluorophenyl)-2-hydroxyethyl)-amino)-2-methylpropyl)cyclohexyl)acetamide hydrochloride

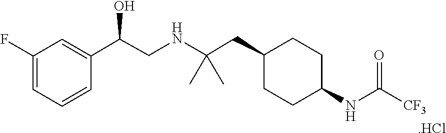 [SMILES: CC(C)(C[C@H]1CC[C@@H](NC(=O)C(F)(F)F)CC1)NC[C@H](O)c1cccc(F)c1 ; ".HCl"]

¹H NMR (400 MHz, CD₃OD) δ 7.42 (td, J=7.7, 5.7 Hz, 1H), 7.31-7.22 (m, 2H), 7.11-7.04 (m, 1H), 4.96 (dd, J=10.3, 2.9 Hz, 1H), 3.90-3.78 (m, 1H), 3.20 (dd, J=12.4, 3.0 Hz, 1H), 3.05 (dd, J=12.5, 10.4 Hz, 1H), 1.82-1.75 (m, 1H), 1.75-1.62 (m, 8H), 1.61-1.49 (m, 2H), 1.40 (d, J=6.4 Hz, 6H).

### Example 169: 3-((1S,4s)-4-(2-(((R)-2-(3-Fluorophenyl)-2-hydroxyethyl)amino)-2-methylpropyl)cyclohexyl)-1,1-dimethylurea hydrochloride

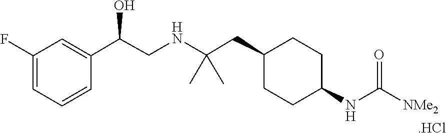 [SMILES: CN(C)C(=O)N[C@H]1CC[C@@H](CC(C)(C)NC[C@H](O)c2cccc(F)c2)CC1 ; ".HCl"]

¹H NMR (400 MHz, CD₃OD) δ 7.42 (td, J=8.1, 5.8 Hz, 1H), 7.30-7.22 (m, 2H), 7.11-7.02 (m, 1H), 4.98 (dd, J=10.3, 2.9 Hz, 1H), 3.70-3.59 (m, 1H), 3.20 (dd, J=12.5, 2.9 Hz, 1H), 3.04 (dd, J=12.4, 10.4 Hz, 1H), 2.90 (s, 6H), 1.79-1.49 (m, 11H), 1.40 (d, J=5.6 Hz, 6H).

### Example 170: N-((1S,4s)-4-(2-(((R)-2-(3-Fluorophenyl)-2-hydroxyethyl)amino)-2-methylpropyl)cyclohexyl)methanesulfonamide hydrochloride

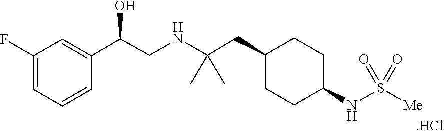 [SMILES: CC(C)(C[C@H]1CC[C@@H](NS(C)(=O)=O)CC1)NC[C@H](O)c1cccc(F)c1 ; ".HCl"]

The title compound was prepared from (R)-2-((1-((1s,4S)-4-aminocyclohexyl)-2-methylpropan-2-yl)amino)-1-(3-fluorophenyl)ethan-1-ol in accordance with the procedure in Example 71, Step (a). The hydrochloride salt was obtained using the general procedure described for Example 154 to 158.

¹H NMR (400 MHz, CD₃OD) δ 7.42 (td, J=7.7, 5.6 Hz, 1H), 7.29-7.22 (m, 2H), 7.10-7.04 (m, 1H), 4.95 (dd, J=10.3, 2.9 Hz, 1H), 3.48 (t, J=4.5 Hz, 1H), 3.19 (dd, J=12.5, 2.9 Hz, 1H), 3.04 (dd, J=12.5, 10.4 Hz, 1H), 2.94 (s, 3H), 1.76-1.69 (m, 4H), 1.69-1.59 (m, 5H), 1.56-1.44 (m, 3H), 1.40 (d, J=6.3 Hz, 6H).

### Example 171: N-((1S,4s)-4-(2-(((R)-2-(3-Fluorophenyl)-2-hydroxyethyl)amino)-2-methylpropyl)cyclohexyl)propane-1-sulfonamide hydrochloride

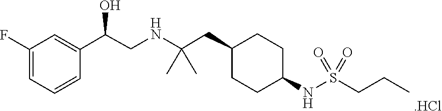 [SMILES: CCCS(=O)(=O)N[C@H]1CC[C@@H](CC(C)(C)NC[C@H](O)c2cccc(F)c2)CC1 ; ".HCl"]

The title compound was prepared from (R)-2-((1-((1s,4S)-4-aminocyclohexyl)-2-methyl-propan-2-yl)amino)-1-(3-fluorophenyl)ethan-1-ol and 1-propanesulfonyl chloride in accordance with the procedure in Example 161.

¹H NMR (400 MHz, CDCl₃), δ, ppm: 9.94 (bs, 1H), 8.06 (bs, 1H), 7.39-7.28 (m, 1H), 7.25-7.18 (m, 2H), 7.05-6.95 (m, 1H), 5.91-5.58 (m, 1H), 5.52-5.33 (m, 1H), 4.65-4.33 (m, 1H), 3.52-3.40 (m, 1H), 3.30-3.13 (m, 1H), 3.07-2.84 (m, 3H), 1.86-1.55 (m, 11H), 1.45 (d, J=16.2 Hz, 8H), 1.40-1.22 (m, 2H), 1.10-0.98 (m, 3H)

### Example 172: 1,1,1-Trifluoro-N-((1S,4s)-4-(2-(((R)-2-(3-fluorophenyl)-2-hydroxyethyl)-amino)-2-methylpropyl)cyclohexyl)methanesulfonamide hydrochloride

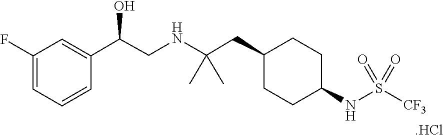 [SMILES: CC(C)(C[C@H]1CC[C@@H](NS(=O)(=O)C(F)(F)F)CC1)NC[C@H](O)c1cccc(F)c1 ; ".HCl"]

The title compound was prepared from (R)-2-((1-((1s,4S)-4-aminocyclohexyl)-2-methylpropan-2-yl)amino)-1-(3-fluorophenyl)ethan-1-ol in accordance with the procedure in Example 71, Step (a), using trifluoromethanesulfonic anhydride instead of methanesulfonyl chloride, with the exception that the addition of the trifluoromethanesulfonic anhydride was made at −18° C. and that the reaction mixture was stirred at −18° C. for 1 h before workup. The hydrochloride was obtained by purification using reverse phase chromatography as described in Example 159.

¹H NMR (400 MHz, CD₃OD), δ, ppm: 7.46-7.37 (m, 1H), 7.31-7.21 (m, 2H), 7.11-7.03 (m, 1H), 4.95 (dd, J=10.4, 3.0 Hz, 1H), 3.67-3.57 (m, 1H), 3.20 (dd, J=12.4, 3.0 Hz, 1H), 3.04 (dd, J=12.4, 10.4 Hz, 1H), 1.80-1.61 (m, 10H), 1.57-1.44 (m, 2H), 1.40 (d, J=6.7 Hz, 6H).

### Example 173: N-((1S,4s)-4-(2-(((R)-2-(3-Fluorophenyl)-2-hydroxyethyl)amino)-2-methylpropyl)cyclohexyl)cyclopropanesulfonamide hydrochloride

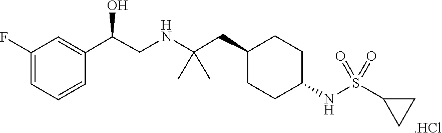 [SMILES: CC(C)(C[C@H]1CC[C@H](NS(=O)(=O)C2CC2)CC1)NC[C@H](O)c1cccc(F)c1 ; ".HCl"]

The title compound was prepared from (R)-2-((1-((1s,4S)-4-aminocyclohexyl)-2-methylpropan-2-yl)amino)-1-(3-fluorophenyl)ethan-1-ol and cyclopropanesulfonyl chloride in accordance with the procedure in Example 161.

¹H NMR (400 MHz, CD₃OD), δ, ppm: 7.46-7.38 (m, 1H), 7.31-7.19 (m, 2H), 7.13-7.01 (m, 1H), 4.94 (dd, J=10.4, 3.0 Hz, 1H), 3.56-3.48 (m, 1H), 3.19 (dd, J=12.5, 3.0 Hz, 1H), 3.04 (dd, J=12.5, 10.4 Hz, 1H), 2.56-2.49 (m, 1H), 1.80-1.70 (m, 4H), 1.70-1.57 (m, 5H), 1.57-1.44 (m, 2H), 1.40 (d, J=6.3 Hz, 6H), 1.07-0.95 (m, 4H).

### Example 174: tert-Butyl ((1S,4s)-4-(2-(((R)-2-(5-fluoropyridin-3-yl)-2-hydroxyethyl)-amino)-2-methylpropyl)cyclohexyl)carbamate

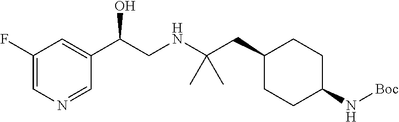 [SMILES: CC(C)(C[C@H]1CC[C@@H](NC(=O)OC(C)(C)C)CC1)NC[C@H](O)c1cncc(F)c1]

The title compound was prepared from (R)-3-fluoro-5-(oxiran-2-yl)pyridine and tert-butyl ((1s,4s)-4-(2-amino-2-methylpropyl)cyclohexyl)carbamate in accordance with the procedure in Example 1, Step (e) followed by crystallization from MeCN.

¹H NMR (400 MHz, CDCl₃) δ 8.41-8.33 (m, 2H), 7.48 (dt, J=9.2, 2.0 Hz, 1H), 4.70-4.52 (m, 2H), 3.65 (s, 1H), 2.93 (dd, J=12.0, 3.8 Hz, 1H), 2.51 (dd, J=12.0, 8.8 Hz, 1H), 1.66-1.51 (m, 7H), 1.44 (s, 9H), 1.33-1.20 (m, 4H), 1.07 (d, J=1.0 Hz, 6H).

### Example 175: (R)-2-((1-((1s,4S)-4-Aminocyclohexyl)-2-methylpropan-2-yl)amino)-1-(5-fluoropyridin-3-yl)ethan-1-ol

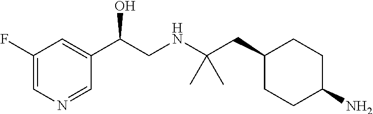 [SMILES: CC(C)(C[C@H]1CC[C@@H](N)CC1)NC[C@H](O)c1cncc(F)c1]

The title compound was prepared from tert-butyl ((1S,4s)-4-(2-(((R)-2-(3-fluorophenyl)-2-hydroxyethyl)amino)-2-methylpropyl)cyclohexyl)carbamate in accordance with the procedure in Example 123, Step (c).

¹H NMR (400 MHz, CDCl₃) δ 8.41-8.32 (m, 2H), 7.53-7.44 (m, 1H), 4.63 (dd, J=8.7, 3.8 Hz, 1H), 2.92 (dd, J=12.0, 3.8 Hz, 1H), 2.89-2.83 (m, 1H), 2.52 (dd, J=12.0, 8.8 Hz, 1H), 1.64-1.54 (m, 3H), 1.53-1.46 (m, 2H), 1.45-1.35 (m, 4H), 1.34-1.30 (m, 2H), 1.06 (s, 6H).

### Example 176: N-((1S,4s)-4-(2-(((R)-2-(5-Fluoropyridin-3-yl)-2-hydroxyethyl)amino)-2-methylpropyl)cyclohexyl)acetamide

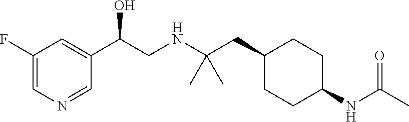 [SMILES: CC(=O)N[C@H]1CC[C@@H](CC(C)(C)NC[C@H](O)c2cncc(F)c2)CC1]

The title compound was prepared from (R)-2-((1-((1s,4S)-4-aminocyclohexyl)-2-methylpropan-2-yl)amino)-1-(3-fluorophenyl)ethan-1-ol in accordance with the procedure in Example 67, Step (f).

¹H NMR (400 MHz, CDCl₃) δ 8.48-8.26 (m, 2H), 7.48 (dt, J=9.4, 2.0 Hz, 1H), 5.56 (d, J=7.6 Hz, 1H), 4.64 (dd, J=8.8, 3.8 Hz, 1H), 4.06-3.78 (m, 1H), 2.91 (dd, J=11.9, 3.8 Hz, 1H), 2.70 (s, 1H), 2.53 (dd, J=12.0, 8.8 Hz, 1H), 1.96 (s, 3H), 1.69-1.48 (m, 7H), 1.31 (s, 2H), 1.28-1.19 (m, 2H), 1.07 (d, J=1.9 Hz, 6H).

### Example 177: N-((1S,4s)-4-(2-(((R)-2-(5-Fluoropyridin-3-yl)-2-hydroxyethyl)amino)-2-methylpropyl)cyclohexyl)methanesulfonamide

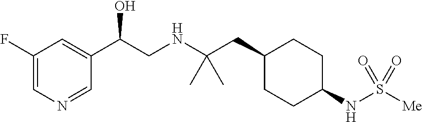 [SMILES: CC(C)(C[C@H]1CC[C@@H](NS(C)(=O)=O)CC1)NC[C@H](O)c1cncc(F)c1]

The title compound was prepared from (R)-2-((1-((1s,4S)-4-aminocyclohexyl)-2-methylpropan-2-yl)amino)-1-(3-fluorophenyl)ethan-1-ol in accordance with the procedure in Example 71, Step (a).

¹H NMR (400 MHz, CDCl₃) δ 8.55-8.22 (m, 2H), 7.50 (dt, J=9.3, 2.4 Hz, 1H), 5.16 (s, 1H), 4.82 (dd, J=9.0, 3.4 Hz, 1H), 3.87 (s, 2H), 3.51 (s, 1H), 3.00-2.95 (m, 1H), 2.95 (s, 3H), 2.62 (dd, J=12.0, 9.1 Hz, 1H), 1.75-1.48 (m, 7H), 1.46-1.38 (m, 2H), 1.38-1.26 (m, 2H), 1.12 (d, J=3.6 Hz, 6H).

### Example 178: N-((1R,4r)-4-(2-(((R)-2-(3-Fluorophenyl)-2-hydroxyethyl)amino)-2-methylpropyl)cyclohexyl)-N-methylacetamide hydrochloride

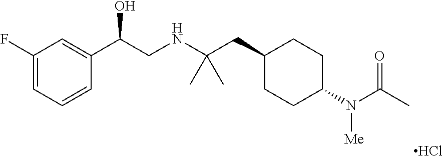 [SMILES: CC(=O)N(C)[C@H]1CC[C@H](CC(C)(C)NC[C@H](O)c2cccc(F)c2)CC1 ; Cl]

### (a) tert-Butyl ((1r,4r)-4-(cyanomethyl)cyclohexyl)(methyl)carbamate

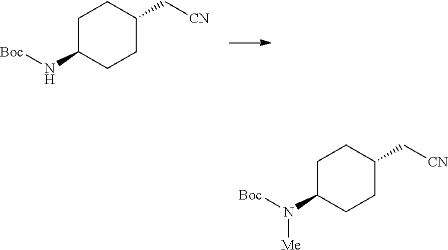 [SMILES: CC(C)(C)OC(=O)N[C@H]1CC[C@H](CC#N)CC1 ; CN(C(=O)OC(C)(C)C)[C@H]1CC[C@H](CC#N)CC1]

NaH (60% in mineral oil, 101 mg, 2.52 mmol) was added to a solution of tert-butyl ((1r,4r)-4-(cyanomethyl)cyclohexyl)carbamate, see Example 85, Step (b), (500 mg, 2.10 mmol) in DMF (10 mL) at 0° C. and the mixture was stirred for 10 min at 0° C. MeI (7.8 μL, 125 μmol) was added at 0° C. and the mixture was stirred at room temperature for 18 h. H₂O was added and the mixture was extracted with EtOAc. The combined extracts were washed with brine, dried (Na₂SO₄) and concentrated. The residue was purified by chromatography to give the sub-title compound (470 mg, 89%).

¹H NMR (400 MHz, CCDl₃), δ, ppm: 4.07-3.62 (m, 1H), 2.71 (s, 3H), 2.25 (d, J=6.8 Hz, 2H), 1.98-1.89 (m, 2H), 1.79-1.71 (m, 2H), 1.68-1.55 (m, 1H), 1.53-1.40 (m, 2H), 1.45 (s, 9H), 1.30-1.16 (m, 2H).

### (b) tert-Butyl ((1r,4r)-4-(2-amino-2-methylpropyl)cyclohexyl)(methyl)carbamate

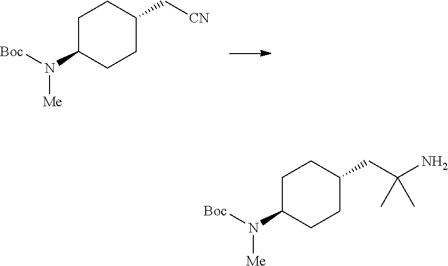 [SMILES: CN(C(=O)OC(C)(C)C)[C@H]1CC[C@H](CC#N)CC1 ; CN(C(=O)OC(C)(C)C)[C@H]1CC[C@H](CC(C)(C)N)CC1]

The sub-title compound was prepared from tert-butyl ((1r,4r)-4-(cyanomethyl)cyclo-hexyl)(methyl)carbamate in accordance with the procedure in Example 13, Step (b). The crude product was purified by chromatography.

### (c) tert-Butyl ((1R,4r)-4-(2-(((R)-2-(3-fluorophenyl)-2-hydroxyethyl)amino)-2-methylpropyl)cyclohexyl)(methyl)carbamate

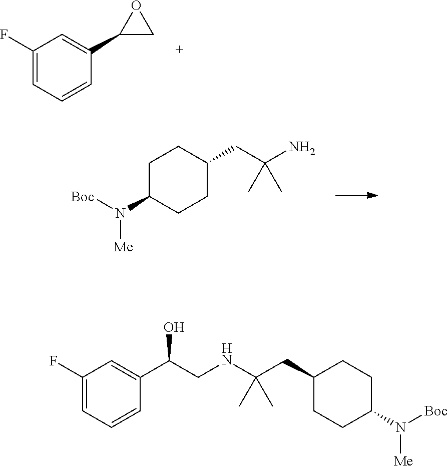 [SMILES: Fc1cccc([C@@H]2CO2)c1 ; CN(C(=O)OC(C)(C)C)[C@H]1CC[C@H](CC(C)(C)N)CC1 ; CN(C(=O)OC(C)(C)C)[C@H]1CC[C@H](CC(C)(C)NC[C@H](O)c2cccc(F)c2)CC1]

The sub-title compound was prepared from (R)-2-(3-fluorophenyl)oxirane and tert-butyl ((1r,4r)-4-(2-amino-2-methylpropyl)cyclohexyl)(methyl)carbamate in accordance with the procedure in Example 1, Step (e).

### (d) (R)-1-(3-fluorophenyl)-2-((2-methyl-1-((1r,4R)-4-(methylamino)cyclohexyl)-propan-2-yl)amino)ethan-1-ol

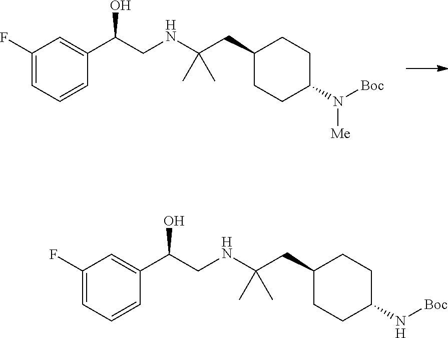 [SMILES: CN(C(=O)OC(C)(C)C)[C@H]1CC[C@H](CC(C)(C)NC[C@H](O)c2cccc(F)c2)CC1 ; CC(C)(C[C@H]1CC[C@H](NC(=O)OC(C)(C)C)CC1)NC[C@H](O)c1cccc(F)c1]

The sub-title compound was prepared from tert-butyl ((1R,4r)-4-(2-(((R)-2-(3-fluoro-phenyl)-2-hydroxyethyl)amino)-2-methylpropyl)cyclohexyl)(methyl)carbamate in accordance with the procedure in Example 67, Step (e).

### (e) N-((1R,4r)-4-(2-(((R)-2-(3-Fluorophenyl)-2-hydroxyethyl)amino)-2-methyl-propyl)cyclohexyl)-N-methylacetamide hydrochloride

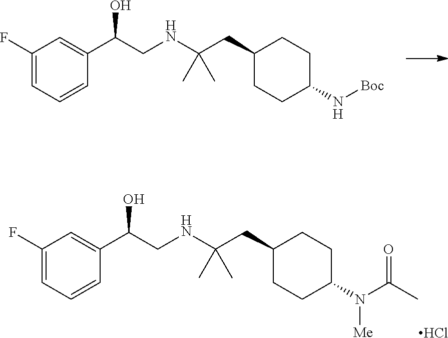 [SMILES: CC(C)(C[C@H]1CC[C@H](NC(=O)OC(C)(C)C)CC1)NC[C@H](O)c1cccc(F)c1 ; CC(=O)N(C)[C@H]1CC[C@H](CC(C)(C)NC[C@H](O)c2cccc(F)c2)CC1 ; Cl]

The title compound was prepared from (R)-1-(3-fluorophenyl)-2-((2-methyl-1-((1r,4R)-4-(methylamino)cyclohexyl)propan-2-yl)amino)ethan-1-ol in accordance with the procedure in Example 67, Step (f) followed by purification by reversed-phase chromatography (HCl (aq, 0.05 M)/MeCN).

¹H NMR (400 MHz, CD₃OD), δ, ppm: 7.46-7.38 (m, 1H), 7.31-7.21 (m, 2H), 7.11-7.03 (m, 1H), 5.00-4.92 (m, 1H), 4.38-4.28 (m, 0.5H), 3.23-3.14 (m, 0.5H), 3.07-2.99 (m, 1H), 2.98-2.84 (m, 3H), 2.24-2.12 (m, 3H), 1.99-1.87 (m, 2H), 1.80-1.70 (m, 2H), 1.67-1.57 (m, 4H), 1.51-1.34 (m, 7H), 1.33-1.19 (m, 2H).

### Example 179: N-((1R,4r)-4-(2-(((R)-2-(3-Fluorophenyl)-2-hydroxyethyl)amino)-2-methylpropyl)cyclohexyl)-N-methylmethanesulfonamide hydrochloride

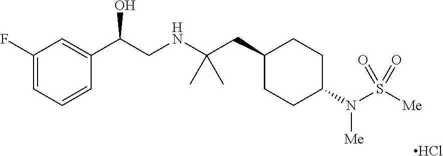 [SMILES: CN([C@H]1CC[C@H](CC(C)(C)NC[C@H](O)c2cccc(F)c2)CC1)S(C)(=O)=O ; Cl]

DIPEA (9.6 μL, 56 μmol), followed by a solution of methanesulfonyl chloride (4.1 μL, 53 μmol) in CH₂Cl₂ (1 mL) were added to a solution of (R)-1-(3-fluorophenyl)-2-((2-methyl-1-((1r,4R)-4-(methylamino)cyclohexyl)propan-2-yl)amino)ethan-1-ol (17 mg, 53 μmol) in CH₂Cl₂ (2 mL) 0° C. The mixture was stirred for 30 min at 0° C., quenched with NaHCO₃ (aq, sat) and extracted with CH₂Cl₂. The combined extracts were dried (Na₂SO₄) and concentrated. The residue was purified twice by reversed-phase chromatography (HCl (aq, 0.05 M)/MeCN) to give the title compound (14 mg, 61%).

¹H NMR (400 MHz, CD₃OD), δ, ppm: 7.45-7.39 (m, 1H), 7.29-7.21 (m, 2H), 7.10-7.04 (m, 1H), 4.94 (dd, J=10.3, 3.0 Hz, 1H), 3.65 (m, 1H), 3.19 (dd, J=12.5, 3.0 Hz, 1H), 3.03 (dd, J=12.5, 10.3 Hz, 1H), 2.86 (s, 3H), 2.78 (s, 3H), 1.96-1.87 (m, 2H), 1.78-1.70 (m, 2H), 1.69-1.55 (m, 4H), 1.48-1.32 (m, 1H), 1.40 (d, J=6.4 Hz, 6H), 1.31-1.19 (m, 2H).

### Example 180: N-((1S,4s)-4-(2-(((R)-2-(3-Fluorophenyl)-2-hydroxyethyl)amino)-2-methylpropyl)cyclohexyl)-N-methylacetamide hydrochloride

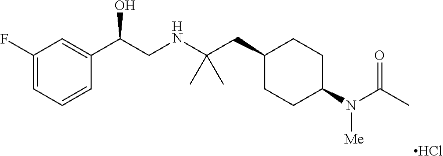 [SMILES: CC(=O)N(C)[C@H]1CC[C@@H](CC(C)(C)NC[C@H](O)c2cccc(F)c2)CC1 ; Cl]

The title compound was prepared in accordance with Example 178, using tert-butyl ((1s,4s)-4-(cyanomethyl)cyclohexyl)carbamate in Step (a), which was prepared from (1s,4s)-4-((tert-butoxycarbonyl)amino)cyclohexane-1-carboxylic acid in accordance with the procedures in Example 67 (a) and (b) and Example 85, Step (a).

¹H NMR (400 MHz, CD₃OD), δ, ppm: 7.47-7.37 (m, 1H), 7.33-7.22 (m, 2H), 7.11-7.03 (m, 1H), 4.99 (d, J=9.7 Hz, 1H), 4.36-4.23 (m, 0.5H), 3.79-3.61 (m, 0.5H), 3.27-3.18 (m, 1H), 3.15-3.03 (m, 1H), 3.02-2.83 (m, 3H), 2.23-2.08 (m, 3H), 1.98-1.89 (m, 1H), 1.87-1.65 (m, 8H), 1.64-1.54 (m, 1H), 1.51-1.44 (m, 1H), 1.44-1.36 (m, 6H)

### Example 182: N-((1S,4s)-4-(2-((®-2-(3-Fluorophenyl)-2-hydroxyethyl)amino)-2-methyl-propyl)cyclohexyl)-N-methylmethanesulfonamide hydrochloride

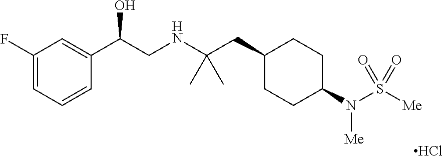 [SMILES: CN([C@H]1CC[C@@H](CC(C)(C)NC[C@H](O)c2cccc(F)c2)CC1)S(C)(=O)=O ; Cl]

The title compound was prepared in accordance with Example 178, Steps (a) to (d), using tert-butyl ((1s,4s)-4-(cyanomethyl)cyclohexyl)carbamate in Step (a) (which was prepared from (1s,4s)-4-((tert-butoxycarbonyl)amino)cyclohexane-1-carboxylic acid, in accordance with the procedures in Example 67 (a) and (b) and Example 85, Step (a)) followed by the procedure in Example 180.

¹H NMR (400 MHz, CD₃OD), δ, ppm: 7.46-7.39 (m, 1H), 7.31-7.23 (m, 2H), 7.11-7.04 (m, 1H), 4.96 (dd, J=10.3, 3.0 Hz, 1H), 3.69-3.60 (m, 1H), 3.23 (dd, J=12.5, 3.0 Hz, 1H), 3.08 (dd, J=12.5, 10.3 Hz, 1H), 2.87 (s, 3H), 2.81 (s, 3H), 1.93-1.87 (m, 1H), 1.82-1.74 (m, 2H), 1.74-1.66 (m, 6H), 1.64-1.56 (m, 2H), 1.39 (d, J=5.4 Hz, 6H).

Examples 182 to 186: The title-compounds were prepared in accordance with the procedures in Example 179 to 182, using (R)-3-fluoro-5-(oxiran-2-yl)pyridine instead of (R)-2-(3-fluorophenyl)oxirane in the appropriate synthetic step.

### Example 182: N-((1R,4r)-4-(2-(((R)-2-(5-Fluoropyridin-3-yl)-2-hydroxyethyl)amino)-2-methylpropyl)cyclohexyl)-N-methylacetamide

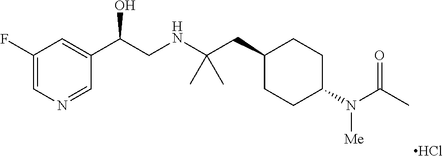 [SMILES: CC(=O)N(C)[C@H]1CC[C@H](CC(C)(C)NC[C@H](O)c2cncc(F)c2)CC1 ; Cl]

¹H NMR (400 MHz, CD₃OD) δ: 8.45-8.40 (m, 1H), 8.37 (d, J=2.9 Hz, 1H), 7.71-7.65 (m, 1H), 4.80 (t, J=6.3 Hz, 1H), 4.33-4.25 (m, 1H), 3.66-3.56 (m, 1H), 2.91-2.78 (m, 3H), 2.76 (d, J=6.7 Hz, 2H), 2.14-2.04 (m, 3H), 1.92-1.81 (m, 2H), 1.71-1.62 (m, 2H), 1.59-1.50 (m, 2H), 1.39-1.28 (m, 3H), 1.23-1.12 (m, 2H), 1.10 (d, J=2.9 Hz, 6H).

### Example 183: N-((1R,4r)-4-(2-(((R)-2-(5-Fluoropyridin-3-yl)-2-hydroxyethyl)amino)-2-methylpropyl)cyclohexyl)-N-methylmethanesulfonamide

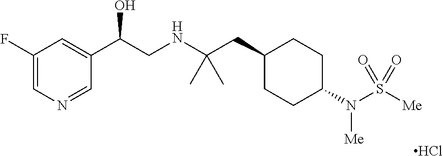 [SMILES: CN([C@H]1CC[C@H](CC(C)(C)NC[C@H](O)c2cncc(F)c2)CC1)S(C)(=O)=O ; Cl]

¹H NMR (400 MHz, CDCl₃) δ: 8.42-8.34 (m, 2H), 7.51-7.45 (m, 1H), 4.64 (dd, J=8.7, 3.8 Hz, 1H), 3.73-3.64 (m, 1H), 2.93 (dd, J=12.0, 3.8 Hz, 1H), 2.82 (s, 3H), 2.78 (s, 3H), 2.52 (dd, J=12.0, 8.7 Hz, 1H), 1.89-1.81 (m, 2H), 1.77-1.70 (m, 2H), 1.59-1.48 (m, 2H), 1.33-1.24 (m, 3H), 1.22-1.12 (m, 2H), 1.07 (d, J=2.7 Hz, 6H).

### Example 184: N-((1S,4s)-4-(2-(((R)-2-(5-Fluoropyridin-3-yl)-2-hydroxyethyl)amino)-2-methylpropyl)cyclohexyl)-N-methylacetamide

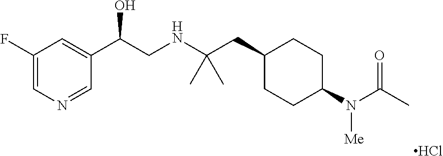 [SMILES: CC(=O)N(C)[C@H]1CC[C@@H](CC(C)(C)NC[C@H](O)c2cncc(F)c2)CC1 ; Cl]

¹H NMR (400 MHz, CD₃OD), δ, ppm: 8.46-8.40 (m, 1H), 8.37 (d, J=2.7 Hz, 1H), 7.71-7.64 (m, 1H), 4.83-4.77 (m, 1H), 4.32-4.25 (m, 0.5H), 3.67-3.56 (m, 0.5H), 2.92-2.79 (m, 3H), 2.79-2.74 (m, 2H), 2.14-2.06 (m, 3H), 1.88-1.82 (m, 1H), 1.79-1.58 (m, 6H), 1.48 (s, 3H), 1.38 (s, 1H), 1.11 (d, J=1.0 Hz, 6H).

### Example 185: N-((1S,4s)-4-(2-(((R)-2-(5-Fluoropyridin-3-yl)-2-hydroxyethyl)amino)-2-methylpropyl)cyclohexyl)-N-methylmethanesulfonamide

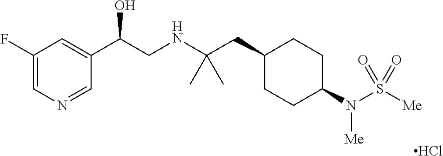 [SMILES: CN([C@H]1CC[C@@H](CC(C)(C)NC[C@H](O)c2cncc(F)c2)CC1)S(C)(=O)=O ; Cl]

¹H NMR (400 MHz, CDCl₃), δ, ppm: 8.43-8.34 (m, 2H), 7.51-7.46 (m, 1H), 4.65 (dd, J=8.8, 3.8 Hz, 1H), 3.73-3.62 (m, 1H), 2.94 (dd, J=12.0, 3.8 Hz, 1H), 2.83 (s, 3H), 2.80 (s, 3H), 2.53 (dd, J=12.0, 8.8 Hz, 1H), 1.89-1.82 (m, 1H), 1.66-1.53 (m, 8H), 1.43-1.30 (m, 2H), 1.08 (d, J=1.9 Hz, 6H).

### Example 186: (1R,4r)-4-(2-(((R)-2-(3-Fluorophenyl)-2-hydroxyethyl)amino)-2-methyl-propyl)-N,N-dimethylcyclohexane-1-sulfonamide hydrochloride

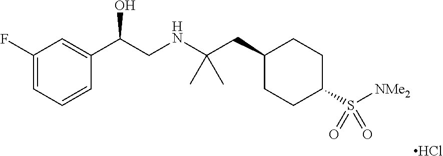 [SMILES: CN(C)S(=O)(=O)[C@H]1CC[C@H](CC(C)(C)NC[C@H](O)c2cccc(F)c2)CC1 ; Cl]

### (a) (1r,4r)-4-(2-((tert-Butoxycarbonyl)amino)-2-methylpropyl)cyclohexyl methanesulfonate

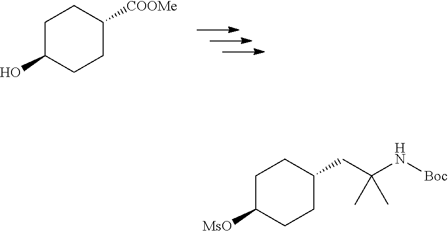 [SMILES: COC(=O)[C@H]1CC[C@H](O)CC1 ; CC(C)(C[C@H]1CC[C@H](OS(C)(=O)=O)CC1)NC(=O)OC(C)(C)C]

The sub-title compound was prepared from methyl (1r,4r)-4-hydroxycyclohexane-1-carboxylate in accordance with the procedures in Example 49, Step (a), Example 1, Step (b) and (c), Example 85, Step (a), Example 13, Step (a) and (b) and Example 111, Step (c).

### (b) S-((1r,4r)-4-(2-((tert-Butoxycarbonyl)amino)-2-methylpropyl)cyclohexyl) ethanethioate

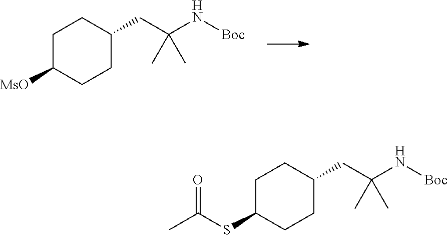 [SMILES: CC(C)(C[C@H]1CC[C@H](OS(C)(=O)=O)CC1)NC(=O)OC(C)(C)C ; CC(=O)S[C@H]1CC[C@H](CC(C)(C)NC(=O)OC(C)(C)C)CC1]

Potassium thioacetate (231 mg, 2.03 mmol) was added to a solution of (1r,4r)-4-(2-((tert-butoxycarbonyl)amino)-2-methylpropyl)cyclohexyl methanesulfonate (256 mg, 0.74 mmol) in DMF (8 mL) at rt. The mixture was stirred at 80° C. for 16 h and cooled to rt. H₂O was added and the mixture was extracted with CH₂Cl₂. The combined extracts were washed with H₂O and brine, dried (Na₂SO₄) and concentrated. The residue was purified by chromatography to give the sub-title compound (162 mg, 67%).

### (c) tert-Butyl (1-((1r,4r)-4-(chlorosulfonyl)cyclohexyl)-2-methylpropan-2-yl)-carbamate

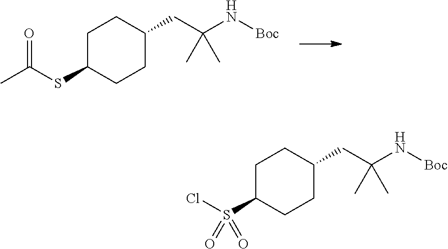 [SMILES: CC(=O)S[C@H]1CC[C@H](CC(C)(C)NC(=O)OC(C)(C)C)CC1 ; CC(C)(C[C@H]1CC[C@H](S(=O)(=O)Cl)CC1)NC(=O)OC(C)(C)C]

HCl (aq, 2 M, 0.6 mL) was added to a solution of N-chlorosuccinimide (1.12 g, 8.43 mmol) in MeCN (7.2 mL). The mixture was cooled to 0° C. and a solution of S-((1s,4s)-4-(2-((tert-butoxycarbonyl)amino)-2-methylpropyl)cyclohexyl)ethanthioate (694 mg, 2.11 mmol) in MeCN (4.8 mL) was added. The mixture was stirred at 0° C. for 30 min, quenched with H₂O, and extracted with EtOAc. The combined extracts were washed with water and brine, dried (Na₂SO₄) and concentrated. The residue was purified by chromatography to give the sub-title compound (584 mg, 78%)

### (d) tert-Butyl (1-((1r,4r)-4-(N,N-dimethylsulfamoyl)cyclohexyl)-2-methylpropan-2-yl)carbamate and tert-Butyl (1-((1s,4s)-4-(N,N-dimethylsulfamoyl)cyclohexyl)-2-methyl-propan-2-yl)carbamate

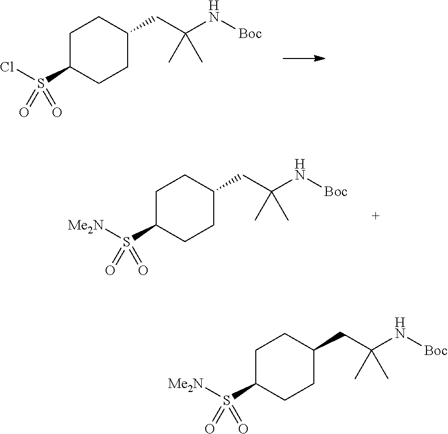 [SMILES: CC(C)(C[C@H]1CC[C@H](S(=O)(=O)Cl)CC1)NC(=O)OC(C)(C)C ; CN(C)S(=O)(=O)[C@H]1CC[C@H](CC(C)(C)NC(=O)OC(C)(C)C)CC1 ; CN(C)S(=O)(=O)[C@H]1CC[C@@H](CC(C)(C)NC(=O)OC(C)(C)C)CC1]

Dimethylamine (2 M in THF, 3.18 ml, 6.36 mmol) was added to an ice-cooled solution of tert-butyl (1-((1s,4s)-4-(chlorosulfonyl)cyclohexyl)-2-methylpropan-2-yl)carbamate (0.225 g, 0.636 mmol) in THF (1 mL). The mixture was stirred at 0° C. for 1 h. NaHCO₃ (aq, sat) was added and the mixture was extracted with CH₂Cl₂. The combined extracts were washed with H₂O and brine, dried (Na₂SO₄) and concentrated. The residue was purified by chromatography followed by chiral chromatography (Daicel Chiralpak IC, 50% iPrOH/heptane, flowrate 40 mL/min) to give the sub-title compounds; 80 mg (35%) for the trans-isomer and 75 mg (33%) for the cis-isomer.

### (e) (1r,4r)-4-(2-Amino-2-methylpropyl)-N,N-dimethylcyclohexane-1-sulfonamide

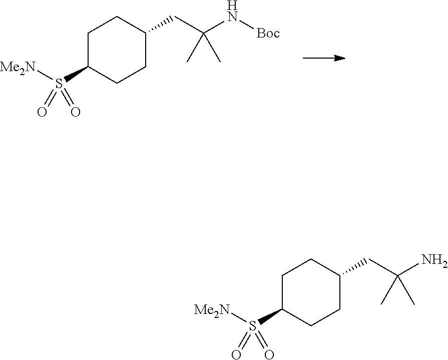 [SMILES: CN(C)S(=O)(=O)[C@H]1CC[C@H](CC(C)(C)NC(=O)OC(C)(C)C)CC1 ; CN(C)S(=O)(=O)[C@H]1CC[C@H](CC(C)(C)N)CC1]

TFA (0.85 mL, 11.0 mmol) was added to a solution of tert-butyl (1-((1r,4r)-4-(N,N-dimethylsulfamoyl)cyclohexyl)-2-methylpropan-2-yl)carbamate (80 mg, 0.221 mmol) in CH₂Cl₂ (3 mL) at rt. The mixture was stirred at rt for 30 min and concentrated. The residue was partitioned between CH₂Cl₂ and H₂O and the aq phase was collected and made alkaline with NaHCO₃(aq, sat) and extracted with 25% iPrOH in CH₂Cl₂. The combined extracts were dried (Na₂SO₄) and concentrated to give the sub-title compound (55 mg, 95%).

### (f) (1R,4r)-4-(2-(((R)-2-(3-Fluorophenyl)-2-hydroxyethyl)amino)-2-methylpropyl)-N,N-dimethylcyclohexane-1-sulfonamide hydrochloride

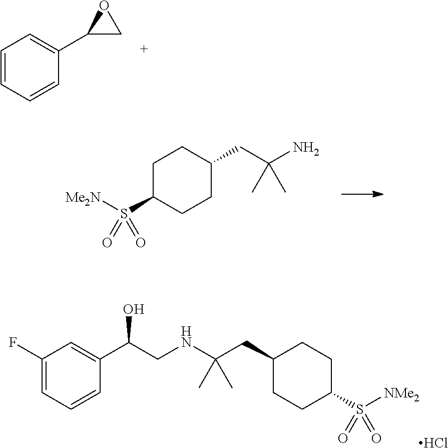 [SMILES: c1ccc([C@@H]2CO2)cc1 ; CN(C)S(=O)(=O)[C@H]1CC[C@H](CC(C)(C)N)CC1 ; CN(C)S(=O)(=O)[C@H]1CC[C@H](CC(C)(C)NC[C@H](O)c2cccc(F)c2)CC1 ; Cl]

(R)-2-(3-Fluorophenyl)oxirane (16 mg, 120 μmol) was added to a solution of (1r,4r)-4-(2-amino-2-methylpropyl)-N,N-dimethylcyclohexane-1-sulfonamide (25 mg, 95 μmol) in iPrOH (0.2 mL) at rt. The mixture was stirred at 80° C. for 16 h and concentrated. The residue was purified by chromatography and the obtained free base was dissolved in CH₂Cl₂ (0.5 mL). HCl (4 M in dioxane, 50 μL) was added and the mixture was diluted with CH₂Cl₂ and added to silica-gel. The silica-gel was washed with pentane and then with iPrOH. The iPrOH washings were concentrated to give the title compound (16 mg, 38%).

¹H NMR (400 MHz, CDCl₃) δ: 7.36-7.28 (m, 1H), 7.24-7.16 (m, 2H), 6.99 (tdd, J=8.4, 2.5, 1.1 Hz, 1H), 5.33 (dd, J=10.3, 2.1 Hz, 1H), 3.18 (dd, J=12.1, 2.1 Hz, 1H), 2.96-2.78 (m, 8H), 2.13-1.99 (m, 2H), 1.99-1.83 (m, 2H), 1.71-1.53 (m, 4H), 1.52-1.43 (m, 4H), 1.42 (s, 3H), 1.11-0.95 (m, 2H).

### Example 187: (1R,4r)-4-(2-(((R)-2-(5-Fluoropyridin-3-yl)-2-hydroxyethyl)amino)-2-methylpropyl)-N,N-dimethylcyclohexane-1-sulfonamide dihydrochloride

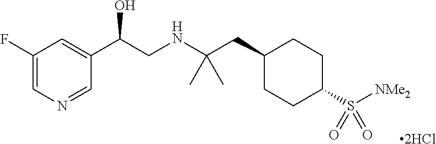 [SMILES: CN(C)S(=O)(=O)[C@H]1CC[C@H](CC(C)(C)NC[C@H](O)c2cncc(F)c2)CC1 ; "·2HCl"]

The title compound was prepared in accordance with the procedures in Example 186, using (R)-3-fluoro-5-(oxiran-2-yl)pyridine in the last step.

¹H NMR (400 MHz, CD₃OD) δ: 8.56 (s, 2H), 7.88 (d, J=8.7 Hz, 1H), 5.05 (dd, J=10.1, 2.6 Hz, 1H), 3.23-3.19 (m, 1H) (overlapping with CD₃OD signal), 3.13-2.97 (m, 2H), 2.83 (s, 6H), 2.11-1.97 (m, 2H), 1.95-1.82 (m, 2H), 1.63-1.49 (m, 4H), 1.48-1.40 (m, 1H), 1.36 (s, 3H), 1.34 (s, 3H), 1.18-1.06 (m, 2H).

### Example 188: (1S,4s)-4-(2-(((R)-2-(3-fluorophenyl)-2-hydroxyethyl)amino)-2-methyl-propyl)-N,N-dimethylcyclohexane-1-sulfonamide hydrochloride

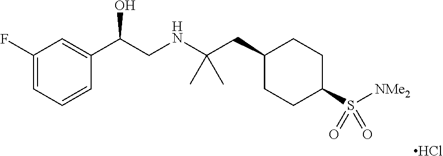 [SMILES: CN(C)S(=O)(=O)[C@H]1CC[C@@H](CC(C)(C)NC[C@H](O)c2cccc(F)c2)CC1 ; Cl]

The title compound was prepared in accordance with the procedures in Example 186, using tert-butyl (1-((1s,4s)-4-(N,N-dimethylsulfamoyl)cyclohexyl)-2-methylpropan-2-yl)-carbamate, see Example 186, Step (d), in Step (e).

¹H NMR (400 MHz, CDCl₃) δ: 7.35-7.27 (m, 1H), 7.24-7.16 (m, 2H), 7.03-6.92 (m, 1H), 5.35 (dd, J=10.4, 2.0 Hz, 1H), 3.18 (dd, J=12.1, 2.1 Hz, 1H), 3.05-2.96 (m, 1H), 2.96-2.83 (m, 7H), 1.97-1.89 (m, 1H), 1.89-1.79 (m, 4H), 1.79-1.65 (m, 4H), 1.65-1.51 (m, 2H), 1.47 (s, 3H), 1.44 (s, 3H).

### Example 189: (1S,4s)-4-(2-(((R)-2-(5-Fluoropyridin-3-yl)-2-hydroxyethyl)amino)-2-methylpropyl)-N,N-dimethylcyclohexane-1-sulfonamide dihydrochloride

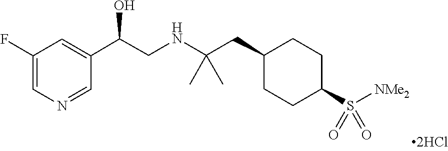 [SMILES: CN(C)S(=O)(=O)[C@H]1CC[C@@H](CC(C)(C)NC[C@H](O)c2cncc(F)c2)CC1]

The title compound was prepared in accordance with the procedures in Example 188, using (R)-3-fluoro-5-(oxiran-2-yl)pyridine in the last step.

¹H NMR (400 MHz, CD₃OD) δ: 8.77 (s, 2H), 8.29-8.11 (m, 1H), 5.28-5.18 (m, 1H), 3.44-3.35 (m, 1H), 3.28-3.13 (m, 2H), 2.92 (s, 6H), 1.94-1.79 (m, 5H), 1.79-1.63 (m, 6H), 1.43 (s, 6H).

### Example 190: (1R,4r)-4-(2-(((R)-2-(3-Fluorophenyl)-2-hydroxyethyl)amino)-2-methyl-propyl)-N-methylcyclohexane-1-sulfonamide hydrochloride

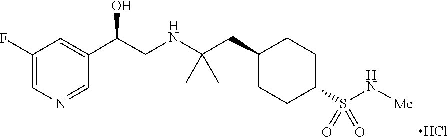 [SMILES: CNS(=O)(=O)[C@H]1CC[C@H](CC(C)(C)NC[C@H](O)c2cncc(F)c2)CC1 ; Cl]

### (a) tert-Butyl (2-methyl-1-((1r,4r)-4-(N-methylsulfamoyl)cyclohexyl)propan-2-yl)-carbamate and tert-butyl (2-methyl-1-((1s,4s)-4-(N-methylsulfamoyl)cyclohexyl)propan-2-yl)carbamate

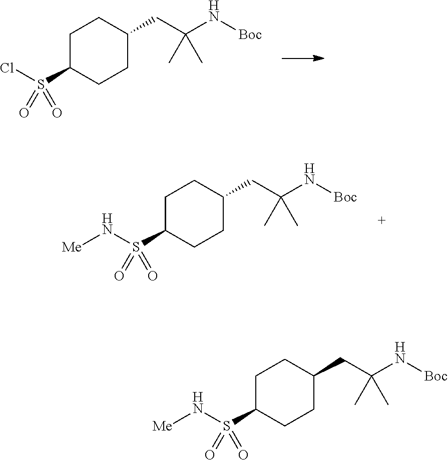 [SMILES: CC(C)(C[C@H]1CC[C@H](S(=O)(=O)Cl)CC1)NC(=O)OC(C)(C)C ; CNS(=O)(=O)[C@H]1CC[C@H](CC(C)(C)NC(=O)OC(C)(C)C)CC1 ; CNS(=O)(=O)[C@H]1CC[C@@H](CC(C)(C)NC(=O)OC(C)(C)C)CC1]

40% Dimethylamine (aq, 40%, 0.10 ml, 1.19 mmol) was added to an ice-cooled solution of tert-butyl (1-((1s,4s)-4-(chlorosulfonyl)cyclohexyl)-2-methylpropan-2-yl)carbamate, see Example 187, Step (c), (0.21 g, 0.59 mmol) in THF (1 mL). The mixture was stirred at 0° C. for 1 h. NaHCO₃(aq, sat) was added and the mixture was extracted with CH₂Cl₂. The combined extracts were washed with water and brine, dried (Na₂SO₄) and concentrated. The residue was purified by chromatography followed by chiral chromatography (Daicel Chiralpak IG, 10% iPrOH/heptane, flowrate 40 mL/min) to give the sub-title compounds; 94 mg (46%) for the trans-isomer and 78 mg (38%) for the cis-isomer.

### (b) (1R,4r)-4-(2-(((R)-2-(3-Fluorophenyl)-2-hydroxyethyl)amino)-2-methylpropyl)-N-methylcyclohexane-1-sulfonamide

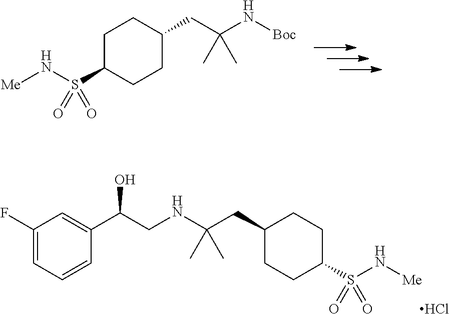 [SMILES: CNS(=O)(=O)[C@H]1CC[C@H](CC(C)(C)NC(=O)OC(C)(C)C)CC1 ; CNS(=O)(=O)[C@H]1CC[C@H](CC(C)(C)NC[C@H](O)c2cccc(F)c2)CC1 ; Cl]

The title compound was prepared from tert-butyl (2-methyl-1-((1r,4r)-4-(N-methylsulfamoyl)cyclohexyl)propan-2-yl)carbamate in accordance with the procedures in Example 186, Steps (e) and (f).

¹H NMR (400 MHz, CDCl₃) δ: 9.78 (br. s, 1H), 7.95 (br. s, 1H), 7.36-7.27 (m, 1H), 7.24-7.15 (m, 2H), 6.98 (tdd, J=8.4, 2.6, 1.1 Hz, 1H), 5.76 (br. s, 1H), 5.38 (d, J=10.1 Hz, 1H), 4.63 (q, J=5.1 Hz, 1H), 3.19 (d, J=11.9 Hz, 1H), 3.03-2.88 (m, 1H), 2.88-2.74 (m, 4H), 2.19-2.09 (m, 2H), 2.01-1.89 (m, 2H), 1.71-1.54 (m, 5H), 1.48 (s, 3H), 1.44 (s, 3H), 1.14-1.03 (m, 2H).

### Example 191: (1R,4r)-4-(2-(((R)-2-(5-Fluoropyridin-3-yl)-2-hydroxyethyl)amino)-2-methylpropyl)-N-methylcyclohexane-1-sulfonamide dihydrochloride

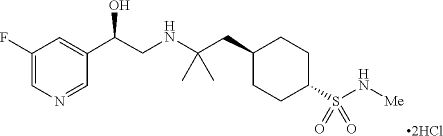 [SMILES: CNS(=O)(=O)[C@H]1CC[C@H](CC(C)(C)NC[C@H](O)c2cncc(F)c2)CC1 ; "·2HCl"]

The title compound was prepared in accordance with the procedures in Example 190, using (R)-3-fluoro-5-(oxiran-2-yl)pyridine in the last step.

¹H NMR (400 MHz, CD₃OD) δ: 8.88 (br. s, 2H), 8.37 (d, J=8.4 Hz, 1H), 5.25 (dd, J=10.0, 2.8 Hz, 1H), 3.39 (dd, J=12.6, 3.0 Hz, 1H), 3.19 (dd, J=12.6, 10.0 Hz, 1H), 2.98 (tt, J=12.3, 3.4 Hz, 1H), 2.71 (s, 3H), 2.20-2.11 (m, 2H), 2.03-1.94 (m, 2H), 1.70-1.53 (m, 4H), 1.53-1.47 (m, 1H), 1.45 (s, 3H), 1.43 (s, 3H), 1.27-1.14 (m, 2H).

### Example 192: (1S,4s)-4-(2-(((R)-2-(3-Fluorophenyl)-2-hydroxyethyl)amino)-2-methyl-propyl)-N-methylcyclohexane-1-sulfonamide hydrochloride

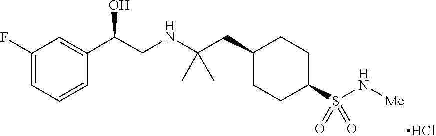 [SMILES: CNS(=O)(=O)[C@H]1CC[C@@H](CC(C)(C)NC[C@H](O)c2cccc(F)c2)CC1 ; Cl]

The title compound was prepared in accordance with the procedures in Example 191, using tert-butyl (2-methyl-1-((1s,4s)-4-(N-methylsulfamoyl)cyclohexyl)propan-2-yl)carbamate, see Example 190, Step (a), in Step (b).

¹H NMR (400 MHz, CD₃OD) δ: 7.47-7.39 (m, 1H), 7.33-7.22 (m, 2H), 7.08 (tdd, J=8.3, 2.6, 1.0 Hz, 1H), 4.96 (dd, J=10.3, 2.9 Hz, 1H), 3.22 (dd, J=12.4, 3.0 Hz, 1H), 3.15-3.02 (m, 2H), 2.72 (s, 3H), 2.00-1.90 (m, 2H), 1.90-1.81 (m, 3H), 1.81-1.61 (m, 6H), 1.41 (s, 3H), 1.40 (s, 3H).

### Example 193: (1S,4s)-4-(2-(((R)-2-(5-Fluoropyridin-3-yl)-2-hydroxyethyl)amino)-2-methylpropyl)-N-methylcyclohexane-1-sulfonamide dihydrochloride

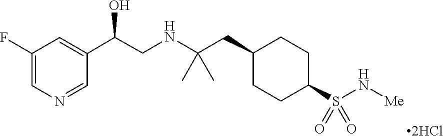 [SMILES: CNS(=O)(=O)[C@H]1CC[C@@H](CC(C)(C)NC[C@H](O)c2cncc(F)c2)CC1 ; "·2HCl"]

The title compound was prepared in accordance with the procedures in Example 193, using (R)-3-fluoro-5-(oxiran-2-yl)pyridine in the last step.

¹H NMR (300 MHz, CDCl₃) δ: 9.13-8.80 (m, 2H), 8.11-7.97 (m, 1H), 5.77 (d, J=9.1 Hz, 1H), 5.40 (s, 1H), 3.67 (dd, J=12.1, 2.4 Hz, 1H), 3.55-3.33 (m, 2H), 3.30 (s, 3H), 2.52-2.29 (m, 5H), 2.28-2.16 (m, 4H), 2.12-2.00 (m, 2H), 1.86 (s, 3H), 1.84 (s, 3H).

### Example 194: (1R,4r)-4-(2-(((R)-2-(3-Fluorophenyl)-2-hydroxyethyl)amino)-2-methylpropyl)cyclohexane-1-sulfonamide hydrochloride

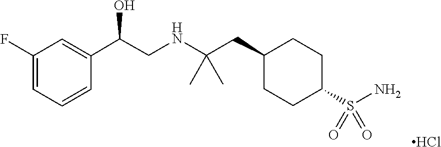 [SMILES: CC(C)(C[C@H]1CC[C@H](S(N)(=O)=O)CC1)NC[C@H](O)c1cccc(F)c1 ; Cl]

### (a) tert-Butyl (2-methyl-1-((1r,4r)-4-sulfamoylcyclohexyl)propan-2-yl)carbamate and tert-Butyl (2-methyl-1-((1s,4s)-4-sulfamoylcyclohexyl)propan-2-yl)carbamate

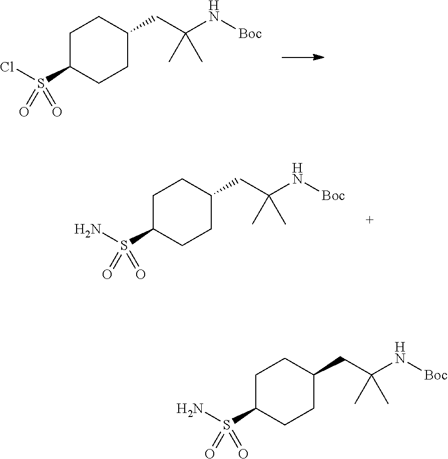 [SMILES: CC(C)(C[C@H]1CC[C@H](S(=O)(=O)Cl)CC1)NC(=O)OC(C)(C)C ; CC(C)(C[C@H]1CC[C@H](S(N)(=O)=O)CC1)NC(=O)OC(C)(C)C ; CC(C)(C[C@H]1CC[C@@H](S(N)(=O)=O)CC1)NC(=O)OC(C)(C)C]

NH₃ (1 M in THF, 3.0 ml, 3.0 mmol) was added to a solution of tert-butyl (1-((1s,4s)-4-(chlorosulfonyl)cyclohexyl)-2-methylpropan-2-yl)carbamate (133 mg, 0.38 mmol) in THF (0.5 mL) at −78° C. The mixture was stirred at −78° C. for 3 h, allowed to warm to rt and diluted with CH₂Cl₂, washed with H₂O and brine, dried (Na₂SO₄) and concentrated. The residue was purified by chromatography followed by chiral chromatography (Daicel Chiralpak IH, 20% iPrOH/MTBE, flowrate 20 mL/min) to give the sub-title compounds; 45 mg (36%) for the trans-isomer and 45 mg (36%) for the cis-isomer.

### (b) (1R,4r)-4-(2-(((R)-2-(3-Fluorophenyl)-2-hydroxyethyl)amino)-2-methyl-propyl)cyclohexane-1-sulfonamide

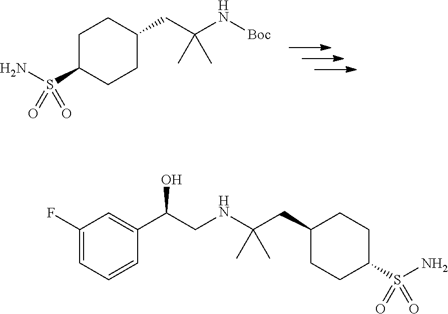 [SMILES: CC(C)(C[C@H]1CC[C@H](S(N)(=O)=O)CC1)NC(=O)OC(C)(C)C ; CC(C)(C[C@H]1CC[C@H](S(N)(=O)=O)CC1)NC[C@H](O)c1cccc(F)c1]

The title compound was prepared from tert-butyl (2-methyl-1-((1r,4r)-4-sulfamoyl-cyclohexyl)propan-2-yl)carbamate in accordance with the procedures in Example 186, Steps (e) and (f).

NMR (400 MHz, CD₃OD) δ: 7.48-7.38 (m, 1H), 7.33-7.21 (m, 2H), 7.13-7.03 (m, 1H), 4.97 (dd, J=10.3, 2.9 Hz, 1H), 3.20 (dd, J=12.5, 3.0 Hz, 1H), 3.05 (dd, J=12.5, 10.3 Hz, 1H), 2.88 (tt, J=12.2, 3.5 Hz, 1H), 2.29-2.20 (m, 2H), 2.04-1.94 (m, 2H), 1.64 (d, J=5.0 Hz, 2H), 1.63-1.54 (m, 2H), 1.53-1.45 (m, 1H), 1.43 (s, 3H), 1.41 (s, 3H), 1.28-1.14 (m, 2H).

### Example 195: (1R,4r)-4-(2-(((R)-2-(5-Fluoropyridin-3-yl)-2-hydroxyethyl)amino)-2-methylpropyl)cyclohexane-1-sulfonamide dihydrochloride

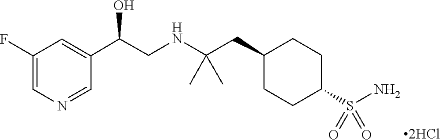 [SMILES: CC(C)(C[C@H]1CC[C@H](S(N)(=O)=O)CC1)NC[C@H](O)c1cncc(F)c1 ; "·2HCl"]

The title compound was prepared in accordance with the procedures in Example 194, using (R)-3-fluoro-5-(oxiran-2-yl)pyridine in the last step.

¹H NMR (400 MHz, CD₃OD) δ: 8.56-8.29 (m, 2H), 7.81-7.66 (m, 1H), 4.96-4.90 (m, 1H), 2.98-2.78 (m, 3H), 2.27-2.14 (m, 2H), 2.02-1.90 (m, 2H), 1.63-1.49 (m, 2H), 1.49-1.37 (m, 3H), 1.22 (s, 3H), 1.21 (s, 3H), 1.17-1.07 (m, 2H).

### Example 196: (1S,4s)-4-(2-(((R)-2-(3-Fluorophenyl)-2-hydroxyethyl)amino)-2-methylpropyl)cyclohexane-1-sulfonamide hydrochloride

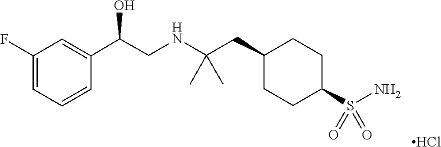 [SMILES: CC(C)(C[C@H]1CC[C@@H](S(N)(=O)=O)CC1)NC[C@H](O)c1cccc(F)c1 ; Cl]

The title compound was prepared in accordance with the procedures in Example 195, using tert-butyl (2-methyl-1-((1s,4s)-4-sulfamoylcyclohexyl)propan-2-yl)carbamate, see Example 196, Step (a), in Step (b).

¹H NMR (400 MHz, CD₃OD) δ: 7.47-7.37 (m, 1H), 7.33-7.20 (m, 2H), 7.07 (tdd, J=8.9, 2.7, 1.0 Hz, 1H), 4.98 (dd, J=10.3, 2.9 Hz, 1H), 3.21 (dd, J=12.5, 2.9 Hz, 1H), 3.05 (dd, J=12.5, 10.3 Hz, 1H), 2.96 (tt, J=9.8, 4.1 Hz, 1H), 2.06-1.95 (m, 2H), 1.93-1.79 (m, 3H), 1.79-1.70 (m, 4H), 1.69-1.63 (m, 2H), 1.41 (s, 3H), 1.40 (s, 3H).

### Example 197: (1S,4s)-4-(2-(((R)-2-(5-Fluoropyridin-3-yl)-2-hydroxyethyl)amino)-2-methylpropyl)cyclohexane-1-sulfonamide dihydrochloride

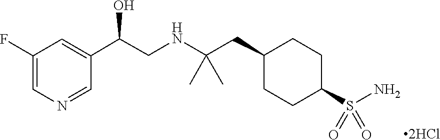 [SMILES: CC(C)(C[C@H]1CC[C@@H](S(N)(=O)=O)CC1)NC[C@H](O)c1cncc(F)c1 ; "·2HCl"]

The title compound was prepared in accordance with the procedures in Example 196, using (R)-3-fluoro-5-(oxiran-2-yl)pyridine in the last step.

¹H NMR (400 MHz, CD₃OD) δ: 9.09 (br s, 2H), 7.82-7.62 (m, 1H), 4.98 (dd, J=9.4, 1.6 Hz, 1H), 3.20-3.16 (m, 1H), 3.05 (dd, J=12.5, 10.0 Hz, 1H), 2.86 (tt, J=9.7, 4.1 Hz, 1H), 1.95-1.86 (m, 2H), 1.82-1.65 (m, 4H), 1.64 (d, J=4.9 Hz, 2H), 1.60-1.51 (m, 3H), 1.31 (s, 3H), 1.30 (s, 3H).

### Example 198: N-((1S,4s)-4-(2-(((R)-2-(5-Fluoropyridin-3-yl)-2-hydroxyethyl)amino)-2-methylpropyl)cyclohexyl)propane-1-sulfonamide

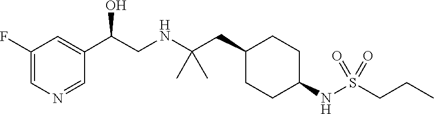 [SMILES: CCCS(=O)(=O)N[C@H]1CC[C@@H](CC(C)(C)NC[C@H](O)c2cncc(F)c2)CC1]

The title compound was prepared from (R)-2-((1-((1s,4S)-4-aminocyclohexyl)-2-methylpropan-2-yl)amino)-1-(5-fluoropyridin-3-yl)ethan-1-ol, see Example 175, and propane-1-sulfonyl fluoride in accordance with the procedure in Example 161.

¹H NMR (400 MHz, CDCl₃) δ 8.40-8.35 (m, 2H), 7.51-7.46 (m, 1H), 4.64 (dd, J=8.8, 3.8 Hz, 1H), 4.45-4.25 (m, 1H), 3.59-3.49 (m, 1H), 3.01-2.95 (m, 2H), 2.93 (dd, J=12.0, 3.8 Hz, 1H), 2.52 (dd, J=12.0, 8.7 Hz, 1H), 2.4-2.0 (br s, 2H), 1.91-1.77 (m, 2H), 1.75-1.47 (m, 9H), 1.42-1.17 (m, 2H), 1.12-1.01 (m, 9H).

### Example 199: 1,1,1-Trifluoro-N-((1S,4s)-4-(2-(((R)-2-(5-fluoropyridin-3-yl)-2-hydroxy-ethyl)amino)-2-methylpropyl)cyclohexyl)methanesulfonamide dihydrochloride

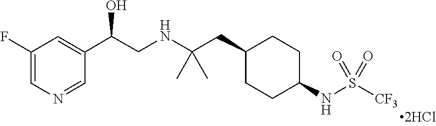 [SMILES: CC(C)(C[C@H]1CC[C@@H](NS(=O)(=O)C(F)(F)F)CC1)NC[C@H](O)c1cncc(F)c1]

The title compound was prepared from (R)-2-((1-((1s,4S)-4-aminocyclohexyl)-2-methyl-propan-2-yl)amino)-1-(5-fluoropyridin-3-yl)ethan-1-ol, see Example 175, and trifluoro-methanesulfonic anhydride in accordance with the procedure in Example 173.

¹H NMR (400 MHz, CD₃OD) δ 8.94-8.80 (m, 2H), 8.48-8.42 (m, 1H), 5.29 (dd, J=9.8, 2.7 Hz, 1H), 3.66-3.56 (m, 1H), 3.42 (dd, J=12.6, 2.9 Hz, 1H), 3.21 (dd, J=12.5, 10.0 Hz, 1H), 1.82-1.62 (m, 9H), 1.60-1.47 (m, 2H), 1.43 (s, 3H), 1.42 (s, 3H).

### Example 200: N-((1S,4s)-4-(2-(((R)-2-(3-Fluorophenyl)-2-hydroxyethyl)amino)-2-methylpropyl)cyclohexyl)ethanesulfonamide hydrochloride

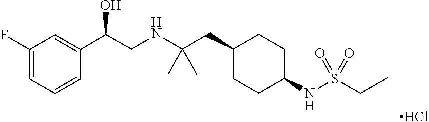 [SMILES: CCS(=O)(=O)N[C@H]1CC[C@@H](CC(C)(C)NC[C@H](O)c2cccc(F)c2)CC1 ; Cl]

The title compound was prepared from (R)-2-((1-((1s,4S)-4-aminocyclohexyl)-2-methyl-propan-2-yl)amino)-1-(5-fluoropyridin-3-yl)ethan-1-ol, see Example 175, and ethane-sulfonyl chloride in accordance with the procedure in Example 161.

¹H NMR (400 MHz, CD₃OD) δ 7.46-7.38 (m, 1H), 7.30-7.20 (m, 2H), 7.12-7.04 (m, 1H), 4.94 (dd, J=10.4, 3.0 Hz, 1H), 3.46-3.40 (m, 1H), 3.19 (dd, J=12.5, 3.0 Hz, 1H), 3.09-2.99 (m, 3H), 1.75-1.58 (m, 9H), 1.56-1.45 (m, 2H), 1.40 (d, J=6.3 Hz, 6H), 1.32 (t, J=7.3 Hz, 3H).

### Example 201: N-((1R,4r)-4-(2-(((R)-2-(5-fluoropyridin-3-yl)-2-hydroxyethyl)amino)-2-methylpropyl)cyclohexyl)ethanesulfonamide

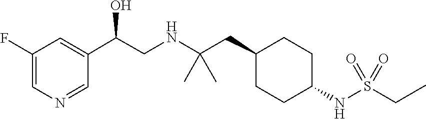 [SMILES: CCS(=O)(=O)N[C@H]1CC[C@H](CC(C)(C)NC[C@H](O)c2cncc(F)c2)CC1]

The title compound was prepared from (R)-2-((1-((1r,4R)-4-aminocyclohexyl)-2-methyl-propan-2-yl)amino)-1-(5-fluoropyridin-3-yl)ethan-1-ol, see Example 85, and ethane-sulfonyl chloride in accordance with the procedure in Example 161, but without the salt formation.

¹H NMR (400 MHz, CD₃OD) δ 8.45-8.41 (m, 1H), 8.38 (d, J=2.9 Hz, 1H), 7.71-7.65 (m, 1H), 4.81 (dd, J=7.5, 5.0 Hz, 1H), 3.14-3.04 (m, 1H), 2.91 (d, J=6.4 Hz, 2H), 2.84-2.72 (m, 2H), 2.26-2.11 (m, 1H), 2.01-1.89 (m, 2H), 1.87-1.75 (m, 2H), 1.45-1.22 (m, 5H), 1.16-1.04 (m, 13H).

### Example 202: N-((1R,4r)-4-(2-(((R)-2-(5-fluoropyridin-3-yl)-2-hydroxyethyl)amino)-2-methylpropyl)cyclohexyl)-2-methylpropane-1-sulfonamide

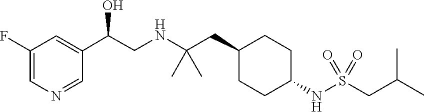 [SMILES: CC(C)CS(=O)(=O)N[C@H]1CC[C@H](CC(C)(C)NC[C@H](O)c2cncc(F)c2)CC1]

The title compound was prepared from (R)-2-((1-((1r,4R)-4-aminocyclohexyl)-2-methyl-propan-2-yl)amino)-1-(5-fluoropyridin-3-yl)ethan-1-ol, see Example 85, and isobutane-sulfonyl chloride in accordance with the procedure in Example 161, but without the salt formation.

¹H NMR (400 MHz, CD₃OD) δ 8.45-8.42 (m, 1H), 8.38 (d, J=2.9 Hz, 1H), 7.71-7.65 (m, 1H), 4.81 (dd, J=7.5, 5.0 Hz, 1H), 3.16-3.03 (m, 1H), 2.91 (d, J=6.4 Hz, 2H), 2.84-2.73 (m, 2H), 2.26-2.13 (m, 1H), 2.01-1.88 (m, 2H), 1.86-1.74 (m, 2H), 1.44-1.23 (m, 5H), 1.20-1.00 (m, 13H).

### BIOLOGICAL EXAMPLES

L6-myoblasts were grown in Dulbecco's Modified Eagle's Medium (DMEM) containing 1 g/L glucose supplemented with 10% fetal bovine serum, 2 mM L-Glutamine, 50 U/mL penicillin, 50 μg/mL streptomycin and 10 mM HEPES. Cells were plated at 1×10⁵ cells per mL in 24-well plates. After reaching 90% confluence the cells were grown in medium containing 2% FBS for 7 days where upon cells differentiated into myotubes.

### Biological Example 1: Glucose Uptake

Differentiated L6-myotubes were serum-starved overnight in medium containing 0.5% fatty-acid free BSA and stimulated with an agonist, with a final concentration of 1×10⁻⁵ M. After 1 h 40 min the cells were washed with warm glucose free medium or PBS twice and another portion of agonist was added to the glucose free medium. After 20 min the cells were exposed to 50 nM ³H-2-deoxyglucose for 10 min before washed with ice cold glucose free medium or PBS three times and lysed with 0.2 M NaOH, 400 μL/well, for 1 h at 60° C. The cell lysate was mixed with 4 mL scintillation buffer (Emulsifier Safe, Perkin Elmer) and the radioactivity was detected in a β-counter (Tri-Carb 4810TR, Perkin Elmer). The activity for each compound is compared to that of isoproterenol. If a compound shows activity of more than 75% of that of isoproterenol, the activity is denoted with +++, if it is between 75 and 50% it is denoted with ++; if it is between 50 and 25% it is denoted with +; if it less than 25% it is denoted with −.

### Biological Example 2: Measurement of Intracellular cAMP Levels

Differentiated cells were serum-starved overnight and stimulated with an agonist, final concentration 1×10⁻⁵ M, for 15 min in stimulation buffer (HBSS supplemented with 1% BSA, 5 mM HEPES and 1 mM IBMX, pH 7.4). The medium was aspirated and 100 μL of 95% EtOH was added to each well of the 24-well plate and cells were kept at −20° C. overnight. The EtOH was allowed to evaporate and 500 μL of lysis buffer (1% BSA, 5 mM HEPES and 0.3% Tween-20, pH 7.4) was added to each well. The plate was kept at −80° C. for 30 min and then at −20° C. until the day of detection when the samples were thawed. Intracellular cAMP levels were detected using an alpha screen cAMP kit (6760635D from Perkin Elmer). The activity for each compound is compared to that of isoproterenol. If a compound shows activity of more than 75% of that of isoproterenol, the activity is denoted with +++, if it is between 75 and 50% it is denoted with ++; if it is between 50 and 25% it is denoted with +; if it less than 25% it is denoted with −.

### Biological Example 3: Glucose Uptake in the Presence of the β₂-Antagonist ICI-118,551

Differentiated L6-myotubes were serum-starved overnight in medium containing 0.5% fatty-acid free BSA and were incubated with the β₂-adrenergic receptor antagonist ICI-118,551 at a final concentration of 1×10⁻⁵ M for 30 min. The cells were stimulated with a compound of the invention, at a final concentration of 1×10⁻⁵ M. After 1 h 40 min the cells were washed twice with warm, glucose free medium or PBS and additional portions of the compound of the invention and the antagonist were added. After 20 min the cells were exposed to 50 nM ³H-2-deoxyglucose for 10 min before washed with ice cold glucose free medium or PBS three times and lysed with 0.2 M NaOH, 400 μL/well, for 1 h at 60° C. The cell lysate was mixed with 4 mL scintillation buffer (Emulsifier Safe, Perkin Elmer) and the radioactivity was detected in a β-counter (Tri-Carb 4810TR, Perkin Elmer). The activity for each compound is compared to that of isoproterenol. If a compound shows activity of more than 75% of that of isoproterenol, the activity is denoted with +++, if it is between 75 and 50% it is denoted with ++; if it is between 50 and 25% it is denoted with +; if it less than 25% it is denoted with −.

Using the assays described in Biological Examples 1, 2 and 3 the following results were obtained.

| Compound | Biological | Biological | Biological |
|---|---|---|---|
| example no. | example 1 | example 2 | example 3 |
| 1 | + + | − | |
| 2 | + + | − | |
| 3 | + + | − | |
| 4 | + + | − | |
| 5 | − | − | |
| 6 | − | − | |
| 7 | + + + | − | |
| 8 | + + + | − | |
| 9 | + + | − | |
| 10 | + + + | − | |
| 11 | + | − | |
| 12 | + + | − | |
| 13 | + + + | − | |
| 14 | + + + | − | |
| 15 | + + + | − | |
| 16 | + + | − | |
| 17 | + | − | |
| 18 | − | − | |
| 19 | + + + | + + | |
| 20 | + + + | − | |
| 21 | + + | − | |
| 22 | + | − | |
| 23 | + + + | + | |
| 24 | + + + | − | |
| 25 | + | − | |
| 26 | + | − | |
| 27 | + + + | − | |
| 28 | + | − | |
| 29 | + | − | |
| 30 | + | − | |
| 31 | + | − | |
| 32 | − | − | |
| 33 | + + | − | |
| 34 | + + | − | |
| 35 | + + | − | |
| 36 | + | − | |
| 37 | + + | − | |
| 38 | + + | − | |
| 39 | + + | − | |
| 40 | + + | − | |
| 41 | + + | − | |
| 42 | + + | − | |
| 43 | + + | − | |
| 44 | + | − | |
| 45 | + | − | |
| 46 | + | − | |
| 47 | − | − | |
| 48 | + | − | |
| 49 | + + | − | |
| 50 | + + + | − | |
| 51 | + + + | − | |
| 52 | + + + | − | |
| 53 | + | − | |
| 54 | + | − | |
| 55 | + | − | |
| 56 | + + | − | |
| 57 | + + | − | |
| 58 | + | − | |
| 59 | + | − | |
| 60 | + + | − | |
| 61 | + | − | |
| 62 | + + + | − | |
| 63 | + | − | |
| 64 | + + + | − | |
| 65 | + + + | + | |
| 66 | + + | − | |
| 67 | + | − | |
| 68 | + | − | |
| 69 | + + + | − | |
| 70 | + | − | |
| 71 | + + | − | |
| 72 | + | − | |
| 73 | + + | − | |
| 74 | + | − | |
| 75 | + + | − | |
| 76 | + + | − | |
| 77 | + + | − | |
| 78 | + + | − | |
| 79 | − | − | |
| 80 | | | |
| 81 | + + + | − | |
| 82 | | | |
| 83 | + | − | |
| 84 | | | |
| 85 | + + + | − | − |
| 86 | | | |
| 87 | + + + | − | |
| 88 | | | |
| 89 | + + + | − | |
| 90 | | | |
| 91 | | | |
| 92 | + | − | |
| 93 | + + + | − | |
| 94 | + + | − | |
| 95 | + + + | | |
| 96 | + + | | |
| 97 | + | + | |
| 98 | + | − | |
| 99 | + + + | − | |
| 100 | + + + | − | |
| 101 | − | − | |
| 102 | | | |
| 103 | + + + | − | |
| 104 | + + + | − | |
| 105 | + | − | |
| 106 | + | − | |
| 107 | + + + | − | − |
| 108 | + + | − | |
| 109 | + + | − | |
| 110 | − | − | |
| 111 | + + + | + | |
| 112 | + + + | + | − |
| 113 | + | − | |
| 114 | + + + | − | |
| 115 | + + + | − | |
| 116 | + + + | − | − |
| 117 | − | − | |
| 118 | + | − | |
| 119 | + + + | − | |
| 120 | + + + | − | |
| 121 | + + | − | |
| 122 | + + + | + + | |
| 123 | + + + | − | |
| 124 | + + | − | |
| 125 | + + + | − | |
| 126 | + + | − | |
| 127 | + + | − | |
| 128 | + + | − | |
| 129 | + + + | + | |
| 130 | + + | − | |
| 131 | + + | − | |
| 132 | + + + | + | |
| 133 | + + | − | |
| 134 | + + | − | |
| 135 | + + | − | |
| 136 | + + | − | |
| 137 | + + | − | |
| 138 | + + | − | |
| 139 | + + + | + | |
| 140 | + + + | − | |
| 141 | + + + | − | |
| 142 | + + + | − | |
| 143 | + + + | − | |
| 144 | + + | − | |
| 145 | + + + | − | |
| 146 | + + | − | |
| 147 | + + | − | |
| 148 | + + + | − | |
| 149 | + + | − | |
| 150 | + + + | − | |
| 151 | + + + | − | |
| 152 | + + | − | |
| 153 | + + + | + | |
| 154 | + + + | + | |
| 155 | + + + | + + | |
| 156 | + + + | + + + | |
| 157 | + + + | + | |
| 158 | + + + | + + + | |
| 159 | + + + | + | |
| 160 | + + + | − | |
| 161 | + + | − | |
| 162 | + + + | − | |
| 163 | + + + | − | |
| 164 | + + + | − | |
| 165 | + + + | − | |
| 166 | + + + | − | |
| 167 | + + | − | |
| 168 | + + + | − | |
| 169 | + + + | + | |
| 170 | + + + | − | |
| 171 | + + + | − | |
| 172 | + + | − | |
| 173 | + + | − | |
| 174 | + + + | − | |
| 175 | + + | − | |
| 176 | + + + | − | |
| 177 | + + + | − | |
| 178 | + + + | − | |
| 179 | + + | − | |
| 180 | + + | − | |
| 181 | + + | − | |
| 182 | + + + | + | |
| 183 | + + + | − | |
| 184 | + + + | − | |
| 185 | + + + | − | |
| 186 | + + + | + + | |
| 187 | + + + | + | |
| 188 | + + + | + | |
| 189 | + + + | + + | |
| 190 | + + + | + | |
| 191 | + + | − | |
| 192 | + + + | − | |
| 193 | + + + | − | |
| 194 | + + + | − | |
| 195 | + + + | − | |
| 196 | + + + | − | |
| 197 | + + + | − | |
| 198 | + + + | − | |
| 199 | + + + | − | |
| 200 | | | |
| 201 | | | |
| 202 | | | |

1. A compound of formula I 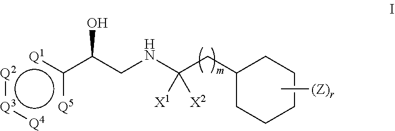
or a pharmaceutically acceptable salt thereof, wherein:
the ring comprising Q¹ to Q⁵ represents:
phenyl optionally substituted with one or more Y¹, or
a 5- or 6-membered heteroaryl optionally substituted with one or more Y²;
X¹ and X² each independently represent H or C₁₋₃ alkyl optionally substituted with one or more fluoro;
each Y¹ independently represents halo, R^{a1}, —CN or —N₃;
each Y² independently represent halo, R^{a2}, —CN or —N₃;
each Z independently represents halo, R^{a3}, —CN, —N₃, —N(R^{b3})R^{c3}, —OR^{d3}, —S(O)ₚR^{e3}, —S(O)_{q}N(R^{f3})R^{g3} or —N(R^{h3})S(O)ₜRⁱ³;
each R^{a1}, R^{a2}, R^{a3}, R^{e3} and Rⁱ³ represents C₁₋₆ alkyl, C₂₋₆ alkenyl, or C₂₋₆ alkynyl each optionally substituted by one or more groups independently selected from halo and G¹;
each R^{b3}, R^{c3}, R^{d3}, R^{f3}, R^{g3} and R^{h3} independently represents
H,
C₁₋₆ alkyl, C₂₋₆ alkenyl, C₂₋₆ alkynyl each optionally substituted by one or more groups independently selected from halo and G²;
or alternatively any of R^{b3} and R^{c3}, and/or R^{f3} and R^{g3} may be linked together to form, together with the nitrogen atom to which they are attached, a 4- to 6-membered ring, which ring optionally contains one further heteroatom and which ring optionally is substituted by one or more groups independently selected from halo, C₁₋₃ alkyl optionally substituted by one or more halo, and ═O;
each G¹ and G² independently represents R^{a4}, —CN, —N₃, —N(R^{b4})R^{c4}, —OR^{d4}, —S(O)ₚR^{e4}, —S(O)_{q}N(R^{f4})R^{g4}, or ═O;
each R^{a4} independently represents phenyl or 5- or 6-membered heteroaryl, each optionally substituted by one or more group selected from halo, —CN or C₁₋₃ alkyl optionally substituted by one or more halo or —CN;
each R^{b4}, R^{c4}, R^{d4}, R^{f4} and R^{g4} independently represents H, or C₁₋₆ alkyl, C₂₋₆ alkenyl or C₂₋₆ alkynyl optionally substituted by one or more halo or —CN;
each R^{e4} independently represents C₁₋₆ alkyl, C₂₋₆ alkenyl, C₂₋₆ alkynyl each optionally substituted by one or more halo or —CN;
or alternatively any of R^{b4} and R^{c4} and/or R^{f4} and R^{g4} may be linked together to form, together with the nitrogen atom to which they are attached, a 4- to 6-membered ring, which ring optionally contains one further heteroatom and which ring optionally is substituted by one or more groups independently selected from halo, C₁₋₃ alkyl optionally substituted by one or more halo, and ═O;
each p independently represents 0, 1 or 2;
each q independently represents 1 or 2;
each t independently represents 1 or 2;
m represents 0 to 3; and
r represents 0 to 6.

2. The compound according to claim 1, wherein each Y¹ and Y² independently represents R^{a1}, halo or —CN.

3. The compound according to any one of the preceding claims, wherein each Y¹ and Y² independently represents F.

4. The compound according to any one of the preceding claims, wherein the ring comprising Q¹ to Q⁵ represents:
phenyl optionally substituted with one or more Y¹, or
a 6-membered heteroaryl optionally substituted with one or more Y².

5. The compound according to any one of the preceding claims, wherein the ring comprising Q¹ to Q⁵ represents:
phenyl optionally substituted with one or more Y¹, or
pyridyl optionally substituted with one or more Y².

6. The compound according to any one of the preceding claims, wherein r represents 1.

7. The compound according to any one of the preceding claims, each Z independently represents R^{a3}, —N(R^{b3})R^{c3}, —OR^{d3}, —N(R^{h3})S(O)ₜRⁱ³, —CN, —S(O)ₚR^{e3}, or —S(O)_{q}N(R^{f3})R^{g3}.

8. The compound according to any one of the preceding claims, wherein each Z independently represents —OMe, —OBn, —OH, —OCH₂COOEt, —OCH₂COOH, —CN, —NH₂, —NHC(O)OtBu, —NHC(O)Me, —NHC(O)tBu, —NHC(O)(c-Bu), —NHC(O)(c-Pr), —NHC(O)CF₃, —NHC(O)NMe₂, —NHC(O)Ph, —NHS(O)₂Me, —NHS(O)₂CF₃, —NHS(O)₂(n-Pr), —NHS(O)₂(c-Pr), —NHS(O)₂(i-Bu), —NHS(O)₂(Et), —N(Me)C(O)Me, —N(Me)S(O)₂Me, —S(O)₂NHMe, —S(O)₂NMe₂, —C(O)OH or —C(O)OMe.

9. The compound according to any one of the preceding claims, wherein either X¹ and X² each both represent H or both represent methyl.

10. The compound according to any of the preceding claims, wherein m represents 0 to 2.

11. A compound as defined in any one of the preceding claims for use in medicine.

12. A pharmaceutical composition comprising a compound as defined in any one of claims 1 to 10, and optionally one or more pharmaceutically acceptable adjuvant, diluent and/or carrier.

13. A compound as defined in any one of claims 1 to 10, for use in the treatment of hyperglycaemia or a disorder characterized by hyperglycaemia.

14. The use of a compound as defined in any one of claims 1 to 10, for the manufacture of a medicament for the treatment of hyperglycaemia or a disorder characterized by hyperglycaemia.

15. A method of treating hyperglycaemia or a disorder characterized by hyperglycaemia comprising administering to a patient in need thereof a therapeutically effective amount of a compound as defined in any one of claims 1 to 10.

16. The compound for use, method or use according to any one of claims 13 to 15, wherein the hyperglycaemia or disorder characterised by hyperglycaemia is, or is characterised by, the patient displaying severe insulin resistance.

17. The compound for use, method or use according to any one of claims 13 to 16, wherein the disorder characterised by hyperglycaemia is selected from the group consisting of Type 2 diabetes, Rabson-Mendenhall syndrome, Donohue's syndrome (leprechaunism), Type A and Type B syndromes of insulin resistance, the HAIR-AN (hyperandrogenism, insulin resistance, and acanthosis nigricans) syndromes, pseudoacromegaly, and lipodystrophy.

18. A compound as defined in anyone of claims 1 to 10, for use in the treatment of a non-alcoholic fatty liver disease.

19. The use of a compound as defined in any one of claims 1 to 10, in the manufacture of a medicament for the treatment or prevention of a non-alcoholic fatty liver disease.

20. A method of treating or preventing a non-alcoholic fatty liver disease as defined in comprising administering to a patient in need thereof a therapeutically effective amount of a compound as defined in any one of claims 1 to 10.

21. A compound as defined in anyone of claims 1 to 10, for use in treating a disease or disorder the treatment of which is mediated by activation of the β₂ adrenergic receptor.

22. The use of a compound as defined in any one of claims 1 to 10, in the manufacture of a medicament for use in treating a disease or disorder the treatment of which is mediated by activation of the β₂ adrenergic receptor.

23. A method of treating a disease or disorder the treatment of which is mediated by activation of the β₂ adrenergic receptor comprising administering to a patient in need thereof a therapeutically effective amount of a compound as defined in any one of claims 1 to 10.
